(19) United States
(12) Patent Application Publication
(10) Pub. No.: US 2023/0014226 A1
(43) Pub. Date: Jan. 19, 2023

(54) NEW COMPOUNDS AND METHODS

(71) Applicant: BenevolentAI Bio Limited, London (GB)

(72) Inventors: Rebecca Paul, London (GB); Mark Rackham, London (GB); Yi Mok, London (GB); Marton Vass, London (GB); Joshua Meyers, London (GB); Andrew Cronin, London (GB); Ajay Mandal, London (GB)

(21) Appl. No.: 17/622,543

(22) PCT Filed: Jun. 24, 2020
(86) PCT No.: PCT/GB2020/051526

§ 371 Date: Dec. 23, 2021

(30) Foreign Application Priority Data
Jun. 24, 2019 (GB) 1909036.4
Sep. 20, 2019 (GB) 1913565.6

(51) Int. Cl. C07D 487/04; C07D 471/04; C07D 401/12; C07D 413/12; C07D 498/04; C07D 401/14; A61P 25/28; A61P 25/16

(52) U.S. Cl. CPC C07D 487/04; C07D 471/04; C07D 401/12; C07D 413/12; C07D 498/04; C07D 401/14; A61P 25/28; A61P 25/16

(57) ABSTRACT

The present invention relates to compounds of Formula (I) or a pharmaceutically acceptable salt, solvate, hydrate, tautomer, optical isomer, N-oxide, and/or prodrug thereof. The present invention also relates to pharmaceutical compositions comprising the compounds of the invention, and to their use in the treatment or prevention of medical conditions in which inhibition of c-ABL is beneficial. (I)

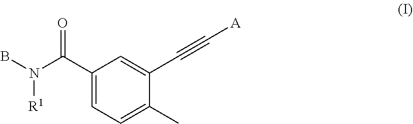

### FIELD OF THE INVENTION

The present invention relates to compounds of Formula (I), and in particular Formula (II), which are inhibitors of c-ABL. The invention also relates to pharmaceutical compositions comprising those compounds, and to their use in the treatment or prevention of medical conditions in which inhibition of c-ABL is beneficial. Such medical conditions include neurodegenerative diseases and cancer.

### BACKGROUND

ABL1 (Abelson Murine Leukaemia Viral Oncogene Homolog 1) is a protein that exhibits tyrosine kinase enzymatic activity and is associated with various cell functions. In humans, this protein is encoded by the ABL1 gene located on chromosome 9. The version of the ABL1 gene found within the mammalian genome is denoted c-Abl.

Philadelphia chromosome is a genetic abnormality in chromosome 22 formed by the t(9,22) reciprocal chromosome translocation, resulting in a fusion gene denoted BCR-ABL1. This fusion gene contains the ABL1 gene from chromosome 9 and part of the BCR gene. The tyrosine kinase activity of the ABL1 protein is normally tightly regulated, however, the BCR domains in the fusion gene result in constitutive activation of the ABL1 kinase. However, the binding domains of BCR-ABL and c-ABL are identical.

Activation of c-Abl has been implicated in various diseases, notably cancer. For instance, the presence of the BCR-ABL mutation is strongly linked to chronic myeloid leukaemia (CML). It is also found in some instances of acute lymphocytic leukaemia (ALL) and acute lymphoblastic leukaemia (ALL). Nilotinib and Ponatinib are both c-Abl inhibitors that have been used in the treatment of chronic myeloid leukaemia (CML) and acute lymphocytic leukaemia (ALL). The range of leukaemias that may be treated by c-ABL inhibition include chronic myeloid leukaemia (CML), acute lymphoblastic leukaemia (ALL), acute myelogenous leukaemia (AML), mixed-phenotype acute leukaemia (MPAL), and central nervous system (CNS) metastases thereof.

Activation of c-Abl has also been implicated in neurodegenerative diseases. Neurodegenerative diseases may be characterised by progressive degeneration and ultimate death of neurons. Particular neurodegenerative diseases include amyotrophic lateral sclerosis (ALS) and Parkinson's disease (PD).

ALS is a fatal neurodegenerative disease caused by the progressive degeneration of motor neurons. It has been reported that c-Abl signalling activation contributes to neuronal apoptosis and that c-Abl inhibitors can prevent motor neuron death [Rojas et al. Frontiers in Cellular Neuroscience, 2015, 9, 203; Imamura et al. Science Translational Medicine, 2017].

Parkinson's disease (PD) is a progressive neurodegenerative disorder caused by a selective loss of dopaminergic neurons in the substantia nigra pars compacta. It has been reported that c-Abl is activated in the brain of patients with PD and that c-Abl inhibition can protect against dopamine neuronal loss [Pagan et al. Pharmacology Research & Perspectives, 2019; Karuppagounder et al. Scientific Reports, 2014, 4, 4874].

Activation of c-Abl has also been implicated in a wide range of other diseases including, but not limited to, prion diseases, viral infections, diabetes, inflammatory diseases such as pulmonary fibrosis, and skeletal or muscular dystrophies.

Viral infections can be mediated by ABL1 kinase activity, as in the case of pox-viruses and the Ebola virus. Gleevec® and Tasigna® have been shown to stop the release of Ebola viral particles from infected cells, in vitro (see for instance WO 2007/002441; Mayra et al. Productive Replication of Ebola Virus Is Regulated by the ABL1 Tyrosine Kinase Science translational medicine 2012, 4, 123ra24). Inhibition of the ABL kinase can therefore be expected to reduce the pathogen's ability to replicate.

In prion disease models, Gleevec® showed beneficial effects. It delayed prion neuroinvasion by inhibiting prion propagation from the periphery to the CNS (Yun et al. The tyrosine kinase inhibitor imatinib mesylate delays prion neuroinvasion by inhibiting prion propagation in the periphery J Neurovirol. 2007, 13, 328-37). Gleevec® and ABL deficiency induced cellular clearance of PrPSc in prion-infected cells (Ertmer et al. The tyrosine kinase inhibitor STI571 induces cellular clearance of PrPSc in prion-infected cells J. Biol. Chem. 2004 279, 41918-27). Therefore, ABL1 inhibitors represent a valid therapeutic approach for the treatment of prion diseases, such as Creutzfeldt-Jacob disease (CJD).

X-linked recessive Emery-Dreifuss muscular dystrophy is caused by mutations of emerin, a nuclear-membrane protein with roles in nuclear architecture, gene regulation and signalling. A study has shown that emerin is tyrosine-phosphorylated directly by ABL1 in cell models, and that the phosphorylation status of emerin changes emerin binding to other proteins such as BAF. This, in turn, may explain the mislocalization of mutant emerin from nuclear to cytosolic compartments and consequently changes in downstream effector and signal integrator for signalling pathway(s) at the nuclear envelope (Tifft et al. Tyrosine phosphorylation of nuclear-membrane protein emerin by SRC, ABL1 and other kinases J. Cell Sci. 2009, 122, 3780-90). Changes in emerin-lamin interactions during both mitosis and interphase are of relevance for the pathology of muscular dystrophies. In addition, results from another study demonstrate that Gleevec® attenuates skeletal muscle dystrophy in mdx mice (Huang et al. Imatinib attenuates skeletal muscle dystrophy in mdx mice FASEB J. 2009, 23, 2539-48). Therefore, ABL1 inhibitors also represent therapeutic approaches for treatment of skeletal and muscular dystrophies.

Furthermore, ABL1 kinase plays a role in inflammation and oxidative stress, two mechanisms that are implicated in a variety of human diseases ranging from acute CNS diseases, such as stroke and traumatic brain or spinal cord injuries, chronic CNS diseases, such as Alzheimer's, Parkinson's, Huntington's and motoneuron diseases, to non-CNS inflammatory and autoimmune diseases, such as diabetes, pulmonary fibrosis.

For example, Gleevec® prevents fibrosis in different preclinical models of systemic sclerosis and induces regression of established fibrosis (Akhmetshina et al. Treatment with imatinib prevents fibrosis in different preclinical models of systemic sclerosis and induces regression of established fibrosis Arthritis Rheum. 2009, 60, 219-24) and it shows antifibrotic effects in bleomycin-induced pulmonary fibrosis in mice (Aono et al. Imatinib as a novel antifibrotic agent in bleomycin-induced pulmonary fibrosis in mice Am. J. Respir. Crit. Care Med. 2005, 171, 1279-85). Another study showed that both imatinib and nilotinib attenuated bleomycin-induced acute lung injury and pulmonary fibrosis in mice (Rhee et al. Effect of nilotinib on bleomycin-induced acute lung injury and pulmonary fibrosis in mice. Respiration 2011, 82, 273-87). Although in these studies the authors were focusing on the implication the mechanism related to PDGFRs, of interest, in the study by Rhee et al. (Respiration. 2011, 82, 273-87), nilotinib which is a more potent c-ABL inhibitor than imatinib showed superior therapeutic antifibrotic effects, thus supporting the therapeutic applicability of c-ABL inhibitors for treatment of human diseases with pulmonary inflammation. In another study, exposure of mice to hyperoxia increased ABL1 activation which is required for dynamin 2 phosphorylation and reactive oxygen species production and pulmonary leak (Singleton et al. Dynamin 2 and c-Abl are novel regulators of hyperoxia-mediated NADPH oxidase activation and reactive oxygen species production in caveolin-enriched microdomains of the endothelium J. Biol. Chem. 2009, 284, 34964-75).

In view of the above there is an unmet need for new compounds that may be used in the treatment and prevention of medical conditions in which inhibition of c-ABL is beneficial, such as neurodegenerative diseases (i.e. ALS and PD) and cancer (especially leukaemias).

### BRIEF DESCRIPTION OF THE FIGURES

FIG. 1 depicts UV chromatograms showing that Ponatinib forms a glutathione adduct (peak at 4.33 min) following incubation with recombinant CYP1A1 and human liver cytosol. That adduct is believed to be responsible (at least in part) for the toxicity associated with Ponatinib.

FIG. 2 depicts UV chromatograms showing that Example 25 does not form a glutathione adduct following incubation with recombinant CYP1A1 and human liver cytosol.

FIG. 3 shows the effect of exemplar compounds at rescuing motor neuron survival in the presence of ALS patient iAstrocytes.

FIG. 4 shows the effect of exemplar compounds at reducing α-synuclein levels in ReNCell VM neuronal cells overexpressing this protein.

### DISCLOSURE OF THE INVENTION

Surprisingly, it has been found that compounds of Formula (I) may inhibit c-ABL and therefore treat or prevent the above medical conditions. Further, they have certain beneficial properties leading to increased potential for use as a drug compared to known compounds. This may be in terms of their efficacy, free brain level at Cₘₐₓ, solubility, selectivity profiles, such as kinase selectivity, low hERG inhibitory activity, safety profile, and/or other notable pharmacokinetic properties.

Consequently, the invention relates to a compound of Formula (I),

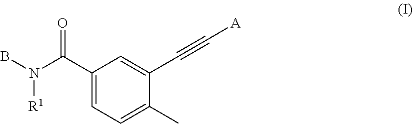

or a pharmaceutically acceptable salt, solvate, hydrate, tautomer, optical isomer, N-oxide, and/or prodrug thereof, wherein
A is an unsubstituted pyridyl;
B is a substituted 5-membered heteroaryl; and
R¹ is H or is selected from the group consisting of
(i) C₁-C₆ alkyl, C₂-C₆ alkenyl, and C₂-C₆ alkynyl, each of which is optionally substituted with one or more substituents independently selected from —NR^{a}R^{b}, —OR^{c}, halo, and oxo; and
(ii) C₆-C₁₀ aryl, C₁-C₉ heteroaryl, C₁-C₉ heterocycle, each of which is optionally substituted with one or more substituents independently selected from halo and C₁-C₆ alkyl, wherein the C₁-C₆ alkyl is optionally substituted with one or more halo atoms,
   wherein
   each R^{a} and R^{b} are independently selected from H and C₁-C₆ alkyl wherein the C₁-C₆ alkyl is optionally substituted with one or more halo atoms, or R^{a} and R^{b}, can be taken together with the nitrogen atom to which they are attached to form a 5- or 6-membered saturated, partially saturated, or unsaturated ring; and each R^{c} is independently selected from H and C₁-C₆ alkyl wherein the C₁-C₆ alkyl is optionally substituted with one or more halo atoms.

These compounds are compounds of the invention.

It is highly preferred that R¹ is H. In this case, the compounds of the invention are of Formula (II).

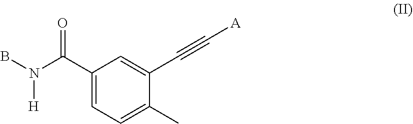

As used herein, the term “pyridyl” is a monovalent radical of pyridine. The pyridyl may be ortho-, meta-, or para-substituted, i.e. group A may be one of the following three groups.

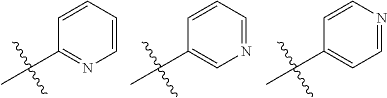

It is preferable that the pyridyl of group A is meta- or para-substituted, i.e. one of the following groups.

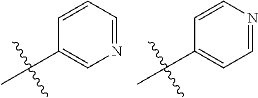 [SMILES: *c1cccnc1 ; *c1ccncc1]

Most preferably, group A is

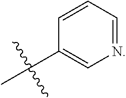 [SMILES: *c1cccnc1]

Without wishing to be bound by theory, the surprising beneficial properties of the compounds of the invention may be attributed, in part, to the pyridyl group. It has been unexpectedly found that compounds that comprise an unsubstituted pyridyl group have increased blood-brain barrier penetration making them particularly useful in the treatment of certain diseases and conditions. It is noted that blood-brain barrier penetration is unpredictable and is established empirically. Overcoming the challenges associated with delivering therapeutic agents to specific regions of the brain presents a major challenge to treatment of most brain disorders.

As used herein, “5-membered heteroaryl” is an aromatic monocyclic hydrocarbon ring in which at least one, such as 1, 2, 3 or 4, ring atom is a heteroatom. Examples of 5-membered heteroaryls useful as group B include, but are not limited to, substituted pyrrole, pyrazole, imidazole, triazole, tetrazole, isoxazole, oxadiazole, and thiazole. It is preferred that the 5-membered heteroaryl is a substituted pyrazole, imidazole, triazole, tetrazole, isoxazole oxadiazole, or thiazole, more preferably a substituted pyrazole, triazole or imidazole group, most preferably a substituted pyrazole or imidazole group.

Preferable examples of 5-membered heteroaryls include

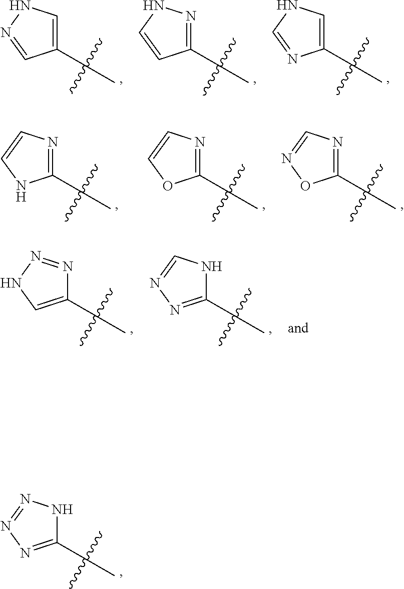

or a tautomer thereof, and each group being substituted. More preferably they include

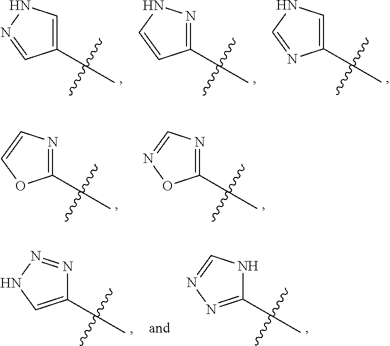

or a tautomer thereof, and each group being substituted.

In a preferred feature of the invention, the 5-membered heteroaryl of group B is substituted with one or more substituents independently selected from the group consisting of
(i) C₁-C₆ alkyl, C₂-C₆ alkenyl, C₂-C₆ alkynyl, and C₁-C₆ alkoxy, each of which is optionally substituted with one or more substituents independently selected from —NR^{d}R^{e}, —OR^{f}, halo, and oxo;
(ii) halo, —CN, —C(O)NR^{g}R^{h}, —C(O)ORⁱ, —C(O)Rⁱ, and —ORⁱ; and
(iii) C₆-C₁₀ aryl, C₁-C₉ heteroaryl, and C₁-C₉ heterocycle, each of which is optionally substituted with one or more substituents independently selected from halo and C₁-C₆ alkyl, wherein the C₁-C₆ alkyl is optionally substituted with one or more halo atoms,
   wherein R^{d}, R^{e}, R^{g}, and R^{h} are independently selected from H and C₁-C₆ alkyl wherein the C₁-C₆ alkyl is optionally substituted with one or more halo atoms, or R^{d} and R^{e}, and/or R^{g} and R^{h}, can be taken together with the nitrogen atom to which they are attached to form a 5- or 6-membered saturated, partially saturated, or unsaturated ring, which ring is optionally substituted with one or more groups selected from halo and C₁-C₃ alkyl, wherein the C₁-C₃ alkyl is optionally substituted with one or more halo atoms; and
   wherein R^{f} and Rⁱ are independently selected from H and C₁-C₆ alkyl, wherein the C₁-C₆ alkyl is optionally substituted with one or more halo atoms.

In a more preferable feature, the 5-membered heteroaryl of group B is substituted with one or more substituents independently selected from the group consisting of
(i) C₁-C₆ alkyl optionally substituted with one or more substituents selected from —NR^{d}R^{e}, —OR^{f}, halo, and oxo;
(ii) halo, —C(O)NR^{g}R^{h}, —C(O)ORⁱ; and
(iii) —ORⁱ.

In a more preferable feature, the 5-membered heteroaryl of group B is substituted with one or more substituents independently selected from the group consisting of
(i) C₁-C₆ alkyl optionally substituted with one or more substituents selected from halo, and —OR^{f};
(ii) halo, —C(O)ORⁱ; and
(iii) —ORⁱ.

Without wishing to be bound by theory, it may be particularly advantageous to include a hydrophobic group as a substituent on the 5-membered heteroaryl of group B, as this may increase interaction with a hydrophobic pocket of c-Abl, thereby increasing binding affinity. It is most preferable that the 5-membered heteroaryl of group B is substituted with a C₁-C₆ alkyl, isopropyl group or t-butyl group, more preferably an isopropyl group or t-butyl group, most preferably a t-butyl group. The substituent is preferably located at the 3- or 4-position relative to the point of attachment to the reminder of the compound as shown by the t-butyl group in the following two examples.

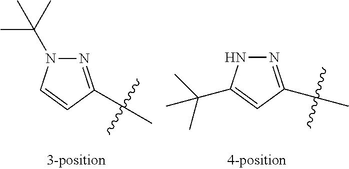

In addition to the above-mentioned preferred features of the invention, an alkylene group may be attached to two adjacent atoms of the 5-membered heteroaryl of group B to form a 5-, 6-, or 7-membered (preferably 5- or 6-membered) unsaturated, partially saturated, or saturated ring which is fused to the 5-membered heteroaryl of group B. Preferably it is a partially saturated or saturated ring which is fused to the 5-membered heteroaryl of group B. This fused bicyclic ring system is a specific aspect of the 5-membered heteroaryl of group B. The “alkylene group” in this instance is a linear chain diradical of C₃, C₄, or C₅ alkyl in which each radical is located at each terminus of the alkyl chain.

The fused bicyclic ring system of group B optionally has one or two carbon atoms of the alkylene group independently replaced with a heteroatom. When the heteroatom is nitrogen, then said nitrogen may be substituted with C₁-C₆ alkyl, or —C(O)O—(C₁-C₆ alkyl) wherein the C₁-C₆ alkyl is optionally substituted with one or more halo atoms. When the heteroatom is sulfur, then said sulfur may form a thionyl or sulfonyl group, such as in the following two examples of group B.

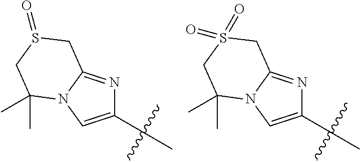 [SMILES: *c1cn2c(n1)CS(=O)CC2(C)C ; *c1cn2c(n1)CS(=O)(=O)CC2(C)C]

The carbon atoms of the alkylene group of the fused bicyclic ring system of group B may optionally be substituted with one or more substituents independently selected from halo, —C(O)O—(C₁-C₆ alkyl), C₁-C₆ alkyl, and oxo, preferably C₁-C₆ alkyl and oxo, wherein said C₁-C₆ alkyl is optionally independently substituted with one or more halo atoms.

Further, two hydrogen atoms attached to the same carbon of the alkylene group of the fused bicyclic ring system of group B may be optionally replaced with carbon atoms which, together with the carbon atom to which they are attached, form a C₃-C₆ cyclic alkyl group (i.e. forming a spiro motif), wherein said cyclic alkyl group is optionally substituted with one or more halo atoms, and/or one carbon is replaced with a heteroatom, preferably O or N.

When the compound comprises the fused bicyclic ring system of group B (such as those mentioned above) it is most preferable that the alkylene group forms a partially saturated or saturated ring with the 5-membered heteroaryl.

Exemplary fused bicyclic ring systems of group B include

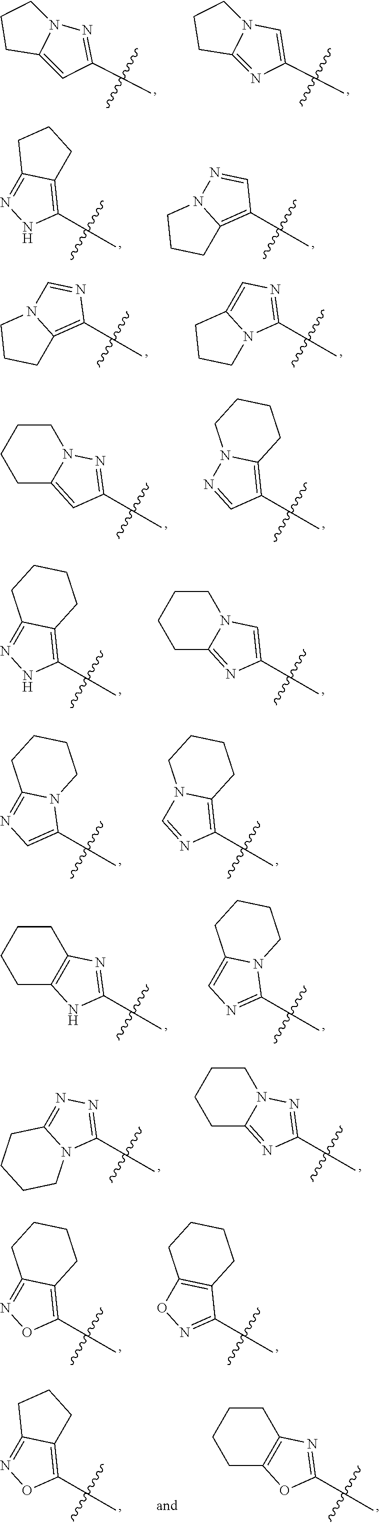

or tautomers thereof, each of which may be optionally substituted as outlined above.

In the case where one or more carbon atoms of the alkylene group has been replaced with a heteroatom, for instance N, O, or S, suitable examples include

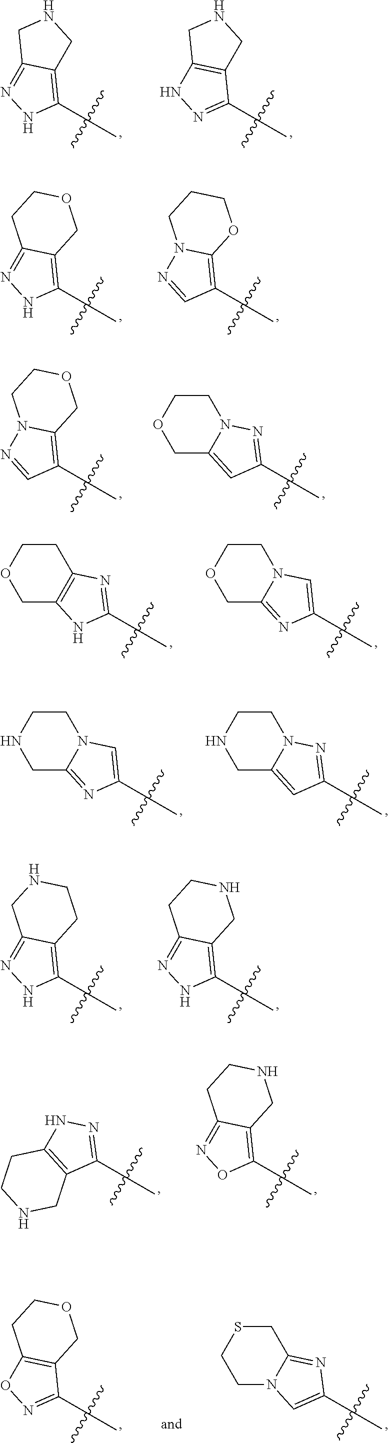

or tautomers thereof, each of which may be optionally substituted as outlined above.

Suitable examples of the above-mentioned spiro motif are found in the following examples of group B

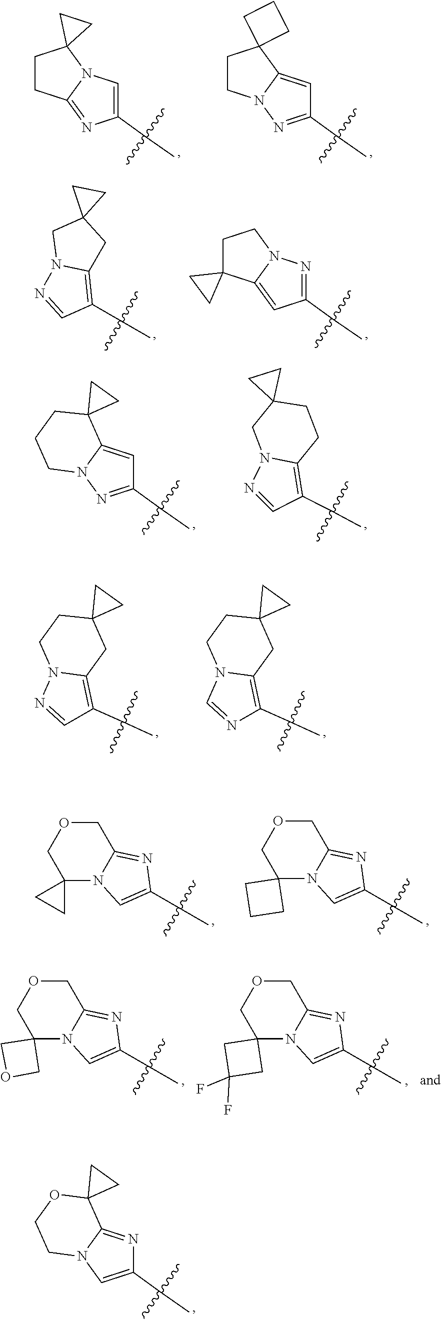

or tautomers thereof, each of which may be optionally substituted as outlined above.

Without wishing to be bound by theory, when the alkylene group is attached at the 2- and 3-positions of the 5-membered heteroaryl of group B, as in the group

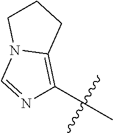 [SMILES: *c1ncn2c1CCC2]

the alkylene group may have increased interaction with a hydrophobic pocket of c-Abl thereby increasing binding affinity. When the alkylene group is attached at the 3- and 4-positions of the 5-membered heteroaryl of group B, as in

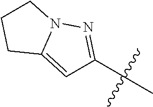 [SMILES: *c1cc2n(n1)CCC2]

it may be particularly advantageous to include a further hydrophobic substituent on the alkylene group to increase interaction with the hydrophobic pocket and thereby increase binding affinity to c-Abl. Preferably, the hydrophobic group is located alpha- to a bridgehead atom (as shown below). The hydrophobic group is preferably a C₁-C₆ alkyl group, more preferably a di-C₁-C₆ alkyl group (i.e. two C₁-C₆ alkyl groups substituted on the same atom), most preferably forming a gem-dimethyl group such as in the following example of group B.

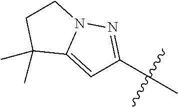 [SMILES: *c1cc2n(n1)CCC2(C)C]

It is especially preferred that group B does not contain any N—H groups. That is, it is preferable that all nitrogen atoms in group B are tri-substituted, for instance they may be tertiary amines. For the avoidance of doubt, this does not include N—H bonds formed between a nitrogen atom of group B and a pharmaceutically acceptable salt (such as HCl). Compounds that do not comprise an N—H as part of group B may have increased interaction with a hydrophobic pocket of c-Abl thereby further increasing binding affinity.

More preferably, the 5-membered heteroaryl of group B is substituted with one or more substituents independently selected from the group consisting of
(i) C₁-C₆ alkyl optionally substituted with one or more substituents selected from halo, and —OR^{f};
(ii) halo, —C(O)ORⁱ; and
(iii) —ORⁱ,
   and/or wherein an alkylene group is attached to two adjacent atoms of the 5-membered heteroaryl of group B to form a 5- or 6-membered partially saturated or saturated ring,
   optionally wherein one or more of the carbon atoms of the alkylene group is substituted with one or more substituents independently selected from halo, C₁-C₆ alkyl and oxo, wherein said alkyl groups are optionally independently substituted with one or more halo atoms; and/or
   optionally wherein two hydrogen atoms attached to the same carbon of the alkylene group are replaced with carbon atoms which, together with the carbon atom to which they are attached, form a C₃-C₆ cyclic alkyl group, wherein said cyclic alkyl group is optionally substituted with one or more halo atoms.

In a particularly preferred feature of the invention, the compound is a compound of formula (I), preferably formula (II),

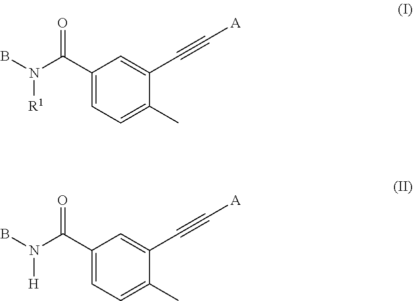

wherein

### A is

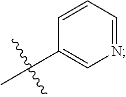 [SMILES: *c1cccnc1]

B is selected from the group consisting of

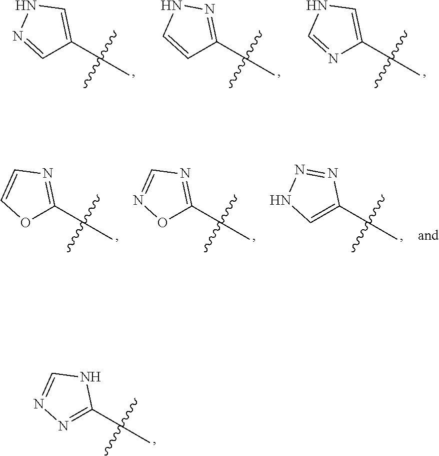

and is substituted with one or more substituents independently selected from the group consisting of
(i) C₁-C₆ alkyl optionally substituted with one or more substituents selected from halo, and —OR^{f};
(ii) halo, —C(O)ORⁱ; and
(iii) —ORⁱ
   and/or wherein an alkylene group is attached to two adjacent atoms of the 5-membered heteroaryl of group B to form a 5-, 6-membered partially saturated or saturated ring, which is fused to the 5-membered heteroaryl of group B,
   optionally wherein one or more of the carbon atoms of the alkylene group is substituted with one or more substituents independently selected from halo, C₁-C₆ alkyl and oxo, wherein said alkyl groups are optionally independently substituted with one or more halo atoms; and/or
   optionally wherein two hydrogen atoms attached to the same carbon of the alkylene group are replaced with carbon atoms which, together with the carbon atom to which they are attached, form a C₃-C₆ cyclic alkyl group, wherein said cyclic alkyl group is optionally substituted with one or more halo atoms.

In one aspect, the compounds of the present invention comprise the above-mentioned fused bicyclic ring system of group B. These particular compounds may be defined as being compounds of Formula (I), preferably Formula (II), with group B being selected from optionally substituted group (V) and optionally substituted group (W)

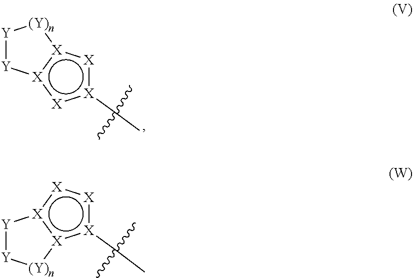

wherein each X and Y is independently selected from C, S, O, and N,
at least one X is N;
at least two X are C;
at least two Y are C;
n=1, 2 or 3, preferably 1 or 2;
wherein
each X is optionally independently substituted with one or more substituents selected from halo, —CN, —C(O)OH, —C(O)O—(C₁-C₆ alkyl), and C₁-C₆ alkyl, preferably halo, —C(O)OH, —C(O)O—(C₁-C₆ alkyl), and C₁-C₆ alkyl, most preferably C₁-C₆ alkyl, wherein said alkyl groups are optionally substituted with one or more halo atoms; and
each Y is optionally independently substituted with one or more substituents selected from halo, —C(O)OH, —C(O)O—(C₁-C₆ alkyl), C₁-C₆ alkyl, and oxo, preferably halo, —C(O)O—(C₁-C₆ alkyl), C₁-C₆ alkyl, and oxo, most preferably C₁-C₆ alkyl and oxo, wherein said alkyl groups are optionally substituted with one or more halo atoms; and/or
optionally wherein two hydrogen atoms attached to the same Y are replaced with carbon atoms which, together with the carbon atom to which they are attached, form a C₃-C₆ cyclic alkyl group, wherein said cyclic alkyl group is optionally substituted with one or more halo atoms.

In this particular aspect, it is preferred that the compounds of the present invention are selected from Formulae (II-V) or (II-W), wherein X and Y are as defined above.

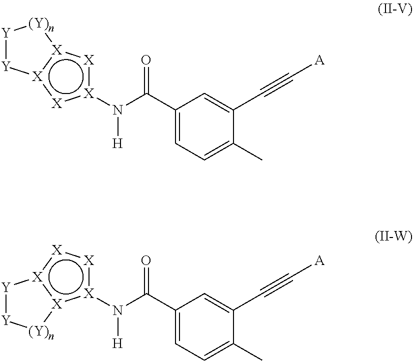

Preferred examples of the fused bicyclic ring system of group B are those listed below, or a tautomer thereof, with each group being optionally substituted as outlined above. These specific examples of the fused bicyclic ring system of group B preferably form the compounds of Formulae (II-V) and (II-W).

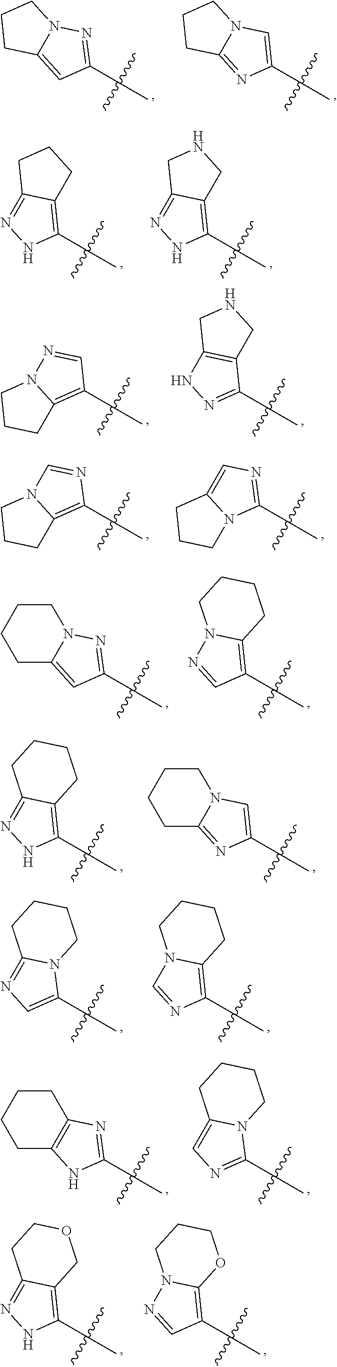

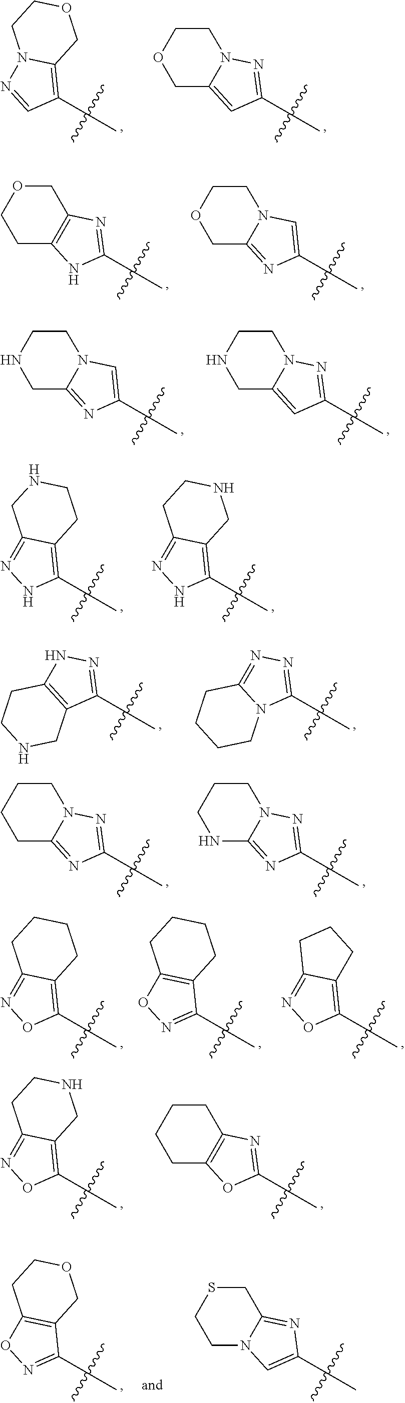

Especially preferred examples of the fused bicyclic ring system of group B are those listed below, or a tautomer thereof, with each group being optionally substituted as outlined above. These specific examples of the fused bicyclic ring system of group B are especially preferred to form the compounds of Formulae (II-V) and (II-W).

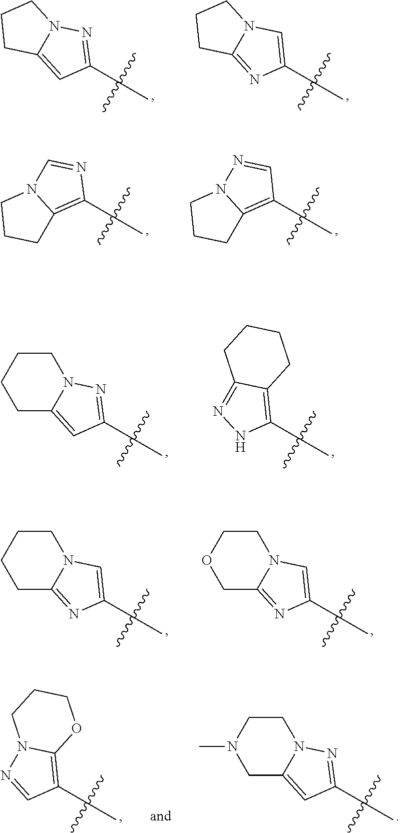

Particular compounds of the invention are those listed below.
4-methyl-N-{4H,5H,6H,7H-pyrazolo[1,5-a]pyridin-2-yl}-3-[2-(pyridin-3-yl)ethynyl]benzamide;
4-methyl-N-(2-methyl-4,5,6,7-tetrahydro-2H-indazol-3-yl)-3-[2-(pyridin-3-yl)ethynyl]benzamide;
4-methyl-N-[1-(propan-2-yl)-1H-1,2,3-triazol-4-yl]-3-[2-(pyridin-3-yl)ethynyl]benzamide;
4-methyl-3-[2-(pyridin-3-yl)ethynyl]-N-[1-(2,2,2-trifluoroethyl)-1H-pyrazol-3-yl]benzamide;
4-methyl-3-[2-(pyridin-3-yl)ethynyl]-N-[1-(3,3,3-trifluoropropyl)-1H-pyrazol-4-yl]benzamide:
N-(1-tert-butyl-1H-pyrazol-3-yl)-4-methyl-3-[2-(pyridin-3-yl)ethynyl]benzamide;
N-(5-cyclopropyl-1-methyl-1H-pyrazol-3-yl)-4-methyl-3-[2-(pyridin-3-yl)ethynyl]benzamide;
4-methyl-N-[5-methyl-1-(2,2,2-trifluoroethyl)-1H-pyrazol-3-yl]-3-[2-(pyridin-3-yl)ethynyl]benzamide;
N-(1-cyclobutyl-1H-pyrazol-4-yl)-4-methyl-3-[2-(pyridin-3-yl)ethynyl]benzamide;
N-(1-tert-butyl-1H-pyrazol-4-yl)-4-methyl-3-[2-(pyridin-3-yl)ethynyl]benzamide;
N-(3-tert-butyl-1-methyl-1H-pyrazol-5-yl)-4-methyl-3-[2-(pyridin-3-yl)ethynyl]benzamide;
N-[1-(2,2-dimethylpropyl)-1H-pyrazol-4-yl]-4-methyl-3-[2-(pyridin-3-yl)ethynyl]benzamide;
N-[1-(cyclopropylmethyl)-1H-pyrazol-4-yl]-4-methyl-3-[2-(pyridin-3-yl)ethynyl]benzamide;
N-[5-(difluoromethyl)-1-methyl-1H-pyrazol-3-yl]-4-methyl-3-[2-(pyridin-3-yl)ethynyl]benzamide;
4-methyl-N-[5-methyl-1-(2-methylpropyl)-1H-pyrazol-4-yl]-3-[2-(pyridin-3-yl)ethynyl]benzamide;
N-[1-(2,2-difluorocyclopropyl)-1H-pyrazol-4-yl]-4-methyl-3-[2-(pyridin-3-yl)ethynyl]benzamide;
N-[1-(1-cyclopropylethyl)-1H-pyrazol-4-yl]-4-methyl-3-[2-(pyridin-3-yl)ethynyl]benzamide;
N-(1-cyclobutyl-5-methyl-1H-pyrazol-4-yl)-4-methyl-3-[2-(pyridin-3-yl)ethynyl]benzamide;
4-methyl-N-[1-(propan-2-yl)-1H-1,2,4-triazol-3-yl]-3-[2-(pyridin-3-yl)ethynyl]benzamide;
4-methyl-N-[1-(2-methylpropyl)-1H-pyrazol-4-yl]-3-[2-(pyridin-3-yl)ethynyl]benzamide;
N-[5-(difluoromethoxy)-1-methyl-1H-pyrazol-3-yl]-4-methyl-3-[2-(pyridin-3-yl)ethynyl]benzamide:
N-[1-(2,2-difluoroethyl)-5-methyl-1H-pyrazol-4-yl]-4-methyl-3-[2-(pyridin-3-yl)ethynyl]benzamide;
4-methyl-N-[5-methyl-1-(propan-2-yl)-1H-pyrazol-4-yl]-3-[2-(pyridin-3-yl)ethynyl]benzamide;
N-(1-isopropylpyrazol-4-yl)-4-methyl-3-[2-(3-pyridyl)ethynyl]benzamide;
N-(1-isopropylimidazol-4-yl)-4-methyl-3-[2-(3-pyridyl)ethynyl]benzamide;
N-(4,4-dimethyl-5,6-dihydropyrrolo[1,2-b]pyrazol-2-yl)-4-methyl-3-[2-(3-pyridyl)ethynyl]benzamide;
N-[1-(cyclopropylmethyl)imidazol-4-yl]-4-methyl-3-[2-(3-pyridyl)ethynyl]benzamide;
4-Methyl-3-[2-(3-pyridyl)ethynyl]-N-[1-(2,2,2-trifluoroethyl)imidazol-4-yl]benzamide;
4-methyl-3-[2-(3-pyridyl)ethynyl]-N-[5-(trifluoromethyl)-6,7-dihydro-5H-pyrrolo[1,2-a]imidazol-2-yl]benzamide;
4-Methyl-3-[2-(3-pyridyl)ethynyl]-N-spiro[6,7-dihydropyrrolo[1,2-a]imidazole-5,1′-cyclopropane]-2-yl-benzamide;
4-methyl-N-(1-propylimidazol-4-yl)-3-[2-(3-pyridyl)ethynyl]benzamide;
N-(1-cyclopropylimidazol-4-yl)-4-methyl-3-[2-(3-pyridyl)ethynyl]benzamide;
4-methyl-3-[2-(3-pyridyl)ethynyl]-N-[5-(trifluoromethyl)-5,6,7,8-tetrahydroimidazo[1,2-a]19yridine-2-yl]benzamide;
N-(4-tert-butyl-1,3-oxazol-2-yl)-4-methyl-3-[2-(pyridin-3-yl)ethynyl]benzamide;
4-methyl-N-[4-(propan-2-yl)-1,3-oxazol-2-yl]-3-[2-(pyridin-3-yl)ethynyl]benzamide;
4-methyl-N-[2-(propan-2-yl)-2H-1,2,3,4-tetrazol-5-yl]-3-[2-(pyridin-3-yl)ethynyl]benzamide;
N-(1-tert-butyl-1H-1,2,4-triazol-3-yl)-4-methyl-3-[2-(pyridin-3-yl)ethynyl]benzamide;
N-(1-tert-butylimidazol-4-yl)-4-methyl-3-[2-(3-pyridyl)ethynyl]benzamide;
N-(5,5-dimethyl-6,8-dihydroimidazo[2,1-c][1,4]oxazin-2-yl)-4-methyl-3-[2-(3-pyridyl)ethynyl]benzamide;
N-(6,6-dimethyl-5,7-dihydropyrrolo[1,2-c]imidazol-1-yl)-4-methyl-3-[2-(3-pyridyl)ethynyl]benzamide;
4-methyl-3-[2-(3-pyridyl)ethynyl]-N-spiro[5,6-dihydropyrrolo[1,2-b]pyrazole-4,1′-cyclobutane]-2-yl-benzamide;
N-(1-cyclopentylimidazol-4-yl)-4-methyl-3-[2-(3-pyridyl)ethynyl]benzamide;
N-[1-(difluoromethyl)-5-methyl-1H-pyrazol-4-yl]-4-methyl-3-[2-(pyridin-3-yl)ethynyl]benzamide;
N-(5,5-dimethyl-6,7-dihydropyrrolo[1,2-a]imidazol-2-yl)-4-methyl-3-[2-(3-pyridyl)ethynyl]benzamide;
4-methyl-N-[4-(2-methylpropyl)-1,3-oxazol-2-yl]-3-[2-(pyridin-3-yl)ethynyl]benzamide;
4-methyl-N-[3-(propan-2-yl)-1,2,4-oxadiazol-5-yl]-3-[2-(pyridin-3-yl)ethynyl]benzamide;
N-[1-(butan-2-yl)-1H-pyrazol-3-yl]-4-methyl-3-[2-(pyridin-3-yl)ethynyl]benzamide;
4-methyl-3-[2-(pyridin-3-yl)ethynyl]-N-[1-(2,2,2-trifluoroethyl)-1H-pyrazol-4-yl]benzamide;
N-{6,6-dimethyl-5H,6H,7H-pyrazolo[3,2-b][1,3]oxazin-3-yl}-4-methyl-3-[2-(pyridin-3-yl)ethynyl]benzamide;
4-methyl-N-(1-propyl-1H-pyrazol-4-yl)-3-[2-(pyridin-3-yl)ethynyl]benzamide;
N-(3-cyclobutyl-1-methyl-1H-pyrazol-5-yl)-4-methyl-3-[2-(pyridin-3-yl)ethynyl]benzamide;
N-[3-methoxy-1-(2,2,2-trifluoroethyl)-1H-pyrazol-4-yl]-4-methyl-3-[2-(pyridin-3-yl)ethynyl]benzamide;
N-[1-(2,2-difluoroethyl)-1H-pyrazol-4-yl]-4-methyl-3-[2-(pyridin-3-yl)ethynyl]benzamide;
N-[1-(2,2-difluoroethyl)-5-methyl-1H-pyrazol-3-yl]-4-methyl-3-[2-(pyridin-3-yl)ethynyl]benzamide;
4-methyl-N-[5-methyl-1-(propan-2-yl)-1H-pyrazol-3-yl]-3-[2-(pyridin-3-yl)ethynyl]benzamide;
N-(5-isopropyl-6,7-dihydro-5H-pyrrolo[1,2-a]imidazol-2-yl)-4-methyl-3-[2-(3-pyridyl)ethynyl]benzamide;
N-(5-isopropyl-6,8-dihydro-5H-imidazo[2,1-c][1,4]oxazin-2-yl)-4-methyl-3-[2-(3-pyridyl)ethynyl]benzamide;
4-methyl-N-[4-methyl-1-(propan-2-yl)-1H-pyrazol-3-yl]-3-[2-(pyridin-3-yl)ethynyl]benzamide;
N-[5-(difluoromethyl)-1-methyl-1H-1,2,3-triazol-4-yl]-4-methyl-3-[2-(pyridin-3-yl)ethynyl]benzamide;
4-methyl-N-(5-methyl-6,7-dihydro-5H-pyrrolo[1,2-c]imidazol-1-yl)-3-[2-(3-pyridyl)ethynyl]benzamide;
4-methyl-N-[1-methyl-3-(2-methylpropyl)-1H-pyrazol-5-yl]-3-[2-(pyridin-3-yl)ethynyl]benzamide;
N-(1-cyclopropyl-1H-pyrazol-4-yl)-4-methyl-3-[2-(pyridin-3-yl)ethynyl]benzamide;
4-methyl-3-[2-(3-pyridyl)ethynyl]-N-spiro[5,6-dihydropyrrolo[1,2-b]pyrazole-4,1′-cyclopropane]-2-yl-benzamide;
N-(1-isobutylimidazol-4-yl)-4-methyl-3-[2-(3-pyridyl)ethynyl]benzamide;
N-(4-ethyl-5,6-dihydro-4H-pyrrolo[1,2-b]pyrazol-2-yl)-4-methyl-3-[2-(3-pyridyl)ethynyl]benzamide;
4-methyl-N-[1-(propan-2-yl)-1H-pyrazol-3-yl]-3-[2-(pyridin-3-yl)ethynyl]benzamide;
4-methyl-N-(5-methyl-5,6,7,8-tetrahydroimidazo[1,2-a]pyridin-2-yl)-3-[2-(3-pyridyl)ethynyl]benzamide;
N-[1-(cyclobutylmethyl)-1H-pyrazol-4-yl]-4-methyl-3-[2-(pyridin-3-yl)ethynyl]benzamide;
N-(1-cyclobutylimidazol-4-yl)-4-methyl-3-[2-(3-pyridyl)ethynyl]benzamide;
N-[3-(cyclopropylmethyl)-1-methyl-1H-pyrazol-5-yl]-4-methyl-3-[2-(pyridin-3-yl)ethynyl]benzamide;
4-methyl-N-[3-methyl-1-(2,2,2-trifluoroethyl)-1H-pyrazol-4-yl]-3-[2-(pyridin-3-yl)ethynyl]benzamide;
N-[1-(2,2-difluoroethyl)-3-methyl-1H-pyrazol-4-yl]-4-methyl-3-[2-(pyridin-3-yl)ethynyl]benzamide;
N-[1-(2,2-difluorocyclopropyl)-1H-pyrazol-3-yl]-4-methyl-3-[2-(pyridin-3-yl)ethynyl]benzamide;
4-methyl-N-[3-methyl-1-(2-methylpropyl)-1H-pyrazol-4-yl]-3-[2-(pyridin-3-yl)ethynyl]benzamide:
N-[1-(2,2-difluoroethyl)-1H-pyrazol-3-yl]-4-methyl-3-[2-(pyridin-3-yl)ethynyl]benzamide;
4-methyl-N-[4-methyl-1-(2,2,2-trifluoroethyl)-1H-pyrazol-3-yl]-3-[2-(pyridin-3-yl)ethynyl]benzamide;
N-(1-cyclobutyl-3-methyl-1H-pyrazol-4-yl)-4-methyl-3-[2-(pyridin-3-yl)ethynyl]benzamide;
N-[1-(2-fluoroethyl)imidazol-4-yl]-4-methyl-3-[2-(3-pyridyl)ethynyl]benzamide;
4-methyl-N-[3-methyl-1-(propan-2-yl)-1H-pyrazol-4-yl]-3-[2-(pyridin-3-yl)ethynyl]benzamide;
4-methyl-N-[1-(2-methylpropyl)-1H-pyrazol-3-yl]-3-[2-(pyridin-3-yl)ethynyl]benzamide;
4-methyl-N-{5-methyl-4-oxo-4H,5H,6H,7H-pyrazolo[1,5-a]pyrazin-2-yl}-3-[2-(pyridin-3-yl)ethynyl]benzamide;
4-methyl-N-(5-methyl-6,7-dihydro-5H-pyrrolo[1,2-a]imidazol-2-yl)-3-[2-(3-pyridyl)ethynyl]benzamide;
N-[1,4-dimethyl-3-(trifluoromethyl)-1H-pyrazol-5-yl]-4-methyl-3-[2-(pyridin-3-yl)ethynyl]benzamide;
N-(3-cyclopropyl-1-methyl-1H-pyrazol-5-yl)-4-methyl-3-[2-(pyridin-3-yl)ethynyl]benzamide;
4-methyl-N-[1-methyl-3-(propan-2-yl)-1H-1,2,4-triazol-5-yl]-3-[2-(pyridin-3-yl)ethynyl]benzamide;
N-[1-(difluoromethyl)-1H-pyrazol-4-yl]-4-methyl-3-[2-(pyridin-3-yl)ethynyl]benzamide;
N-[1-(2,2-difluoroethyl)-4-methyl-1H-pyrazol-3-yl]-4-methyl-3-[2-(pyridin-3-yl)ethynyl]benzamide;
4-methyl-N-(4-oxo-6,7-dihydro-5H-pyrazolo[1,5-a]pyridin-2-yl)-3-[2-(3-pyridyl)ethynyl]benzamide;
4-methyl-N-(5-methyl-6,8-dihydro-5H-imidazo[2,1-c][1,4]oxazin-2-yl)-3-[2-(3-pyridyl)ethynyl]benzamide;
N-[4-chloro-1-(propan-2-yl)-1H-pyrazol-3-yl]-4-methyl-3-[2-(pyridin-3-yl)ethynyl]benzamide;
N-(3-cyclopropyl-1-ethyl-1H-pyrazol-5-yl)-4-methyl-3-[2-(pyridin-3-yl)ethynyl]benzamide:
4-methyl-N-[5-methyl-1-(2-methylpropyl)-1H-pyrazol-3-yl]-3-[2-(pyridin-3-yl)ethynyl]benzamide;
N-[1-(1-methoxypropan-2-yl)-1H-pyrazol-3-yl]-4-methyl-3-[2-(pyridin-3-yl)ethynyl]benzamide;
N-[1-(cyclobutylmethyl)imidazol-4-yl]-4-methyl-3-[2-(3-pyridyl)ethynyl]benzamide;
4-Methyl-3-[2-(3-pyridyl)ethynyl]-N-spiro[6,7-dihydro-5H-pyrazolo[1,5-a]pyridine-4,1′-cyclopropane]-2-yl-benzamide;
N-(6,6-dimethyl-5,8-dihydroimidazo[2,1-c][1,4]oxazin-2-yl)-4-methyl-3-[2-(3-pyridyl)ethynyl]benzamide;
N-(4,4-difluoro-6,7-dihydro-5H-pyrazolo[1,5-a]pyridin-2-yl)-4-methyl-3-[2-(3-pyridyl)ethynyl]benzamide;
N-[1-(2-fluoroethyl)-1H-pyrazol-4-yl]-4-methyl-3-[2-(pyridin-3-yl)ethynyl]benzamide;
4-methyl-N-{4H,5H,6H,7H-pyrazolo[1,5-a]pyridin-3-yl}-3-[2-(pyridin-3-yl)ethynyl]benzamide;
4-methyl-3-[2-(pyridin-3-yl)ethynyl]-N-(4,5,6,7-tetrahydro-1,2-benzoxazol-3-yl)benzamide;
4-methyl-3-[2-(pyridin-3-yl)ethynyl]-N-(4,5,6,7-tetrahydro-2,1-benzoxazol-3-yl)benzamide;
N-(5,5-difluoro-4,5,6,7-tetrahydro-1,2-benzoxazol-3-yl)-4-methyl-3-[2-(pyridin-3-yl)ethynyl]benzamide; or
N-(5-tert-butyl-1,3,4-oxadiazol-2-yl)-4-methyl-3-[2-(pyridin-3-yl)ethynyl]benzamide.

The compounds of the invention may include isotopically-labelled and/or isotopically-enriched forms of the compounds. The compounds of the invention herein may contain unnatural proportions of atomic isotopes at one or more of the atoms that constitute such compounds. Examples of isotopes that can be incorporated into the disclosed compounds include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl.

The compounds of the invention may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

Throughout the present disclosure, a given chemical formula or name shall also encompass all pharmaceutically acceptable salts, solvates, hydrates, N-oxides, and/or prodrug forms thereof. It is to be understood that the compounds of the invention include any and all hydrates and/or solvates of the compound formulas. It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates and/or solvates.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein can be asymmetric (e.g. having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis- and trans-geometric isomers of the compounds of the present invention are described and may be isolated as a mixture of isomers or as separated isomeric forms.

In the case of the compounds which contain an asymmetric carbon atom, the invention relates to the D form, the L form, and D,L mixtures and also, where more than one asymmetric carbon atom is present, to the diastereomeric forms. Those compounds of the invention which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

The term “prodrugs” refers to compounds that may be converted under physiological conditions or by solvolysis to a biologically active compound of the invention. A prodrug may be inactive when administered to a subject in need thereof, but is converted in vivo to an active compound of the invention. Prodrugs are typically rapidly transformed in vivo to yield the parent compound of the invention, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2nd Ed., Elsevier Academic Press (2004), page 498 to 549). Prodrugs of a compound of the invention may be prepared by modifying functional groups, such as a hydroxy, amino or mercapto groups, present in a compound of the invention in such a way that the modifications are cleaved, either in routine manipulation or in vivo, to the parent compound of the invention. Examples of prodrugs include, but are not limited to, acetate, formate and succinate derivatives of hydroxy functional groups or phenyl carbamate derivatives of amino functional groups.

Another object of the present invention relates to the compounds of the invention for use in therapy.

The compounds of the invention are useful as inhibitors of c-ABL. As such, they are useful in the treatment or prevention of medical conditions (conditions or diseases) in which inhibition of c-ABL is beneficial. There is therefore provided a method of for the treatment or prevention of a disease or condition responsive to c-ABL inhibition comprising administering a therapeutically effective amount of a compound of the invention to a subject. Whilst the compounds of the invention may be suitable to prevent a range of diseases and conditions, it is preferable that they are used to treat said diseases and conditions. Therefore, it is preferred that the method is for the treatment of a disease or condition, and therefore the method comprises administering a therapeutically effective amount of a compound of the invention to a subject in need thereof.

The term “treatment” as used herein may include prophylaxis of the named disorder or condition, or amelioration or elimination of the disorder once it has been established. The term “prevention” refers to prophylaxis of the named disorder or condition.

The range of diseases and conditions treatable or preventable by c-ABL inhibition is well known. The compounds of the invention therefore may be used to treat or prevent this range of diseases or conditions. This includes neurodegenerative disorders, cancers, prion diseases, viral infections, diabetes, inflammatory diseases such as pulmonary fibrosis, or a skeletal or muscular dystrophy. Preferably, the disease is a neurodegenerative disorder or a cancer.

Treatable or preventable neurodegenerative disorders include, but are not limited to, Alzheimer disease, Down's syndrome, frontotemporal dementia, progressive supranuclear palsy, Pick's disease, Niemann-Pick disease, Parkinson's disease, Huntington's disease (HD), dentatorubropallidoluysian atrophy, Kennedy's disease, and spinocerebellar ataxia, fragile X (Rett's) syndrome, fragile XE mental retardation, Friedreich's ataxia, myotonic dystrophy, spinocerebellar ataxia type 8, and spinocerebellar ataxia type 12, Alexander disease, Alper's disease, amyotrophic lateral sclerosis (ALS), ataxia telangiectasia, Batten disease, Canavan disease, Cockayne syndrome, corticobasal degeneration, Creutzfeldt-Jakob disease, ischemia stroke, Krabbe disease, Lewy body dementia, multiple sclerosis, multiple system atrophy, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis.

Of the treatable or preventable neurodegenerative disorders, most notable are amyotrophic lateral sclerosis (ALS) and Parkinson's disease. Most preferably the neurodegenerative disorder is ALS.

Treatable or preventable cancers include, but are not limited to, leukaemia.

Of the treatable or preventable cancers, most notable are chronic myeloid leukaemia (CML), acute lymphoblastic leukaemia (ALL), acute myelogenous leukaemia (AML), and mixed-phenotype acute leukaemia (MPAL), or any central nervous system (CNS) metastases thereof. Most preferably the cancer is CML or ALL.

The invention thus includes the use of the compounds of the invention in the manufacture of a medicament for the treatment or prevention of a disease or condition, such as the above-mentioned neurodegenerative disorders and cancers. The invention also relates to the compounds of the invention for use in the treatment of a disease or condition, such as the above-mentioned neurodegenerative disorders and cancers.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is pre-screened or identified as in need of such treatment by assessment for a relevant marker or indicator of suitability for such treatment.

The invention provides a method of monitoring treatment progress. The method includes the step of determining a level of diagnostic marker (Marker) (e.g. any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

A level of Marker or Marker activity in a subject may be determined at least once. Comparison of Marker levels, e.g., to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, is ELISA, radiolabeling/assay techniques, blotting/chemiluminescence methods, real-time PCR, and the like.

For clinical use, the compounds disclosed herein are formulated into pharmaceutical compositions (or formulations) for various modes of administration. It will be appreciated that compounds of the invention may be administered together with a physiologically acceptable carrier, excipient, and/or diluent (i.e. one, two, or all three of these). The pharmaceutical compositions disclosed herein may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration. Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration. The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds disclosed herein may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

### Definitions

The term “unsubstituted” means that the group to which it refers has no hydrogen atoms substituted for a different group. For instance, “unsubstituted pyridyl” refers to a monovalent radical of pyridine with only hydrogens attached to the ring except for the point at which it is attached to the remainder of the compound.

The term “heteroatom” means O, N, or S. Typically, it is preferred that the heteroatom or heteroatoms in the 5-membered heteroaryl group B is nitrogen.

“Optional” or “optionally” means that the subsequently described event or circumstance may, but need not, occur, and that the description includes instances where the event or circumstance occurs and instances in which it does not.

The term “C₁-C₆ alkyl” denotes a straight, branched or cyclic or partially cyclic alkyl group having from 1 to 6 carbon atoms, i.e. 1, 2, 3, 4, 5 or 6 carbon atoms. For the “C₁-C₆ alkyl” group to comprise a cyclic portion it should be formed of 3 to 6 carbon atoms. For parts of the range “C₁-C₆ alkyl” all subgroups thereof are contemplated, such as C₁-C₅ alkyl, C₁-C₄ alkyl, C₁-C₃ alkyl, C₁-C₂ alkyl, C₁ alkyl, C₂-C₆ alkyl, C₂-C₅ alkyl, C₂-C₄ alkyl, C₂-C₃ alkyl, C₂ alkyl, C₃-C₆ alkyl, C₃-C₅ alkyl, C₃-C₄ alkyl, C₃ alkyl, C₄-C₆ alkyl, C₄-C₅ alkyl, C₄ alkyl, C₅-C₆ alkyl, C₅ alkyl, and C₆ alkyl. Examples of “C₁-C₆ alkyl” include methyl, ethyl, n-propyl, isopropyl, cyclopropyl, n-butyl, isobutyl, sec-butyl, t-butyl, cyclobutyl, cyclopropylmethyl, and straight, branched or cyclic or partially cyclic pentyl and hexyl etc.

When a term denotes a range, for instance “1 to 6 carbon atoms” in the definition of C₁-C₆ alkyl, each integer is considered to be disclosed, i.e. 1, 2, 3, 4, 5 and 6.

The term “C₂-C₆ alkenyl” denotes a straight, branched or cyclic or partially cyclic alkyl group having at least one carbon-carbon double bond, and having from 2 to 6 carbon atoms. The alkenyl group may comprise a ring formed of 3 to 6 carbon atoms. For parts of the range “C₂-C₆ alkenyl” all subgroups thereof are contemplated, such as C₂-C₅ alkenyl, C₂-C₄ alkenyl, C₂-C₃ alkenyl, C₂ alkenyl, C₃-C₆alkenyl, C₃-C₅ alkenyl, C₃-C₄ alkenyl, C₃ alkenyl, C₄-C₆ alkenyl, C₄-C₅ alkenyl, C₄ alkenyl, C₅-C₆ alkenyl, C₅ alkenyl, and C₆ alkenyl. Examples of “C₂-C₆ alkenyl” include 2-propenyl, 2-butenyl, 3-butenyl, 2-methyl-2-propenyl, 2-hexenyl, 5-hexenyl, 2,3-dimethyl-2-butenyl.

The term “C₂-C₆ alkynyl” denotes a straight, branched or cyclic or partially cyclic alkyl group having at least one carbon-carbon triple bond, and having from 2 to 6 carbon atoms. The alkynyl group may comprise a ring formed of 3 to 6 carbon atoms. For parts of the range “C₂-C₆ alkynyl” all subgroups thereof are contemplated, such as C₂-C₅ alkynyl, C₂-C₄ alkynyl, C₂-C₃ alkynyl, C₂ alkynyl, C₃-C₆alkynyl, C₃-C₅ alkynyl, C₃-C₄ alkynyl, C₃ alkynyl, C₄-C₆ alkynyl, C₄-C₅ alkynyl, C₄ alkynyl, C₅-C₆ alkynyl, C₅ alkynyl, and C₆ alkynyl. Examples of “C₂-C₆ alkynyl” include 2-propynyl, 2-butynyl, 3-butynyl, 2-pentynyl, 3-methyl-4-pentynyl, 2-hexynyl, 5-hexynyl etc.

The term “C₁-C₆ alkoxy” denotes —O—(C₁-C₆alkyl) in which a C₁-C₆ alkyl group is as defined above and is attached to the remainder of the compound through an oxygen atom. Examples of “C₁-C₆ alkoxy” include methoxy, ethoxy, n-propoxy, isopropoxy, n-butoxy, isobutoxy, sec-butoxy, t-butoxy and straight- and branched-chain pentoxy and hexoxy.

The term “halo” means a halogen atom, and is preferably, F, Cl, Br and I, more preferably F and Cl, and most preferably F.

The term “oxo” denotes a double bond to an oxygen atom (═O). This typically forms a ketone or aldehyde group.

The term “C₆-C₁₀ aryl” denotes an aromatic monocyclic or fused bicyclic hydrocarbon ring comprising 6 to 10 ring atoms. Examples of “C₆-C₁₀ aryl” groups include phenyl, indenyl, naphthyl, and naphthalene.

The term “C₁-C₉ heteroaryl” denotes an aromatic monocyclic or fused bicyclic heteroaromatic ring system having 5 to 10 ring atoms in which 1 to 9 of the ring atoms are carbon and one or more of the ring atoms are selected from nitrogen, sulphur, and oxygen. Examples of “C₁-C₉ heteroaryl” include furyl, pyrrolyl, thienyl, oxazolyl, isoxazolyl, imidazolyl, thiazolyl, isothiazolyl, pyridinyl, pyrimidinyl, tetrazolyl, quinazolinyl, indolyl, indolinyl, isoindolyl, isoindolinyl, pyrazolyl, pyridazinyl, pyrazinyl, quinolinyl, quinoxalinyl, thiadiazolyl, benzofuranyl, 2,3-dihydrobenzofuranyl, 1,3-benzodioxolyl, 1,4-benzodioxinyl, 2,3-dihydro-1,4-benzodioxinyl, benzothiazolyl, benzimidazolyl, benzothiadiazolyl, benzotriazolyl and chromanyl.

The term “C₁-C₉ heterocycle” denotes a non-aromatic monocyclic or fused bicyclic ring system having 5 to 10 ring atoms containing 1 to 9 carbon atoms and one or more of the ring atoms are selected from nitrogen, sulphur, and oxygen. When present, the sulfur atom may be in an oxidized form (i.e. the diradicle of S═O or the diradical of O═S═O). The ring system may be fully saturated or partially unsaturated. Examples of “C₁-C₉ heterocycle” include piperidinyl, tetrahydropyranyl, tetrahydrofuranyl, oxetanyl, azepinyl, azetidinyl, pyrrolidinyl, morpholinyl, imidazolinyl, imidazolidinyl, thiomorpholinyl, pyranyl, dioxanyl, piperazinyl, homopiperazinyl, and 5,6-dihydro-4H-1,3-oxazin-2-yl.

“An effective amount” refers to an amount of a compound of the invention that confers a therapeutic effect on the treated subject. The therapeutic effect may be objective (i.e. measurable by some test or marker) or subjective (i.e. subject gives an indication of or feels an effect).

As used herein, the terms “administration” or “administering” mean a route of administration for a compound disclosed herein. Exemplary routes of administration include, but are not limited to, oral, intravenous, intraperitoneal, intraarterial, and intramuscular. The preferred route of administration can vary depending on various factors, e.g. the components of the pharmaceutical composition comprising a compound disclosed herein, site of the potential or actual disease and severity of disease.

The terms “subject” and “patient” are used herein interchangeably. They refer to a human or another mammal (e.g., mouse, rat, rabbit, dog, cat, cattle, swine, sheep, horse or primate) that can be afflicted with or is susceptible to a disease or disorder but may or may not have the disease or disorder. It is preferred that the subject is human.

Compounds of the invention may be disclosed by the name or chemical structure. If a discrepancy exists between the name of a compound and its associated chemical structure, then the chemical structure prevails.

The invention will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilise the present invention to its fullest extent. All references and publications cited herein are hereby incorporated by reference in their entirety.

### Preparation of Compounds of the Invention

The compounds of formula (I) disclosed herein may be prepared by, or in analogy with, conventional methods. Appropriate reaction conditions for the individual reaction steps are known to a person skilled in the art. The necessary starting materials for preparing the compounds of formula (I) are either commercially available, or may be prepared by methods known in the art.

The compounds of formula (I) may possess one or more chiral carbon atoms, and they may therefore be obtained in the form of optical isomers, e.g., as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

Particular experimental procedures for examples of the invention are described below. The processes may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The chemicals used in the synthetic routes delineated herein may include, for example, solvents, reagents, catalysts, and protecting group and deprotecting group reagents. Examples of protecting groups are t-butoxycarbonyl (Boc), benzyl and trityl(triphenylmethyl). The methods described below may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The invention will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilize the present invention to its fullest extent. All references and publications cited herein are hereby incorporated by reference in their entirety.

### EXAMPLES AND INTERMEDIATE COMPOUNDS

### Experimental Methods

All reagents were commercial grade and were used as received without further purification, unless otherwise specified. Reagent grade solvents were used in all cases.

LC-MS and UPLC data was recorded under the following conditions:

### Method A

Waters Aquity system BEH-C18, 1.7 μm, 2.1′ 50 mm, 40° C., 0.5 μL injection, 0.4 mL/min. 0% MeCN (+0.1% aq. NH₃+5% water) in water (+0.1% NH₃+5% MeCN) for 0.2 min, 0-100% over 3.3 min, hold for 1 min, re-equilibrate 1.0 min, 200-400 nm.

### Method B

MeCN:H₂O (99:1), 0.1% formic acid; flow rate 3 mL/min; injection volume 0.5 μL; Ionization mode Electrospray ionization (ESI); Scan range m/z 83-600; DAD 215 nm, 254 nm; positive/negative mode. LC-MS data was recorded on one of the following systems: Agilent 1100 Series LC/MSD system with DAD\ELSD Alltech 2000ES and Agilent LC\MSD VL (G1956B), SL (G1956B) mass-spectrometer; Agilent 1200 Series LC/MSD system with DAD\ELSD Alltech 3300 and Agilent LC\MSD G6130A, G6120B mass-spectrometer; Agilent Technologies 1260 Infinity LC/MSD system with DAD\ELSD Alltech 3300 and Agilent LC\MSD G6120B mass-spectrometer; or Agilent Technologies 1260 Infinity II LC/MSD system with DAD\ELSD G7102A 1290 Infinity II and Agilent LC\MSD G6120B mass-spectrometer.

### Method C

LC-MS Phenomenex Kinetex XB-C18, 1.7 μm, 2.1×50 mm, 40° C., 0.8 mL/min, 5-100% MeCN (+0.085% TFA) in water (+0.1% TFA) over 1.2 min, hold for 0.2 min, re-equilibrate 0.6 min, 200-300 nm.

### Method D

Agilent 1100 (quaternary pump) XBridge-C18, 5 μm, 4.6×50 mm, 25° C., 2 mL/min, 5 μL injection, 5% MeCN in water (+10 mM ammonium formate), gradient 5-95% over 3.5 min, hold for 1 min, 200-400 nm.

### Method E

Waters Aquity system CSH-C18, 1.7 μm, 2.1×50 mm, 40° C., 0.5 μL injection, 0.4 mL/min. 0% MeCN (+0.1% formic acid+5% water) in water (+0.1% formic acid+5% MeCN) for 0.2 min, 0-100% over 3.3 min, hold for 1 min, 200-400 nm.

### Method F

Waters Aquity system BEH-C18, 1.7 μm, 2.1×50 mm, 40° C., 0.5 μL injection, 0.4 mL/min. 0% MeCN (+0.1% aq. NH₃+5% water) in water (+0.1% NH3+5% MeCN) for 0.2 min, 0-100% over 3.3 min, hold for 1 min, reequilibrate 1.0 min, 200-400 nm.

### Method G

Waters Aquity system BEH-C18, 1.7 μm, 2.1′ 50 mm, 40° C., 0.5 μL injection, 0.4 mL/min. 50% MeCN (+0.1% aq. NH₃+5% water) in water (+0.1% NH₃+5% MeCN) for 0.2 min, 50-100% over 1.8 min, hold for 2.5 min, 200-400 nm.

### Method H

Phenomenex Kinetex XB-C18, 1.7 μm, 2.1×100 mm, 40° C., 0.5 mL/min, 5% MeCN (+0.085% TFA) in water (+0.1% TFA) for 0.7 min, 5-100% over 8.0 min, hold for 0.3 min, reequilibrate 1.0 min. 200-300 nm.

### Method I

Phenomenex Kinetex XB-C18, 1.7 μm, 2.1×50 mm, 40° C., 0.8 mL/min, 5% MeCN (+0.085% TFA) in water (+0.1% TFA) for 0.7 min, 5-100% over 3.0 min, hold for 0.3 min, reequilibrate 1.0 min. 200-300 nm.

### Method J

Phenomenex Kinetex XB-C18, 1.7 μm, 2.1×50 mm, 40° C., 0.8 mL/min, 5% MeCN (+0.085% TFA) in water (+0.1% TFA) for 1.0 min, 5-100% over 3.0 min, hold for 0.2 min, reequilibrate 0.8 min. 200-300 nm

### Intermediate 1

### Methyl 4-methyl-3-(pyridin-3-ylethynyl)benzoate

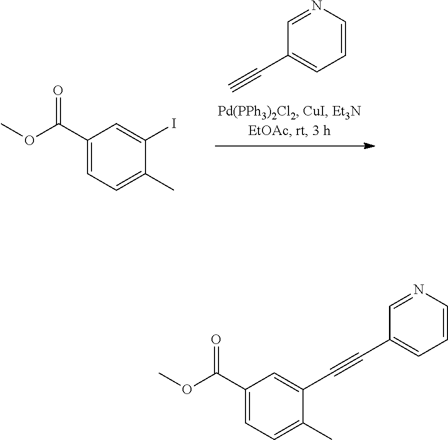

To a degassed solution of methyl 3-iodo-4-methylbenzoate (5.5 g, 20 mmol, 1 eq), 3-ethynylpyridine (2.1 g, 20 mmol, 1 eq) and triethylamine (6.1 g, 60 mmol, 3 eq) in EtOAc (87 mL) was added copper (I) iodide (190 mg, 1 mmol, 0.05 eq) and Pd(PPh₃)₂Cl₂ (702 mg, 1 mmol, 0.05 eq) and the reaction stirred at rt under nitrogen for 3 h. The solid was removed by filtration and the solution concentrated in vacuo, the crude material was purified by normal phase chromatography EtOAc/heptane (1:1) to afford methyl 4-methyl-3-(pyridin-3-ylethynyl)benzoate (4.3 g, 85%) as a pale yellow solid. UPLC (Method A) 3.44 min, 99%, [M+H]⁺=252.2.

### Intermediate 2

### 4-Methyl-3-(pyridin-3-ylethynyl)benzoic acid

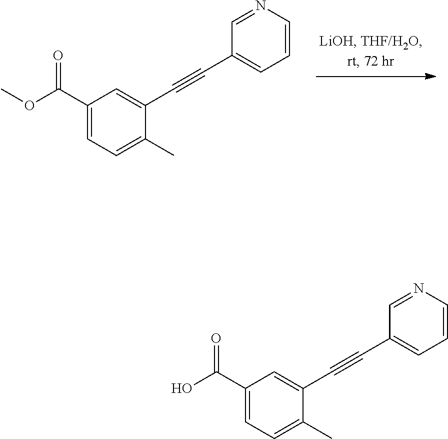 [SMILES: COC(=O)c1ccc(C)c(C#Cc2cccnc2)c1 ; Cc1ccc(C(=O)O)cc1C#Cc1cccnc1]

To a solution of Intermediate 1 (2.80 g, 11.0 mmol, 1.0 eq) in THF (40 mL) was added a solution of lithium hydroxide monohydrate (0.69 g, 16.5 mmol, 1.5 eq) in water (10 mL) and the reaction stirred at rt under nitrogen over 72 h. The reaction was acidified to pH ˜5 by addition of 2M HCl (8.5 mL) and stirred for 1 h. The solid was collected by filtration, washed with tert-butyl methyl ether (2×30 mL), and dried in vacuo to afford 4-Methyl-3-(pyridin-3-ylethynyl)benzoic acid (2.00 g, 77%) as an off white solid. UPLC (Method A) 1.97 min, 98%, [M+H]⁺=238.2.

### Intermediate 3

### 1-Isopropyl-4-nitro-1H-imidazole

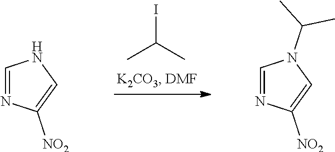

To a solution of 4-nitro-1H-imidazole (0.80 g, 7.1 mmol, 1.0 eq) in DMF (35 mL) was added K₂CO₃ (1.96 g, 14.2 mmol, 2.0 eq) and 2-iodopropane (0.85 mL, 8.5 mmol, 1.2 eq) and the reaction was stirred at 50° C. overnight. The reaction mixture was cooled to rt, quenched with H₂O (150 mL) and diluted with EtOAc (150 mL). The phases were separated and the aqueous extracted EtOAc (2×100 mL). Combined organics were dried (MgSO₄) and concentrated in vacuo (azeotroped with toluene). The crude material was purified by normal phase chromatography and tert-butyl methyl ether/heptane (1:1) followed by 100% EtOAc to isolate the major product as a yellow oil, which crystallised on standing. The crystals were azeotroped with toluene (×2) to afford 1-Isopropyl-4-nitro-1H-imidazole (2.76 g, 84%) as crystalline pale-yellow needles. UPLC (Method A) 1.96 min, 100%, [M+H]⁺=156.1.

### Intermediate 4

### 1-Isopropyl-1H-imidazol-4-amine hydrochloride

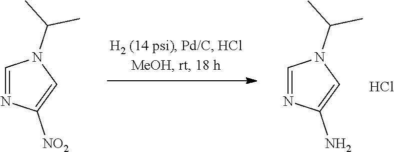

To a solution of isopropyl-4-nitro-1H-imidazole (930 mg, 6 mmol, 1 eq) in methanol (50 mL) was added palladium on carbon (100 mg, 10% w/w) and the reaction was stirred at rt under an atmosphere of hydrogen (1 atm/14 psi) overnight. The suspension was filtered through a pad of dicalite and to the resulting solution was added 2M HCl (3 mL) and the mixture concentrated in vacuo to afford 1-Isopropyl-1H-imidazol-4-amine hydrochloride (assumed quant.). UPLC (Method A) 1.36 min, 85%, [M+H]⁺=126.1.

### Intermediate 5

### 6-Hydroxy-4,4-dimethyl-3-oxohexanenitrile

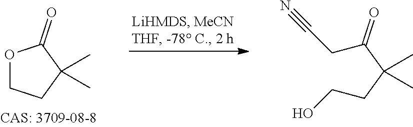

A solution of lithium bis(trimethylsilyl)amide (1M in THF) (38.5 mL, 38.5 mmol, 2.2 eq) in THF (29 mL) was cooled to −78° C. under nitrogen after which a solution of 3,3-dimethyldihydrofuran-2(3H)-one (2.0 g, 27.5 mmol, 1.0 eq) and MeCN (1.83 mL, 35.0 mmol, 2.0 eq) in THF (20 mL) was added dropwise. The reaction was stirred at −78° C. for 15 min then warmed to rt and stirred for a further 2 h. The reaction was quenched with sat. aq. NH₄Cl, diluted with EtOAc, the phases separated and the aqueous extracted with EtOAc. Combined organics were washed with sat. NaCl, dried (MgSO₄) and concentrated in vacuo to afford 6-hydroxy-4,4-dimethyl-3-oxohexanenitrile crude (assumed quant.) as an oil.

### Intermediate 6

### 3-(3-Amino-1H-pyrazol-5-yl)-3-methylbutan-1-ol

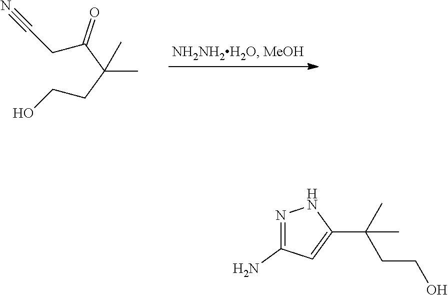

To a solution of intermediate 5 (2.72 g, 17.5 mmol, 1.0 eq) in MeOH (25 mL) was added hydrazine hydrate (1.64 mL, 26.3 mmol, 1.5 eq) and the reaction heated at 60° C. for 64 h. The reaction was cooled to rt and CO₂ was bubbled through the mixture for 1 h. The reaction was decanted from the off-white precipitate and the solution concentrated in vacuo. The resulting residue was purified by normal phase chromatography 1-10% MeCOH/DCM (stained with KMnO₄) to afford 3-(3-amino-1H-pyrazol-5-yl)-3-methylbutan-1-ol (1.18 g, 40%) as a clear oil. LCMS (Method D) 1.36 min, 100%, [M+H]⁺=170.10.

### Intermediate 7

### 4,4-Dimethyl-4H,5H,6H-pyrrolo[1,2-b]pyrazol-2-amine

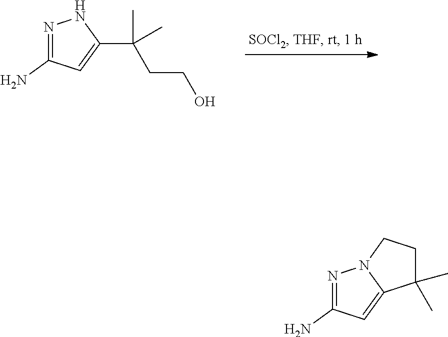

To a solution of Intermediate 6 (1.18 g, 7.0 mmol, 1 eq) in THF (38 mL) was added SOCl₂ (2.53 mL, 34.9 mmol, 5 eq) dropwise, and the reaction stirred at rt for 1 h. The reaction was poured into 28% NH₄OH solution on ice and diluted with DCM. The phases were separated, the aqueous extracted with DCM and combined organics washed with sat. NaCl, dried (MgSO₄) and concentrated in vacuo. The crude material was purified by normal phase chromatography 1-10% MeCOH/DCM to afford 4,4-dimethyl-4H,5H,6H-pyrrolo[1,2-b]pyrazol-2-amine (334 mg, 29% yield) as a yellow oil. LCMS (Method D) 1.87 min, 90%, [M+H]⁺=152.10.

### Intermediate 8

### Methyl 2-[2-oxo-5-(trifluoromethyl)pyrrolidin-1-yl]acetate

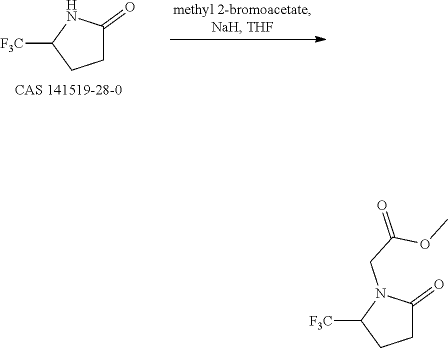

To a suspension of 5-(trifluoromethyl)pyrrolidin-2-one (500 mg, 3.3 mmol, 1.0 eq) in anhydrous THF (20 mL) under a N₂ atmosphere at 0° C. was added 60% NaH (157 mg, 3.9 mmol, 1.2 eq) in small portions with constant stirring. The reaction was allowed to warm to rt and stirred for 30 min before addition of methyl 2-bromoacetate (550 mg, 3.6 mmol, 1.1 eq) and further stirring at rt for 18 h. The reaction was quenched with sat. NH₄Cl (5 mL) and the volatiles removed in vacuo. The residue was partitioned between EtOAc (25 mL) and H₂O (15 mL) and the organic layer dried (MgSO₄) and concentrated in vacuo. The residue was purified by normal phase chromatography 30-100% tert-butyl methyl ether/heptane to afford methyl 2-[2-oxo-5-(trifluoromethyl)pyrrolidin-1-yl]acetate (400 mg, 54%) as a colourless oil. 1H-NMR (400 MHz, CDCl₃) δ_{H} 4.64 (d, J=18.2 Hz, 1H), 4.28-4.20 (m, 1H), 3.78 (d, J=18.2 Hz, 1H), 3.74 (s, 3H), 2.64-2.50 (m, 1H), 2.48-2.31 (m, 2H), 2.24-2.13 (m, 1H) ppm. 19F-NMR (373 MHz, chloroform-D) δ_{F} −75.4 (d, J=7.0 Hz, 3F) ppm.

### Intermediate 9

### 2-(2-Oxo-5-(trifluoromethyl)pyrrolidin-1-yl)acetamide

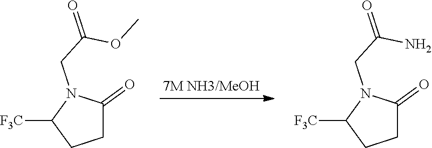

Methyl 2-(2-oxo-5-(trifluoromethyl)pyrrolidin-1-yl)acetate (400 mg, 1.8 mmol, 1.0 eq) was dissolved in NH₃ (7M in MeCOH, 5 mL) and stirred at rt for 4 h in a sealed tube. The reaction was concentrated in vacuo to afford 2-(2-oxo-5-(trifluoromethyl)pyrrolidin-1-yl)acetamide (374 mg, 100%) as a white solid. 1H-NMR (400 MHz, CDCl₃) δ_{H} 5.78 (s, 1H), 5.42 (s, 1H), 4.42 (d, J=17.0 Hz, 1H), 4.33-4.22 (m, 1H), 3.79 (d, J=16.3 Hz, 1H), 2.65-2.50 (m, 1H), 2.49-2.32 (m, 2H), 2.24-2.14 (m, 1H) ppm.

### Intermediate 10

### 5-(Trifluoromethyl)-6,7-dihydro-3H-pyrrolo[1,2-a]imidazol-2(5H)-one

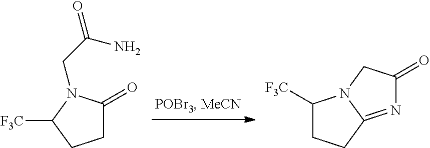 [SMILES: NC(=O)CN1C(=O)CCC1C(F)(F)F ; O=C1CN2C(=N1)CCC2C(F)(F)F]

To a suspension of Intermediate 9 (400 mg, 1.9 mmol, 1 eq) in MeCN (10 mL) was added phosphorus(V)oxybromide (1.64 g, 5.7 mmol, 3 eq) and the reaction heated at 70° C. under N₂ for 2 h. The volatiles were removed in vacuo and the residue stirred with H₂O (20 mL), basified with solid K₂CO₃, extracted with EtOAc (3×20 mL), dried (MgSO₄) and concentrated in vacuo. The residue was purified by normal phase chromatography 0.2:2:98 NH₃:MeOH:DCM to afford 5-(Trifluoromethyl)-6,7-dihydro-3H-pyrrolo[1,2-a]imidazol-2(5H)-one (240 mg, 50%) as a colourless oil. 1H-NMR (400 MHz, CDCl₃) δ_{□} 4.83 (d, J=17.6 Hz, 1H), 4.21 (m, 1H), 3.90 (d, J=17.6 Hz, 1H), 2.65-2.50 (m, 1H), 2.50-2.32 (m, 2H), 2.32-2.20 (m, 1H). 19F-NMR (373 MHz, chloroform-D) δ_{F} −75.2 (d, J=6.3 Hz, 3F) ppm.

### Intermediate 11

### 2-Bromo-5-(trifluoromethyl)-6,7-dihydro-5H-pyrrolo[1,2-a]imidazole

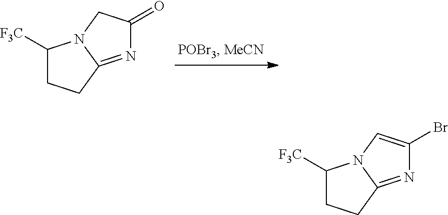 [SMILES: O=C1CN2C(=N1)CCC2C(F)(F)F ; FC(F)(F)C1CCc2nc(Br)cn21]

To a suspension of Intermediate 10 (300 mg, 1.6 mmol, 1 eq) in MeCN (4 mL) was added phosphorus(V)oxybromide (1.34 g, 4.7 mmol, 3 eq) and the reaction heated in a sealed tube at 100° C. for 18 h. The volatiles were removed in vacuo and the residue stirred with H₂O (20 mL), basified with solid K₂CO₃ and extracted EtOAc (3×20 mL), dried (MgSO₄) and concentrated in vacuo. The residue was purified by normal phase chromatography 0.2:2:98 NH₃:MeOH:DCM to afford 2-Bromo-5-(trifluoromethyl)-6,7-dihydro-5H-pyrrolo[1,2-a]imidazole (160 mg, 24%) as a colourless oil. UPLC (Method A) 2.61 min, 89%, [M+H]⁺=255.0, 257.0.

### Intermediate 12

### 4-Methyl-3-(pyridin-3-ylethynyl)benzamide

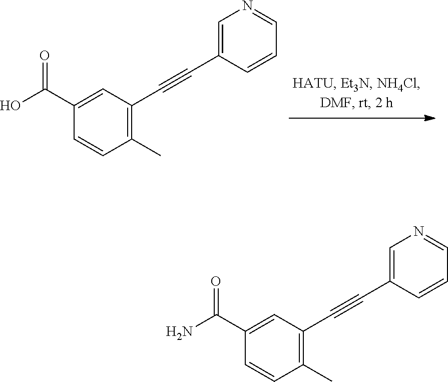 [SMILES: Cc1ccc(C(=O)O)cc1C#Cc1cccnc1 ; Cc1ccc(C(N)=O)cc1C#Cc1cccnc1]

To a solution of 4-methyl-3-(pyridin-3-ylethynyl)benzoic acid (1.50 g, 6.3 mmol, 1.0 eq), ammonium chloride (1.01 g, 19.0 mmol, 3.0 eq) and triethylamine (2.67 mL, 19.6 mmol, 3.1 eq) in DMF (20 mL) was added (1-[bis(dimethylamino)methylene]-1H-1,2,3-triazolo[4,5-b]pyridinium 3-oxid hexafluorophosphate (3.12 g, 8.2 mmol, 1.3 eq), the reaction stirred at rt for 2 h. The reaction mixture was stirred with H₂O (100 mL) and tert-butyl methyl ether (100 mL) and the solid was collected by filtration and dried in vacuo to afford 4-Methyl-3-(pyridin-3-ylethynyl)benzamide (1.0 g, 67%) as a colourless solid. UPLC (Method A) 2.59 min, 100%, [M+H]⁺=237.2.

### Intermediate 13

### Methyl 2-(5-oxo-4-azaspiro[2.4]heptan-4-yl)acetate

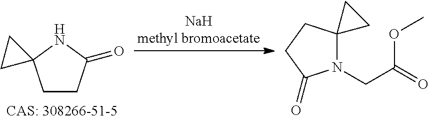

To a suspension of NaH (60% in oil, 216 mg, 5.40 mmol, 1.2 eq) in THF (8 mL), under N₂ and at ca. 15° C. (chilled water bath), was added a solution of 4-azaspiro[2.4]heptan-5-one (500 mg, 4.50 mmol, 1.0 eq) in THF (5 mL) dropwise and the reaction stirred at 15° C. for 30 min. To the reaction was added a solution of methyl bromoacetate (516 μL, 5.40 mmol, 1.2 eq) in THF (1.5 mL) dropwise and the reaction stirred at rt for 1 h. The reaction was quenched with sat. NH₄Cl (60 mL), diluted with EtOAc (30 mL), the phases separated and the aqueous extracted with EtOAc (3×30 mL). Combined organics were washed with brine (50 mL), dried (Na₂SO₄) and concentrated in vacuo to afford methyl 2-(5-oxo-4-azaspiro[2.4]heptan-4-yl)acetate (824 mg, assumed quant.) as pale yellow-orange oil. 1H NMR (400 MHz, CDCl₃) δ_{H} 3.72 (s, 3H), 3.70 (s, 2H), 2.58 (t, J=8.2 Hz, 2H), 2.14 (t, J=8.2 Hz, 2H), 0.80 (t, J=6.7 Hz, 2H), 0.61 (t, J=6.7 Hz, 2H) ppm.

### Intermediate 14

### 2-(5-Oxo-4-azaspiro[2.4]heptan-4-yl)acetamide

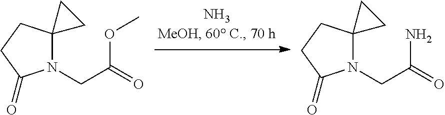

A solution of Intermediate 13 (821 mg, 4.48 mmol, 1 eq) in NH₃ (7M in MeCOH, 10 mL, 15 eq) was heated at 60° C. for 70 h. The reaction was cooled to rt and was concentrated in vacuo to afford 2-(5-oxo-4-azaspiro[2.4]heptan-4-yl)acetamide (753 mg, assumed quant) as a pale-yellow sticky residue. 1H NMR (400 MHz, DMSO-d6) δ_{H} 7.23 (br. s, 1H), 7.03 (br. s, 1H), 3.41 (s, 2H), 2.38 (t, J=8.2 Hz, 2H), 2.05 (t, J=8.2 Hz, 2H), 0.82 (t, J=6.5 Hz, 2H), 0.51 (t, J=6.5 Hz, 2H) ppm.

### Intermediate 15

### 2′-Bromo-6′,7′-dihydrospiro[cyclopropane-1,5′-pyrrolo[1,2-a]imidazole]

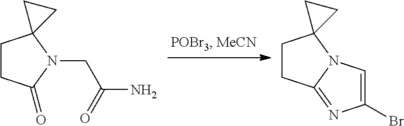 [SMILES: NC(=O)CN1C(=O)CCC12CC2 ; Brc1cn2c(n1)CCC21CC1]

To a suspension of Intermediate 14 (400 mg, 2.38 mmol, 1 eq) in MeCN (2 mL) was added phosphorus(V)oxybromide (2.73 g, 9.51 mmol, 4 eq) and the reaction heated at 85° C. for 3.5 h. The reaction was cooled to rt, poured into H₂O (50 mL) and extracted with DCM (2×40 mL). The aqueous layer was basified with K₂CO₃ (to pH=9), extracted with DCM (40 mL) and combined organics washed with K₂CO₃ (3% wt/wt aq sol, 100 mL), brine (150 mL), dried (Na₂SO₄) and concentrated in vacuo. The residue was purified by normal phase chromatography (dry loaded) in 0-15% MeCOH/DCM to afford 2′-Bromo-6′,7′-dihydrospiro[cyclopropane-1,5′-pyrrolo[1,2-a]imidazole] (94 mg, 19%) as a brown solid. UPLC (Method E) 2.05 min, 100%, [M+H]⁺=213.0, 215.0.

### Intermediate 16

### Methyl 4-bromo-1-cyclopropyl-1H-imidazole

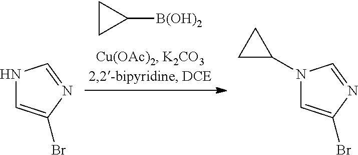 [SMILES: Brc1c[nH]cn1 ; OB(O)C1CC1 ; Brc1cn(C2CC2)cn1]

To a solution of 4-bromo-1H-imidazole (2.00 g, 13.6 mmol, 1 eq) in DCE (100 mL), under nitrogen was added cyclopropylboronic acid (2.34 g, 27.2 mmol, 2 eq), copper (II) acetate (2.47 g, 13.6 mmol, 1 eq), 2,2′-bipyridine (2.13 g, 13.6 mmol, 1 eq) and potassium carbonate (3.76 g, 27.2 mmol, 2 eq) and the suspension heated at 70° C. for 3 h. The reaction was cooled to rt and poured into H₂O (100 mL). The phases were separated, and the organic phase was washed with 1M HCl sol. (100 mL) and sat. Na₂CO₃ (100 mL), the aqueous was basified with K₂CO₃ (to pH=10) and extracted with DCM (100 mL) and with DCM/isopropanol (9:1, 2×100 mL). Combined organics were washed with brine, dried (MgSO₄) and concentrated in vacuo. The residue was purified by normal phase chromatography (dry loaded) 10-50% EtOAc/DCM to afford methyl 4-bromo-1-cyclopropyl-1H-imidazole (195 mg, 7%) as a pale-yellow oil. UPLC (Method F) 2.26 min, 100%, [M+H]⁺=187.0, 189.0.

### Intermediate 17

### 3-Ethynylimidazo[1,2-a]pyridine

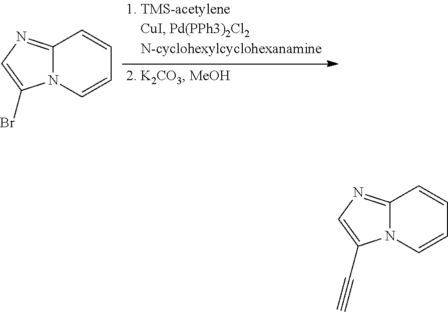

Ethynyltrimethylsilane (1.41 mL, 10.20 mmol, 2.0 eq) and N-cyclohexylcyclohexanamine (1.21 mL, 6.09 mmol, 1.2 eq) were added to a solution of 3-bromoimidazo[1,2-a]pyridine (1.00 g, 5.08 mmol, 1.0 eq), bis(triphenylphosphine)palladium(II) dichloride (89.1 mg, 127 μmol, 0.025 eq) and copper(I) iodide (33.8 mg, 178 μmol, 0.035 eq) in MeCN (10 mL), the solution sparged with N₂ for 10 min and then heated to 80° C. for 5 min to form a very thick slurry. The reaction was cooled to rt, diluted with MeCN (40 mL) and re-heated at 80° C. for 3 h. The reaction mixture was then concentrated in vacuo and the residue partitioned between DCM (100 mL) and H₂O (100 mL). The aqueous layer was extracted with DCM (100 mL) and the organic layers combined, dried (MgSO₄) and concentrated in vacuo. The residue was dissolved in MeCOH (20 mL), K₂CO₃ (701 mg, 5.08 mmol) was added and the reaction was stirred for 30 min at rt. The reaction mixture was filtered and concentrated in vacuo. The residue was purified by column chromatography (normal phase, [24 g], RediSep silica gel, 35-60 μm (230-400 mesh), 35 mL per min, gradient 0% to 100% EtOAc in iso-hexanes). The product was dried in a vacuum oven at 50° C. for 3 h to give 3-ethynylimidazo[1,2-a]pyridine (473 mg, 66%) as a brown solid. LC-MS (Method C) 0.50 min, [M+H]⁺=143.0.

### Intermediate 18

### N-(5-tert-butylisoxazol-3-yl)-3-iodo-4-methyl-benzamide

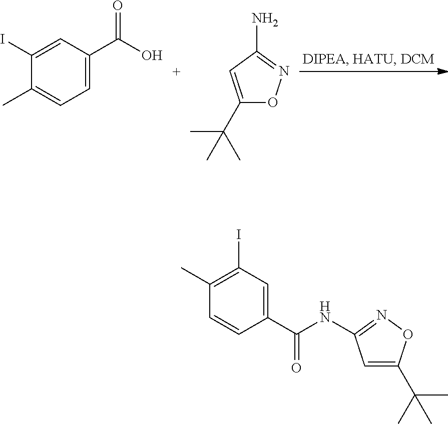

DIPEA (1.32 mL, 7.63 mmol, 2 eq) was added to a solution of 3-iodo-4-methyl-benzoic acid (1.00 g, 3.82 mmol, 1 eq), 3-amino-5-tert-butylisoxazole (535 mg, 3.82 mmol, 1 eq) and HATU (1.45 g, 3.82 mmol, 1 eq) in DCM (20 mL) and the reaction stirred at reflux for 96 h. The mixture was partitioned between DCM (50 mL) and H₂O (50 mL) and the aqueous layer extracted with DCM (50 mL). Combined organics were dried (MgSO₄) and concentrated in vacuo. The residue was purified by column chromatography (normal phase, [40 g], RediSep silica gel, 35-60 μm (230-400 mesh), 40 mL per min, gradient 0% to 100% EtOAc in iso-hexanes). The product was dried in a vacuum oven at 60° C. for 2 h to give N-(5-tert-butylisoxazol-3-yl)-3-iodo-4-methyl-benzamide (767 mg, 51%) as a white solid. LC-MS (Method C) 1.47 min, [M+H]⁺=385.2.

### Intermediate 19

### 1-(cyclobutylmethyl)-4-iodo-imidazole

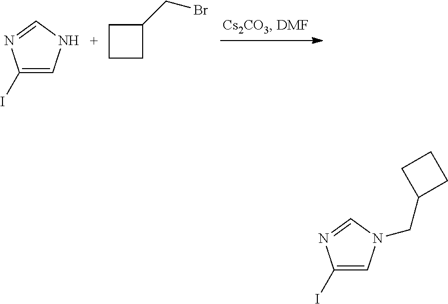 [SMILES: Ic1c[nH]cn1 ; BrCC1CCC1 ; Ic1cn(CC2CCC2)cn1]

To a solution of iodoimidazole (0.90 g, 4.6 mmol, 1.0 eq) in DMF (9 mL) under N₂ at rt was added Cs₂CO₃ (4.54 g, 13.9 mmol, 3.0 eq) followed by bromomethylcyclobutane (0.63 mL, 5.5 mmol, 1.2 eq) and the resulting suspension heated to 60° C. for 1 h. The white suspension was then cooled to rt and the volatiles removed in vacuo. To the residue was added H₂O (50 mL) and EtOAc (25 mL), the phases separated and the aqueous extracted with EtOAc (2×25 mL). Combined organics were washed with brine (50 mL), dried over Na₂SO₄ and concentrated in vacuo. The crude mixture was combined with the crude from a 100 mg scale reaction, and the combined residues were purified by column chromatography (Biotage Isolera, 100% EtOAc to EtOAc:DCM, 1:1 over 10 CV) to yield the title product (819 mg, 61%) as a colourless oil. Structure confirmed by NOESY. UPLC (Method A) 2.88 min, 100%, [M+H]⁺=263.0.

### Intermediate 20

### 3-[1-(3-Hydroxypropyl)cyclopropyl]-3-oxo-propanenitrile

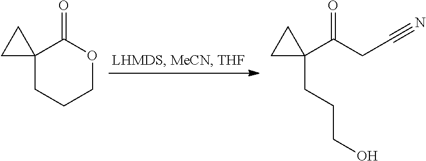 [SMILES: O=C1OCCCC12CC2 ; N#CCC(=O)C1(CCCO)CC1]

LHMDS (1.5 M in THF, 11.6 mL, 17.3 mmol) diluted with dry THF (20 mL) was cooled to −78° C. and MeCN (0.82 mL, 15.8 mmol) added dropwise. The resulting solution was stirred for 1 h before addition of a solution of 5-oxaspiro[2.5]octan-4-one (995 mg, 7.9 mmol) in dry THF (5 mL) dropwise. The reaction was stirred at −78° C. for 2 h and then warmed to rt where it was quenched by addition of the reaction mixture into saturated NH₄Cl solution (130 mL) and extracted with DCM (3×60 mL). The combined organics were dried (MgSO₄), filtered and concentrated in vacuo to yield the crude product (1.63 g, >100%) as a red/brown oil, which was used without further purification in the next step.

### Intermediate 21

### 3-[1-(3-Amino-1H-pyrazol-5-yl)cyclopropyl]propan-1-ol

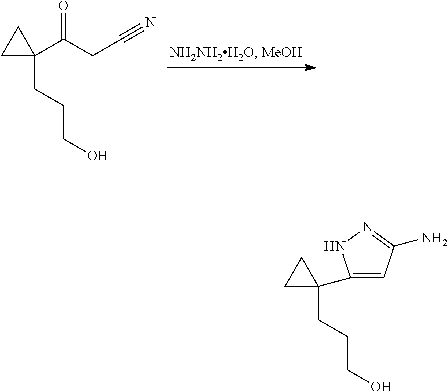 [SMILES: N#CCC(=O)C1(CCCO)CC1 ; Nc1cc(C2(CCCO)CC2)[nH]n1]

Hydrazine monohydrate (1.2 mL, 25.0 mmol) was added to a solution of Intermediate 20 (1.39 g, 8.3 mmol) in MeCOH (25 mL) dropwise in an autoclave (150 mL). The resulting reaction mixture was then heated at 120° C. for 18 h. The mixture was then allowed to cool to rt before addition of dry ice over a period of 10 min. The clear solution was decanted and the solvent removed in vacuo to afford the crude product (1.60 g) as an orange oil. This was purified by column chromatography on silica (MeOH/DCM, 1:99 to 1:9) to yield the title compound (1.10 g, 73%) as an orange oil. UPLC (Method A) 1.72 min, 94.6%, [M+H]⁺=182.1.

### Intermediate 22

### 5-[1-(3-Chloropropyl)cyclopropyl]-1H-pyrazol-3-amine

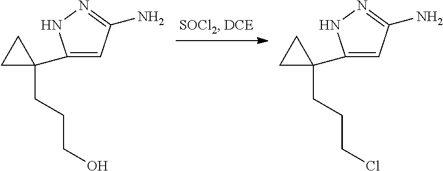 [SMILES: Nc1cc(C2(CCCO)CC2)[nH]n1 ; Nc1cc(C2(CCCCl)CC2)[nH]n1]

Thionyl chloride (150 μL, 1.99 mmol) was added to a solution of Intermediate 21 (300 mg, 1.66 mmol) in 1,2-dichloroethane (5.0 mL) at rt. The reaction mixture was then heated at 90° C. for 1 h. The reaction was cooled to rt and combined with an identical reaction carried out on a 1.17 mmol scale. To the mixture was added aq. potassium carbonate (2M, 15 mL) and the layers separated. The aqueous phase was extracted with DCM (2×20 mL) and combined organics dried (Na₂SO₄) and concentrated in vacuo to give the crude title compound (474 mg) as a brown oil, which was used in the next step without further purification.

### Intermediate 23

### Spiro[6,7-dihydro-5H-pyrazolo[1,5-a]pyridine-4,1′-cyclopropane]-2-amine

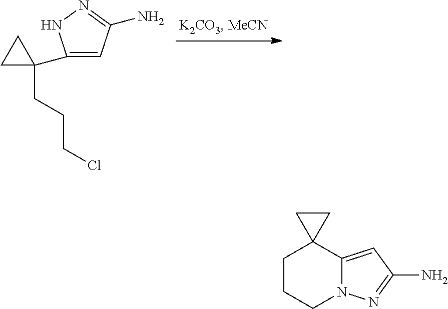 [SMILES: Nc1cc(C2(CCCCl)CC2)[nH]n1 ; Nc1cc2n(n1)CCCC21CC1]

Intermediate 22 (474 mg, 2.37 mmol) and potassium carbonate (656 mg, 4.75 mmol) in MeCN (20 mL) were heated at 80° C. overnight. The reaction was allowed to cool to rt and the solvent removed in vacuo. To the resulting residue was added H₂O (20 mL) and DCM (20 mL) and the phases separated. The aqueous phase was washed with DCM (2×20 mL) and combined organics dried under reduced pressure to afford the crude product. This was purified by column chromatography on silica (MeOH/DCM, 1:99 to 1:9) to yield the title product (260 mg, 67%) as a brown solid which was used in the next step without further purification. UPLC (Method A) 2.29 min, 81.4%, [M+H]⁺=164.1.

### Intermediate 24

### 2-Chloro-N-(2-hydroxy-2-methyl-propyl)acetamide

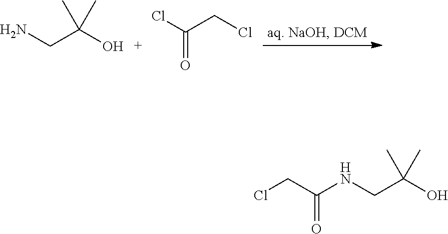

To 1-amino-2-methyl-propan-2-ol (5.00 g, 56.1 mmol) in DCM (80 mL) and 2M NaOH (39.3 mL, 78.5 mmol) at 0° C. was added chloroacetyl chloride (5.4 mL, 67.3 mmol) dropwise. The reaction was stirred for 2 h at rt, the layers were separated, the organic layer dried over MgSO₄ and the solvent removed in vacuo to give 2-chloro-N-(2-hydroxy-2-methyl-propyl)acetamide (5.60 g, 60%) as a colourless oil. ¹H NMR (400 MHz, CDCl₃): δ_{H} 7.02 (s, 1H), 4.13 (m, 3H), 3.35 (d, J=6.1 Hz, 2H), 1.30-1.25 (m, 6H) ppm.

### Intermediate 25

### 6,6-Dimethylmorpholin-3-one

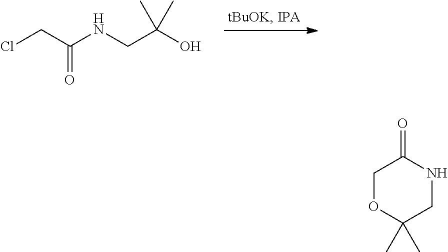

To Intermediate 24 (5.60 g, 33.8 mmol) in IPA (40 mL) at 0° C. under N₂ was added tert-butoxypotassium (7.59 g, 67.6 mmol) and the reaction mixture warmed to rt over 16 h. The reaction was neutralised with HCl solution (2 M) to pH 7 and the organic solvent removed under reduced pressure. The resulting aqueous phase was extracted with DCM (3×20 mL). The combined organics were dried over MgSO₄ and the solvent removed under reduced pressure to give the crude product (1.99 g) as a yellow oil. This was purified by column chromatography (manual column, normal phase, silica gel 40-63 μm/230-400 mesh, 60 Å, 0% to 10% MeCOH in EtOAc) to afford 6,6-dimethylmorpholin-3-one as a crystalline white solid (832 mg, 19.1%). ¹H NMR (400 MHz, CDCl₃): δ_{H} 6.39 (s, 1H), 4.18 (s, 2H), 3.25 (d, J=2.4 Hz, 2H), 1.33 (s, 6H) ppm.

### Intermediate 26

### Methyl 2-(2,2-dimethyl-5-oxo-morpholin-4-yl)acetate

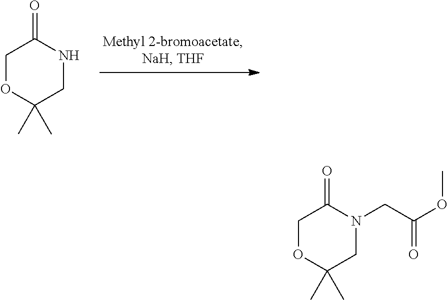

To a stirred solution of Intermediate 25 (832 mg, 6.44 mmol) in THF (25 mL) under N₂ at rt was added NaH (60% in mineral oil, 258 mg, 6.44 mmol) in portions. The reaction was stirred at rt for 40 min followed by addition of methyl 2-bromoacetate (608 μL, 6.44 mmol) dropwise at rt and the reaction stirred at rt for 16 h. The reaction was carefully diluted with water (20 mL) and the layers separated. The aqueous phase was extracted with EtOAc (3×10 mL). The combined organics were washed with brine (20 mL), dried (MgSO₄) and concentrated in vacuo to give crude product (1.20 g) as a pink oil. The residue was purified by column chromatography (manual column, normal phase, silica gel 40-63 μm/230-400 mesh, 60 Å, 70% to 100% EtOAc in heptane) to afford the title compound (726 mg, 56%) as a colourless oil. ¹H NMR (400 MHz, CDCl₃): δ_{H} 4.22 (s, 2H), 4.15 (s, 2H), 3.75 (s, 3H), 3.28 (s, 2H), 1.36 (s, 6H) ppm.

### Intermediate 27

### 2-(2,2-Dimethyl-5-oxo-morpholin-4-yl)acetic acid

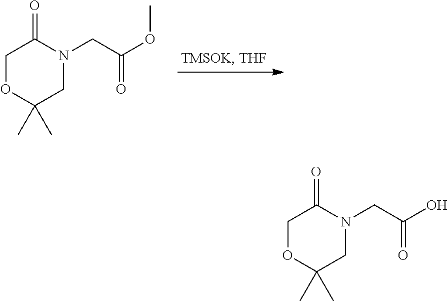

To a stirred solution of Intermediate 26 (726 mg, 3.61 mmol) in THF (20 mL) was added TMSOK (669 mg, 4.69 mmol) at rt followed by stirring for 16 h. The reaction was diluted with TBME (30 mL), filtered under vacuum and the filtrate discarded. The filter cake was dissolved in water (10 mL), acidified to pH 2 using 2M HCl, saturated by addition of solid NaCl and extracted with EtOAc (8×10 mL). The combined organics were dried (MgSO₄) and concentrated in vacuo to give the title compound (378 mg, 56%) as a white solid. ¹H NMR (400 MHz, CDCl₃): δ_{H} 4.24 (s, 2H), 4.17 (s, 2H), 3.31 (s, 2H), 1.36 (s, 6H) ppm.

### Intermediate 28

### 2-(2,2-Dimethyl-5-oxo-morpholin-4-yl)acetamide

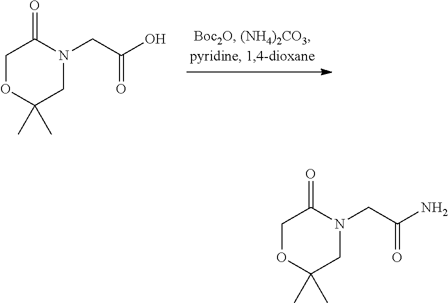

To a stirred solution of Intermediate 27 (378 mg, 2.02 mmol) in 1,4-dioxane (10 mL) at rt was added pyridine (81 μL, 1.01 mmol), ammonium carbonate (194 mg, 2.02 mmol) and Boc₂O (617 mg, 2.83 mmol) and the reaction stirred for 16 h at rt. The reaction was concentrated in vacuo and the residue was purified by column chromatography (manual column, normal phase, silica gel 40-63 μm/230-400 mesh, 60 Å, 0% to 20% MeCOH in EtOAc) to afford the title compound (349 mg, 92.8%) as a white solid. ¹H NMR (400 MHz, CDCl₃): δ_{H} 6.20 (s, 1H), 5.38 (s, 1H), 4.22 (s, 2H), 4.02 (s, 2H), 3.35 (s, 2H), 1.34 (s, 6H) ppm.

### Intermediate 29

### 2-Bromo-6,6-dimethyl-5,8-dihydroimidazo[2,1-c][1,4]oxazine

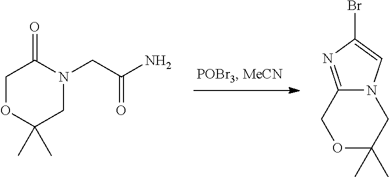

To a microwave vial containing Intermediate 28 (160 mg, 0.86 mmol) in MeCN (4.0 mL) was added POBr₃ (1.48 g, 5.16 mmol). The vial was then heated to 120° C. in a Biotage initiator for 35 min. The reaction was then added dropwise to a stirring solution of H₂O (10 mL) and DCM (10 mL) at 0° C. Solid K₂CO₃ was then added to basify the aqueous layer to pH 10. The organic layer was separated and the aqueous extracted with 4:1 DCM:IPA (5×10 mL). The combined organic layers were dried over MgSO₄, filtered and concentrated to give the crude product (310 mg). The crude residue was purified by silica column chromatography (0-60% EtOAc in heptane) to afford the title compound (35.0 mg, 17.6%) as a brown oil. This was used in the next step without further purification. UPLC (Method A) 2.44 min, 28.3%, [M+H]⁺=231.0/233.0.

### Intermediate 30

### Diethyl 1-(4-ethoxy-4-oxo-butyl)pyrazole-3,5-dicarboxylate

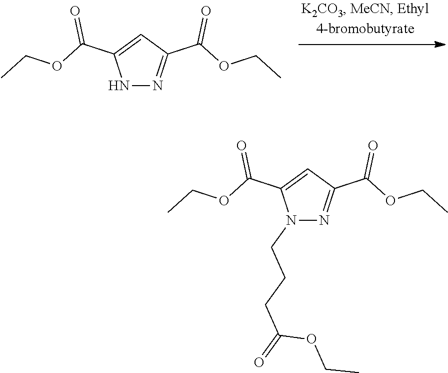

According to a literature procedure (Pfizer EP1241170A2 (2002), the contents of which are incorporated herein), to a solution of diethyl-3,5-pyrazoledicarboxylate (10.00 g, 47.1 mmol, 1.0 eq) in MeCN (100 mL) was added K₂CO₃ (6.51 g, 47.1 mmol, 1.0 eq) followed by ethyl 4-bromobutyrate (9.19 g, 47.1 mmol, 1.0 eq) and the mixture heated at 80° C. for 3.5 h. The resulting white suspension was cooled to rt and allowed to stand overnight. The solvent was removed in vacuo and the residue combined with aq. NH₄Cl (100 mL) and EtOAc (100 mL) and the phases separated. The aqueous layer was extracted with EtOAc (1×50 mL) and combined organics were washed with aq. NaHCO₃ (100 mL), aq. NH₄Cl (100 mL), dried (Na₂SO₄), filtered and the solvent removed in vacuo to yield the title compound (15.77 g, quant.) as a pale yellow oil. LC-MS (Method D) 2.89 min, 98%, [M+H]⁺=327.2.

### Intermediate 31

### Diethyl 4-oxo-6,7-dihydro-5H-pyrazolo[1,5-a]pyridine-2,5-dicarboxylate

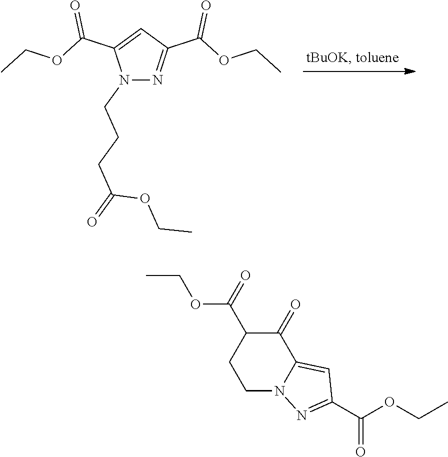

To a solution of Intermediate 30 (10.0 g, 30.6 mmol, 1 eq) in toluene (100 mL) was added dropwise a solution of t-BuOK in THF (1.6 M in THF, 21.1 mL, 33.7 mmol, 1.1 eq) over 5 min, during which time the temperature rose steadily to 30° C. to generate a precipitate in a pale orange solution. The mixture was stirred at rt for 25 min and then at 90° C. for 3 h to generate a thick slurry. The mixture was then allowed to cool to rt and stirred overnight. The slurry was diluted with EtOAc (150 mL), poured into aq. NH₄Cl (200 mL) and 2 M HCl (50 mL) was added to acidify the aqueous layer. The layers were separated and the aqueous extracted with EtOAc (2×80 mL). Combined organics were washed with aqueous NHaCl (2×200 mL), dried (Na₂SO₄) and filtered. The solvent was removed in vacuo to afford the title compound (7.72 g, 90%) as a pale yellow solid. UPLC (Method A) 1.87 min, 99%, [M+H]⁺=281.2.

### Intermediate 32

### 4-Oxo-6,7-dihydro-5H-pyrazolo[1,5-a]pyridine-2-carboxylic acid

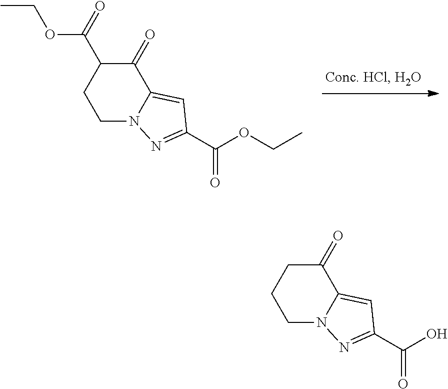 [SMILES: CCOC(=O)c1cc2n(n1)CCC(C(=O)OCC)C2=O ; O=C(O)c1cc2n(n1)CCCC2=O]

A suspension of Intermediate 31 (5.38 g, 19.20 mmol) and conc. HCl/H₂O (2:1, 90 mL) was heated at 100° C. for 6 h, cooled to rt and the solvent removed in vacuo. The yellow residue was dissolved in MeCN:THF (1:4) and the solvent removed in vacuo and this process repeated (×1), and then subsequently repeated with MeCN to afford the title compound (3.44 g, 99%) as a pale yellow solid. UPLC (Method E) 1.55 min, 97%, no ionisation observed.

### Intermediate 33

### Methyl 4-oxo-6,7-dihydro-5H-pyrazolo[1,5-a]pyridine-2-carboxylate

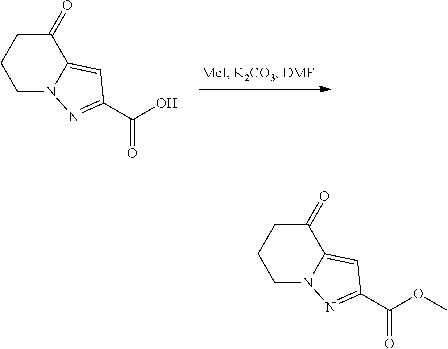

To a solution of 4-oxo-6,7-dihydro-5H-pyrazolo[1,5-a]pyridine-2-carboxylic acid (1.83 g, 10.1 mmol, 1.0 eq) in DMF (25 mL) was added K₂CO₃ (2.81 g, 20.3 mmol, 2.0 eq) to form a suspension. To this suspension was added iodomethane (0.76 mL, 12.2 mmol, 1.2 eq) and stirring continued at rt overnight. To the resulting black mixture was added aq. NH₄Cl (30 mL) and the mixture extracted with EtOAc (3×30 mL). Combined organics were washed with aq. sodium thiosulfate (30 mL), aq. NaHCO₃ (30 mL), aq. NH₄Cl (2×30 mL), dried (Na₂SO₄), filtered and the solvent removed in vacuo to yield the title compound (1.36 g, 69%) as an off white solid.

### Intermediate 34

### Methyl 4,4-difluoro-6,7-dihydro-5H-pyrazolo[1,5-a]pyridine-2-carboxylate

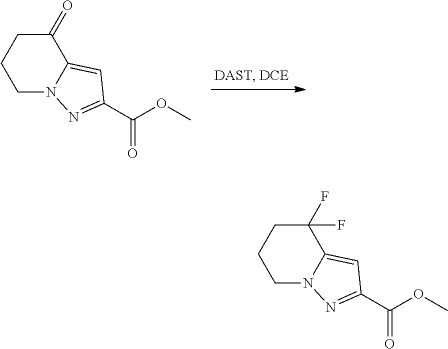

Two identical reactions were set up in 10 mL microwave vials: To a solution of Intermediate 33 (0.67 g, 3.4 mmol, 1 eq) in DCE (3.5 mL) was added DAST (4.5 mL, 34.3 mmol, 10 eq) dropwise and the mixture stirred at rt for 2-3 min, sealed and then stirred at rt for 5 days. The two microwave vials were uncapped and the contents of each vial diluted with DCM (10 mL). The reaction mixtures were each pipetted into stirred aq. NaHCO₃ (100 mL) over 10 min. Additional DCM (20 mL) was added and the mixture stirred for 1-2 h. The phases were separated and combined organics were washed with aq. NaHCO₃ (2×100 mL), dried (MgSO₄), filtered and the solvent removed in vacuo. The residue was purified by column chromatography on silica (100% DCM to EtOAc/DCM, 1:99) to afford the title compound (650 mg, 44%) as a yellow oil. UPLC (Method A) 2.58 min, 84%, [M+H]⁺=217.1.

### Intermediate 35

### 4,4-Difluoro-6,7-dihydro-5H-pyrazolo[1,5-a]pyridine-2-carboxylic acid

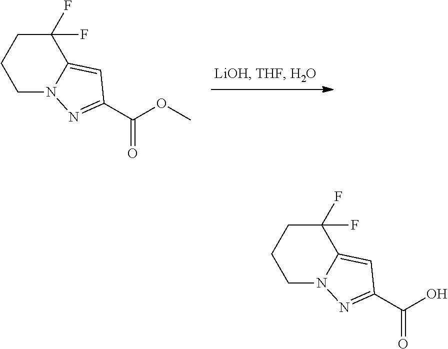

To a solution of Intermediate 34 (735 mg, 3.40 mmol, 1 eq) in THF (10 mL) was added LiOH (163 mg, 6.80 mmol, 2 eq) and H₂O (0.5 mL) and the solution stirred at rt overnight. The resulting yellow solution was acidified to pH 5 using 2M HCl and the solvent partially removed under reduced pressure. The oily residue was diluted with H₂O (6 mL) and 2M HCl (1 mL) and the resulting thick white precipitate sonicated, filtered, washed on the filter paper with H₂O (2×10 mL) and dried in vacuo to furnish the title compound (551 mg, 80%) as a white powder. UPLC (Method E) 2.37 min, 92%, [M+H]⁺=203.1.

### Intermediate 36

### Benzyl N-(4,4-difluoro-6,7-dihydro-5H-pyrazolo[1,5-a]pyridin-2-yl)carbamate

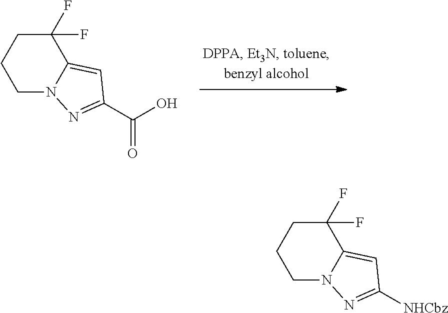 [SMILES: O=C(O)c1cc2n(n1)CCCC2(F)F ; O=C(Nc1cc2n(n1)CCCC2(F)F)OCc1ccccc1]

To a suspension of Intermediate 35 (169 mg, 0.84 mmol, 1 eq) in toluene (10 mL) and Et₃N (233 μL, 1.67 mmol, 2 eq) was added benzyl alcohol (434 μL, 4.18 mmol, 5 eq) and DPPA (359 μL, 1.67 mmol, 2 eq) and the reaction heated to 90° C. overnight. The resulting orange reaction mixture was diluted with EtOAc (40 mL), washed with H₂O (10 mL), sat. aq. NaHCO₃ (10 mL) and brine (10 mL), dried (Na₂SO₄), filtered and concentrated to an orange oil. The crude oil was purified by column chromatography (toluene/EtOAc, 9:1 to 4:1 to 1:1) to yield the desired product (145 mg, 56%) as an off white solid. UPLC (Method A) 3.20 min, 80%, [M+H]⁺=308.

### Intermediate 37

### 4,4-Difluoro-6,7-dihydro-5H-pyrazolo[1,5-a]pyridin-2-amine

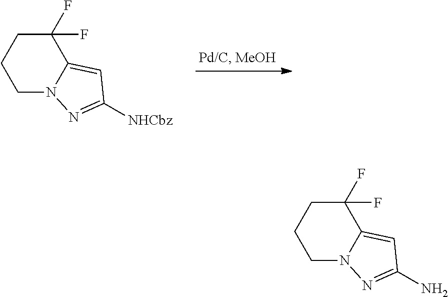 [SMILES: O=C(Nc1cc2n(n1)CCCC2(F)F)OCc1ccccc1 ; Nc1cc2n(n1)CCCC2(F)F]

To a suspension of Intermediate 36 (145 mg, 0.47 mmol, 1 eq) in MeCOH (10 mL) was added 10% Pd/C (50 mg) and the reaction stirred under 1 atm H₂ at rt for 16 h. The mixture was then filtered through dicalcite and the filtrate concentrated under reduced pressure to yield the title compound (78 mg, 95%) as a brown oil, which was used in the next step without further purification. UPLC (Method A) 2.10 min, 72%, [M+H]⁺=174.

### Intermediate 38

### Methyl 3-ethynyl-4-methyl-benzoate

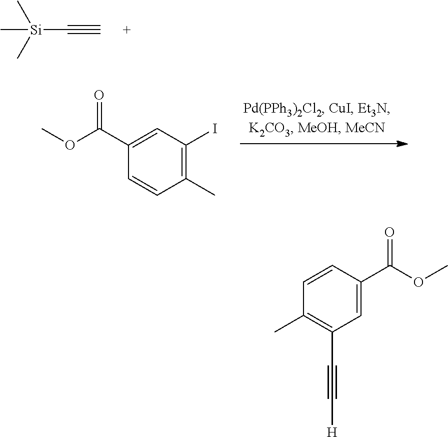

A solution of methyl 3-iodo-4-methylbenzoate (6.00 g, 21.7 mmol), ethynyltrimethylsilane (7.52 mL, 54.3 mmol) and Et₃N (9.09 mL, 65.2 mmol) in MeCN (50 mL) was degassed with N₂ for 15 min. CuI (207 mg, 1.09 mmol) and Pd(PPh₃)₂Cl₂ (763 mg, 1.09 mmol) were added and the mixture was degassed with N₂ for 5 min then stirred under N₂ for 45 min. The reaction mixture was concentrated in vacuo, the sample was redissolved in MeCOH (50 mL), K₂CO₃ (3.00 g, 21.7 mmol) was added and the mixture was stirred for 1 h at rt. An additional portion of K₂CO₃ (3.00 g, 21.7 mmol) was added after 15 min. The reaction mixture was concentrated in vacuo and the sample was redissolved in EtOAc (75 mL) and water (75 mL). The aqueous phase was extracted with EtOAc (2×35 mL) and the combined organic phases were washed with brine (50 mL) and concentrated in vacuo. The sample was purified by column chromatography (C18 reverse phase, [(86 g)], RediSep C18-derivatized silica, 40-63 μm (230-400 mesh), 60 mL per min, gradient 10% to 100% MeCOH in 10% MeOH/H₂O) and dried in a vacuum oven at 60° C. for 18 h to give methyl 3-ethynyl-4-methyl-benzoate (1.40 g, 37%) as a dark black solid. UPLC (Method J) 2.76 min, 100%.

### Intermediate 39

### 3-Ethynyl-4-methyl-benzoic acid

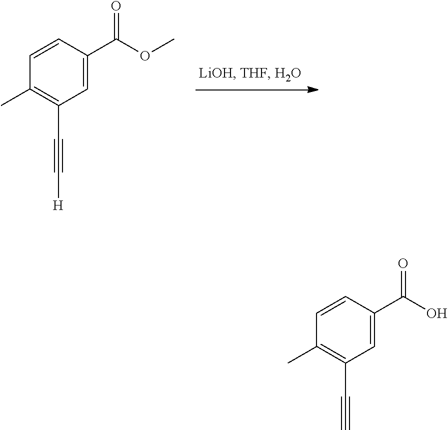

Intermediate 39 (700 mg, 4.0 mmol) and LiOH monohydrate (512 mg, 11.9 mmol) were dissolved in THF:H₂O (10 mL, 1:1) and the reaction stirred at rt for 2 h. A further portion of LiOH monohydrate (512 mg, 11.9 mmol) was added and the reaction stirred at room temperature for 3 d. The THF was removed in vacuo and the residue acidified with 1M HCl. The product was extracted with EtOAc (4×50 mL), dried (MgSO₄) and concentrated in vacuo to give 3-ethynyl-4-methyl-benzoic acid (200 mg, 29%) as a light brown solid. LC-MS (Method 1) 2.27 min, no ionisation observed.

### Intermediate 40

### 3-Ethynyl-4-methyl-N-[1-(2-morpholinoethyl)pyrazol-3-yl]benzamide

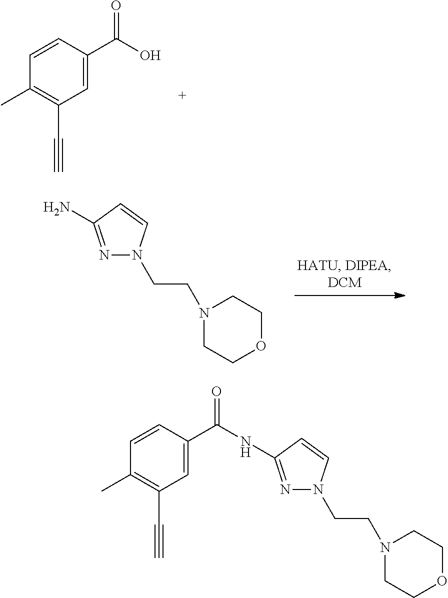

1-[2-(Morpholin-4-yl)ethyl]-1H-pyrazol-3-amine (251 mg, 1.28 mmol), Intermediate 39 (200 mg, 1.16 mmol, 93% pure), DIPEA (202 μL, 1.16 mmol) and HATU (574 mg, 1.51 mmol) were dissolved in DCM (40 mL) and the reaction stirred at rt for 18 h. The mixture was washed with sat. aq. NaHCO₃ (30 mL), dried (MgSO₄) and concentrated in vacuo. The residue was purified by column chromatography (normal phase, [24 g], RediSep silica gel, 35-60 μm (230-400 mesh), 35 mL per min, gradient 0% to 100% EtOAc in iso-hexanes then 0% to 20% MeCOH in DCM [residue loaded in DCM]) to give 3-ethynyl-4-methyl-N-[1-(2-morpholinoethyl)pyrazol-3-yl]benzamide (377 mg, 92%) as a light brown solid. LC-MS (Method 1) 2.04 min, [M+H]⁺=339.2.

### Intermediate 41

### 6-Fluoroisoquinolin-3-amine

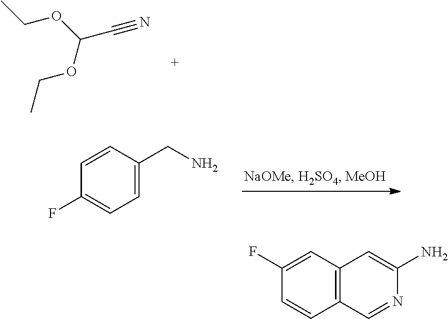

To MeCOH (15 mL) was added 2,2-diethoxyacetonitrile (3.00 g, 23.2 mmol) and a methanolic solution of NaOMe (0.25 g, 4.7 mmol in 1 mL MeCOH) and the reaction stirred at rt for 24 h. 4-Fluorobenzylamine (2.39 mL, 20.9 mmol) was then added and the reaction stirred for a further 24 h at rt. The reaction mixture was concentrated in vacuo and the vessel was cooled to 0° C. before addition of concentrated sulfuric acid (15.0 mL) and further stirring at rt for 24 h. The reaction was neutralised to pH 7 using 4M KOH, the product extracted using DCM (3×150 mL), dried (MgSO₄) and concentrated in vacuo. The residue was purified by column chromatography (normal phase, [80 g], RediSep silica gel, 35-60 μm (230-400 mesh), 60 mL per min, gradient 0% to 100% EtOAc in iso-hexanes [residue loaded in DCM]) to give 6-fluoroisoquinolin-3-amine (292 mg, 7.8%) as a brown solid. LC-MS (Method 1) 1.38 min, [M+H]⁺=163.0.

### Intermediate 42

### 6-Fluoro-4-iodo-isoquinolin-3-amine

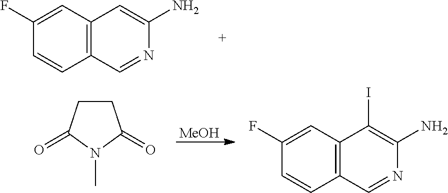

Intermediate 41 (292 mg, 1.66 mmol, 92% pure) and NIS (347 mg, 1.54 mmol) were dissolved in MeCOH (75 mL) and the reaction was stirred at rt for 3 h. A further portion of NIS (187 mg, 0.83 mmol) was then added and the reaction was stirred at room temperature for a further 18 h. The solvents were removed in vacuo and the residue purified by column chromatography (normal phase, [40 g], RediSep silica gel, 35-60 μm (230-400 mesh), 40 mL per min, gradient 0% to 100% EtOAc in iso-hexanes [residue loaded in DCM]) to give 6-fluoro-4-iodo-isoquinolin-3-amine (296 mg, 57%) as a brown solid. LC-MS (Method 1) 2.15 min, [M+H]⁺=289.

### Intermediate 43

### Methyl 4-methyl-3-(2-trimethylsilylethynyl)benzoate

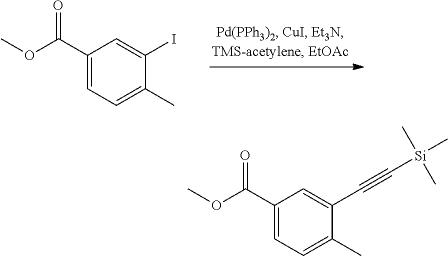

A solution of methyl 3-iodo-4-methylbenzoate (50.0 g, 0.18 mol), Et₃N (55.0 g, 0.54 mol) and ethynyltrimethylsilane (23.1 g, 0.24 mol) in EtOAc (700 mL) was degassed using three cycle of vacuum/N₂. Bis(triphenylphosphine)palladium(II) dichloride (1.27 g, 1.81 mmol) and CuI (0.35 g, 1.81 mmol) were added and the reaction stirred at rt under N₂ for 2 h. The reaction was reduced to dryness in vacuo and the dark brown solid purified by column chromatography (100% heptane to heptane/EtOAc, 9:1) to yield the title compound (44.6 g, 95.1%) as a pale yellow solid. UPLC (Method A) 4.26 min, 95%, mass ion not detected.

### Intermediate 44

### Methyl 3-(2-imidazo[1,2-b]pyridazin-3-ylethynyl)-4-methyl-benzoate

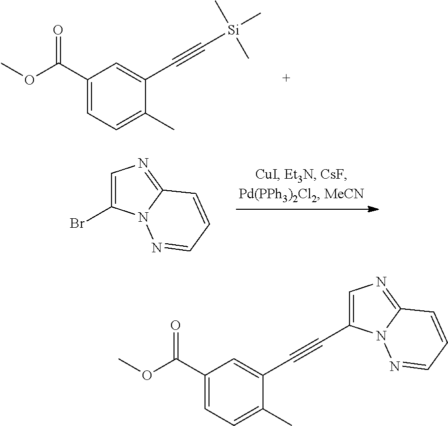

A mixture of Intermediate 43 (318 mg, 1.29 mmol), 3-bromoimidazo[1,2-b]pyridazine (307 mg, 1.55 mmol), triethylamine (0.54 mL, 3.87 mmol), copper(I) iodide (25 mg, 0.13 mmol), bis(triphenylphosphine)palladium(II) dichloride (91 mg, 0.13 mmol) and caesium fluoride (392 mg, 2.58 mmol) in MeCN (4.0 mL) in a microwave tube was degassed with N2 for 2 min and heated at 100° C. in a microwave for 2 h. The reaction mixture was combined with two further batches of the reaction (1.29 and 0.81 mmol) with DCM (50 mL) and H₂O (50 mL) added to the mixture. The layers were separated and the aqueous phase extracted with DCM (2×50 mL). Combined organics were concentrated and the crude product purified on silica gel (DCM/MeOH, 99:1 to 9:1) and recrystallised with MeCN (15 mL) to yield the title compound (238 mg, 24%) as a yellow solid. UPLC (Method A), 3.21 min, 99%, [M+H]⁺=292.2.

### Intermediate 45

### 3-(2-Imidazo[1,2-b]pyridazin-3-ylethynyl)-4-methyl-benzoic acid

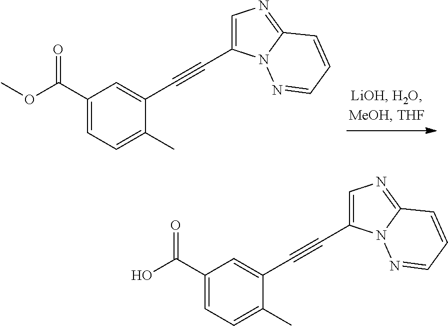 [SMILES: COC(=O)c1ccc(C)c(C#Cc2cnc3cccnn23)c1 ; Cc1ccc(C(=O)O)cc1C#Cc1cnc2cccnn12]

To a solution of Intermediate 44 (238 mg, 0.82 mmol) in MeOH (10 mL) and THF (10 mm) at rt was added LOH.H₂O (103 mg, 2.45 mmol) in H₂O (2.0 m L) and the resulting mixture stirred at rt overnight. The solvent was then removed in vacuo and H₂O (10 mL) added to the residue, which was acidified aq. HCl to pH 1-2. The solvent was removed in vacuo to give the title compound (320 mg) as a brown solid, which was used in the next step without further purification. UPLC (Method A), 1.87 min, 99%, [M+H]⁺=278.2.

### Intermediate 46

### 6′-Nitrospiro[1,3-dithiolane-2,1′-indane]

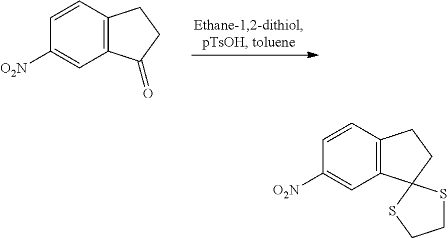 [SMILES: O=C1CCc2ccc([N+](=O)[O-])cc21 ; O=[N+]([O-])c1ccc2c(c1)C1(CC2)SCCS1]

Ethane-1,2-dithiol (1.68 g, 17.9 mmol, 1.10 eq), 6-nitroindan-1-one (2.88 g, 16.2 mmol, 1.00 eq), p-toluene sulfonic acid (0.56 g, 3.3 mmol, 0.20 eq) and toluene (15 mL) were heated under Dean Stark conditions at 100° C. for 24 h. The reaction was then allowed to cool to rt and the toluene removed in vacuo. The residue was purified by column chromatography on silica (EtOAc/heptane, 1:9) to yield the title product (4.05 g, 98%) as a yellow oil. UPLC (Method A) 1.71 min, 100%, no ionisation observed.

### Intermediate 47

### 2-Bromo-1,1-difluoro-6-nitro-indane

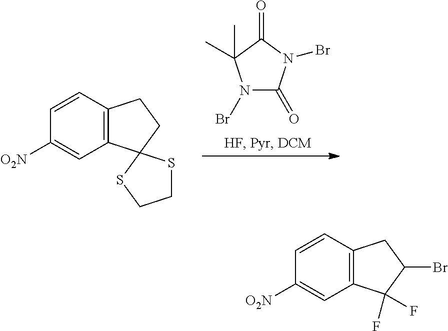 [SMILES: O=[N+]([O-])c1ccc2c(c1)C1(CC2)SCCS1 ; CC1(C)C(=O)N(Br)C(=O)N1Br ; O=[N+]([O-])c1ccc2c(c1)C(F)(F)C(Br)C2]

To a solution of 1,3-dibromo-5,5-dimethyl-imidazolidine-2,4-dione (18.1 g, 63.2 mmol) in anhydrous DCM (90 mL) at −70° C. was added HF-pyridine (18.8 mL, 70%) dropwise and the reaction stirred at −70° C. for 30 min before addition of a solution of Intermediate 46 (4.00 g, 15.8 mmol) in DCM (10 mL) dropwise and further stirring for 4 h. The mixture was then allowed to warm to rt and stirred overnight. To the dark brown mixture was added NaOH (2 M, 50 mL) and NaHSO₃ (3 M, 5.0 mL) and the phases separated. The aqueous phase was extracted with DCM (2×80 mL) and the combined organics were concentrated in vacuo to give the crude product (4.02 g) as an orange oil. This was purified on silica gel (EtOAc/heptane, 1:9 to 3:7) to give 2-bromo-1,1-difluoro-6-nitro-indane (3.63 g) as a yellow oil, which was used in the next step without further purification. UPLC (Method G), 1.30 min, 80%, no ionisation.

### Intermediate 48

### 1,1-Difluoro-6-nitro-indene

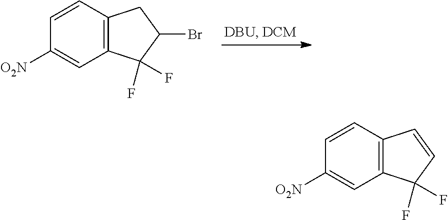 [SMILES: O=[N+]([O-])c1ccc2c(c1)C(F)(F)C(Br)C2 ; O=[N+]([O-])c1ccc2c(c1)C(F)(F)C=C2]

DBU (3.3 mL, 22.2 mmol) was added to a solution of Intermediate 47 (3.63 g, 13.1 mmol) in anhydrous DCM (50 mL) at rt for 2 h. To the mixture was then added aq. HCl (50 mL, 2M) and the phases separated. The aqueous phase was washed with DCM (2×50 mL) and combined organics concentrated in vacuo to give the crude product (3.01 g) as a purple solid. The crude product was purified on silica (EtOAc/heptane, 1:9 to 3:7) to yield 1,1-difluoro-6-nitro-1H-indene (2.62 g) as a yellow solid, which was used in the next step without further purification. UPLC (Method G) 0.99 min, 83%, no ionisation.

### Intermediate 49

### 1,1-Difluoro-6-nitro-indane

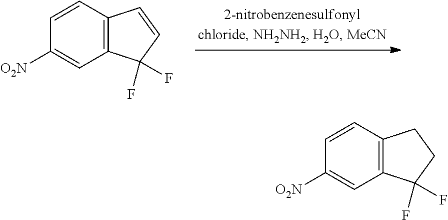 [SMILES: O=[N+]([O-])c1ccc2c(c1)C(F)(F)C=C2 ; O=[N+]([O-])c1ccc2c(c1)C(F)(F)CC2]

Hydrazine monohydrate (2.58 mL, 53.2 mmol) was added to a solution of Intermediate 48 (2.62 g, 13.3 mmol) and 2-nitrobenzenesulfonyl chloride (5.89 g, 26.6 mmol) in MeCN (60 mL) at 0° C. dropwise to give a yellow suspension. The reaction mixture was allowed to warm to rt, resulting in the formation of a clear solution, which was stirred at rt for 72 h. The MeCN was removed in vacuo at 30° C. and H₂O (80 mL) was added to the reaction mixture to dissolve the precipitate. The crude product was extracted with EtOAc (3×50 mL) and combined organics were concentrated in vacuo. The crude product was purified on silica using EtOAc/heptane (1:9 to 3:7) to yield 1,1-difluoro-6-nitro-indane (2.00 g, 76% yield) as a yellow oil which solidified on standing. UPLC (Method G), 1.12 min, 99.6%, no ionisation.

### Intermediate 50

### 3-bromo-1,1-difluoro-6-nitro-indane

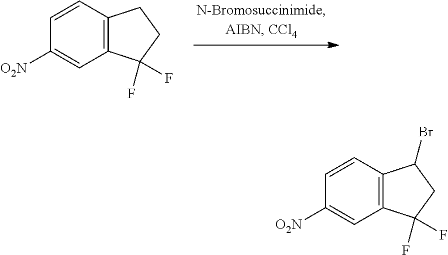 [SMILES: O=[N+]([O-])c1ccc2c(c1)C(F)(F)CC2 ; O=[N+]([O-])c1ccc2c(c1)C(F)(F)CC2Br]

Intermediate 49 (500 mg, 2.51 mmol), AIBN (41 mg, 0.25 mmol) and NBS (536 mg, 3.01 mmol) in carbon tetrachloride (30 mL) were heated at reflux (90° C.) overnight. This reaction was then repeated on an additional batch of material and both reactions combined after cooling to rt. Silica gel (10.0 g) was added to the crude mixture and the solvent removed in vacuo. The crude product was purified on silica (EtOAc/heptane, 3:97), to yield 3-bromo-1,1-difluoro-6-nitro-indane (788 mg, 56%) as a yellow oil, which contained 16% starting material by UPLC and was used without further purification. UPLC (Method G) 1.39 min, 83%, no ionisation.

### Intermediate 51

### 1-(3,3-difluoro-5-nitro-indan-1-yl)-4-methyl-piperazine

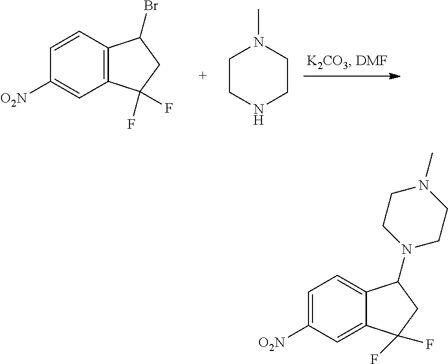

1-Methylpiperazine (568 mg, 5.7 mmol) was added to Intermediate 50 (788 mg, 2.8 mmol) and potassium carbonate (783 mg, 5.7 mmol) in DMF (10 mL) at rt and the resulting mixture stirred at rt for 4 h. To the reaction was then added H₂O (100 mL) and EtOAc (3×50 mL) and the solvent removed in vacuo from the combined organics to yield a purple oil. This was purified on silica (MeOH/DCM, 1:99 to 1:9) to yield the title product (100 mg, 12%) as a dark green solid. UPLC (Method A) 3.07 min, 78%, [M+H]⁺=298.2.

### Intermediate 52

### 3,3-difluoro-1-(4-methylpiperazin-1-yl)indan-5-amine

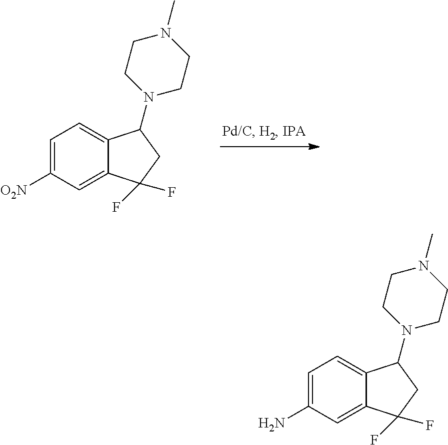 [SMILES: CN1CCN(C2CC(F)(F)c3cc([N+](=O)[O-])ccc32)CC1 ; CN1CCN(C2CC(F)(F)c3cc(N)ccc32)CC1]

Pd/C (15.0 mg, 10% wt) was added to Intermediate 51 (100 mg, 0.34 mmol) in IPA (5 mL). The resulting reaction mixture was hydrogenated at rt at 1 atm for 3 h. The reaction mixture was filtered through Celite and washed with IPA (5 mL). The solvent was removed in vacuo to give crude 3,3-difluoro-1-(4-methylpiperazin-1-yl)indan-5-amine (103 mg) as a grey solid, which was taken on directly to the next step. UPLC (Method A) 2.31 min, 67%, [M+H]⁺=268.2.

### Intermediate 53

### Methyl 2-(3,3-dimethyl-5-oxo-morpholin-4-yl)acetate

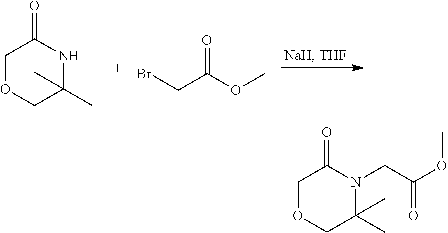

To a N₂ purged stirring solution of 5,5-dimethylmorpholin-3-one (2.00 g, 15.5 mmol) in anhydrous THF (70 mL) at rt was added NaH (60% in oil) (619 mg, 15.5 mmol) portionwise and the reaction stirred at rt for 50 min. Methyl 2-bromoacetate (1.46 mL, 15.5 mmol) was added dropwise and the reaction stirred for a further 4 h before the addition of water (5.0 mL) dropwise to the reaction and subsequent pouring of the reaction into water (40 mL). The resulting solution was extracted using EtOAc (6×10 mL), the combined organics washed with brine (30 mL), dried over MgSO₄ and concentrated to give the crude product (4.0 g) as a cloudy oil. The crude product was purified by silica column chromatography (20-90% EtOAc in heptane) to afford methyl 2-(3,3-dimethyl-5-oxo-morpholin-4-yl)acetate (1.60 g, 46%) as a colourless oil. ¹H NMR (400 MHz, CDCl₃): δ_{H} 4.24 (s, 2H), 4.04 (s, 2H), 3.75 (s, 3H), 3.66 (s, 2H), 1.26 (s, 6H) ppm.

### Intermediate 54

### 2-(3,3-dimethyl-2-oxo-1-piperidyl)acetamide

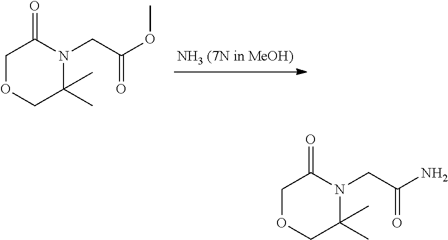

A solution of Intermediate 53 (2.40 g, 11.9 mmol) in 7N NH₃/MeOH (100 mL) was stirred overnight at 100° C. in a sealed Parr hydrogenator vessel. The reaction was concentrated, split across two microwave vials, each vial was diluted with 7N NH₃/MeOH (10 mL) and heated to 110° C. in a Biotage initiator for 1.5 h. The reaction was concentrated and purified by silica column chromatography (0-20% MeCOH in EtOAc) to give the desired product (119 mg, 5%) as a colourless oil.

### Intermediate 55

### 2-bromo-8,8-dimethyl-6,7-dihydro-5H-imidazo[1,2-a]pyridine

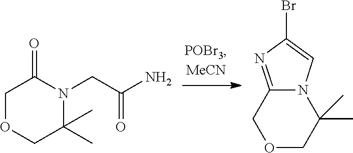

To two microwave vials each containing Intermediate 54 (243 mg, 1.31 mmol) in MeCN (5.0 mL) was added POBr₃ (2.25 g, 7.83 mmol) and both vials heated to 120° C. in a Biotage initiator for 35 min. The reactions were then both added dropwise to a stirring solution of H₂O (40 mL) and DCM (40 mL) at 0° C. The solution was then carefully basified to pH10 with the addition of solid K₂CO₃. The biphasic solution was extracted with 1:9 IPA:DCM (7×10 mL), the combined organic layers washed with brine (30 mL) dried over MgSO₄, filtered and concentrated to a brown residue. Purification by silica chromatography (40-100% EtOAc in heptane) afforded the title compound as a brown crystalline solid (215 mg, 36%). UPLC (Method A) 2.41 min, 17%, [M+H]⁺=233.0/231.0.

### Intermediate 56

### 6,6-Dimethyl-5,7-dihydropyrrolo[1,2-c]imidazole

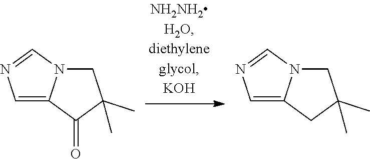

Hydrazine monohydrate (0.13 mL, 2.76 mmol) was added to a stirred solution of 6,6-dimethyl-5H-pyrrolo[1,2-c]imidazol-7-one (83.0 mg, 0.55 mmol) dissolved in diethylene glycol (5.0 mL, 0.55 mmol). The resulting solution was heated using a Biotage Initiator microwave reactor at 180° C. for 60 min. The reaction was then allowed to cool to rt, and the flask was unsealed before KOH (217 mg, 3.87 mmol) was carefully added to the mixture. The flask was resealed and the resulting suspension was heated using a Biotage Initiator microwave reactor at 220° C. for 120 min. The reaction mixture was acidified to pH 5 with dilute aqueous HCl (2 M), and the solvent removed in vacuo. The resulting crude residue was triturated with DCM/MeOH (1:1), filtered and the solvent removed in vacuo to yield the title compound (40 mg, 53%) containing significant DEG, which was taken directly on to the next step.

### Intermediate 57

### 1,3-Diiodo-6,6-dimethyl-5,7-dihydropyrrolo[1,2-c]imidazole

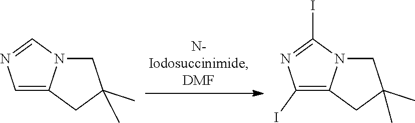

Intermediate 56 (17.0 mg, 0.07 mmol) and N-iodosuccinimide (35.2 mg, 0.16 mmol) were dissolved in DMF (0.7 mL) at rt and the reaction heated at 70° C. for 2 h. The mixture was cooled to rt, diluted with water (2 mL), extracted with DCM (3×5 mL), and the layers separated. The combined organics were dried over MgSO₄, filtered and concentrated in vacuo to give crude product (29.0 mg), which was purified by column chromatography (manual column, normal phase, SilaFlash®P60 silica gel 40-63 μm/230-400 mesh, 60 Å, [silica/crude=30/1], 0-5% MeCOH in DCM) to yield the title compound (11 mg, 40%) as an off-yellow solid. LC-MS (Method D) 2.82 min, 37%, [M+H]⁺=388.7.

### Intermediate 58

### 1-Iodo-6,6-dimethyl-5,7-dihydropyrrolo[1,2-c]imidazole

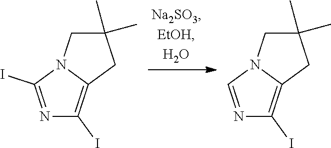

To a stirred solution of Intermediate 57 (50.0 mg, 0.13 mmol) in EtOH (5.0 mL) was added a solution of Na₂SO₃ (81.2 mg, 0.64 mmol) in H₂O (5.0 mL) and the solution stirred for 35 min at 60° C. The reaction was allowed to cool down to rt and the volatiles were removed in vacuo. The aqueous was extracted with EtOAc (3×5 mL), combined organic phases dried over Na₂SO₄, filtered and concentrated in vacuo to yield 1-iodo-6,6-dimethyl-5,7-dihydropyrrolo[1,2-c]imidazole (54 mg) which was taken on crude to the next step. UPLC (Method A) 2.77 min, 86%, [M+H]⁺=263.0.

### Intermediate 59

### 2-(5-Hydroxy-6-oxaspiro[3.4]octan-5-yl)acetonitrile

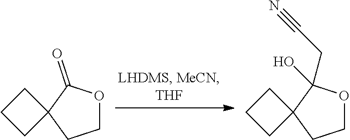 [SMILES: O=C1OCCC12CCC2 ; N#CCC1(O)OCCC12CCC2]

To a solution of LHMDS (1M in THF, 14.7 mL, 14.7 mmol, 2.2 eq.) in anhydrous THF (20 mL) at −78° C. was added a solution of the starting lactone (840 mg, 6.7 mmol, 1.0 eq) and MeCN (0.69 mL, 13.3 mmol, 2.0 eq) in THF (6.6 mL) dropwise. The reaction was stirred at −78° C. for 30 min and then at rt for 2 h. To the reaction mixture was added sat. aq. NH₄Cl (10 mL) and the reaction stirred at rt for 5 min under N₂. The reaction mixture was partitioned between sat. aq. NH₄Cl (20 mL) and DCM (20 mL) and the phases were separated. The aqueous phase was washed with DCM (3×50 mL) and combined organics dried over Na₂SO₄ and the solvent removed in vacuo. The crude residue was purified by column chromatography (Silicycle 40 g, EtOAc in heptane, 0 to 40% over 15 CV) to yield the desired product (640 mg, 57%) as a colourless oil. UPLC (Method A) 1.85 min, 98%, [M−H]⁻=166.1.

### Intermediate 60

### 2-[1-(3-Amino-1H-pyrazol-5-yl)cyclobutyl]ethanol

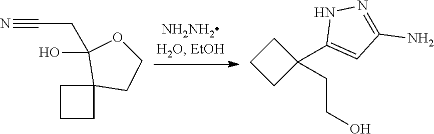 [SMILES: N#CCC1(O)OCCC12CCC2 ; Nc1cc(C2(CCO)CCC2)[nH]n1]

Intermediate 59 (490 mg, 2.93 mmol) was dissolved in EtOH (5.0 mL) and hydrazine monohydrate (213 μL, 4.38 mmol) was added. The resulting solution was stirred at 60° C. for 3 days. The reaction was cooled to rt and CO₂ was bubbled through for 1 h. The reaction was concentrated and MecOH (10 mL) added and the resulting white solid was filtered off. The filtrate was concentrated to give the crude title product (526 mg) as a brown oil. This was used in the next step without further purification.

### Intermediate 61

### Spiro[5,6-dihydropyrrolo[1,2-b]pyrazole-4,1′-cyclobutane]-2-amine

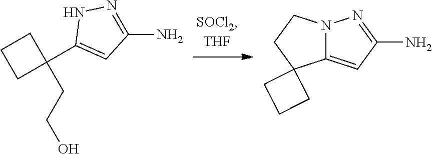 [SMILES: Nc1cc(C2(CCO)CCC2)[nH]n1 ; Nc1cc2n(n1)CCC21CCC1]

To a solution of Intermediate 60 (used crude, 354 mg, 1.95 mmol) in dry THF (10 mL) was added dropwise SOCl₂ (708 μL, 9.77 mmol) and the resulting mixture stirred at rt for 3 h. The reaction was carefully poured into a 1:1 mixture of NH₄OH (28% aq.) and ice (total 60 mL) and the aqueous extracted three times with DCM (3×50 mL). The combined organic phases were washed with brine (60 mL), dried over Na₂SO₄ and concentrated to dryness to give crude spiro[5,6-dihydropyrrolo[1,2-b]pyrazole-4,1′-cyclobutane]-2-amine (200 mg, 24%) as a brown oil, which was used without further purification. LC-MS (Method D) 2.38 min, 38.2%, [M+H]⁺=264.0.

### Intermediate 62

### 1-Cyclopentyl-4-iodo-imidazole

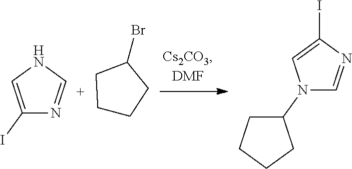

To an N₂ purged stirring solution of 4-Iodo-1H-imidazole (1.00 g, 5.2 mmol) and Cs₂CO₃ (5.07 g, 15.5 mmol) in DMF (10 mL) at rt was added dropwise cyclopentyl bromide (663 μL, 6.2 mmol) and the reaction stirred at 60° C. for 16 h. The temperature was then increased to 80° C. and the reaction further stirred for 3 h. The solvent was removed in vacuo and the crude solid diluted with H₂O (40 mL) and EtOAc (30 mL) followed by sonication. The organic layer was then separated and the aqueous extracted using EtOAc (4×10 mL). The combined organics were washed with brine (30 mL), dried (MgSO₄) and concentrated to a brown/orange oil (1.41 g). The crude was purified by silica chromatography (20-70% EtOAc in heptane) to afford 1-cyclopentyl-4-iodo-imidazole (874 mg, 65%). ¹H NMR (400 MHz, CDCl₃: δH 7.41 (s, 1H), 7.02 (s, 1H), 4.47-4.40 (m, 1H), 2.20-2.14 (m, 2H), 1.86-1.68 (m, 6H) ppm.

### Intermediate 63

### Methyl 2-(2,2-dimethyl-5-oxo-pyrrolidin-1-yl)acetate

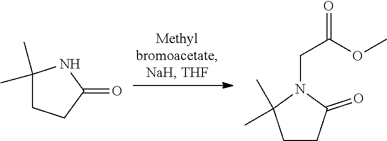

To an ice-cold solution of 5,5-dimethylpyrrolidin-2-one (10.0 g, 88 mmol) in dry THF (300 mL) was added methyl bromoacetate (10.0 mL, 106 mmol) followed by NaH (3.9 g, 97 mmol) portionwise and the reaction stirred at 0° C. for 1.5 h under N₂. The reaction was then carefully poured into sat. aq. NH₄Cl (300 mL) and extracted with EtOAc (3×100 mL). The combined organic phases were dried over Na₂SO₄, filtered and concentrated in vacuo to yield methyl 2-(2,2-dimethyl-5-oxo-pyrrolidin-1-yl)acetate (21.2 g) as a yellow oil which was taken on crude to the next step of the reaction. ¹H NMR (400 MHz, CDCl₃) δ 3.90 (s, 2H), 3.72 (s, 3H), 2.44 (t, J=8 Hz, 2H), 1.93 (t, J=8 Hz, 2H), 1.20 (s, 6H) ppm.

### Intermediate 64

### 2-(2,2-dimethyl-5-oxo-pyrrolidin-1-yl)acetamide

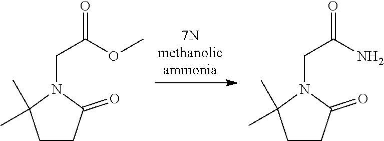

Intermediate 63 (32.7 g, 177 mmol) was dissolved in NH₃ (7N in MeCOH, 200 mL) and transferred into a Hasteloid bomb. The reaction vessel was sealed and heated to 60° C. overnight. The reaction was allowed to cool to rt and the solvent removed in vacuo to yield a mixture of SM and product (7:3) by ¹H NMR (35.9 g, 119%) as a yellow oil. The crude mixture was dissolved in NH₃ (7N in MeCOH, 600 mL) and transferred to a 1 L bomb, sealed and heated to 80° C. overnight. The reaction was allowed to cool to rt and the solvent removed in vacuo to yield methyl 2-(2,2-dimethyl-5-oxo-pyrrolidin-1-yl)acetate (33.0 g) which was taken on crude to the next step.

### Intermediate 65

### 2-bromo-5,5-dimethyl-6,7-dihydropyrrolo[1,2-a]imidazole

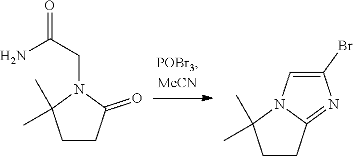

This reaction was divided into 8 different batches, all carried out in the same way. To a MW vial was added crude Intermediate 64 (2.0 g, 11.8 mmol), POBr₃ (10.1 g, 35.3 mmol) and MeCN (10 mL). The reaction mixture was sealed and heated to 70° C. overnight. The 8 batches were allowed to cool to rt and poured into water (1 L). The aqueous was basified with K₂CO₃ (120 g) to pH10 and was extracted with EtOAc (3×1 L). The combined organic phases were dried over Na₂SO₄, filtered and concentrated to dryness. The residue was purified by NP chromatography (Silica, EtOAc/DCM 0 to 50%) to yield 2-bromo-5,5-dimethyl-6,7-dihydropyrrolo[1,2-a]imidazole (11.6 g, 56%) as a yellow oil which crystallised on standing. UPLC (Method A) 2.60 min, 99%, [M+H]⁺=215.1.

### Intermediate 66

### Methyl 2-(2-isopropyl-5-oxo-pyrrolidin-1-yl)acetate

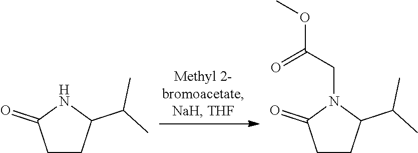

To a cooled (0° C.) suspension of 5-isopropylpyrrolidin-2-one (500 mg, 3.9 mmol, 1.0 eq) in dry THF (15 mL) under N₂ was added NaH (60% in mineral oil, 142 mg, 5.9 mmol, 1.5 eq) portionwise and the mixture stirred at rt for 30 min after which methyl 2-bromoacetate (751 mg, 4.9 mmol, 1.3 eq) was added and the mixture stirred at rt for 18 h. The reaction was quenched with sat. NH₄Cl (5 mL) and the volatiles removed in vacuo. The residue was partitioned between EtOAc (25 mL) and H₂O (15 mL) and the organic phase dried over MgSO₄ and concentrated in vacuo. The residue was purified by normal phase chromatography (100% tert-butyl methyl ether) to afford the title compound (350 mg, 45%) as a colourless oil. 1H-NMR (396 MHz, chloroform-D) δ 4.48 (d, J=17.6 Hz, 1H), 3.74-3.66 (m, 4H), 3.57 (d, J=17.6 Hz, 1H), 2.38 (t, J=8.5 Hz, 2H), 2.04-1.92 (m, 2H), 1.80-1.71 (m, 1H), 0.91 (d, J=6.7 Hz, 3H), 0.76 (d, J=6.7 Hz, 3H) ppm.

### Intermediate 67

### 2-(2-Isopropyl-5-oxo-pyrrolidin-1-yl)acetamide

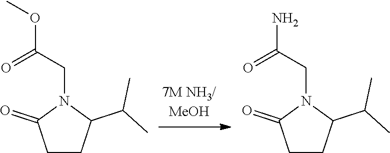

Intermediate 66 (350 mg, 1.76 mmol, 1 eq) was dissolved in 7 N methanolic NH₃ (5 mL) and stirred at rt in a sealed tube for 48 h. The reaction was concentrated in vacuo to afford the title compound (320 mg, 99%) as a colourless gum. 1H-NMR (396 MHz, chloroform-D) δ 6.53 (s, 1H), 5.40 (s, 1H), 3.94 (d, J=15.1 Hz, 1H), 3.82 (d, J=15.1 Hz, 1H), 3.68-3.64 (m, 1H), 2.41-2.37 (m, 2H), 2.18-2-07 (m, 1H), 2.06-1.95 (m, 1H), 1.87-1.75 (m, 1H), 0.92 (d, J=7.3 Hz, 3H), 0.77 (d, J=6.7 Hz, 3H) ppm.

### Intermediate 68

### 2-Bromo-5-isopropyl-6,7-dihydro-5H-pyrrolo[1,2-a]imidazole

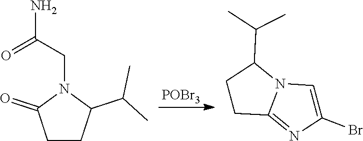

Intermediate 67 (310 mg, 1.68 mmol, 1 eq) and phosphorus(V) oxybromide (1.93 g, 6.73 mmol, 4 eq) were sealed in a tube and heated at 100° C. for 1 h. The reaction was cooled to rt and poured into water (25 mL), the insoluble material was removed by filtration and the solution basified with K₂CO₃, extracted with EtOAc (2×20 mL), the combined organics dried (MgSO₄) and concentrated in vacuo to afford the title compound (310 mg, 80%) as a brown gum. UPLC (Method A): 2.84 min, 80%, [M+H]⁺=229.0/231.0.

### Intermediate 69

### 2-bromo-5-isopropyl-6,8-dihydro-5H-imidazo[2,1-c][1,4]oxazine

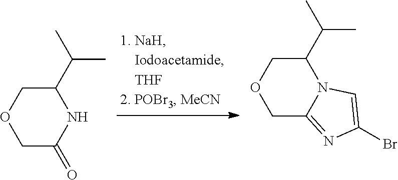

To a solution of 5-isopropylmorpholin-3-one (1.25 g, 8.7 mmol, 1.0 eq) in THF (15 mL) was added NaH (60% in mineral oil, 0.45 g, 11.4 mmol, 1.3 eq) portionwise over 5 min and the reaction stirred at rt for 10 min before addition of a solution of iodoacetamide (1.78 g, 9.6 mmol, 1.1 eq) in THF (10 mL) dropwise over 10 min. The reaction was stirred at rt for 2 h before addition of a further portion of iodoacetamide (0.32 g, 1.9 mmol, 0.2 eq) and further stirring at rt for 2 h. A further portion of NaH (0.25 g, 1.7 mmol, 0.2 eq) was added and the reaction mixture stirred at rt for a further 16 h, quenched by addition of 3 drops of water and then concentrated in vacuo to give a pale orange foamy solid. This was suspended in MeCN (20 mL), phosphoryl tribromide (7.22 g, 25.2 mmol, 3.0 eq) added and the reaction mixture heated at 95° C. for 3 h. The reaction mixture was then concentrated in vacuo and partitioned between saturated NaHCO₃ solution (100 mL) and DCM (200 mL). The organic layer was concentrated in vacuo, absorbed onto silica and purified by normal phase chromatography 0-30% EtOAc/DCM to give the title compound (282 mg, 14%) as an orange brown oily residue, which was used directly in the next step. UPLC (Method A): 2.73 min, 60%, [M+H]⁺=245.0/247.0.

### Intermediate 70

### 4-(1H-imidazol-4-yl)but-3-yn-2-ol

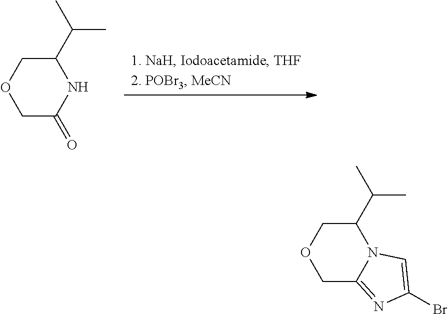

A suspension of 4-iodo-1H-imidazole (190 mg, 0.98 mmol, 1.00 eq), CuI (9 mg, 0.05 mmol, 0.05 eq), Pd(Ph₃P)₂Cl₂ (34 mg, 0.05 mmol, 0.05 eq) and but-3-yn-2-ol (70 mg, 1.00 mmol, 2.00 eq) in MeCN (3 mL) was degassed with N₂, after which triethylamine (410 μL, 2.94 mmol, 3.00 eq.) was added. The reaction was heated at 100° C. in a sealed tube for 3 h, and then concentrated in vacuo. The crude material was dissolved in H₂O (10 mL), filtered and purified by ion-exchange (Dowex W50X), washed with water and eluted with 20% NH₃/H₂O to afford the title compound (610 mg, 87%) as a brown gum. UPLC (Method A): 0.91 min, 89%, [M+H]⁺=137.1.

### Intermediate 71

### 4-(1H-imidazol-4-yl)butan-2-ol

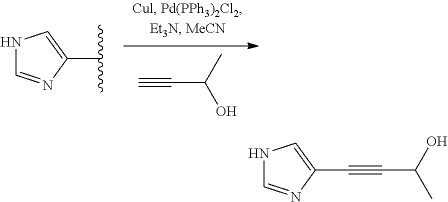

A solution of Intermediate 70 (610 mg, 4.48 mmol, 1 eq) in MeCOH (25 mL) was split into 5 vials. To each vial was added 10% Pd/C (40 mg) and ammonium formate (566 mg, 8.96 mmol, 10 eq) portionwise. The reactions were heated at 55° C. for 3 h and then at reflux for 1 h. The reactions were combined, filtered through a pad of Celite and concentrated in vacuo. The crude material was dissolved in H₂O, purified by ion-exchange (Dowex W50X), washed with water and eluted with 20% NH₃/H₂O to afford the title compound (410 mg, 65%) as a brown gum. UPLC (Method A): 0.89 min, 88.7%, [M+H]⁺=141.1.

### Intermediate 72

### 4-(3-Chlorobutyl)-1H-imidazole

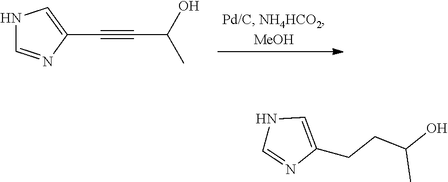

A solution of Intermediate 71 (410 mg, 2.92 mmol, 1 eq) in thionyl chloride (5 mL) was heated at 80° C. for 5 min, cooled to rt and the reaction concentrated in vacuo to afford the title compound (464 mg, 100%, 2.92 mmol). UPLC (Method A): 2.36 min, 83%, [M+H]⁺=159.1/161.1.

### Intermediate 73

### 5-Methyl-6,7-dihydro-5H-pyrrolo[1,2-c]imidazole

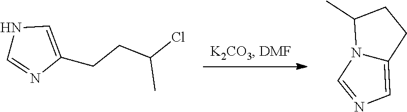

To a solution of Intermediate 72 (0.46 g, 2.9 mmol, 1 eq) in DMF (15 mL) was added K₂CO₃ (2.02 g, 14.6 mmol, 5 eq) and the mixture heated at 100° C. overnight. The reaction was concentrated in vacuo, the crude material dissolved in EtOAc (2×15 mL), the solids removed by filtration and the solution concentrated in vacuo to afford the title compound (410 mg, assumed quant) as a gum. UPLC (Method A): 1.95 min, 83%, [M+H]⁺=123.1.

### Intermediate 74

### 1,3-Diiodo-5-methyl-6,7-dihydro-5H-pyrrolo[1,2-c]imidazole

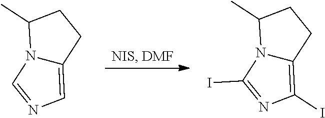

To a solution of Intermediate 73 (100 mg, 0.8 mmol, 1.0 eq) in DMF (10 mL) was added N-iodosuccinimide (405 mg, 1.8 mmol, 2.2 eq) and the reaction heated at 75° C. under N₂ for 2 h. The reaction was concentrated in vacuo, diluted with DCM (10 mL) and H₂O (10 mL), the phases separated and the aqueous extracted with DCM (2×10 mL). The combined organics were dried (MgSO₄) and concentrated in vacuo. The crude material was purified by normal phase chromatography 0.5:5:95 NH₃/MeOH/DCM to afford the title compound (157 mg, 51%) as a brown gum. UPLC (Method A): 2.98 min, 22%, [M+H]⁺=375.0.

### Intermediate 75

### 1-Iodo-5-methyl-6,7-dihydro-5H-pyrrolo[1,2-c]imidazole

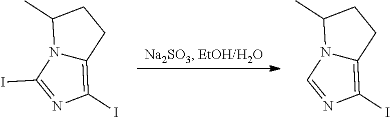

To a solution of Intermediate 74 (150 mg, 0.4 mmol, 1 eq) in EtOH (10 mL) was added a solution of sodium sulfite (253 mg, 2.0 mmol, 5 eq) in H₂O (10 mL) and the reaction heated at 60° C. for 15 min. The EtOH was removed in vacuo and the aqueous extracted with EtOAc (3×10 mL), the combined organics dried (MgSO₄) and the solvent removed in vacuo to afford the title compound (75 mg, 75%) as a pale brown gum. UPLC (Method A): 2.52 min, 54%, [M+H]⁺=249.1.

### Intermediate 76

### 3-[1-(2-Hydroxyethyl)cyclopropyl]-3-oxo-propanenitrile

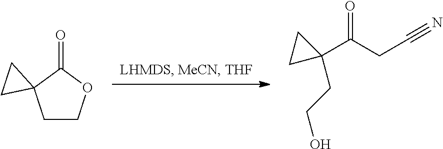 [SMILES: O=C1OCCC12CC2 ; N#CCC(=O)C1(CCO)CC1]

To a solution of LHMDS (1M in THF, 9.8 mmol, 2.2 eq.) in THF (22 mL) cooled to −78° C. was added a solution of 5-oxaspiro[2.4]heptan-4-one (500 mg, 4.5 mmol, 1.0 eq) and MeCN (0.47 mL, 8.9 mmol, 2.0 eq) in THF (2 mL) dropwise and the reaction stirred at −78° C. for 15 min and subsequently warmed to rt over 1 h. The reaction was quenched by dropwise addition of sat. aq. NH₄Cl solution (20 mL) and washed with EtOAc (3×30 mL). Combined organics were dried over MgSO₄ and concentrated in vacuo to yield the title compound (468 mg, 69%) as a colourless oil, which was used in the next step without further purification. 1H-NMR (396 MHz, CDCl₃) δ 3.92-3.87 (m, 2H), 3.65 (t, J=5.4 Hz, 2H), 1.88 (t, J=5.4 Hz, 2H), 1.32-1.27 (m, 2H), 0.83-0.79 (m, 2H) ppm.

### Intermediate 77

### 2-[1-(3-Amino-1H-pyrazol-5-yl)cyclopropyl]ethanol

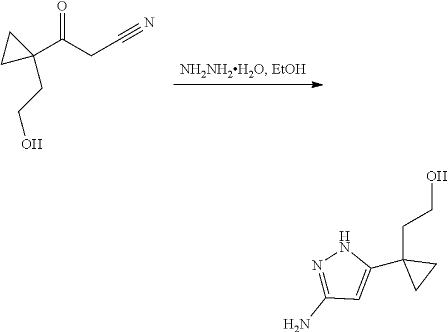 [SMILES: N#CCC(=O)C1(CCO)CC1 ; Nc1cc(C2(CCO)CC2)[nH]n1]

To a solution of Intermediate 76 (468 mg, 3.1 mmol, 1.0 eq) in EtOH (4 mL) was added hydrazine hydrate (4.45 mL, 4.6 mmol, 1.5 eq) and the reaction heated to 90° C. for 5 h in a sealed vial. The reaction was then concentrated in vacuo and the crude residue purified by normal phase column chromatography, 0-15% MeCOH/DCM to afford the title compound (340 mg, 73%) as a pale yellow gum, which was taken on directly to the next step.

### Intermediate 78

### Spiro[5,6-dihydropyrrolo[1,2-b]pyrazole-4,1′-cyclopropane]-2-amine

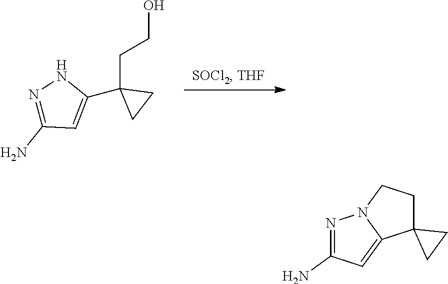 [SMILES: Nc1cc(C2(CCO)CC2)[nH]n1 ; Nc1cc2n(n1)CCC21CC1]

To a stirred solution of Intermediate 77 (340 mg, 2.0 mmol) in THF (11 mL) was added SOCl₂ (0.74 mL, 10.2 mmol) dropwise over 1 min and the reaction stirred at rt for 45 min. The reaction was poured slowly into a stirred solution of NH₄OH (35% solution in H₂O) and ice (8 g) (Vol NH₃OH=5/3×volume THF, mass ice=Vol NH₃OH/2.5) and stirred for 5 min. The solution was extracted with DCM (3×20 mL), combined organics were dried over MgSO₄ and concentrated in vacuo. The crude material was purified by normal phase column chromatography (Biotage Isolera, 25 g, SiliaSep silica gel 40-63 μm/230-400 mesh, 60 Å, residue [loaded in DCM], 0-10% Methanol in DCM to afford spiro[5,6-dihydropyrrolo[1,2-b]pyrazole-4,1′-cyclopropane]-2-amine (46.0 mg, 15%) as a colourless glass. 1H-NMR (396 MHz, chloroform-d4) δ 4.95 (s, 1H), 4.13-4.02 (m, 2H), 3.52 (br s, 2H), 2.51-2.43 (m, 2H), 1.00-0.95 (m, 2H), 0.92-0.86 (m, 2H) ppm.

### Intermediate 79

### 4-Iodo-1-isobutyl-imidazole

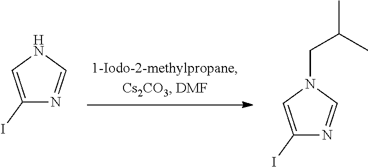

To a solution of iodoimidazole (1.0 g, 5.2 mmol, 1.0 eq) in DMF (10 mL) was added caesium carbonate (5.0 g, 15.5 mmol, 3.0 eq) and 1-iodo-2-methylpropane (710 μL, 6.2 mmol, 1.2 eq) and the mixture heated at 60° C. for 2 h. A further portion of 1-iodo-2-methylpropane (237 μL, 2.1 mmol, 0.4 eq) was added and heating continued for a further 3 h. The reaction was then cooled to rt and the volatiles removed in vacuo. The residue was diluted with water (50 mL) and EtOAc (25 mL), the phases separated and the aqueous phase extracted with EtOAc (2×25 mL). Combined organics were washed with brine (50 mL), dried (Na₂SO₄) and the solvent removed in vacuo. The residue was purified by normal phase chromatography, 0-20% EtOAc/DCM to afford the title compound (687 mg, 53%) as a yellow oil. UPLC (Method A) 2.74 min, 99%, [M+H]⁺=251.0.

### Intermediate 80

### 2-(3-Ethyl-2-hydroxy-tetrahydrofuran-2-yl)acetonitrile

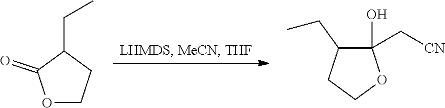

A solution of LHMDS (1M in THF, 17.3 mL, 17.3 mmol) in anhydrous THF (20 mL) was cooled to −78° C. and MeCN (821 μL, 15.8 mmol) was added dropwise. The resulting solution was stirred for 30 min before adding dropwise a solution of 3-ethyltetrahydrofuran-2-one (900 mg, 7.9 mmol) in dry THF (5.0 mL) and the reaction stirred at −78° C. for 5 h. The reaction was transferred via cannula to a round bottom flask containing MeCOH (100 mL) and the resulting solution carefully neutralised to pH 8 using 1 M HCl. The mixture was extracted with DCM (3×100 mL) and combined organics were washed with brine (200 mL), dried over Na₂SO₄ and concentrated in vacuo to yield the crude product as a pale brown oil. The crude residue was purified by column chromatography (Biotage Isolera, normal phase, 25 g, SiliaSep silica gel 40-63 μm/230-400 mesh, 60 Å, residue loaded in DCM, 0 to 40% EtOAc in Heptane, product not UV active) to yield 2-(3-ethyl-2-hydroxy-tetrahydrofuran-2-yl)acetonitrile (702 mg, 57%) as a pale yellow oil, which was used without further purification.

### Intermediate 81

### 3-(3-Amino-1H-pyrazol-5-yl)pentan-1-ol

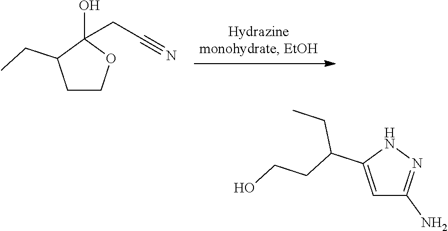

To a solution of Intermediate 80 (600 mg, 3.87 mmol) in EtOH (19 mL) was added NH₂NH₂.H₂O (282 μL, 5.80 mmol) and the resulting solution stirred at 60° C. for 20 h. The reaction was cooled to τt and CO₂ (dry ice) was bubbled through for 1 h. The reaction mixture was decanted and the filtrate was concentrated to a crude yellow oil (575 mg). The material was purified by column chromatography (Biotage Isolera, normal phase, 40 g, SiliaSep silica gel 40-63 μm, 230-400 mesh, 60 Å, [residue loaded in DCM (0.5 mL)]0-10% MeCOH in DCM to afford 3-(3-amino-1H-pyrazol-5-yl)pentan-1-ol (260 mg, 33%) as a pale pink gum. UPLC (Method A) 1.56 min, 84%, [M−H]⁻=168.1.

### Intermediate 82

### 4-Ethyl-5,6-dihydro-4H-pyrrolo[1,2-b]pyrazol-2-amine

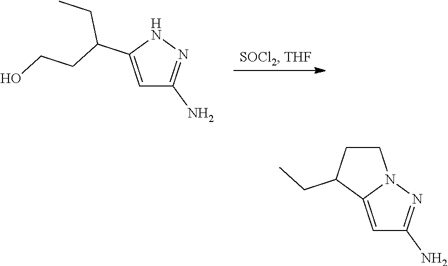

To a solution of Intermediate 81 (260 mg, 1.54 mmol) in THF (7.7 mL) was added SOCl₂ (0.56 mL, 7.68 mmol) dropwise and the reaction stirred under N₂ at rt. After 4 h a further portion of SOCl₂ (0.56 mL, 7.68 mmol) was added to the reaction with further stirring at rt for 1 h. The reaction was then concentrated in vacuo and the residue dissolved in DCM (20 mL) and heated at reflux for 2 h. The reaction was cooled to rt, SOCl₂ (0.56 mL, 7.68 mmol) was added and the mixture stirred at rt for 2 h before the solvent was removed in vacuo. The crude material was taken on directly to the next step.

### Intermediate 86

### 4-Methyl-N-(6-methyl-2-pyridyl)benzenesulfonamide

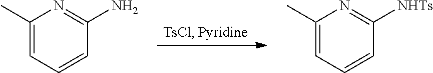

To a solution of 6-methylpyridin-2-amine (1.00 g, 9.3 mmol, 1.0 eq) in pyridine (5 mL) was added p-toluenesulfonyl chloride (2.29 g, 12.2 mmol, 1.3 eq) and the reaction stirred at 80° C. for 4 h, then rt overnight. The reaction was quenched with H₂O (25 mL), diluted with DCM (20 mL), the phases were separated and the aqueous extracted with DCM (3×10 mL). The combined organics were extracted with phosphate buffer (pH 7, 3×10 mL), dried (Na₂SO₄) and concentrated in vacuo. The crude material was purified by normal phase chromatography eluting with 2-10% EtOAc/heptane to afford the title compound (1.60 g, 66%) as a white sticky oil. UPLC (Method A): 2.17 min, 100%, [M+H]⁺=263.1.

### Intermediate 87

### 2-[(6E)-2-Methyl-6-(p-tolylsulfonylimino)-1-pyridyl]acetamide

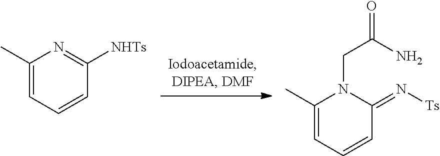

To a solution of Intermediate 86 (867 mg, 3.31 mmol, 1.0 eq) in DMF (2 mL) was added N,N-diisopropylethylamine (0.63 mL, 3.64 mmol, 1.1 eq) and the reaction stirred at rt for 15 min before addition of a solution of iodoacetamide (672 mg, 3.64 mmol, 1.1 eq) in DMF (1.5 mL) and the reaction stirred at rt overnight. The reaction was concentrated in vacuo, the oil was suspended in water (8 mL) and the solids were collected by filtration and triturated from tert-butyl methyl ether (assisted by sonication, ×3) to afford the title compound (668 mg, 63%) as a brown solid. UPLC (Method A): 2.18 min, 88%, [M+H]⁺=320.2.

### Intermediate 88

### 2,2,2-Trifluoro-N-(5-methylimidazo[1,2-a]pyridin-2-yl)acetamide

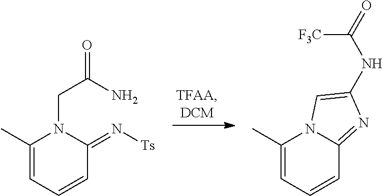 [SMILES: Cc1ccc(S(=O)(=O)/N=c2\cccc(C)n2CC(N)=O)cc1 ; Cc1cccc2nc(NC(=O)C(F)(F)F)cn12]

To a suspension of Intermediate 87 (0.67 g, 2.1 mmol, 1.0 eq) in DCM (2 mL) was added trifluoroacetic anhydride (1.37 mL, 9.9 mmol, 4.7 eq) and the reaction stirred at rt overnight. The reaction was concentrated in vacuo, dissolved in DCM (10 mL) and washed with sat. aq. NaHCO₃ (3×10 mL) and phosphate buffer (pH 7, 3×10 mL), the organics were dried (Na₂SO₄) and concentrated in vacuo.

The crude material was purified by normal phase chromatography 3-10% acetone/DCM to afford the title compound (315 mg, 62%) as a pale yellow solid. UPLC (Method A): 1.43 min, 99%, [M+H]⁺=244.

### Intermediate 89

### 2,2,2-Trifluoro-N-(5-methyl-5,6,7,8-tetrahydroimidazo[1,2-a]pyridin-2-yl)acetamide

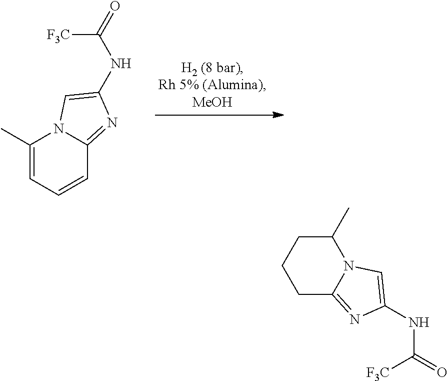 [SMILES: Cc1cccc2nc(NC(=O)C(F)(F)F)cn12 ; CC1CCCc2nc(NC(=O)C(F)(F)F)cn21]

A solution of Intermediate 88 (200 mg, 0.82 mmol, 1 eq) in MeOH (8 mL) was prepared in an autoclave. R^{h} (5% on alumina, 1.69 g, 0.82 mmol, 1 eq) was added. The autoclave was purged and refilled with N₂ (×3) and subsequently purged and loaded with H₂ (8 atm). The reaction was stirred at rt for 18 h, concentrated in vacuo and the crude material purified by normal phase chromatography (10% acetone/DCM) to afford the title compound (136 mg, 55%) as a white solid. UPLC (Method A): 2.28 min, 97%, [M+H]⁺=248.2.

### Intermediate 90

### 5-Methyl-5,6,7,8-tetrahydroimidazo[1,2-a]pyridin-2-amine

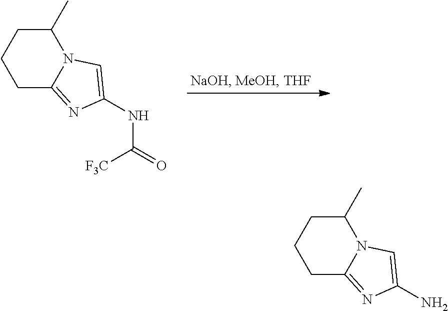 [SMILES: CC1CCCc2nc(NC(=O)C(F)(F)F)cn21 ; CC1CCCc2nc(N)cn21]

A suspension of Intermediate 89 (136 mg, 0.45 mmol, 1 eq) and NaOH (180 mg, 4.49 mmol, 10 eq) in THF (0.50 mL) and MeCOH (0.50 mL) was heated by microwave irradiation at 80° C. for 1 h. The reaction was concentrated in vacuo and H₂O (1 mL) was added. The pH was adjusted to pH 7 by dropwise addition of 2 M HCl and the resulting solution was freeze dried overnight and used directly in the next step, assumed quantitative. UPLC (Method A): 1.84 min, 85%, [M+H]⁺=152.1.

### Intermediate 94

### 2-(3-Methyl-5-oxo-morpholin-4-yl)acetamide

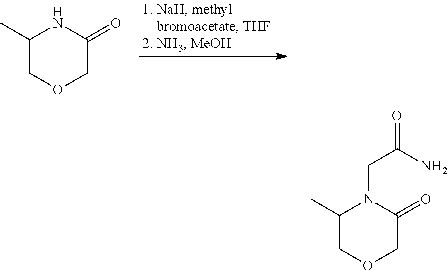

To a suspension of sodium hydride (60% in mineral oil, 267.5 mg, 6.7 mmol, 1.1 eq) in THF (4 mL), under N₂, at ˜15° C. (chilled water bath), was added a solution of 5-methylmorpholin-3-one (700.0 mg, 6.1 mmol, 1.0 eq) in THF (4 mL) dropwise and the reaction mixture stirred at 15° C. for 30 min. To the solution was then added methyl bromoacetate (640 μL, 6.7 mmol, 1.1 eq) dropwise and the reaction further stirred at room temperature for 1 h. The reaction was quenched with sat. NH₄Cl (60 mL), diluted with EtOAc (30 mL), the phases separated and the aqueous extracted with EtOAc (2×30 mL). The combined organics were dried (Na₂SO₄) and concentrated in vacuo. The crude residue was purified by normal phase chromatography, EtOAc/DCM 1:1 to afford methyl 2-(3-methyl-5-oxo-morpholin-4-yl)acetate (858 mg, 75%) as a pale-yellow oil. 1H NMR (CDCl₃, 400 MHz) δ_{H} 4.49 (m, 1H), 4.23 (m, 2H), 3.94 (m, 1H), 3.84-3.75 (m, 4H), 3.69 (m, 1H), 3.59-3.51 (m, 1H), 1.29 (m, 3H) ppm.

A solution of methyl 2-(3-methyl-5-oxo-morpholin-4-yl)acetate (429.0 mg, 2.3 mmol, 1.0 eq) in NH₃ (7M in MeCOH, 5 mL, 15.0 eq) was heated to 60° C. in a sealed vial for 63 h. The reaction mixture was cooled to rt and concentrated in vacuo. The residue was triturated with DCM/tert-butyl methyl ether (ca. 10 mL, 3:7) to afford the title compound (270 mg, 34%) as a milky gummy residue. 1H NMR (400 MHz, DMSO-d6) δ_{H} ppm, 7.33 (br. s, 1H), 7.05 (br. s, 1H), 4.12 (d, J=16.3 Hz, 1H), 4.04 (d, J=1.2 Hz, 2H), 3.85 (dd, J=11.5, 3.6 Hz, 1H), 3.66-3.58 (m, 2H), 3.53-3.47 (m, 1H), 1.16 (d, J=6.7 Hz, 3H) ppm.

### Intermediate 95

### 2-Bromo-5-methyl-6,8-dihydro-5H-imidazo[2,1-c][1,4]oxazine

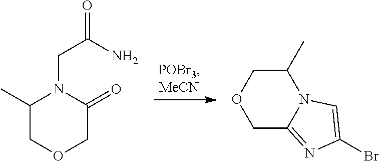

To a suspension of Intermediate 94 (270.0 mg, 1.57 mmol, 1 eq) in MeCN (1 mL) was added phosphorus(V)oxybromide (2.25 g, 7.84 mmol, 5 eq) and the vial sealed and heated at 90° C. for 3 h. The reaction was cooled to rt, poured into H₂O (40 mL), diluted with DCM (40 mL) and the phases were separated. The aqueous phase was extracted with DCM (30 mL), neutralised with solid K₂CO₃ (to pH 9) and further extracted with DCM (30 mL). The combined organics were diluted with water (40 mL) and basified with solid K₂CO₃ (to pH 9). The phases were separated, the organic phase dried (Na₂SO₄) and the solvent removed in vacuo. The crude residue was purified by normal phase chromatography (dry loaded) 0-100% EtOAc/DCM followed by 0-15% MeCOH/DCM and the resulting residue was triturated with acetone (2 mL) to afford the title compound (100 mg, 29%) as an orange solid. UPLC (Method E) 2.02 min, 100%, [M+H]⁺=217.0/219.0.

### Intermediate 99

### Tert-butyl N-(4-oxo-6,7-dihydro-5H-pyrazolo[1,5-a]pyridin-2-yl)carbamate

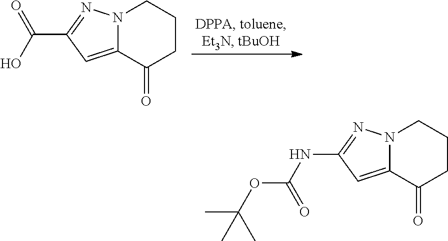 [SMILES: O=C(O)c1cc2n(n1)CCCC2=O ; CC(C)(C)OC(=O)Nc1cc2n(n1)CCCC2=O]

To a suspension of Intermediate 98 (1.54 g, 8.5 mmol, 1.0 eq) in dry toluene (20 mL) was added triethylamine (1.43 mL, 10.2 mmol, 1.2 eq) followed by anhydrous t-BuOH (10.0 mL, 104.0 mmol, 12.0 eq). To the solution was added diphenyl phosphoryl azide (2.21 mL, 10.2 mmol, 1.2 eq) and the reaction stirred at 80° C. overnight. The reaction was cooled to rt, diluted with EtOAc (45 mL) and H₂O (45 mL), the phases separated and the aqueous extracted with EtOAc (25 mL). The combined organics were washed with sat. aq. NaHCO₃ (30 mL), H₂O (30 mL), dried (Na₂SO₄) and concentrated in vacuo. The residue was purified through a short pad of silica 0-10% EtOAc/DCM to afford the title compound (1.17 g, 54%) as a white solid. UPLC (Method A) 2.68 min, 88%, [M−H]⁻=250.2.

### Intermediate 100

### 2-Amino-6,7-dihydro-5H-pyrazolo[1,5-a]pyridin-4-one

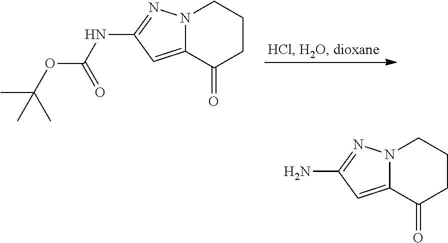 [SMILES: CC(C)(C)OC(=O)Nc1cc2n(n1)CCCC2=O ; Nc1cc2n(n1)CCCC2=O]

To a solution of Intermediate 99 (190 mg, 0.76 mmol, 1 eq) in dioxane (4 mL) was added HCl (4 mL, 4 M in dioxane) followed by one drop of water from a glass pipette and the reaction stirred at rt overnight. The solvent was removed in vacuo and the solid was suspended in DCM (10 mL). Sat. aq. NaHCO₃ (10 mL) was added and stirred until a solution formed, the phases were separated and the aqueous extracted with DCM (2×5 mL). The combined organics were dried (phase separator) and concentrated in vacuo to afford the title compound (81 mg, 71%) as a yellow oil. UPLC (Method A) 0.80 min, 56%, [M+H]⁺=152.1.

### Intermediate 101

### Methyl 2-(2-methyl-5-oxo-pyrrolidin-1-yl)acetate

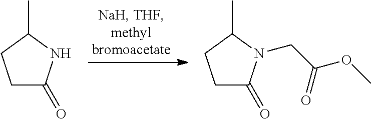

To an ice-cold solution of anhydrous THF (60 mL) was added sodium hydride (60% in mineral oil, 2.22 g, 55.4 mmol, 1.1 eq) portionwise. To the grey slurry was added a solution of 5-methylpyrrolidin-2-one (5.00 g, 50.4 mmol, 1.0 eq) in dry THF (20 mL) dropwise over 10 min, keeping the temperature below 7° C. After a further 5 min, additional THF (40 mL) was added and the reaction stirred for a further 45 min before addition of methyl bromoacetate (5.7 mL, 60.5 mmol, 1.2 eq) in THF (10 mL). The reaction was allowed to warm to rt and stirred for 1.5 h. The reaction was poured into NH₄Cl aq. sat. solution (100 mL), extracted with EtOAc (3×80 mL), washed with water (2×100 mL), aq. NH₄Cl (2×100 mL), dried over Na₂SO₄, filtered and concentrated under reduced pressure to afford the title compound (4.81 g, 56%) as a colourless oil. UPLC (Method A): 1.86 min, 66%, [M+H]⁺=172.1.

### Intermediate 102

### 2-(2-Methyl-5-oxo-pyrrolidin-1-yl)acetamide

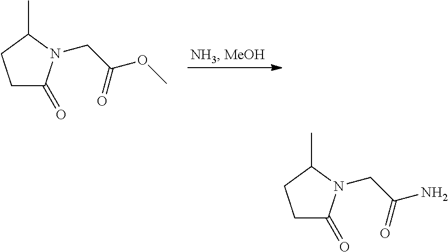

A solution of Intermediate 101 (3.80 g, 22.2 mmol, 1 eq) in ammonia (7 M in methanol, 40 mL, 13 eq) was stirred at rt for 48 h. The reaction mixture was concentrated in vacuo and re-dissolved in MeCOH (40 mL) and MeCN (10 mL) and washed with heptane. The MeCOH/MeCN layer was concentrated in vacuo and the residue was triturated with MeCN (30 mL) and stored at 5° C. for 3-4 h. The solvent was decanted from the insoluble impurities and evaporated under reduced pressure to afford the title compound (2.16 g, 62%) as a colourless oil. ¹H NMR (DMSO-d6, 396 MHz): δ 7.29 (br s, 1H), 7.01 (br s, 1H) 3.88 (m, 1H), 3.63 (m, 1H), 3.51 (m, 1H), 2.08-2.21 (m, 3H), 1.47 (m, 1H), 1.07 (d, J=6.1 Hz, 3H) ppm.

### Intermediate 103

### 2-Bromo-5-methyl-6,7-dihydro-5H-pyrrolo[1,2-a]imidazole

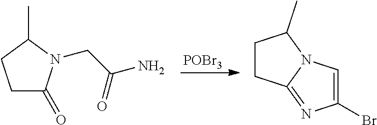

Intermediate 102 (2.00 g, 12.8 mmol, 1 eq) and POBr₃ (7.34 g, 25.6 mmol, 2 eq) were heated at 80° C. in a sealed vial for 3 h, and the reaction was then cooled to rt overnight. The mixture was taken up in DCM/H₂O (3×10 mL), the phases separated, the aqueous basified with K₂CO₃ (3-4 g) and extracted with DCM (2×30 mL). Combined organics were washed with aq. K₂CO₃ solution (20 mL) and H₂O (20 mL), dried (Na₂SO₄) and concentrated in vacuo. The residue was purified through a short pad of silica (0-50% DCM/EtOAc) to afford the title compound (1.15 g, 45%) as an amber oil, which crystallized on standing. UPLC (Method A): 2.42 min, 96%, [M+H]⁺=201.1/203.1.

### Intermediates 104A and 104B

### 1-(2-Fluoroethyl)-5-iodo-imidazole (Intermediate 104A) & 1-(2-fluoroethyl)-4-iodo-imidazole (Intermediate 104B)

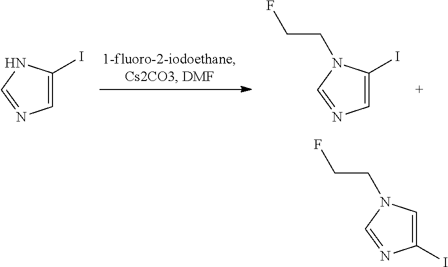

To a solution of 4-iodo-1H-imidazole (0.93 g, 4.77 mmol, 1.0 eq) in DMF (9.5 mL) was added caesium carbonate (4.66 g, 14.31 mmol, 3.0 eq) and 1-fluoro-2-iodo-ethane (1.00 g, 5.72 mmol, 1.2 eq). The reaction was stirred at rt for 3 h, concentrated in vacuo and the residue was dissolved in EtOAc (100 mL). The organic layer was washed with water (3×50 mL), brine (50 mL), dried (MgSO₄) and concentrated in vacuo. The residue was purified by normal phase chromatography, 20% EtOAc/DCM to afford an inseparable mixture of isomers (749 mg, 65%) as a colourless oil. UPLC (Method A): 2.03 min, 77%, [M+H]⁺=240.9; UPLC (Method A): 2.07 min, 22%, no mass ion observed.

### Intermediate 105

### 6-(Trifluoromethyl)piperidin-2-one

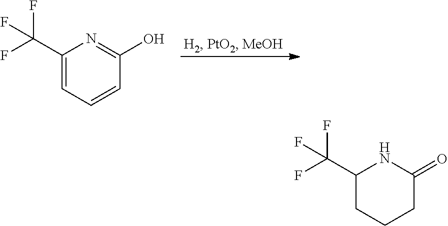 [SMILES: Oc1cccc(C(F)(F)F)n1 ; O=C1CCCC(C(F)(F)F)N1]

The starting pyridone (1.50 g, 9.20 mmol, 1 eq) was dissolved in dry MeCOH (90 mL) and placed in a bomb. PtO₂ (300 mg, 20% wt) was added. The reaction vessel was sealed and the atmosphere was purged with H₂ (3×). The reaction was stirred at rt for 16 h under 10 bar of H₂. The reaction mixture was filtered through Dicalite and washed thoroughly with MeCOH (about 200 mL). The filtrate was concentrated to dryness to yield the title compound (1.49 g, 97%) as a colourless solid. 1H NMR (DMSO, 400 MHz) δ_{H} 8.01 (bs, 1H), 4.02-4.09 (m, 1H), 2.13-2.21 (m, 2H), 1.85-1.94 (m, 1H), 1.57-1.78 (m, 3H) ppm. 19F NMR (DMSO, 376 MHz) δ_{F} −75.34 (d, J=8.65 Hz) ppm.

### Intermediate 106

### Methyl 2-[2-oxo-6-(trifluoromethyl)-1-piperidyl]acetate

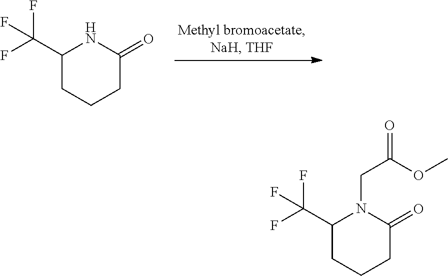 [SMILES: O=C1CCCC(C(F)(F)F)N1 ; COC(=O)CN1C(=O)CCCC1C(F)(F)F]

To a mixture of pyridone and piperidone of Intermediate 105 (ca 67% piperidone) (2.40 g, 14.4 mmol, 1.0 eq) in dry THF (50 mL) was added methyl bromoacetate (1.63 mL, 17.2 mmol, 1.2 eq) under N₂. The mixture was cooled to 0° C. in an ice-bath and NaH (60% in mineral oil, 632 mg, 15.8 mmol, 1.1 eq) was added portionwise. The reaction was stirred for 30 min at 0° C. and 1.5 h at rt. The reaction was carefully poured into sat. aq. NH₄Cl (100 mL) and extracted with EtOAc (3×75 mL). The combined organic phases were dried over Na₂SO₄, filtered and concentrated to dryness to yield a yellow oil. The residue was purified by NP chromatography (Hept/EtOAc 10-75%) to yield the title compound (2.21 g, 64%) as a colourless oil. 1H NMR (CDCl₃, 400 MHz) δ_{H} 4.79 (d, J=17.6 Hz, 1H), 4.00-3.92 (m, 1H), 3.71-3.75 (m, 4H), 2.54-2.49 (m, 2H), 2.17-1.99 (m, 3H), 1.88-1.82 (m, 1H) ppm. 19F NMR (CDCl3, 376 MHz) δ_{F} 78.2 (d, J=6.7 Hz) ppm.

### Intermediate 107

### 2-[2-Oxo-6-(trifluoromethyl)-1-piperidyl]acetamide

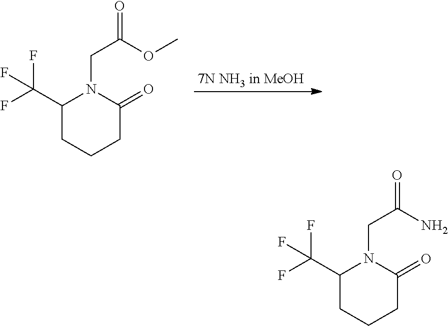 [SMILES: COC(=O)CN1C(=O)CCCC1C(F)(F)F ; NC(=O)CN1C(=O)CCCC1C(F)(F)F]

Intermediate 106 (2.21 g, 9.23 mmol, 1 eq) was solubilized in 7 N methanolic ammonia (30 mL) and the solution divided into 2×15 mL batches, which were each heated in a sealed tube to 60° C. for 24 h in a MW. The two vials were allowed to cool to rt, combined and concentrated to dryness to yield the title compound (1.91 g, 92%) as an off-white solid. 1H NMR (DMSO, 400 MHz) δ_{H} 7.28 (bs, 1H), 6.98 (bs, 1H), 4.34 (d, J=16.3 Hz, 1H), 4.23-4.29 (m, 1H), 3.49 (d, J=16.3 Hz, 1H), 2.29-2.33 (m, 2H), 1.91-2.09 (m, 2H), 1.71-1.83 (m, 2H) ppm. 19F NMR (DMSO, 376 MHz) δ_{F} −70.50 (d, J=7.8 Hz) ppm.

### Intermediate 108

### 2-Bromo-5-(trifluoromethyl)-5,6,7,8-tetrahydroimidazo[1,2-a]pyridine

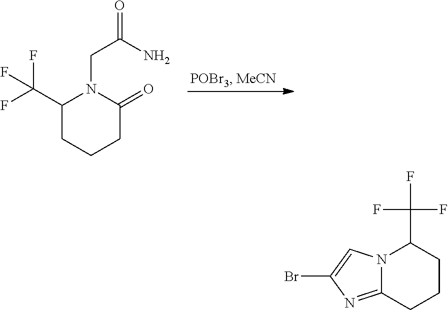 [SMILES: NC(=O)CN1C(=O)CCCC1C(F)(F)F ; FC(F)(F)C1CCCc2nc(Br)cn21]

Intermediate 107 (0.30 g, 1.34 mmol) and POBr₃ (1.53 g, 5.35 mmol) were dissolved in MeCN (3.0 mL) and heated using a Biotage Initiator microwave reactor at 120° C. for 60 min. The mixture was quenched with 10% K₂CO₃ solution (20 mL) and extracted with DCM:IPA (90:10, 2×30 mL), washed with saturated brine solution (20 mL), dried (MgSO₄), concentrated in vacuo and azeotroped with TBME/heptane to give 2-bromo-5-(trifluoromethyl)-5,6,7,8-tetrahydroimidazo[1,2-a]pyridine (279 mg, 78%) as a brown solid, which was used without further purification. UPLC (Method A) 2.70 min, 31%, [M+H]⁺=269.0, 271.0.

### Intermediate 109

### 1-Tert-butyl-2,5-diiodo-imidazole and 1-tert-butyl-2,4-diiodo-imidazole

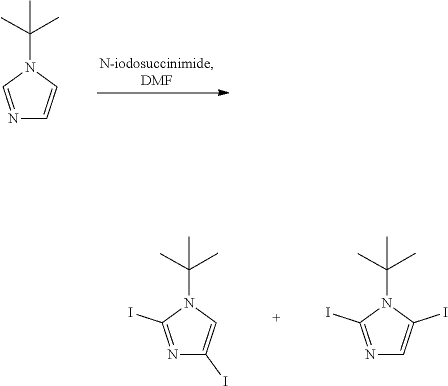

To a stirred solution of 1-tert-butylimidazole (1.00 g, 8.05 mmol) in DMF (20 mL) was added NIS (3.80 g, 16.9 mmol). The reaction was stirred for 24 h at 70° C. The reaction was allowed to cool to rt, diluted with water (40 mL) and extracted with DCM (3×40 mL). The combined organics were washed with Na₂S2O₃ aq. sat. (40 mL), H2O (40 mL) and brine (3×40 mL), dried (Na₂SO₄), filtered and concentrated to dryness. The residue was purified by NP chromatography (acetone/tol 0-10%) to lead to an inseparable mixture of 1-tert-butyl-2,4-diiodo-imidazole and 1-tert-butyl-2,5-diiodo-imidazole (363 mg, 10%, 4:1 ratio). UPLC (Method A) 3.07 min (18%), 3.12 min (67%), [M+H]⁺=376.9.

### Intermediate 110

### 1-Tert-butyl-4-iodo-imidazole and 1-tert-butyl-5-iodo-imidazole

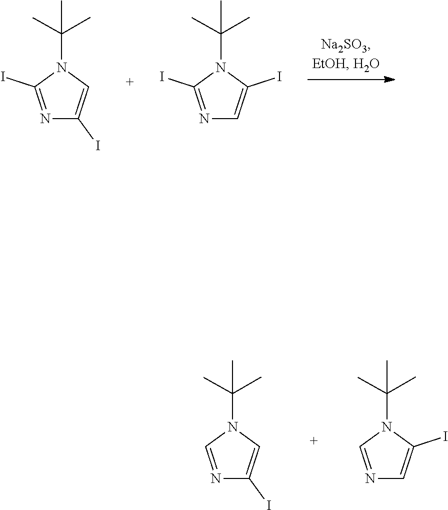

To a stirred solution of the compounds of Intermediate 109 (363 mg, 0.97 mmol) in EtOH (20 mL) was added a solution of Na₂SO₃ (608 mg, 4.83 mmol) in H₂O (20 mL). The reaction was stirred for 30 min at 60° C. The volatiles were removed under reduced pressure and the aqueous was extracted with EtOAc (3×15 mL). The combined organic phases were washed with brine (15 mL), dried (Na₂SO₄), filtered and concentrated to dryness to yield a mixture of 1-tert-butyl-4-iodo-imidazole and 1-tert-butyl-5-iodo-imidazole as an inseparable mixture (278 mg, 59%, 4:1 ratio). LCMS (Method D) 2.54 min, 52%, [M+H]⁺=250.9.

### Intermediate 111

### 3-Bromo-4,4-difluoro-6,7-dihydro-5H-pyrazolo[1,5-a]pyridin-2-amine

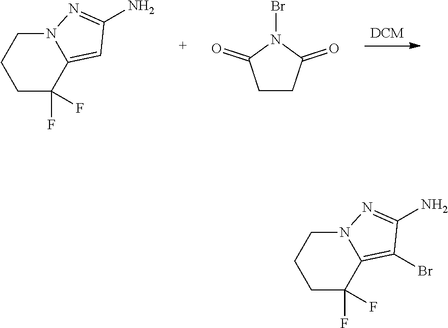 [SMILES: Nc1cc2n(n1)CCCC2(F)F ; O=C1CCC(=O)N1Br ; Nc1nn2c(c1Br)C(F)(F)CCC2]

To a solution of Intermediate 37 (205 mg, 1.18 mmol) in DCM (10 mL) was added N-bromosuccinimide (232 mg, 1.30 mmol) and the mixture stirred at rt for 2.5 h. Sodium thiosulfate solution (20% w/w, 20 mL) was then added and the layers were separated. The aqueous layer was further extracted with DCM (20 mL) and combined organics were washed with aq. NaOH solution (1 M, 20 mL). The organic layer was dried over MgSO₄ before concentration to give the product as a yellow gum. The residue was adsorbed onto silica and purified by column chromatography, manual column, normal phase, silica gel 40-63 μm/230-400 mesh, 60 Å, eluting with 1:1 EtOAc-heptane and 1% Et₃N. The product fractions were combined, redissolved in CH₃CN/H₂O and freeze dried overnight. 3-Bromo-4,4-difluoro-6,7-dihydro-5H-pyrazolo[1,5-a]pyridin-2-amine (248 mg, 78%) was isolated as a pale yellow solid. UPLC (Method F) 2.45 min and 2.63 min, 66.6% and 25.5%, ES+: 252.0/254.0 [M(79Br/81Br)+H]+.

### Intermediate 112

### 4,4-Difluoro-3-methyl-6,7-dihydro-5H-pyrazolo[1,5-a]pyridin-2-amine

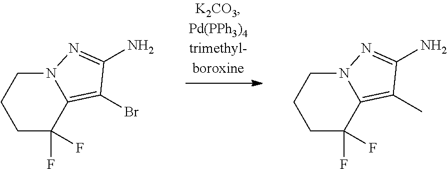

To trimethylboroxine (293 mg, 2.33 mmol) and Intermediate 111 (195 mg, 0.77 mmol) in dry 1,4-dioxane (6.0 mL) was added potassium carbonate (250 mg, 1.81 mmol) and Pd(PPh₃)₄ (90 mg, 0.08 mmol) and the mixture degassed (vacuum/nitrogen purge×3) and heated at 90° C. overnight. The reaction mixture was cooled to rt., diluted with EtOAc (15 mL) and filtered to remove inorganics. The solvent was removed in vacuo to yield crude residue.

A second reaction was set up in parallel. To trimethylboroxine (75 mg, 0.60 mmol) and Intermediate 111 (50 mg, 0.20 mmol) in dry (previously degassed) 1,4-dioxane (2.5 mL) was added potassium carbonate (55 mg, 0.40 mmol) and Pd(PPh₃)₄ (23 mg, 0.02 mmol) and the mixture heated at 90° C. overnight. Additional Pd(PPh₃)₄ (23 mg, 0.02 mmol), trimethylboroxine (75 mg, 0.60 mmol) and 1,4-dioxane (1.0 mL) were added and the reaction continued at 90° C. The reaction was cooled and filtered and washed with EtOAc. The solvent was evaporated, and the residue dissolved in methanol (3.0 mL). The mixture was purified using an SCX-2 (2 g) cartridge eluting with methanol (3CV) followed by 4.5 M NH₃ in MeCOH (3CV) to elute the product. The solvent was evaporated and the resulting residue (20 mg) was shown by UPLC to contain the desired product as a mixture with starting material and the des-methyl impurity.

The crude residues from the two reactions were combined for purification. The combined product was adsorbed onto silica and purified by column chromatography (manual column, normal phase, silica gel 40-63 μm/230-400 mesh, 60 Å), 0% to 3% MeCOH in DCM. The mixture obtained (214 mg) was passed through an SCX-2 (5 g) cartridge eluting with methanol (3 CV) to remove OPPh₃ then eluted with 4.5 M NH₃ in MeCOH (3 CV) to elute the product as a mixture with starting material to afford a yellow oil (130 mg). The yellow oil was dissolved in DCM (1.0 mL) and purified by column chromatography (manual column, normal phase, silica gel 40-63 μm/230-400 mesh, 60 Å) 0% to 2% MeCOH in DCM. Fractions were combined to yield two batches of crude product. The isolated material A (60 mg) contained the desired product (57%), OPPh₃ (27%) and the des-methyl by product (9%) by UPLC. The isolated material B (76 mg) contained product (40%), OPPh₃ (16%) and bromo-starting material (25%). Both the impure batches were taken directly to the next step. UPLC (Method E) 2.13 min, 57%, ES⁺: 188.1 [M+H]⁺.

### Intermediate 113

### 4,4-Difluoro-3-iodo-6,7-dihydro-5H-pyrazolo[1,5-a]pyridin-2-amine

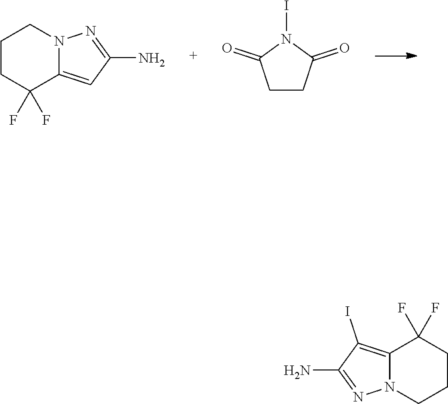 [SMILES: Nc1cc2n(n1)CCCC2(F)F ; O=C1CCC(=O)N1I ; Nc1nn2c(c1I)C(F)(F)CCC2]

To a solution of Intermediate 37 (317 mg, 1.83 mmol) in DCM (15 mL) was added N-iodosuccinimide (618 mg, 2.75 mmol) and the reaction stirred at rt for 72 h under nitrogen. The volatiles were evaporated in vacuo and the residue was purified via column chromatography (manual column, normal phase, silica gel 40-63 μm/230-400 mesh, 60 Å, 4% acetone in DCM with 1% of triethylamine). The fractions containing the product were concentrated and the residue was further purified via column chromatography (manual column, normal phase, silica gel 40-63 μm/230-400 mesh, 60 Å, 2% acetone in DCM with 1% of triethylamine) to provide 4,4-difluoro-3-iodo-6,7-dihydro-5H-pyrazolo[1,5-a]pyridin-2-amine (113 mg, 15%) as a yellow solid. UPLC (Method A) 2.55 min, 72%, ES⁺: 300.0 [M+H]⁺.

### Intermediate 114

### 4,4-Difluoro-3-vinyl-6,7-dihydro-5H-pyrazolo[1,5-a]pyridin-2-amine

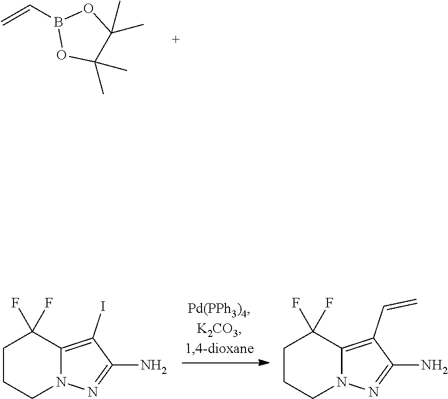

A suspension of Intermediate 113 (113 mg, 0.38 mmol), 2-vinylboronic acid pinacol ester (0.08 mL, 0.45 mmol), Pd(PPh₃)₄ (43.7 mg, 0.04 mmol), K₂CO₃ (157 mg, 1.13 mmol) in a degassed mixture of 1,4-dioxane (4.0 mL) and water (1.0 mL), was heated to 150° C. for 20 min in a sealed vial under MW irradiation. The reaction mixture was quenched with water (10 mL) and extracted with EtOAc (3×10 mL). The combined organics were washed with brine (15 mL), dried over Na₂SO₄ and concentrated in vacuo to yield the crude product. This was purified via column chromatography (manual column, normal phase, silica gel 40-63 μm/230-400 mesh, 60 Å, 10 to 50% EtOAc in heptane) to provide 4,4-difluoro-3-vinyl-6,7-dihydro-5H-pyrazolo[1,5-a]pyridin-2-amine (71.0 mg, 71% yield) as a pale yellow solid. UPLC (Method A) 2.51 min, 75%, ES⁺: 200.1 [M+H]⁺.

### Intermediate 115

### 3-Ethyl-4,4-difluoro-6,7-dihydro-5H-pyrazolo[1,5-a]pyridin-2-amine

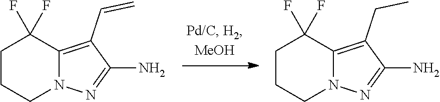

To a solution of Intermediate 114 (71.0 mg, 0.30 mmol) in MeCOH (5.0 mL) in a stainless steel vessel, was added Pd/C (10% purity, 32.2 mg, 0.03 mmol). The vessel was sealed and stirred under an atmosphere of hydrogen (at 4 bar) at rt overnight. The mixture was filtered through a pad of Dicalite, eluting with methanol. The solvent was removed in vacuo to provide 3-ethyl-4,4-difluoro-6,7-dihydro-5H-pyrazolo[1,5′-a]pyridin-2-amine (54.0 mg, 57%) as a white solid. UPLC (Method A) 2.52 min, 64%, ES⁺: 202.1 [M+H]⁺.

### Intermediate 116

### 1-[2-(4-Methylpiperazin-1-yl)ethyl]-5-(trifluoromethyl)pyrazol-3-amine

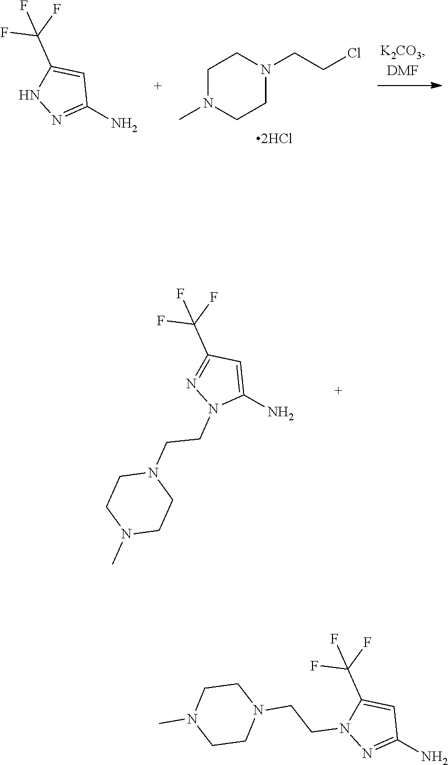

To a solution of 5-(trifluoromethyl)-1H-pyrazol-3-amine (380 mg, 2.52 mmol) in DMF (10 mL) was added potassium carbonate (1.11 g, 8.05 mmol) and the mixture stirred at room temperature for 0.25 h. To this was then added 1-(2-chloroethyl)-4-methyl-piperazine; dihydrochloride (711 mg, 3.02 mmol) and the reaction mixture stirred at rt for 72 h. The reaction was then heated at 55° C. for 24 h. The reaction was concentrated and partitioned between H₂O (40 mL) and TBME (2×40 mL). The combined organic layers were washed with water (2×10 mL), dried over MgSO₄ and concentrated to give an oily residue. The combined aqueous layers were extracted with DCM (3×20 mL) and these layers combined, dried over MgSO₄ and concentrated to give an oily residue. The original TBME extract was purified by column chromatography (manual column, normal phase, silica gel 40-63 μm/230-400 mesh, 60 Å, [residue dry loaded], 3% to 5% MeCOH in DCM containing 1% aqueous ammonia) to give the undesired regioisomer 2-[2-(4-methylpiperazin-1-yl)ethyl]-5-(trifluoromethyl)pyrazol-3-amine (122 mg, 15.9%) as a pale orange solid. The mixed product fractions were combined to give 93 mg of brown oily residue which was combined with the DCM extract from the aqueous layers described earlier. This residue was purified by column chromatography (manual column, normal phase, silica gel 40-63 μm/230-400 mesh, 60 Å, [residue dry loaded], 3% to 5% MeCOH in DCM containing 1% aqueous ammonia) to give some separation, but no clean 1-[2-(4-methylpiperazin-1-yl)ethyl]-5-(trifluoromethyl)pyrazol-3-amine fractions with the desired regioisomer. Mixed product fractions were concentrated to give 1-[2-(4-methylpiperazin-1-yl)ethyl]-5-(trifluoromethyl)pyrazol-3-amine (114 mg, 6.6%) as an off-white solid containing 2-[2-(4-methylpiperazin-1-yl)ethyl]-5-(trifluoromethyl)pyrazol-3-amine as a significant impurity. UPLC (Method A) 2.23 min, 40.3%, ES⁺: 278.1 [M+H]⁺.

### Intermediate 117

### 6-Aminopyridine-2-carbohydrazide

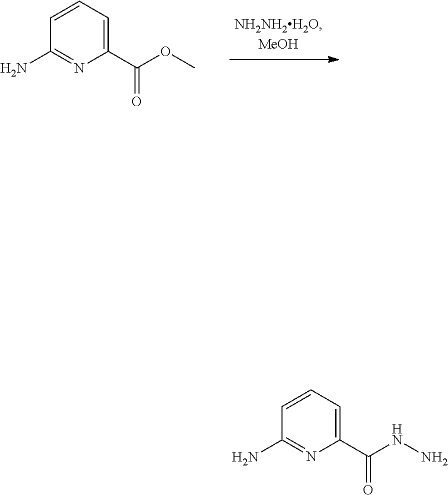

To a solution of methyl 6-aminopyridine-2-carboxylate (152 mg, 1.00 mmol) in MeCOH (2.0 mL) was added hydrazine monohydrate (0.15 mL, 3.00 mmol). The mixture was heated at 78° C. under nitrogen for 3 h. The mixture was cooled to rt and the solvent removed. The resulting solid was washed with a mixture of EtOAc and TBME (1:3, 15 mL) and then with TBME (10 mL). The resulting solid was air-dried to provide 6-aminopyridine-2-carbohydrazide (117 mg, 77%) as an off-white solid, which was used without further purification in the next step.

### Intermediate 118

### N,6-bis[(E)-dimethylaminomethyleneamino]pyridine-2-carboxamide

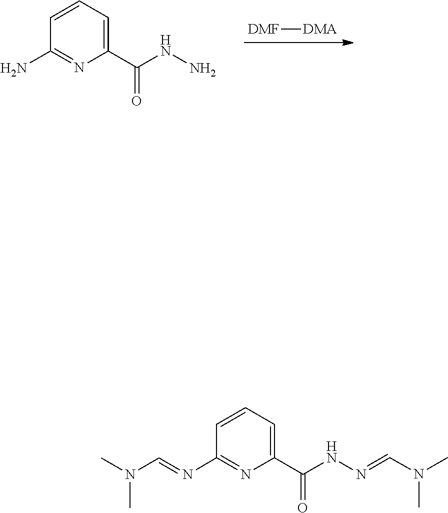

A stirred mixture of Intermediate 117 (117 mg, 0.77 mmol) and DMF-DMA (5.0 mL, 37.60 mmol) was heated at 110° C. for 24 h. The mixture was cooled to r.t. and the volatiles were removed in vacuo. The resulting solid was suspended in TBME. The mixture was put in an ultrasound bath for 5 min, the solvent was removed and the solid was dried to provide N,6-bis[(E)-dimethylaminomethyleneamino]pyridine-2-carboxamide (220 mg, 102%) which was used crude in the next step without further purification. UPLC (Method A) 1.97 min, 93.2%, ES⁺: 263.1 [M+H]⁺.

### Intermediate 119

### 6-(4-Isopropyl-1,2,4-triazol-3-yl)pyridin-2-amine

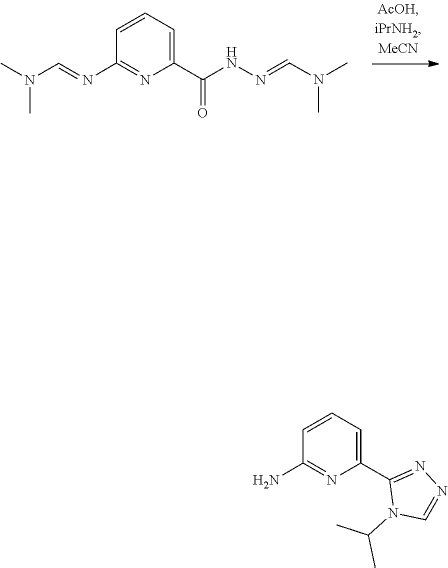

To a stirred solution of Intermediate 118 (220 mg, 0.78 mmol) in a mixture of MeCN (1.0 mL) and AcOH (0.25 mL, 4.44 mmol) was added isopropylamine (0.34 mL, 3.91 mmol). The mixture was stirred at 100° C. under nitrogen for 16 h. The reaction mixture was quenched with water and the pH adjusted to 8 with 2M NaOH aq. solution. The mixture was extracted with EtOAc (3×15 mL) and combined organics washed with brine (10 mL), dried over Na₂SO₄ and concentrated to provide 6-(4-isopropyl-1,2,4-triazol-3-yl)pyridin-2-amine (50 mg, 22%) as a yellow oil, which was taken on directly to the next step. UPLC (Method A) 1.86 min, 68.8%, ES⁺: 204.1 [M+H]⁺.

### Intermediate 120

### [3-[2-(3-Pyridyl)ethynyl]benzoyl]oxypotassium

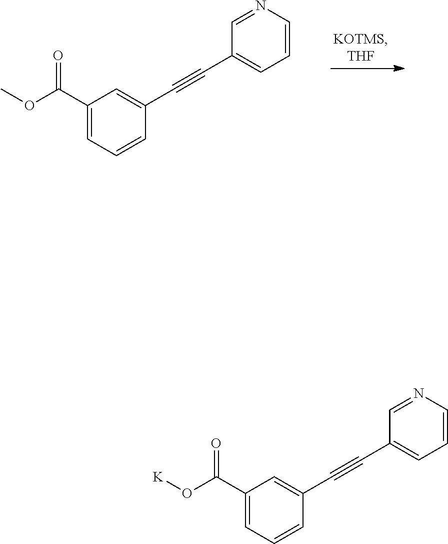

To a solution of methyl 3-[2-(3-pyridyl)ethynyl]benzoate (237 mg, 1.00 mmol) in THF (10 mL) was added KOTMS (385 mg, 3.00 mmol). The reaction was stirred at rt overnight under nitrogen. The volatiles were evaporated in vacuo. The resulting solid was suspended in TBME (20 mL) and put in an ultrasound bath for 5 min. The solvent was removed and the solid was dried to provide [3-[2-(3-pyridyl)ethynyl]benzoyl]oxypotassium (288 mg, 110%) as a light yellow solid, which was used in the next step without further purification. UPLC (Method A) 1.79 min, 100%, ES⁺: 224.1 [M-K+2H]⁺.

### Intermediate 121

### 3-[2-(3-Pyridyl)ethynyl]benzoyl chloride

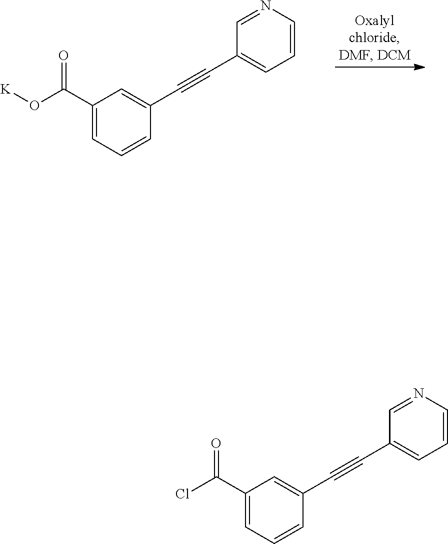

To a suspension of Intermediate 120 (288 mg, 0.99 mmol) in DCM (5.0 mL) was added oxalyl chloride (0.11 mL, 1.29 mmol) and few drops of DMF. The reaction was stirred overnight at rt under nitrogen. Another portion of oxalyl chloride (0.09 mL, 0.99 mmol) was added to the reaction mixture and the reaction was stirred at rt overnight under nitrogen. Another portion of oxalyl chloride (0.09 mL, 0.99 mmol) was added to the reaction mixture and the reaction was stirred at rt under nitrogen for 2 h. The volatiles were evaporated in vacuo. Toluene (10 mL) was added to the reaction mixture and the volatiles were evaporated in vacuo. The residue was dissolved in DCM (10 mL) and filtered. The solid was washed with DCM (10 mL) and the organic phases were combined and concentrated in vacuo to provide a mixture of 3-[2-(3-pyridyl)ethynyl]benzoyl chloride and 3-[2-(3-pyridyl)ethynyl]benzoic acid. To this mixture suspended in DCM (5.0 mL) was added oxalyl chloride (0.11 mL, 1.29 mmol). The reaction was stirred for 2 h at rt under nitrogen. The volatiles were evaporated in vacuo. Toluene (10 mL) was added to the mixture and the volatiles were evaporated in vacuo. The residue was dissolved in DCM (10 mL) and filtered. The solid was washed with DCM (10 mL) and the organic phases were combined and concentrated in vacuo to provide 3-[2-(3-pyridyl)ethynyl]benzoyl chloride (230 mg, 92%) as an orange oil, which was used crude in the next step. UPLC (Method A) 3.23 min, 95.8%, ES⁺: 238.1 [MH-Cl+OMe]⁺.

### Intermediate 122

### Methyl 4-fluoro-3-[2-(3-pyridyl)ethynyl]benzoate

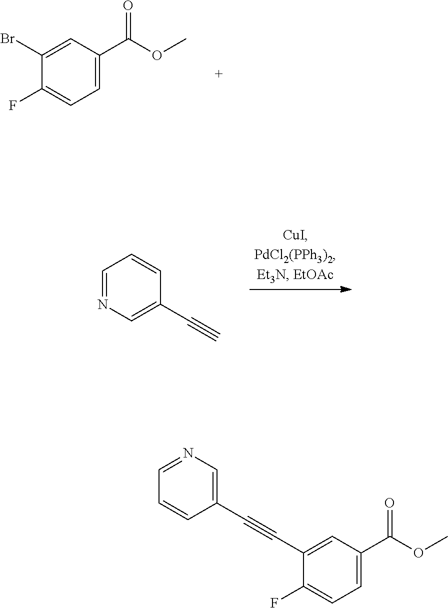

Methyl 3-bromo-4-fluoro-benzoate (1.00 g, 4.30 mmol), 3-ethynylpyridine (665 mg, 6.45 mmol), triethylamine (1.82 mL, 13.1 mmol) and EtOAc (20 mL) were added to a two-neck round bottom flask, degassed with N₂ for 15 min, then CuI (26.8 mg, 0.14 mmol) and Pd(PPh₃)₂Cl₂ (85.0 mg, 0.12 mmol) were added and the resulting mixture was stirred at rt under N₂ overnight. The reaction mixture was allowed to cool to rt, filtered through a celite pad and concentrated. The residue was purified by column chromatography (manual column, normal phase, silica gel 40-63 μm/230-400 mesh, 60 Å, [silica/crude=40/1, residue loaded in DCM], 0% to 20% EtOAc in heptane to give methyl 4-fluoro-3-[2-(3-pyridyl)ethynyl]benzoate (577 mg, 52%) as a pale yellow solid. UPLC (Method A) 3.30 min, 99.0%, ES⁺: 256.1 [M+H]⁺.

### Intermediate 123

### Methyl 4-pyrazol-1-yl-3-[2-(3-pyridyl)ethynyl]benzoate

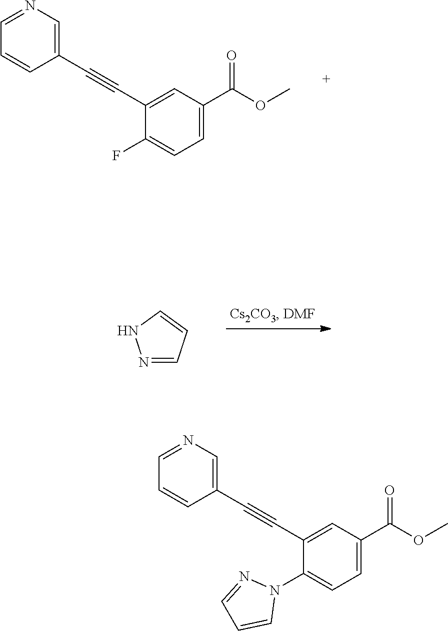 [SMILES: COC(=O)c1ccc(F)c(C#Cc2cccnc2)c1 ; c1cn[nH]c1 ; COC(=O)c1ccc(-n2cccn2)c(C#Cc2cccnc2)c1]

A RBF was charged with methyl Intermediate 122 (208 mg, 0.81 mmol), Cs₂CO₃ (797 mg, 2.44 mmol), pyrazole (56 mg, 0.81 mmol) and DMF (15 mL) and heated to 80° C. for 0.5 h under a N₂ atmosphere. The reaction mixture was allowed to cool to rt, the solvent partially removed, the residue diluted with water (15 mL) and EtOAc (20 mL) and the layers separated. The aqueous layer was extracted with EtOAc (3×30 mL), combined organics were dried over Na₂SO₄, filtered, and concentrated to give a beige solid. The residue was purified by column chromatography (manual column, normal phase, silica gel 40-63 μm/230-400 mesh, 60 Å, [silica/crude=20/1, residue loaded in DCM], 0% to 30% EtOAc in heptane to give methyl 4-pyrazol-1-yl-3-[2-(3-pyridyl)ethynyl]benzoate (177 mg, 65%) as a colourless solid. UPLC (Method A) 3.13 min, 91%, ES⁺: 304.1 [M+H]⁺.

### Intermediate 124

### 4-pyrazol-1-yl-3-[2-(3-pyridyl)ethynyl]benzoic acid

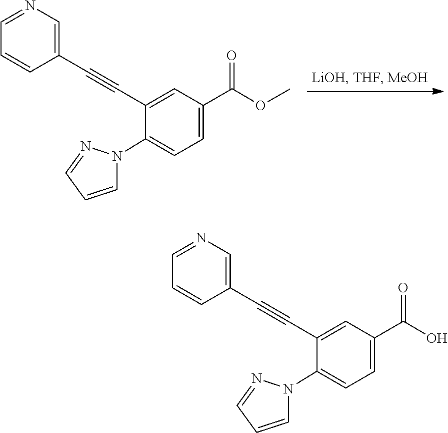 [SMILES: COC(=O)c1ccc(-n2cccn2)c(C#Cc2cccnc2)c1 ; O=C(O)c1ccc(-n2cccn2)c(C#Cc2cccnc2)c1]

To a solution of Intermediate 123 (177 mg, 0.58 mmol) in THF:MeOH:Water (2.0 mL:1.0 mL:1.0 mL) was added LiOH.H₂O (40 mg, 0.95 mmol) and the mixture stirred at rt, under a N₂ atmosphere, overnight. The reaction mixture was partially concentrated, water (5.0 mL) added and the solution acidified using 2N HCl to pH 4. The resultant precipitate was collected on a frit and washed with MTBE (20 mL), and dried to yield 4-pyrazol-1-yl-3-[2-(3-pyridyl)ethynyl]benzoic acid (149 mg, 87.8%). UPLC (Method A) 1.74 min, 99.5%, ES⁺: 290.1 [M+H]⁺.

### General Procedure A

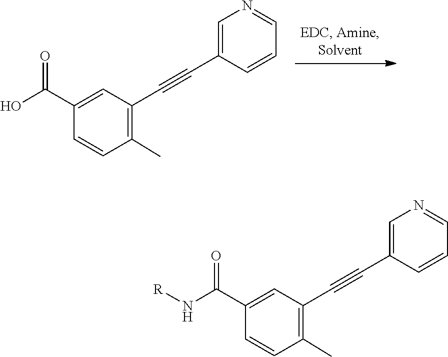

Intermediate 2 (1.1 mmol) and a solution of N-hydroxybenzotriazole in DMSO (100 g/L, 2 mL, 1.5 mmol) were placed in a vial, and an amine (1 mmol; “starting amine”) was added. If the amine was a hydrochloride salt, Et₃N (1 mmol) was also added. The reaction mixture was stirred for 30 min in a shaker, and EDC (1.2 mmol) was added.

After all the reagents were loaded, the vial was sealed and stirred in a shaker for 1 h. If a clear solution was formed, the vial was left at rt for 24 h. Otherwise, the reaction mixture was kept in a sonication bath for 24 h (strong heating should be avoided). If a significant thickening of the reaction mixture was observed rendering stirring ineffective, 0.2 mL of DMSO was added in one portion.

The crude reaction mixture was analyzed by LC-MS (Method B) and then subjected to chromatographic purification.

### General Procedure B

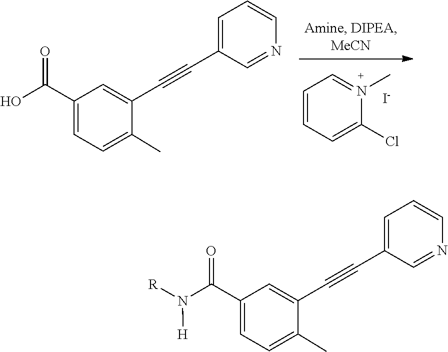

Intermediate 2 (0.10 mmol, 1.0 eq), dry MeCN (0.5 mL), amine (0.10 mmol, 1.0 eq) and DIPEA* (0.25 mmol, 2.5 eq) were placed in a vial. The reaction mixture was stirred for 30 min, and pyridinium salt (0.12 mmol, 1.2 eq) was added. The vial was sealed and the reaction mixture was heated for 6 h at 100° C. After cooling to rt the mixture was evaporated. The residue was dissolved in DMSO, filtered, and the solution was subjected to chromatographic purification. The product was analysed by LC-MS (Method B). * If the amine was purchased as a salt, an additional amount of DIPEA was added to the reaction mixture generate the free base.

### General Procedure C

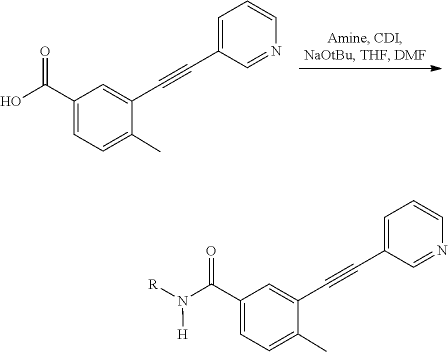

Intermediate 2 (0.1 mmol, 1.0 eq) and CDI (1.0 eq) in DMF (1 mL) were placed in a vial. The reaction mixture was heated with stirring for 1 h at 50° C. Then amine (1.0 eq) and a solution of NaOtBu* in THF (2.0 eq) were added. The vial was sealed and the reaction mixture was heated for 6 h at 60° C. After cooling to rt the mixture was quenched with excess acetic acid and evaporated. The residue was dissolved in DMSO, filtered, and the solution was subjected to chromatographic purification. The product was analysed by LC-MS (Method B). *If the amine was purchased as a salt, an additional amount of NaOtBu was added to the reaction mixture generate the free base.

### General Procedure D

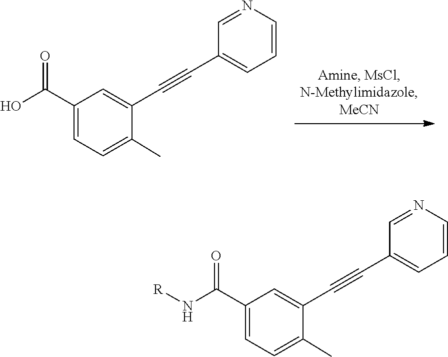

To Intermediate 2 (0.11 mmol, 1.1 eq), N-methylimidazole (2.2 eq) and MeCN (0.7 mL) at rt was carefully added mesyl chloride (1.1 eq) (small exotherm observed). The reaction mixture was stirred for 0.5 h to allow it to cool back to rt. Amine (1.0 eq) was then added, the vial sealed, and the reaction mixture stirred in a shaker for 1 h at rt and heated for 2 h at 100° C. After cooling to rt the solvent was evaporated in vacuo, the residue was dissolved in DMSO, filtered, and the solution was subjected to chromatographic purification. The product was analysed by LC-MS (Method B).

### Examples 1 to 23

Examples 1 to 23 were prepared according to the General procedure A

| | | | | LC-MS |
|---|---|---|---|---|
| | | | LC-MS | Retention |
| | | | m/z | Time |
| Ex. | Compound Name | Structure | [M + H]⁺ | (min) |
| 1 | 4-methyl-N- {4H,5H,6H,7H- pyrazolo[1,5- a]pyridin-2- yl}-3-[2-(pyridin-3- yl)ethynyl]benzamide | 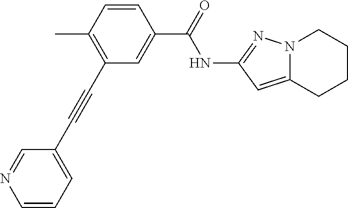 [SMILES: Cc1ccc(C(=O)Nc2cc3n(n2)CCCC3)cc1C#Cc1cccnc1] | 357.2 | 1.312 |
| 2 | 4-methyl-N-(2- methyl-4,5,6,7- tetrahydro-2H- indazol-3- yl)-3-[2-(pyridin-3- yl)ethynyl]benzamide | 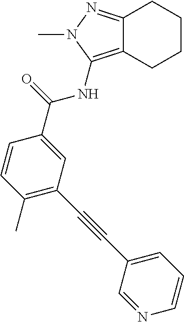 [SMILES: Cc1ccc(C(=O)Nc2c3c(nn2C)CCCC3)cc1C#Cc1cccnc1] | 371.2 | 1.448 |
| 3 | 4-methyl-N-[1- (propan-2-yl)-1H- 1,2,3-triazol-4-yl]-3- [2-(pyridin-3- yl)ethynyl]benzamide | 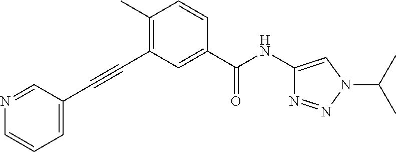 [SMILES: Cc1ccc(C(=O)Nc2cn(C(C)C)nn2)cc1C#Cc1cccnc1] | 346.2 | 1.304 |
| 4 | 4-methyl-3-[2- (pyridin-3-yl)ethynyl]- N-[1-(2,2,2- trifluoroethyl)-1H- pyrazol-3- yl]benzamide | 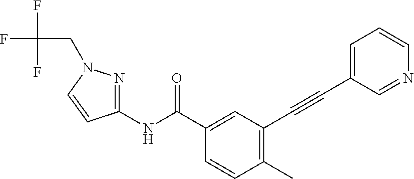 [SMILES: Cc1ccc(C(=O)Nc2ccn(CC(F)(F)F)n2)cc1C#Cc1cccnc1] | 385.2 | 1.41 |
| 5 | 4-methyl-3-[2- (pyridin-3-yl)ethynyl]- N-[1-(3,3,3- trifluoropropyl)-1H- pyrazol-4- yl]benzamide | 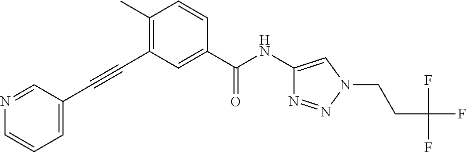 [SMILES: Cc1ccc(C(=O)Nc2cn(CCC(F)(F)F)nn2)cc1C#Cc1cccnc1] | 399.2 | 1.346 |
| 6 | N-(1-tert-butyl-1H- pyrazol-3-yl)-4- methyl-3-[2- (pyridin-3- yl)ethynyl]benzamide | 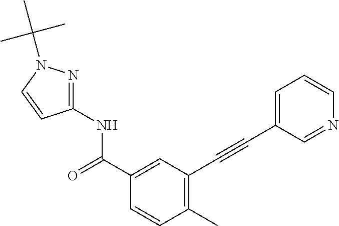 [SMILES: Cc1ccc(C(=O)Nc2ccn(C(C)(C)C)n2)cc1C#Cc1cccnc1] | 359.2 | 1.431 |
| 7 | N-(5-cyclopropyl-1- methyl-1H-pyrazol-3- yl)-4-methyl-3- [2-(pyridin-3- yl)ethynyl]benzamide | 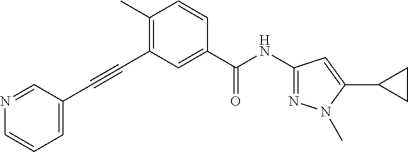 [SMILES: Cc1ccc(C(=O)Nc2cc(C3CC3)n(C)n2)cc1C#Cc1cccnc1] | 357.2 | 1.42 |
| 8 | 4-methyl-N-[5- methyl-1-(2,2,2- trifluoroethyl)-1H- pyrazol-3-yl]-3-[2- (pyridin-3- yl)ethynyl]benzamide | 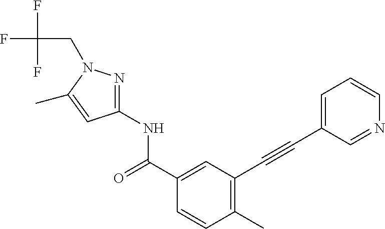 [SMILES: Cc1ccc(C(=O)Nc2cc(C)n(CC(F)(F)F)n2)cc1C#Cc1cccnc1] | 399.2 | 1.41 |
| 9 | N-(1-cyclobutyl-1H- pyrazol-4-yl)-4- methyl-3-[2- (pyridin-3- yl)ethynyl]benzamide | 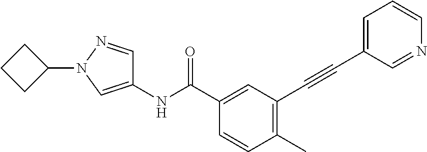 [SMILES: Cc1ccc(C(=O)Nc2cnn(C3CCC3)c2)cc1C#Cc1cccnc1] | 357.2 | 1.363 |
| 10 | N-(1-tert-butyl-1H- pyrazol-4-yl)-4- methyl-3-[2- (pyridin-3- yl)ethynyl]benzamide | 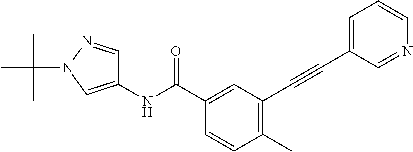 [SMILES: Cc1ccc(C(=O)Nc2cnn(C(C)(C)C)c2)cc1C#Cc1cccnc1] | 359.2 | 1.396 |
| 11 | N-(3-tert-butyl-1- methyl-1H-pyrazol-5- yl)-4-methyl-3- [2-(pyridin-3- yl)ethynyl]benzamide | 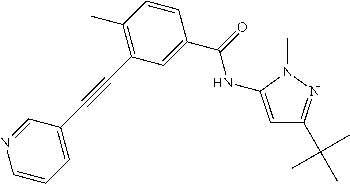 [SMILES: Cc1ccc(C(=O)Nc2cc(C(C)(C)C)nn2C)cc1C#Cc1cccnc1] | 373.2 | 1.424 |
| 12 | N-[1-(2,2- dimethylpropyl)-1H- pyrazol-4-yl]-4- methyl-3-[2-(pyridin-3- yl)ethynyl]benzamide | 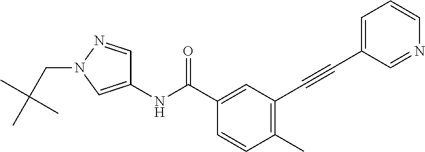 [SMILES: Cc1ccc(C(=O)Nc2cnn(CC(C)(C)C)c2)cc1C#Cc1cccnc1] | 373.2 | 1.446 |
| 13 | N-[1- (cyclopropylmethyl)- 1H-pyrazol-4-yl]-4- methyl-3- [2-(pyridin-3- yl)ethynyl]benzamide | 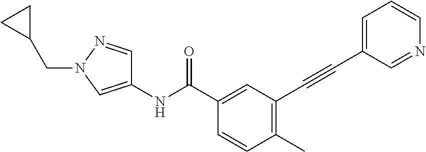 [SMILES: Cc1ccc(C(=O)Nc2cnn(CC3CC3)c2)cc1C#Cc1cccnc1] | 357.2 | 1.329 |
| 14 | N-[5- (difluoromethyl)-1- methyl-1H-pyrazol-3- yl]-4-methyl-3-[2- (pyridin-3- yl)ethynyl]benzamide | 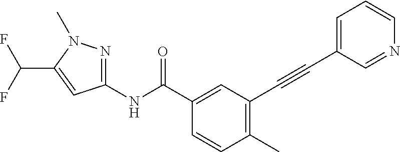 [SMILES: Cc1ccc(C(=O)Nc2cc(C(F)F)n(C)n2)cc1C#Cc1cccnc1] | 367.2 | 1.41 |
| 15 | 4-methyl-N-[5- methyl-1-(2- methylpropyl)-1H- pyrazol-4- yl]-3-[2-(pyridin-3- yl)ethynyl]benzamide | 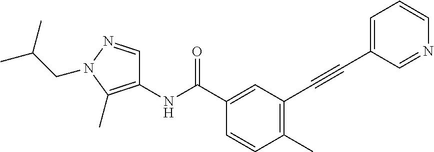 [SMILES: Cc1ccc(C(=O)Nc2cnn(CC(C)C)c2C)cc1C#Cc1cccnc1] | 373.2 | 1.345 |
| 16 | N-[1-(2,2- difluorocyclopropyl)- 1H-pyrazol-4-yl]-4- methyl-3-[2-(pyridin-3- yl)ethynyl]benzamide | 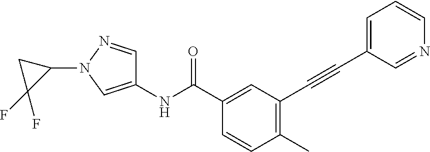 [SMILES: Cc1ccc(C(=O)Nc2cnn(C3CC3(F)F)c2)cc1C#Cc1cccnc1] | 379.2 | 1.25 |
| 17 | N-[1-(1- cyclopropylethyl)-1H- pyrazol-4-yl]-4- methyl-3-[2-(pyridin-3- yl)ethynyl]benzamide | 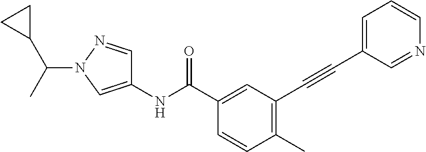 [SMILES: Cc1ccc(C(=O)Nc2cnn(C(C)C3CC3)c2)cc1C#Cc1cccnc1] | 371.2 | 1.384 |
| 18 | N-(1-cyclobutyl-5- methyl-1H-pyrazol-4- yl)-4-methyl-3- [2-(pyridin-3- yl)ethynyl]benzamide | 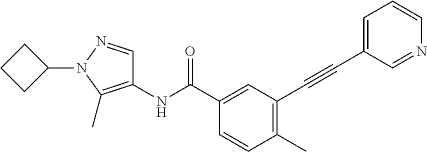 [SMILES: Cc1ccc(C(=O)Nc2cnn(C3CCC3)c2C)cc1C#Cc1cccnc1] | 371.2 | 1.251 |
| 19 | 4-methyl-N-[1- (propan-2-yl)-1H- 1,2,4-triazol-3-yl]-3- [2-(pyridin-3- yl)ethynyl]benzamide | 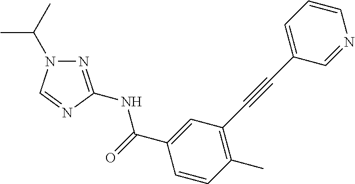 [SMILES: Cc1ccc(C(=O)Nc2ncn(C(C)C)n2)cc1C#Cc1cccnc1] | 346.2 | 1.217 |
| 20 | 4-methyl-N-[1-(2- methylpropyl)-1H- pyrazol-4-yl]-3- [2-(pyridin-3- yl)ethynyl]benzamide | 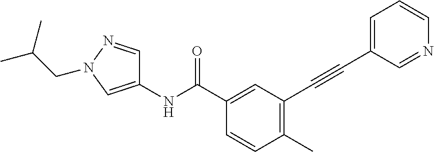 [SMILES: Cc1ccc(C(=O)Nc2cnn(CC(C)C)c2)cc1C#Cc1cccnc1] | 359.2 | 1.431 |
| 21 | N-[5- (difluoromethoxy)-1- methyl-1H-pyrazol-3- yl]-4-methyl-3-[2- (pyridin-3- yl)ethynyl]benzamide | 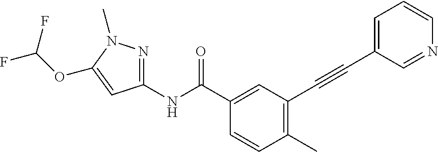 [SMILES: Cc1ccc(C(=O)Nc2cc(OC(F)F)n(C)n2)cc1C#Cc1cccnc1] | 383.2 | 1.41 |
| 22 | N-[1-(2,2- difluoroethyl)-5- methyl-1 H-pyrazol-4- yl]-4-methyl-3-[2- (pyridin-3-yl) ethynyl]benzamide | 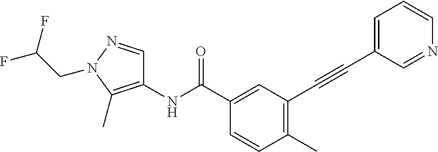 [SMILES: Cc1ccc(C(=O)Nc2cnn(CC(F)F)c2C)cc1C#Cc1cccnc1] | 381 | 1.221 |
| 23 | 4-methyl-N-[5- methyl-1-(propan-2- yl)-1H-pyrazol-4- yl]-3-[2-(pyridin-3- yl)ethynyl]benzamide | 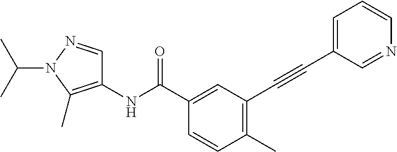 [SMILES: Cc1ccc(C(=O)Nc2cnn(C(C)C)c2C)cc1C#Cc1cccnc1] | 359.2 | 1.358 |

| | | Starting Amine |
|---|---|---|
| Ex. | Starting Amine | CAS Number |
| 1 | 4H,5H,6H,7H-pyrazolo[1,5-a]pyridin-2-amine | 149978-63-2 |
| 2 | 4,5,6,7-tetrahydro-2-methyl-2H-Indazol-3-amine, | 26503-23-1 |
| 3 | 1-(propan-2-yl)-1H-1,2,3-triazol-4-amine | 959237-92-4 |
| 4 | 1H-Pyrazol-3-amine, 1-(2,2,2-trifluoroethyl)- | 947179.47.7 |
| 5 | 1-(3,3,3-trifluoropropyl)-1H-Pyrazol-4-amine | 1245771-72-5 |
| 6 | 1-(1,1-dimethylethyl)-1H-pyrazol-3-amine | 1152980-49-8 |
| 7 | 5-cyclopropyl-1-methyl-1H-Pyrazol-3-amine | 1170238-67-1 |
| 8 | 5-methyl-1-(2,2,2-trifluoroethyl)-1H-Pyrazol-3-amine | 1006486-85-6 |
| 9 | 1-cyclobutyl-1H-pyrazol-4-amine | 1190380-64-3 |
| 10 | 1-tert-Butyl-1H-pyrazol-4-amine | 97421-13-1 |
| 11 | 3-tert-Butyl-1-methyl-1H-pyrazol-5-amine | 118430-73-2 |
| 12 | 1-neopentyl-1H-pyrazol-4-amine | 1240567-28-5 |
| 13 | 1-(cyclopropylmethyl)-1H-pyrazol-4-amine | 1156169-29-7 |
| 14 | 5-(difluoromethyl)-1-methyl-1H-pyrazol-3-amine | 1638927-79-3 |
| 15 | 5-methyl-1-(2-methylpropyl)-1H-pyrazol-4-amine | 405548-39-2 |
| 16 | 1-(2,2-difluorocyclopropyl)-1H-pyrazol-4-amine | 2172602-66-1 |
| 17 | 1-(1-cyclopropylethyl)-1H-pyrazol-4-amine | 1251266-33-7 |
| 18 | 1-cyclobutyl-5-methyl-1H-pyrazol-4-amine | 2137931-45-2 |
| 19 | 1-(propan-2-yl)-1H-1,2,4-triazol-3-amine | 1183827-04-4 |
| 20 | 1-Isobutyl-1H-pyrazol-4-amine | 405548-42-7 |
| 21 | 5-(difluoromethoxy)-1-methyl-1H-pyrazol-3-amine | 149978-55-2 |
| 22 | 1-(2,2-difluoroethyl)-5-methyl-1H-pyrazol-4-amine | 2105567-91-5 |
| 23 | 5-methyl-1-(propan-2-yl)-1H-pyrazol-4-amine | 1006495-81-3 |

### Example 24

### N-(1-isopropylpyrazol-4-yl)-4-methyl-3-[2-(3-pyridyl)ethynyl]benzamide

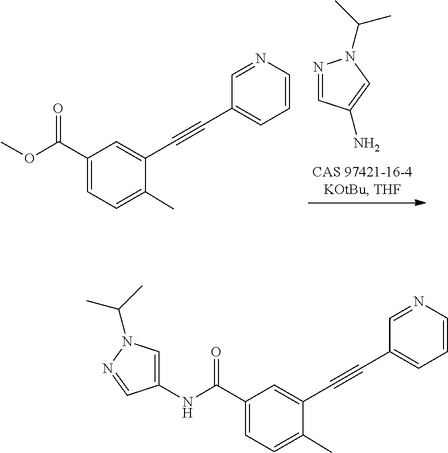

To an ice-cold solution of 1-isopropyl-1H-pyrazol-4-amine (1.00 g, 8.0 mmol, 1.0 eq) and methyl 4-methyl-3-(pyridin-3-ylethynyl)benzoate (2.01 g, 8.0 mmol, 1.0 eq) in THF (40 mL) was added KtOBu (20% wt solution in THF, 5.17 mL, 8.8 mmol, 1.1 eq) dropwise over ˜2 min and the resulting solution stirred at 0° C. for 30 min then allowed to warm to rt for 1 h. The reaction mixture was quenched by addition of H₂O (250 mL) and then concentrated in vacuo to remove ˜20 mL THF. This was then diluted with water (100 mL) and the aqueous extracted with tert-butyl methyl ether (2×200 mL). Combined organics were washed with saturated brine (150 mL), dried over MgSO₄ and concentrated in vacuo to give a purple/brown oily solid. This was combined with further crude product (from an identical 220 mg scale reaction), absorbed onto silica and purified by normal phase chromatography, EtOAc:heptane (1:1) to 100% EtOAc, and the resulting residue triturated with tert-butyl methyl ether to afford N-(1-isopropylpyrazol-4-yl)-4-methyl-3-[2-(3-pyridyl)ethynyl]benzamide (1.28 g, 37%) as a colourless solid. UPLC-MS (Method A) 3.11 min, 100%, [M+H]⁺=345.3.

### Example 25

### N-(1-isopropylimidazol-4-yl)-4-methyl-3-[2-(3-pyridyl)ethynyl]benzamide

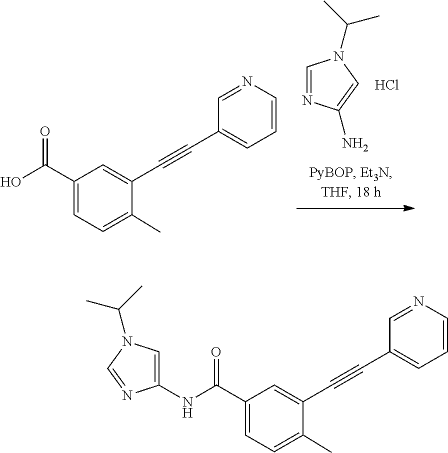

To a suspension of Intermediate 2 (1.42 g, 6 mmol, 1 eq) in tetrahydrofuran (75 mL) was added triethylamine (1.82 g, 18 mmol, 3 eq) and the suspension stirred for 10 min after which Intermediate 4 (assumed 6 mmol, 1 eq) followed by benzotriazol-1-yl-oxytripyrrolidinophosphonium hexafluorophosphate (3.12 g, 6 mmol, 1 eq) were added and the reaction stirred at rt under nitrogen for 18 h. The reaction was quenched by pouring into H₂O (80 mL) and diluted with DCM (80 mL), the phases were separated and the organic phase dried (MgSO₄) and concentrated in vacuo. The crude material was purified by normal phase chromatography (MeOH:DCM, 1:19) and then re-purified by normal phase chromatography (MeOH:DCM, 1:39). The material was crystallised from EtOAc (40 mL) to afford two batches (878 mg, 350 mg). The second batch (350 mg) was taken up in DCM (10 mL) and washed with H₂O (10 mL), dried (MgSO₄) and concentrated in vacuo and the material was then crystallised from EtOAc (7 mL) over the weekend. The resulting solid was washed with EtOAc (0° C., 2 mL) to afford N-(1-isopropylimidazol-4-yl)-4-methyl-3-[2-(3-pyridyl)ethynyl]benzamide (124 mg & 878 mg, 48%). UPLC (Method A) 2.75 min, 99.7%, [M+H]⁺=345.2.

### Example 26

### N-(4,4-dimethyl-5,6-dihydropyrrolo[1,2-b]pyrazol-2-yl)-4-methyl-3-[2-(3-pyridyl)ethynyl]benzamide

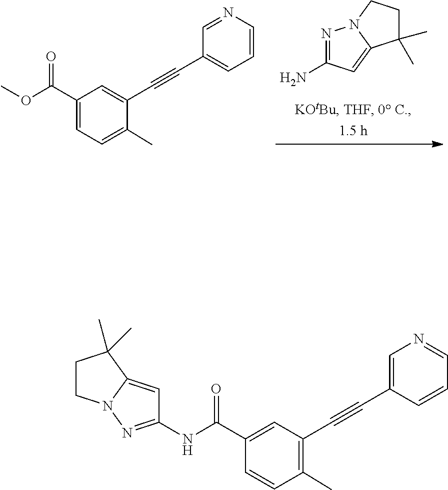 [SMILES: COC(=O)c1ccc(C)c(C#Cc2cccnc2)c1 ; CC1(C)CCn2nc(N)cc21 ; Cc1ccc(C(=O)Nc2cc3n(n2)CCC3(C)C)cc1C#Cc1cccnc1]

A solution of Intermediate 1 (150 mg, 0.60 mmol, 1.0 eq) and Intermediate 7 (108 mg, 0.72 mmol, 1.2 eq) in THF (4.6 mL) was cooled to 0° C. after which KOtBu (20% wt solution in THF) (0.45 mL. 0.72 mmol, 1.2 eq) was added dropwise and stirred at 0° C. for 1.5 h. The reaction was quenched with brine, the phases separated and the aqueous layer extracted with EtOAc (×3), the combined organics were dried (MgSO₄) and concentrated in vacuo. The crude material was purified by reverse phase chromatography 5-80% MeCN/H₂O (0.1% NH₃ modifier) to afford N-(4,4-dimethyl-5,6-dihydropyrrolo[1,2-b]pyrazol-2-yl)-4-methyl-3-[2-(3-pyridyl)ethynyl]benzamide (36 mg, 16%) as an off-white solid. UPLC (Method A) 3.36 min, 98.91, [M+H]⁺=371.30.

### Example 27

### N-[1-(cyclopropylmethyl)imidazol-4-yl]-4-methyl-3-[2-(3-pyridyl)ethynyl]benzamide

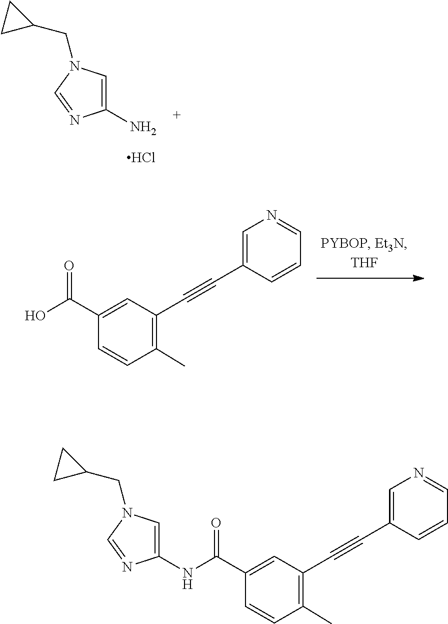 [SMILES: Nc1cn(CC2CC2)cn1 ; Cl ; Cc1ccc(C(=O)O)cc1C#Cc1cccnc1 ; Cc1ccc(C(=O)Nc2cn(CC3CC3)cn2)cc1C#Cc1cccnc1]

To a suspension of Intermediate 2 (169 mg, 0.71 mmol, 1.0 eq) in THF (8 mL) was added triethylamine (398 μL, 2.86 mmol, 4.0 eq) and the reaction stirred at rt for 10 min, before addition of 1-(cyclopropylmethyl)-1H-imidazol-4-amine dihydrochloride (150 mg, 0.71 mmol, 1.0 eq) and (benzotriazol-1-yl-oxytripyrrolidinophosphonium hexafluorophosphate) (409 mg, 0.79 mmol, 1.1 eq) and further stirring at rt for 2 h. Additional 1-(cyclopropylmethyl)-1H-imidazol-4-amine dihydrochloride (15 mg, 0.07 mmol, 0.1 eq) and triethylamine (100 μL, 0.71 mmol, 1.0 eq) were added and the reaction stirred for a further 1 h, then quenched with H₂O (40 mL) and extracted with DCM (3×30 mL). Combined organics were washed with 1 M ammonia (40 mL), H₂O (40 mL) and brine (40 mL), dried (phase separator) and concentrated in vacuo. The crude was absorbed onto silica and purified by normal phase chromatography 10-40% Acetone/DCM and the solid obtained was crystallised from EtOAc to afford N-[1-(cyclopropylmethyl)imidazol-4-yl]-4-methyl-3-[2-(3-pyridyl)ethynyl]benzamide (130 mg, 51%) as a colourless crystalline solid. UPLC (Method A) 3.05 min, 98%, [M+H]⁺=357.2.

### Example 28

### 4-Methyl-3-[2-(3-pyridyl)ethynyl]-N-[1-(2,2,2-trifluoroethyl)imidazol-4-yl]benzamide

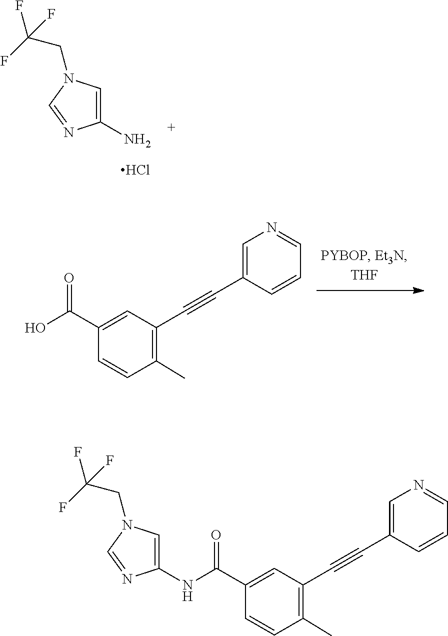 [SMILES: Nc1cn(CC(F)(F)F)cn1 ; Cl ; Cc1ccc(C(=O)O)cc1C#Cc1cccnc1 ; Cc1ccc(C(=O)Nc2cn(CC(F)(F)F)cn2)cc1C#Cc1cccnc1]

To a suspension of Intermediate 2 (118 mg, 0.50 mmol, 1.0 eq) in THF (5 mL) was added triethylamine (207 μL, 1.49 mmol, 3.0 eq) and the reaction stirred at rt for 10 min before addition of 1-(2,2,2-trifluoroethyl)-1H-imidazol-4-amine hydrochloric salt (100 mg, 0.50 mmol, 1.0 eq) and (benzotriazol-1-yl-oxytripyrrolidinophosphonium hexafluorophosphate) (284 mg, 0.55 mmol, 1.1 eq) and further stirring at rt for 7 h. To the reaction was then added 1-(2,2,2-trifluoroethyl)-1H-imidazol-4-amine hydrochloric salt (10 mg, 0.05 mmol, 0.1 eq) and triethylamine (69 μL, 0.50 mmol, 1 eq) and the reaction stirred at rt for another 18 h. The reaction was quenched with water (40 mL), extracted with DCM (3×20 mL) and combined organics were washed with 1M ammonia (40 mL), water (40 mL) and brine (40 mL), dried (phase separator) and concentrated in vacuo. The crude was purified by normal phase chromatography using 4% MeCOH/DCM and the solid crystallised from hot EtOAc overnight. The resulting crystals were washed with EtOAc (×2) and dried under vacuum to give the title compound as colourless solid containing pyrrolidine phosphine oxide. The mother liquors were absorbed onto silica and purified by normal phase chromatography using DCM/Acetone 9/1 to 6/4. The correct fractions were collected and reduced to dryness to give 4-methyl-3-[2-(3-pyridyl)ethynyl]-N-[1-(2,2,2-trifluoroethyl)imidazol-4-yl]benzamide (64 mg, 34%) as colourless solid. UPLC (Method A) 3.04 min, 98%, [M+H]⁺=385.2.

### Example 29

### 4-methyl-3-[2-(3-pyridyl)ethynyl]-N-[5-(trifluoromethyl)-6,7-dihydro-5H-pyrrolo[1,2-a]imidazol-2-yl]benzamide

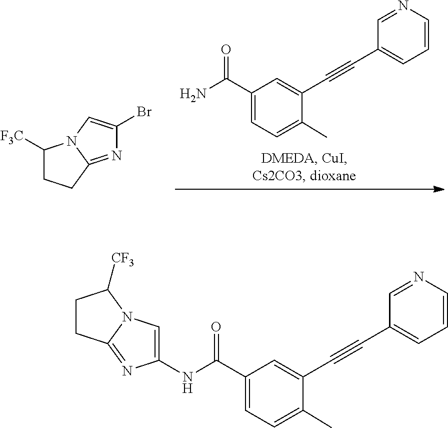 [SMILES: FC(F)(F)C1CCc2nc(Br)cn21 ; Cc1ccc(C(N)=O)cc1C#Cc1cccnc1 ; Cc1ccc(C(=O)Nc2cn3c(n2)CCC3C(F)(F)F)cc1C#Cc1cccnc1]

2-Bromo-5-(trifluoromethyl)-6,7-dihydro-5H-pyrrolo[1,2-a]imidazole Intermediate 11 (160 mg, 0.63 mmol, 1.0 eq), Intermediate 12 (148 mg, 0.63 mmol, 1.0 eq), caesium carbonate (306 mg, 0.94 mmol, 1.5 eq), copper (I) iodide (120 mg, 0.63 mmol, 1.0 eq) and N,N′-dimethylethylenediamine (55 mg, 0.63 mmol, 1.0 eq) were combined in dioxane (4 mL) and DMSO (1 mL), the reaction degassed for 5 min with N₂ and then heated at 100° C. in a sealed tube for 4 h. The mixture was cooled, evaporated under reduced pressure and the residue partitioned between H₂O (10 mL) and EtOAc (2×15 mL). Combined organics were dried (MgSO₄), concentrated in vacuo and the residue purified by normal phase chromatography (dry loaded) 0.2:2:98-0.5:5:95 NH₃:MeOH:DCM and triturated with diethyl ether to afford the title compound (49 mg, 19%) as a white solid. UPLC (Method A): 3.12 min, 100%, [M+H]⁺=411, [M−H]⁻=409.

### Example 30

### 4-Methyl-3-[2-(3-pyridyl)ethynyl]-N-spiro[6,7-dihydropyrrolo[1,2-a]imidazole-5,1′-cyclopropane]-2-yl-benzamide

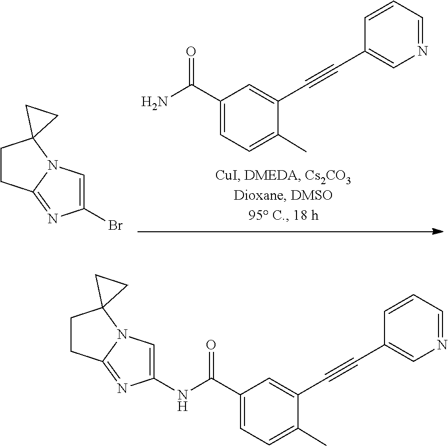 [SMILES: Brc1cn2c(n1)CCC21CC1 ; Cc1ccc(C(N)=O)cc1C#Cc1cccnc1 ; Cc1ccc(C(=O)Nc2cn3c(n2)CCC32CC2)cc1C#Cc1cccnc1]

A solution of Intermediate 15 (90 mg, 0.42 mmol, 1.0 eq) in degassed DMSO (1.5 mL) and degassed dioxane (3.5 mL) was further degassed with nitrogen for ca. 10 min. To the solution was added caesium carbonate (208 mg, 0.63 mmol, 1.5 eq), copper (I) iodide (81 mg, 0.42 mmol, 1.0 eq), N,N′-dimethylethylenediamine (55 μL, 0.51 mmol, 1.2 eq) and Intermediate 12 (100 mg, 0.42 mmol, 1.0 eq), and the mixture heated to 95° C. for 18 h. The reaction was cooled to rt and the volatiles removed in vacuo. The residue was diluted with H₂O (20 mL) and DCM/isopropanol (9:1, 70 mL), basified with (15% sol) NH₄OH (to pH=10), the phases separated and the aqueous extracted DCM/isopropanol (9:1, 2×70 mL). Combined organics were washed with brine (50 mL), dried (Na₂SO₄) and concentrated in vacuo. The residue was purified by reverse phase chromatography 15-20% MeCN/H₂O (0.1% NH₄OH modifier) and freeze dried to afford the title compound (16 mg, 10%) as a beige solid. UPLC (Method F) 3.06 min, 99%, [M+H]⁺=369.3, [M−H]⁻=367.2.

### Example 31

### 4-methyl-N-(1-propylimidazol-4-yl)-3-[2-(3-pyridyl)ethynyl]benzamide

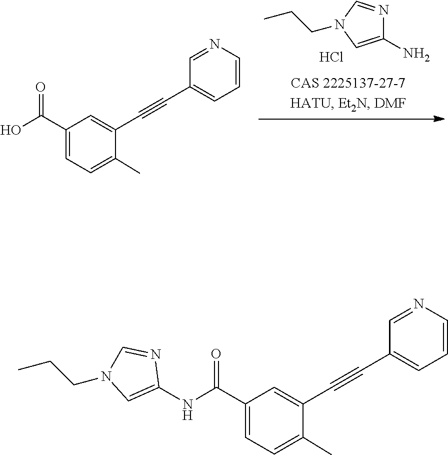

To a suspension of 1-propyl-1H-imidazol-4-amine hydrochloride (100 mg, 0.62 mmol, 1.0 eq), Intermediate 2 (162 mg, 0.68 mmol, 1.1 eq) and (1-[bis(dimethylamino)methylene]-1H-1,2,3-triazolo[4,5-b]pyridinium 3-oxid hexafluorophosphate (282 mg, 0.74 mmol, 1.2 eq) in DMF (4 mL) was added triethylamine (350 μL, 2.47 mmol, 4.0 eq) and the reaction stirred at rt for 10 min. The volatiles were removed in vacuo, the residue partitioned between EtOAc (20 mL) and H₂O (10 mL), the phases separated and the organic washed with 2 M sodium hydroxide (10 mL), dried (MgSO₄) and concentrated in vacuo. The residue was triturated with EtOAc and the solid recrystallised from EtOAc to afford the title compound (50 mg, 24%) as a white solid. UPLC (Method A): 3.10 min, 100%, [M+H]⁺=345.2, [M−H]⁻=343.1.

### Example 32

### N-(1-cyclopropylimidazol-4-yl)-4-methyl-3-[2-(3-pyridyl)ethynyl]benzamide

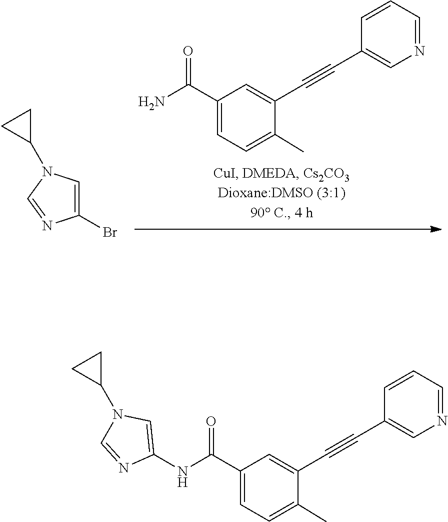 [SMILES: Brc1cn(C2CC2)cn1 ; Cc1ccc(C(N)=O)cc1C#Cc1cccnc1 ; Cc1ccc(C(=O)Nc2cn(C3CC3)cn2)cc1C#Cc1cccnc1]

A solution of Intermediate 16 (150.0 mg, 0.80 mmol, 1.0 eq) in DMSO (2 mL) and dioxane (6 mL) was degassed with nitrogen. To the solution was added caesium carbonate (391.9 mg, 1.20 mmol, 1.5 eq), copper (I) iodide (153.0 mg, 0.80 mmol, 1.0 eq), N,N′-dimethylethylenediamine (150 μL, 0.96 mmol, 1.2 eq) and Intermediate 12 (189.5 mg, 0.80 mmol, 1.0 eq). The resulting reaction mixture was heated to 90° C. for 4 h. The reaction was cooled to rt and the volatiles were removed in vacuo. The residue was diluted with DCM/isopropanol (9:1, 25 mL) and sat. NH₄Cl (50 mL), the phases separated and the aqueous acidified with 2 M HCl (to pH=1). DCM/isopropanol (9:1, 25 mL) was then added and the aqueous basified with K₂CO₃ (to pH=9), the phases separated and the aqueous extracted with DCM/isopropanol (9:1, 25 mL). Combined organics were washed with brine (50 mL), dried (MgSO₄) and concentrated in vacuo. The crude residue was purified by reverse phase chromatography using 15-20% MeCN/H₂O (0.1% NH₄OH modifier) to afford the title compound (98 mg, 36%) as a colourless solid. UPLC (Method F) 2.96 min, 99%, [M+H]⁺=343.2, [M−H]⁻=341.1.

### Example 33

### 4-methyl-3-[2-(3-pyridyl)ethynyl]-N-[5-(trifluoromethyl)-5,6,7,8-tetrahydroimidazo[1,2-a]pyridine-2-yl]benzamide

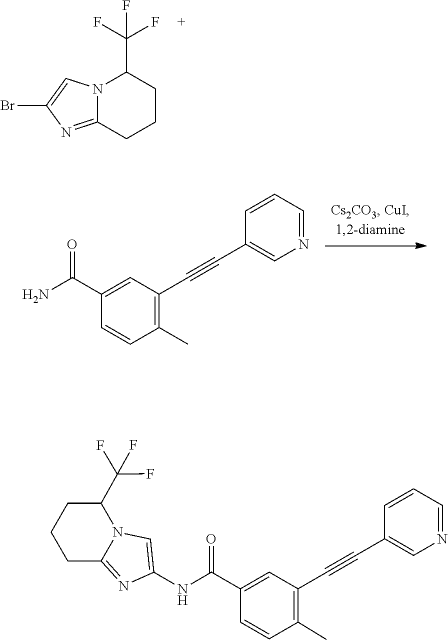 [SMILES: FC(F)(F)C1CCCc2nc(Br)cn21 ; Cc1ccc(C(N)=O)cc1C#Cc1cccnc1 ; Cc1ccc(C(=O)Nc2cn3c(n2)CCCC3C(F)(F)F)cc1C#Cc1cccnc1]

To a degassed solution of Intermediate 108 (250 mg, 0.93 mmol, 1.0 eq), caesium carbonate (454 mg, 1.39 mmol, 1.5 eq), copper (I) iodide (177 mg, 0.93 mmol, 1.0 eq) and trans N,N′-dimethylcyclohexane-1,2-diamine (0.18 mL, 1.11 mmol, 1.2 eq) in 1,4-dioxane/DMSO (12 mL, 3:1) was added Intermediate 12 (220 mg, 0.93 mmol, 1.0 eq) and the reaction heated at 90° C. for 2 h. To the reaction was then added an additional portion of copper (I) iodide (0.5 eq) and trans N,N′-dimethylcyclohexane-1,2-diamine (0.5 eq) and heating continued at 90° C. for a further 1 h. A further portion of copper iodide (0.2 eq) and trans N,N′-dimethylcyclohexane-1,2-diamine (0.2 eq) were added and the reaction heated at 90° C. for a further 3 h. The reaction mixture was partitioned between H₂O (50 mL) and TBME (50 mL) and the aqueous layer extracted with 2-MeTHF (3×30 mL). Combined organics were concentrated in vacuo to give a brown oily residue. This was purified by column chromatography (manual column, normal phase, SilaFlash®P60 silica gel 40-63 μm/230-400 mesh, 60 Å, 0-2% MeCOH in DCM) to give 120 mg of brown oily solid. This was further purified by column chromatography (Biotage Isolera, reverse phase, 12 g, HP-Sphere C18 ULTRA, 25 μm, 35 to 60% MeCN in H₂O, both eluents containing 0.1 Vol % NH₃). Product fractions were concentrated in vacuo to remove solvent, affording a suspension that was filtered and triturated with 1 mL MeCN, giving 4-methyl-3-[2-(3-pyridyl)ethynyl]-N-[5-(trifluoromethyl)-5,6,7,8-tetrahydroimidazo[1,2-a]pyridine-2-yl]benzamide (42 mg, 11%) as a colourless solid. UPLC (Method A) 3.21 min, 100%, [M+H]⁺=425.2, [M−H]⁻=423.2.

The examples in the table below are produced using the general procedure listed therein. The amine used for each example is mentioned in the subsequent table.

| | | | | | LC-MS |
|---|---|---|---|---|---|
| | | | | LC-MS | retention |
| | | | General | m/z | time |
| Ex. | Compound Name | Structure | Procedure | [M + H]⁺ | (min) |
| 34 | N-(4-tert-butyl-1,3- oxazol-2-yl)-4- methyl-3-[2- (pyridin-3- yl)ethynyl]benzamide | 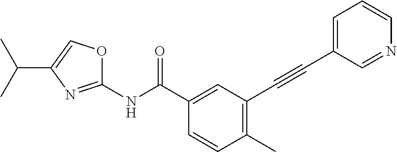 [SMILES: Cc1ccc(C(=O)Nc2nc(C(C)C)co2)cc1C#Cc1cccnc1] | C | 360.1 | 1.33 |
| 35 | 4-methyl-N-[4- (propan-2-yl)-1,3- oxazol-2-yl]-3-[2- (pyridin-3- yl)ethynyl]benzamide | 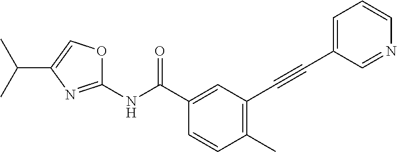 [SMILES: Cc1ccc(C(=O)Nc2nc(C(C)C)co2)cc1C#Cc1cccnc1] | B | 346.2 | 1.385 |
| 36 | 4-methyl-N-[2- (propan-2-yl)-2H- 1,2,3,4-tetrazol-5-yl]- 3-[2-(pyridin-3- yl)ethynyl]benzamide | 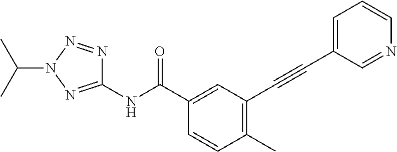 [SMILES: Cc1ccc(C(=O)Nc2nnn(C(C)C)n2)cc1C#Cc1cccnc1] | C | 347.1 | 1.18 |
| 37 | N-(1-tert-butyl-1H- 1,2,4-triazol-3-yl)-4- methyl-3-[2- (pyridin-3- yl)ethynyl]benzamide | 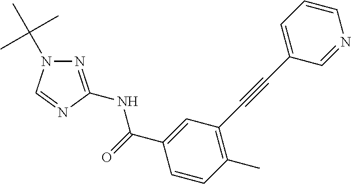 [SMILES: Cc1ccc(C(=O)Nc2ncn(C(C)(C)C)n2)cc1C#Cc1cccnc1] | B | 360.2 | 1.219 |
| 43 | N-[1- (difluoromethyl)-5- methyl-1H-pyrazol-4- yl]-4-methyl-3-[2- (pyridin-3- yl)ethynyl]benzamide | 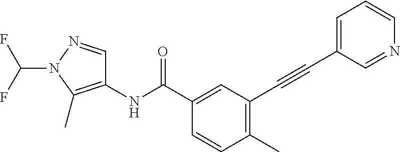 [SMILES: Cc1ccc(C(=O)Nc2cnn(C(F)F)c2C)cc1C#Cc1cccnc1] | A | 367 | 1.33 |
| 45 | 4-methyl-N-[4-(2- methylpropyl)-1,3- oxazol-2-yl]-3-[2- (pyridin-3- yl)ethynyl]benzamide | 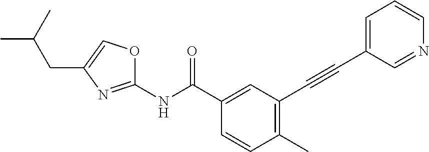 [SMILES: Cc1ccc(C(=O)Nc2nc(CC(C)C)co2)cc1C#Cc1cccnc1] | C | 360 | 1.400 |
| 46 | 4-methyl-N-[3- (propan-2-yl)-1,2,4- oxadiazol-5-yl]-3-[2- (pyridin-3- yl)ethynyl]benzamide | 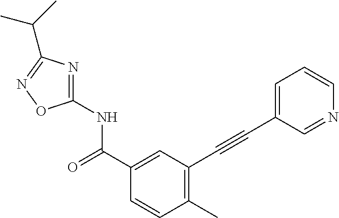 [SMILES: Cc1ccc(C(=O)Nc2nc(C(C)C)no2)cc1C#Cc1cccnc1] | C | 347.2 | 1.286 |
| 47 | N-[1-(butan-2-yl)-1H- pyrazol-3-yl]-4- methyl-3-[2- (pyridin-3- yl)ethynyl]benzamide | 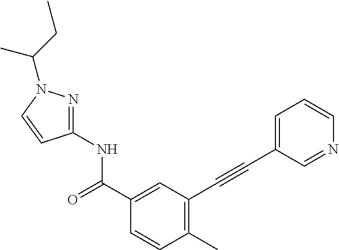 [SMILES: CCC(C)n1ccc(NC(=O)c2ccc(C)c(C#Cc3cccnc3)c2)n1] | A | 359.2 | 1.42 |
| 48 | 4-methyl-3-[2- (pyridin-3-yl)ethynyl]- N-[1-(2,2,2- trifluoroethyl)-1H- pyrazol-4- yl]benzamide | 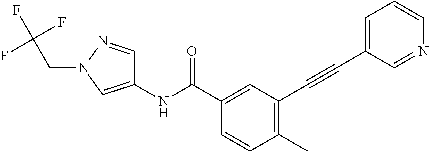 [SMILES: Cc1ccc(C(=O)Nc2cnn(CC(F)(F)F)c2)cc1C#Cc1cccnc1] | A | 385 | 1.329 |
| 49 | N-{6,6-dimethyl- 5H,6H,7H- pyrazolo[3,2- b][1,3]oxazin-3-yl}- 4-methyl-3-[2- (pyridin-3- yl)ethynyl]benzamide | 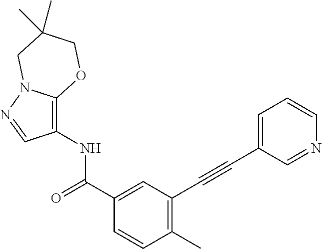 [SMILES: Cc1ccc(C(=O)Nc2cnn3c2OCC(C)(C)C3)cc1C#Cc1cccnc1] | A | 387.2 | 1.171 |
| 50 | 4-methyl-N-(1- propyl-1H-pyrazol-4- yl)-3-[2-(pyridin-3- yl)ethynyl]benzamide | 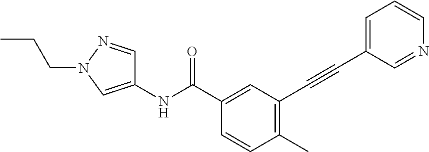 [SMILES: CCCn1cc(NC(=O)c2ccc(C)c(C#Cc3cccnc3)c2)cn1] | A | 345.2 | 1.32 |
| 51 | N-(3-cyclobutyl-1- methyl-1H-pyrazol-5- yl)-4-methyl-3-[2- (pyridin-3- yl)ethynyl]benzamide | 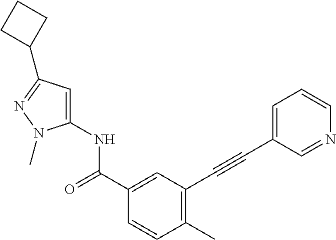 [SMILES: Cc1ccc(C(=O)Nc2cc(C3CCC3)nn2C)cc1C#Cc1cccnc1] | A | 371.2 | 1.374 |
| 52 | N-[3-methoxy-1- (2,2,2-trifluoroethyl)- 1H-pyrazol-4-yl]-4- methyl-3-[2- (pyridin-3- yl)ethynyl]benzamide | 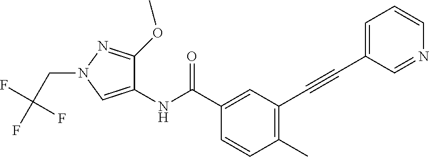 [SMILES: COc1nn(CC(F)(F)F)cc1NC(=O)c1ccc(C)c(C#Cc2cccnc2)c1] | A | 415.2 | 1.44 |
| 53 | N-[1-(2,2- difluoroethyl)-1H- pyrazol-4-yl]-4- methyl-3-[2- (pyridin-3- yl)ethynyl]benzamide | 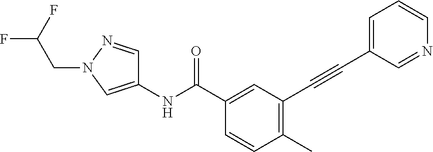 [SMILES: Cc1ccc(C(=O)Nc2cnn(CC(F)F)c2)cc1C#Cc1cccnc1] | A | 367 | 1.262 |
| 54 | N-[1-(2,2- difluoroethyl)-5- methyl-1H-pyrazol-3- yl]-4-methyl-3-[2- (pyridin-3- yl)ethynyl]benzamide | 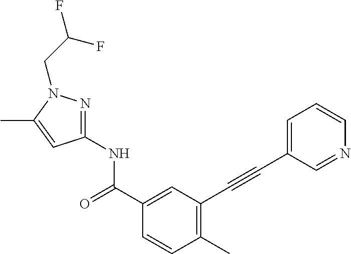 [SMILES: Cc1ccc(C(=O)Nc2cc(C)n(CC(F)F)n2)cc1C#Cc1cccnc1] | A | 381.2 | 1.35 |
| 55 | 4-methyl-N-[5- methyl-1-(propan-2- yl)-1H-pyrazol-3-yl]- 3-[2-(pyridin-3- yl)ethynyl]benzamide | 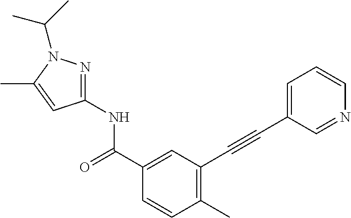 [SMILES: Cc1ccc(C(=O)Nc2cc(C)n(C(C)C)n2)cc1C#Cc1cccnc1] | A | 359.2 | 1.418 |
| 58 | 4-methyl-N-[4- methyl-1-(propan-2- yl)-1H-pyrazol-3-yl]- 3-[2-(pyridin-3- yl)ethynyl]benzamide | 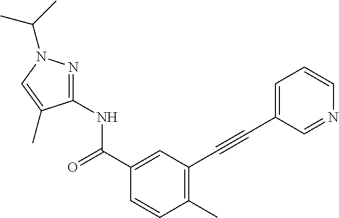 [SMILES: Cc1ccc(C(=O)Nc2nn(C(C)C)cc2C)cc1C#Cc1cccnc1] | A | 359.2 | 1.338 |
| 59 | N-[5- (difluoromethyl)-1- methyl-1H-1,2,3- triazol-4-yl]-4-methyl- 3-[2-(pyridin-3- yl)ethynyl]benzamide | 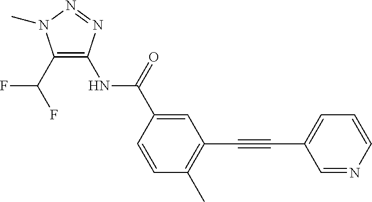 [SMILES: Cc1ccc(C(=O)Nc2nnn(C)c2C(F)F)cc1C#Cc1cccnc1] | A | 368.2 | 1.325 |
| 61 | 4-methyl-N-[1- methyl-3-(2- methylpropyl)-1H- pyrazol-5-yl]-3-[2- (pyridin-3- yl)ethynyl]benzamide | 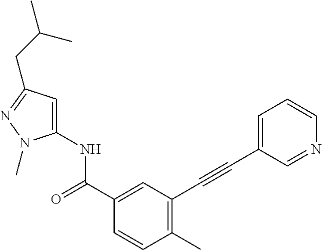 [SMILES: Cc1ccc(C(=O)Nc2cc(CC(C)C)nn2C)cc1C#Cc1cccnc1] | A | 373.2 | 1.435 |
| 62 | N-(1-cyclopropyl-1H- pyrazol-4-yl)-4- methyl-3-[2- (pyridin-3- yl)ethynyl]benzamide | 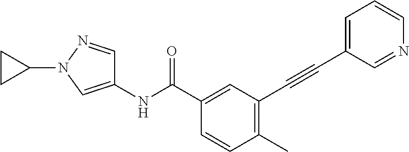 [SMILES: Cc1ccc(C(=O)Nc2cnn(C3CC3)c2)cc1C#Cc1cccnc1] | A | 343.2 | 1.286 |
| 66 | 4-methyl-N-[1- (propan-2-yl)-1H- pyrazol-3-yl]-3-[2- (pyridin-3- yl)ethynyl]benzamide | 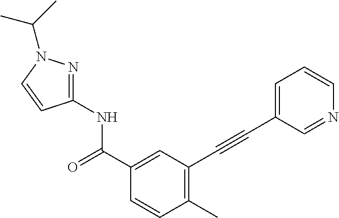 [SMILES: Cc1ccc(C(=O)Nc2ccn(C(C)C)n2)cc1C#Cc1cccnc1] | A | 345.2 | 1.363 |
| 68 | N-[1- (cyclobutylmethyl)- 1H-pyrazol-4-yl]-4- methyl-3-[2- (pyridin-3- yl)ethynyl]benzamide | 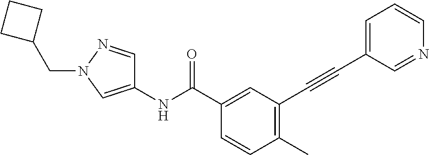 [SMILES: Cc1ccc(C(=O)Nc2cnn(CC3CCC3)c2)cc1C#Cc1cccnc1] | A | 371.2 | 1.388 |
| 70 | N-[3- (cyclopropylmethyl)- 1-methyl-1H-pyrazol- 5-yl]-4-methyl-3-[2- (pyridin-3- yl)ethynyl]benzamide | 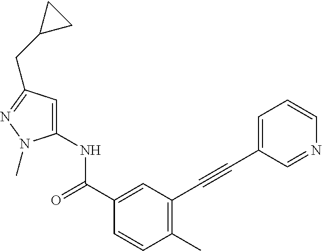 [SMILES: Cc1ccc(C(=O)Nc2cc(CC3CC3)nn2C)cc1C#Cc1cccnc1] | A | 371.2 | 1.289 |
| 71 | 4-methyl-N-[3- methyl-1-(2,2,2- trifluoroethyl)-1H- pyrazol-4-yl]-3-[2- (pyridin-3- yl)ethynyl]benzamide | 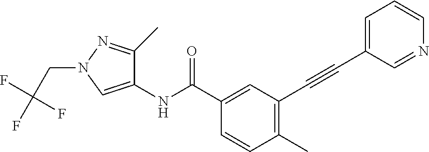 [SMILES: Cc1ccc(C(=O)Nc2cn(CC(F)(F)F)nc2C)cc1C#Cc1cccnc1] | A | 399.2 | 1.403 |
| 72 | N-[1-(2,2- difluoroethyl)-3- methyl-1H-pyrazol-4- yl]-4-methyl-3-[2- (pyridin-3- yl)ethynyl]benzamide | 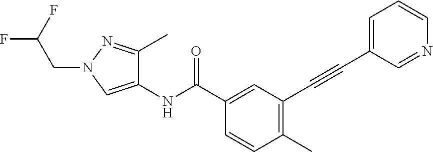 [SMILES: Cc1ccc(C(=O)Nc2cn(CC(F)F)nc2C)cc1C#Cc1cccnc1] | A | 381.2 | 1.305 |
| 73 | N-[1-(2,2- difluorocyclopropyl)- 1H-pyrazol-3-yl]-4- methyl-3-[2- (pyridin-3- yl)ethynyl]benzamide | 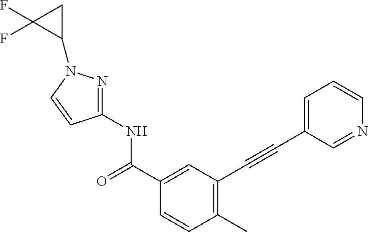 [SMILES: Cc1ccc(C(=O)Nc2ccn(C3CC3(F)F)n2)cc1C#Cc1cccnc1] | A | 379 | 1.349 |
| 74 | 4-methyl-N-[3- methyl-1-(2- methylpropyl)-1H- pyrazol-4-yl]-3-[2- (pyridin-3- yl)ethynyl]benzamide | 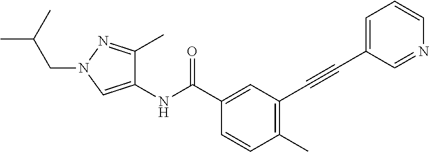 [SMILES: Cc1ccc(C(=O)Nc2cn(CC(C)C)nc2C)cc1C#Cc1cccnc1] | A | 373.2 | 1.396 |
| 75 | N-[1-(2,2- difluoroethyl)-1H- pyrazol-3-yl]-4- methyl-3-[2- (pyridin-3- yl)ethynyl]benzamide | 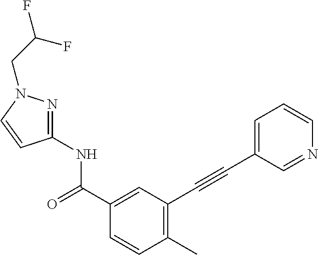 [SMILES: Cc1ccc(C(=O)Nc2ccn(CC(F)F)n2)cc1C#Cc1cccnc1] | A | 367.2 | 1.306 |
| 76 | 4-methyl-N-[4- methyl-1-(2,2,2- trifluoroethyl)-1H- pyrazol-3-yl]-3-[2- (pyridin-3- yl)ethynyl]benzamide | 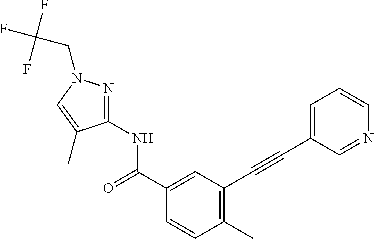 [SMILES: Cc1ccc(C(=O)Nc2nn(CC(F)(F)F)cc2C)cc1C#Cc1cccnc1] | A | 399.2 | 1.354 |
| 77 | N-(1-cyclobutyl-3- methyl-1H-pyrazol-4- yl)-4-methyl-3-[2- (pyridin-3- yl)ethynyl]benzamide | 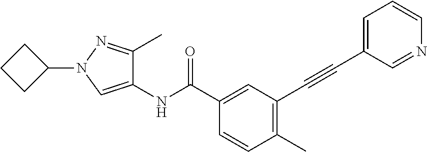 [SMILES: Cc1ccc(C(=O)Nc2cn(C3CCC3)nc2C)cc1C#Cc1cccnc1] | A | 371.2 | 1.356 |
| 79 | 4-methyl-N-[3- methyl-1-(propan-2- yl)-1H-pyrazol-4-yl]- 3-[2-(pyridin-3- yl)ethynyl]benzamide | 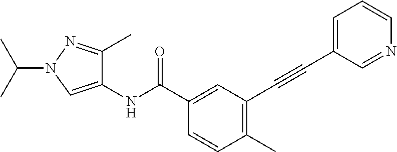 [SMILES: Cc1ccc(C(=O)Nc2cn(C(C)C)nc2C)cc1C#Cc1cccnc1] | A | 359.2 | 1.311 |
| 80 | 4-methyl-N-[1-(2- methylpropyl)-1H- pyrazol-3-yl]-3-[2- (pyridin-3- yl)ethynyl]benzamide | 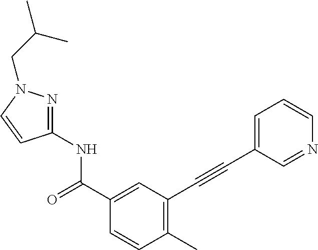 [SMILES: Cc1ccc(C(=O)Nc2ccn(CC(C)C)n2)cc1C#Cc1cccnc1] | A | 359.2 | 1.435 |
| 81 | 4-methyl-N-{5- methyl-4-oxo- 4H,5H,6H,7H- pyrazolo[1,5- a]pyrazin-2-yl}-3-[2- (pyridin-3- yl)ethynyl]benzamide | 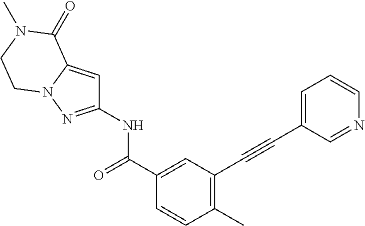 [SMILES: Cc1ccc(C(=O)Nc2cc3n(n2)CCN(C)C3=O)cc1C#Cc1cccnc1] | A | 386.2 | 1.085 |
| 83 | N-[1,4-dimethyl-3- (trifluoromethyl)-1H- pyrazol-5-yl]-4- methyl-3-[2- (pyridin-3- yl)ethynyl]benzamide | 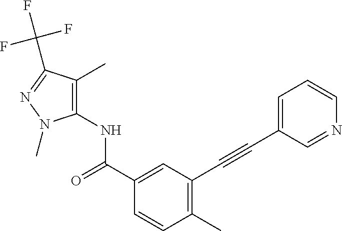 [SMILES: Cc1ccc(C(=O)Nc2c(C)c(C(F)(F)F)nn2C)cc1C#Cc1cccnc1] | B | 399.2 | 1.481 |
| 84 | N-(3-cyclopropyl-1- methyl-1H-pyrazol-5- yl)-4-methyl-3-[2- (pyridin-3- yl)ethynyl]benzamide | 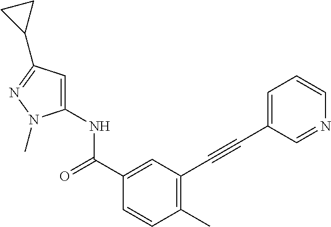 [SMILES: Cc1ccc(C(=O)Nc2cc(C3CC3)nn2C)cc1C#Cc1cccnc1] | A | 357.2 | 1.373 |
| 85 | 4-methyl-N-[1- methyl-3-(propan-2- yl)-1H-1,2,4-triazol- 5-yl]-3-[2-(pyridin-3- yl)ethynyl]benzamide | 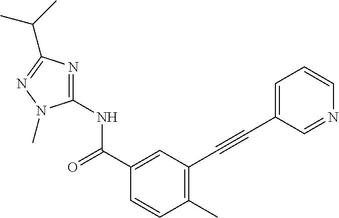 [SMILES: Cc1ccc(C(=O)Nc2nc(C(C)C)nn2C)cc1C#Cc1cccnc1] | B | 360.2 | 1.185 |
| 86 | N-[1- (difluoromethyl)-1H- pyrazol-4-yl]-4- methyl-3-[2- (pyridin-3- yl)ethynyl]benzamide | 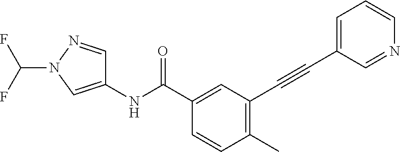 [SMILES: Cc1ccc(C(=O)Nc2cnn(C(F)F)c2)cc1C#Cc1cccnc1] | A | 353.2 | 1.375 |
| 87 | N-[1-(2,2- difluoroethyl)-4- methyl-1H-pyrazol-3- yl]-4-methyl-3-[2- (pyridin-3- yl)ethynyl]benzamide | 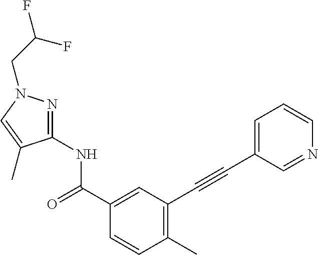 [SMILES: Cc1ccc(C(=O)Nc2nn(CC(F)F)cc2C)cc1C#Cc1cccnc1] | A | 381.2 | 1.282 |
| 90 | N-[4-chloro-1- (propan-2-yl)-1H- pyrazol-3-yl]-4- methyl-3-[2- (pyridin-3- yl)ethynyl]benzamide | 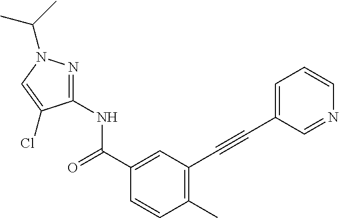 [SMILES: Cc1ccc(C(=O)Nc2nn(C(C)C)cc2Cl)cc1C#Cc1cccnc1] | A | 379.2 | 1.365 |
| 91 | N-(3-cyclopropyl-1- ethyl-1H-pyrazol-5- yl)-4-methyl-3-[2- (pyridin-3- yl)ethynyl]benzamide | 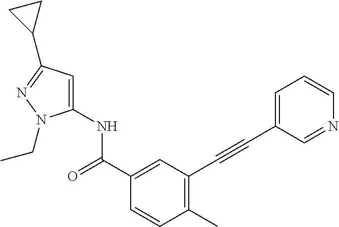 [SMILES: CCn1nc(C2CC2)cc1NC(=O)c1ccc(C)c(C#Cc2cccnc2)c1] | A | 371.2 | 1.346 |
| 92 | 4-methyl-N-[5- methyl-1-(2- methylpropyl)-1H- pyrazol-3-yl]-3-[2- (pyridin-3- yl)ethynyl]benzamide | 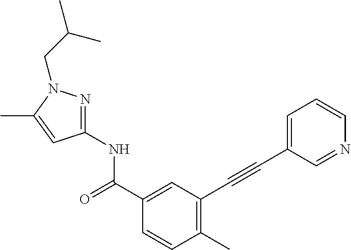 [SMILES: Cc1ccc(C(=O)Nc2cc(C)n(CC(C)C)n2)cc1C#Cc1cccnc1] | A | 373.2 | 1.488 |
| 93 | N-[1-(1- methoxypropan-2- yl)-1H-pyrazol-3-yl]- 4-methyl-3-[2- (pyridin-3- yl)ethynyl]benzamide | 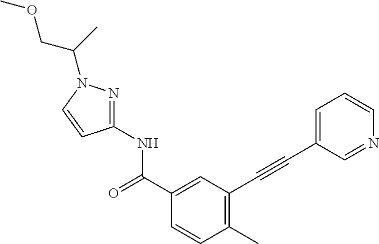 [SMILES: COCC(C)n1ccc(NC(=O)c2ccc(C)c(C#Cc3cccnc3)c2)n1] | A | 375.2 | 1.320 |
| 98 | N-[1-(2-fluoroethyl)- 1H-pyrazol-4-yl]-4- methyl-3-[2- (pyridin-3- yl)ethynyl]benzamide | 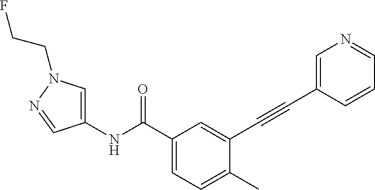 [SMILES: Cc1ccc(C(=O)Nc2cnn(CCF)c2)cc1C#Cc1cccnc1] | A | 349.2 | 1.253 |
| 99 | 4-methyl-N- {4H,5H,6H,7H- pyrazolo[1,5- a]pyridin-3-yl}-3-[2- (pyridin-3- yl)ethynyl]benzamide | 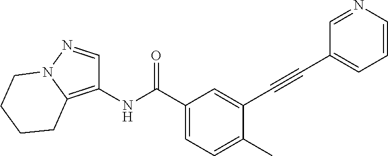 [SMILES: Cc1ccc(C(=O)Nc2cnn3c2CCCC3)cc1C#Cc1cccnc1] | A | 357.2 | 1.258 |
| 100 | 4-methyl-3-[2- (pyridin-3-yl)ethynyl]- N-(4,5,6,7- tetrahydro-1,2- benzoxazol-3- yl)benzamide | 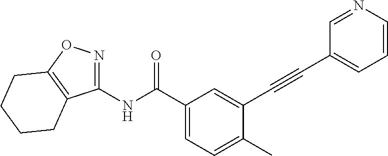 [SMILES: Cc1ccc(C(=O)Nc2noc3c2CCCC3)cc1C#Cc1cccnc1] | D | 358.0 | 1.380 |
| 101 | 4-methyl-3-[2- (pyridin-3-yl)ethynyl]- N-(4,5,6,7- tetrahydro-2,1- benzoxazol-3- yl)benzamide | 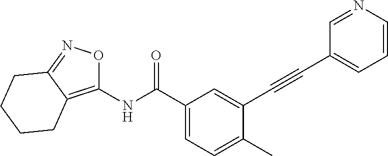 [SMILES: Cc1ccc(C(=O)Nc2onc3c2CCCC3)cc1C#Cc1cccnc1] | C | 358.0 | 1.453 |
| 102 | N-(5,5-difluoro- 4,5,6,7-tetrahydro- 1,2-benzoxazol-3-yl)- 4-methyl-3-[2- (pyridin-3- yl)ethynyl]benzamide | 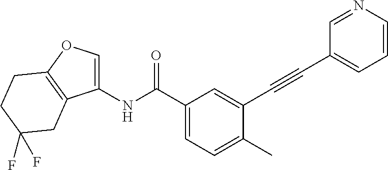 [SMILES: Cc1ccc(C(=O)Nc2coc3c2CC(F)(F)CC3)cc1C#Cc1cccnc1] | D | 394.2 | 1.491 |
| 103 | N-(5-tert-butyl-1,3,4- oxadiazol-2-yl)-4- methyl-3-[2- (pyridin-3- yl)ethynyl]benzamide | 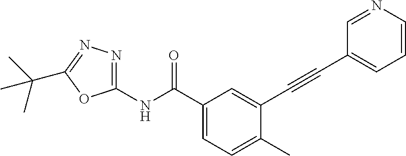 [SMILES: Cc1ccc(C(=O)Nc2nnc(C(C)(C)C)o2)cc1C#Cc1cccnc1] | C | 361.2 | 1.429 |

| | | | Yield |
|---|---|---|---|
| Ex. | Starting Amine | CAS No. | (%) |
| 34 | 4-tert-butyl-1,3-oxazol-2-amine | 97567-79-8 | 33 |
| 35 | 4-(propan-2-yl)-1,3-oxazol-2-amine | 229003-15-0 | 19 |
| 36 | 2-(propan-2-yl)-2H-1,2,3,4-tetrazol-5-amine | 229003-16-1 | 25 |
| 37 | 1-tert-butyl-1H-1,2,4-triazol-3-amine | 1380786-07-1 | 31 |
| 43 | 1-(difluoromethyl)-5-methyl-1H-pyrazol-4-amine | 1245772-66-0 | 45 |
| 45 | 4-(2-methylpropyl)-1,3-oxazol-2-amine | 1782545-44-1 | 28 |
| 46 | 3-(propan-2-yl)-1,2,4-oxadiazol-5-amine | 3874-89-3 | 38 |
| 47 | 1-(butan-2-yl)-1H-pyrazol-3-amine | 1006481-33-9 | 44 |
| 48 | 1-(2,2,2-trifluoroethyl)-1H-pyrazol-4-amine | 919278-39-0 | 54 |
| 49 | 6,6-dimethyl-5H,6H,7H-pyrazolo[3,2-b][1,3]oxazin- | 1708157-62-3 | 35 |
| | 3-amine | | |
| 50 | 1-propyl-1H-pyrazol-4-amine | 1006483-43-7 | 41 |
| 51 | 3-cyclobutyl-1-methyl-1H-pyrazol-5-amine | 92406-41-2 | 38 |
| 52 | 3-methoxy-1-(2,2,2-trifluoroethyl)-1H-pyrazol-4-amine | 1006462-54-9 | 72 |
| 53 | 1-(2,2-difluoroethyl)-1H-pyrazol-4-amine | 1006333-08-9 | 34 |
| 54 | 1-(2,2-difluoroethyl)-5-methyl-1H-pyrazol-3-amine | 1006486-89-0 | 41 |
| 55 | 5-methyl-1-(propan-2-yl)-1H-pyrazol-3-amine | 956440-80-5 | 49 |
| 58 | 4-methyl-1-(propan-2-yl)-1H-pyrazol-3-amine | 1174866-04-6 | 41 |
| 59 | 5-(difluoromethyl)-1-methyl-1H-1,2,3-triazol-4-amine | 1936660-43-3 | 14 |
| 61 | 1-methyl-3-(2-methylpropyn-1H-pyrazol-5-amine | 118430-72-1 | 41 |
| 62 | 1-(cydopropylmethyl)-1H-pyrazol-4-amine | 1156169-29-7 | 45 |
| 66 | 1-(propan-2-yl)-1H-pyrazol-3-amine | 857267-04-0 | 46 |
| 68 | 1-(cyclobutylmethyl)-1H-pyrazol-4-amine | 1225065-25-7 | 57 |
| 70 | 3-(cydopropylmethyl)-1-methyl-1H-pyrazol-5-amine | 1249335-32-7 | 26 |
| 71 | 3-methyl-1-(2,2,2-trifluoroethyl)-1H-pyrazol-4-amine | 1006448-59-4 | 56 |
| 72 | 1-(2,2-difluoroethyl)-3-methyl-1H-pyrazol-4-amine | 1006462-68-5 | 58 |
| 73 | 1-(2,2-difluorocyclopropyl)-1H-pyrazol-3-amine | 2173998-94-0 | 44 |
| 74 | 3-methyl-1-(2-methylpropyl)-1H-pyrazol-4-amine | 405548-38-1 | 59 |
| 75 | 1-(2,2-difluoroethyl)-1H-pyrazol-3-amine | 1006462-38-9 | 53 |
| 76 | 4-methyl-1-(2,2,2-trifluoroethyl)-1H-pyrazol-3-amine | 1174876-14-2 | 63 |
| 77 | 1-cyclobutyl-3-methyl-1H-pyrazol-4-amine | 2137577-49-0 | 49 |
| 79 | 3-methyl-1-(propan-2-yl)-1H-pyrazol-4-amine | 29751-98-2 | 48 |
| 80 | 1-(2-methylpropyl)-1H-pyrazol-3-amine | 1003012-08-5 | 50 |
| 81 | 2-amino-5-methyl-4H,5H,6H,7H-pyrazolo[1,5-a] | 1378683-88-5 | 41 |
| | pyrazin-4-one | | |
| 83 | 1,4-dimethyl-3-(trifluoromethyl)-1H-pyrazol-5-amine | 164668-13-7 | 25 |
| 84 | 3-cyclopropyl-1-methyl-1H-Pyrazol-5-amine | 118430-74-3 | 53 |
| 85 | 1-methyl-3-(propan-2-yl)-1H-1,2,4-triazol-5-amine | 1269152-62-6 | 14 |
| 86 | 14difluoromethyl)-1H-pyrazol-4-amine | 1174309-16-0 | 62 |
| 87 | 1-(2,2-difluoroethyl)-4-methyl-1H-pyrazol-3-amine | 1174875-53-6 | 44 |
| 90 | 4-chloro-1-(propan-2-yl)-1H-pyrazol-3-amine | 1006481-38-4 | 19 |
| 91 | 3-cyclopropyl-1-ethyl-1H-pyrazol-5-amine | 1172505-99-5 | 34 |
| 92 | 5-methyl-1-(2-methylpropyl)-1H-pyrazol-3-amine | 1006470-61-6 | 54 |
| 93 | 1-(1-methoxypropan-2-yl)-1H-pyrazol-3-amine | 1341630-16-7 | 41 |
| 96 | 1-(2-fluoroethy)-1H-pyrazol-4-amine | 1427021-88-2 | 57 |
| 99 | 4H,5H,6H,7H-pyrazolo[1,5-a]pyridin-3-amine | 1196152-11-0 | 45 |
| 100 | 4,5,6,7-tetrahydro-1,2-benzoxazol-3-amine | 1004-64-4 | 26 |
| 101 | 4,5,6,7-tetrahydro-2,1-benzoxazol-3-amine | 13054-47-2 | 32 |
| 102 | 5,5-difluor-4,5,6,7-tetrahydro-1,2-benzoxazol-3-amine | 1783608-43-4 | 40 |
| 103 | 5-tert-butyl-1,3,4-oxadiazol-2-amine | 69741-92-0 | 30 |

### Example 38

### N-(1-tert-butylimidazol-4-yl)-4-methyl-3-[2-(3-pyridyl)ethynyl]benzamide

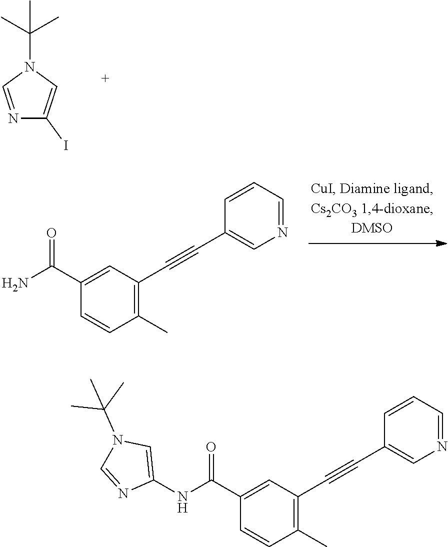

To Intermediate 12 (263 mg, 1.11 mmol) in a microwave vial was added Cs₂CO₃ (905 mg, 2.78 mmol) and CuI (212 mg, 1.11 mmol) and the atmosphere purged and replaced with N₂ (×3). 1,4-Dioxane (7.0 mL) was added and the mixture degassed for 5 min before addition of a solution of 1-tert-butyl-4-iodo-imidazole and 1-tert-butyl-5-iodo-imidazole as an inseparable mixture (Intermediate 110) (278 mg, 1.11 mmol) in DMSO (1.4 mL) followed by trans-N,N′-dimethylcyclohexane-1,2-diamine (175 μL, 1.11 mmol) and the reaction sealed and heated to 100° C. for 2 h. The reaction was allowed to cool to rt, diluted with DCM (15 mL) and washed with sat aq NH₄Cl (10 mL). The aqueous phase was extracted three times with DCM (3×10 mL). The combined organic phases were dried over Na₂SO₄, filtered and concentrated to dryness. The residue was purified by RP chromatography ([Biotage system, 30 g C18 cartridge, loaded in DMSO]0.1% NH₃/MeCN 5 to 80%) and the product freeze-dried to obtain N-(1-tert-butylimidazol-4-yl)-4-methyl-3-[2-(3-pyridyl)ethynyl]benzamide (135 mg, 33%) as a white solid. UPLC (Method A) 3.15 min, 99%, [M+H]⁺=359.3. Structure confirmed by NOESY analysis, with a clear interaction between the 2- and 5-protons on the imidazole and the tBu.

### Example 39

### N-(5,5-dimethyl-6,8-dihydroimidazo[2,1-c][1,4]oxazin-2-yl)-4-methyl-3-[2-(3-pyridyl)ethynyl]benzamide

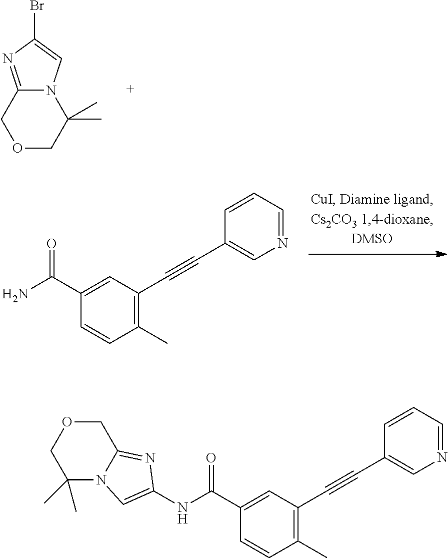 [SMILES: CC1(C)COCc2nc(Br)cn21 ; Cc1ccc(C(N)=O)cc1C#Cc1cccnc1 ; Cc1ccc(C(=O)Nc2cn3c(n2)COCC3(C)C)cc1C#Cc1cccnc1]

To a degassed, stirred solution of Intermediate 55 (307 mg, 1.33 mmol) and Cs₂CO₃ (649 mg, 1.99 mmol) in 1,4-dioxane (9.0 mL) and DMSO (3.0 mL) at rt under N2 was added copper (I) iodide (253 mg, 1.33 mmol) portionwise and trans N,N′-dimethylcyclohexane-1,2-diamine (251 μL, 1.59 mmol) dropwise. Intermediate 12 (314 mg, 1.33 mmol) was then added and the reaction was stirred at 90° C. for 2 h. The reaction was cooled, concentrated, diluted with sat. NH₄Cl (20 mL) and extracted with 1:4 IPA:DCM (7×10 mL). The combined organic layers were dried over MgSO₄, filtered and concentrated to a crude blue/black oil (5.50 g). The crude material was passed through a silica plug (0-20% MeCOH in EtOAc) to afford a green/brown oil (910 mg). This was further purified by reverse phase column chromatography (Biotage Isolera, reverse phase, [60 g], HP-Sphere C18 ULTRA, 25 μm [residue loaded in DMSO], 0-45% MeCN in H₂O, both eluents containing 0.1 Vol % NH₃) to afford the title compound as beige solid (130 mg, 25%). UPLC (Method A) 3.01 min, 100%, [M+H]⁺=387.3.

### Example 40

### N-(6,6-dimethyl-5,7-dihydropyrrolo[1,2-c]imidazol-1-yl)-4-methyl-3-[2-(3-pyridyl)ethynyl]benzamide

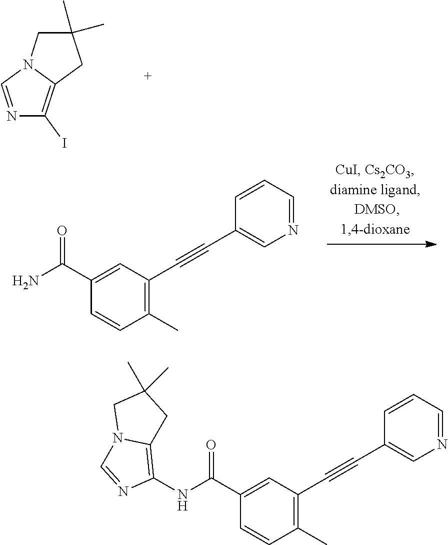

In a microwave vial was added Intermediate 12 (45.1 mg, 0.19 mmol), Cs₂CO₃ (155.0 mg, 0.48 mmol) and CuI (36.3 mg, 0.19 mmol) and the atmosphere purged and replaced by N₂ (×3). 1,4-Dioxane (1.5 mL) was added and the mixture degassed for 5 min, before addition of a solution of Intermediate 58 (50.0 mg, 0.19 mmol) in DMSO (0.4 mL) followed by trans N,N′-dimethylcyclohexane-1,2-diamine (30.1 μL, 0.19 mmol). The reaction was sealed and heated to 100° C. for 2 h. The reaction was allowed to cool to rt, diluted with DCM (15 mL) and washed with sat. aq. NH₄Cl (10 mL). The aqueous phase was extracted with DCM (3×10 mL) and combined organics dried over Na₂SO₄, filtered and concentrated to dryness. The residue was purified by reverse phase chromatography ([Biotage system, 30 g C18 cartridge, loaded in DMSO] 0.1% NH₃/MeCN 5 to 80%). The material was further purified by preparative HPLC (Phenomenex Luna C18, 5 μm, MeCN/H₂O 65/35 isocratic, 5 min run) and the product freeze-dried to yield N-(6,6-dimethyl-5,7-dihydropyrrolo[1,2-c]imidazol-1-yl)-4-methyl-3-[2-(3-pyridyl)ethynyl]benzamide (5.7 mg, 8.0%). UPLC (Method A) 3.11 min, 99.3%, [M+H]⁺=371.3.

### Example 41

### 4-Methyl-3-[2-(3-pyridyl)ethynyl]-N-spiro[5,6-dihydropyrrolo[1,2-b]pyrazole-4,1′-cyclobutane]-2-yl-benzamide

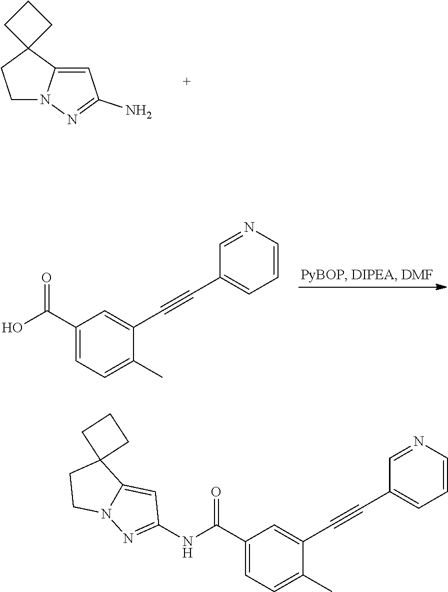

To a solution of Intermediate 2 (145 mg, 0.61 mmol) and Intermediate 61 (used crude, assumed 50%, 200 mg, 0.61 mmol) in DMF (3.0 mL) was added PyBOP (478 mg, 0.92 mmol) followed by DIPEA (320 μL, 1.84 mmol). The reaction mixture was stirred at rt for 1 h before being concentrated and purified by column chromatography (Biotage Isolera, reverse phase, 30 g, HP-Sphere C18 ULTRA, 25 μm [residue loaded in DMF], 0% to 70% MeCOH in H₂O, both eluents containing 0.1 Vol % NH₃) to give crude product (35.0 mg) containing an aromatic impurity, observed by 1H NMR. The crude mixture was triturated with TBME/MeCN (9:1) to give 4-methyl-3-[2-(3-pyridyl)ethynyl]-N-spiro[5,6-dihydropyrrolo[1,2-b]pyrazole-4,1′-cyclobutane]-2-yl-benzamide (14.7 mg, 6%). UPLC (Method A) 3.40 min, 100%, [M+H]⁺=383.3.

### Example 42

### N-(1-cyclopentylimidazol-4-yl)-4-methyl-3-[2-(3-pyridyl)ethynyl]benzamide

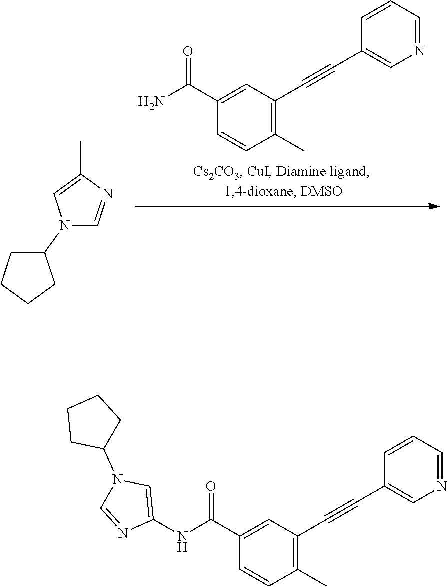 [SMILES: Cc1cn(C2CCCC2)cn1 ; Cc1ccc(C(N)=O)cc1C#Cc1cccnc1 ; Cc1ccc(C(=O)Nc2cn(C3CCCC3)cn2)cc1C#Cc1cccnc1]

An N₂ purged stirring solution of Intermediate 62 (300 mg, 1.15 mmol) and caesium carbonate (560 mg, 1.72 mmol) in 1,4-dioxane (9.0 mL) and DMSO (3.0 mL) at rt was degassed for 15 min followed by portionwise addition of copper (I) iodide (220 mg, 1.14 mmol) and dropwise addition of trans N,N′-dimethylcyclohexane-1,2-diamine (220 μL, 1.37 mmol). Intermediate 12 (270 mg, 1.14 mmol) was then added and the reaction was stirred at 90° C. for 6 h. The reaction was then retreated with copper (I) iodide (110 mg, 0.57 mmol) and trans N,N′-dimethylcyclohexane-1,2-diamine (110 μL, 0.69 mmol) and stirred for a further 16 h. The reaction was cooled, concentrated and the resulting residue was diluted with sat. NH₄Cl (10 mL) and extracted using 1:3 IPA:DCM (5×10 mL). The combined organic layers were dried over MgSO₄, filtered and concentrated to give the crude product as a blue/black oil (3.01 g). The crude product was passed through a silica plug (eluted with 0-20% MeCOH in EtOAc) to give a semi-purified product (1.30 g). Purification by reverse-phase chromatography (Biotage Isolera, reverse phase, [30 g], HP-Sphere C18 ULTRA, 25 μm [residue loaded in DMSO], 10% to 70% MeCN in H₂O, both eluents containing 0.1 Vol % NH₃) gave the purified product as a yellow solid (45 mg). Trituration of the product with TBME (10 mL) and filtration afforded the title compound (25 mg, 6%) as a white solid. UPLC (Method A) 3.24 min, 99%, [M+H]⁺=371.3.

### Example 44

### N-(5,5-dimethyl-6,7-dihydropyrrolo[1,2-a]imidazol-2-yl)-4-methyl-3-[2-(3-pyridyl)ethynyl]benzamide

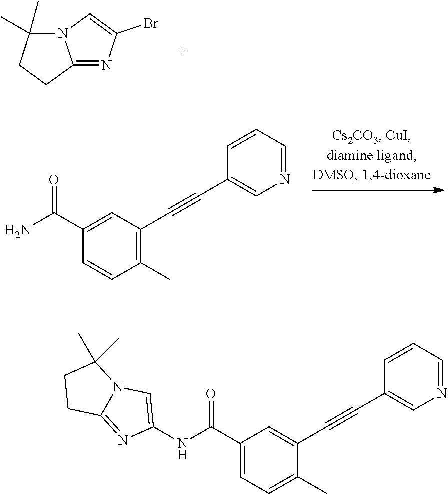 [SMILES: CC1(C)CCc2nc(Br)cn21 ; Cc1ccc(C(N)=O)cc1C#Cc1cccnc1 ; Cc1ccc(C(=O)Nc2cn3c(n2)CCC3(C)C)cc1C#Cc1cccnc1]

To Intermediate 12 (1.33 g, 5.63 mmol), CuI (1.07 g, 5.63 mmol) and Cs₂CO₃ (4.58 g, 14.1 mmol) under an atmosphere of N₂ was added 1,4-dioxane (56 mL) and the mixture degassed for 5 min before addition of a solution of Intermediate 65 (1.21 g, 5.63 mmol) in DMSO (14 mL), followed by trans N,N′-dimethylcyclohexane-1,2-diamine (0.89 mL, 5.63 mmol). The reaction was heated to 100° C. for 2 h, allowed to cool to rt and filtered through Celite. The filtrate was concentrated in vacuo, diluted with DCM (300 mL), washed with NH₄Cl sat aq (3×50 mL), dried over Na₂SO₄, filtered and concentrated in vacuo. The residue was purified by RP chromatography ([Biotage system, C18 30 g cartridge, loaded in DMSO] 0.1% aq NH₃/MeCN 10 to 80%) and the desired product freeze-dried to yield N-(5,5-dimethyl-6,7-dihydropyrrolo[1,2-a]imidazol-2-yl)-4-methyl-3-[2-(3-pyridyl)ethynyl]benzamide (812 mg, 38%) as a colourless solid. UPLC (Method A) 3.11 min, 99%, [M+H]⁺=371.3.

### Example 56

### N-(5-isopropyl-6,7-dihydro-5H-pyrrolo[1,2-a]imidazol-2-yl)-4-methyl-3-[2-(3-pyridyl)ethynyl]benzamide

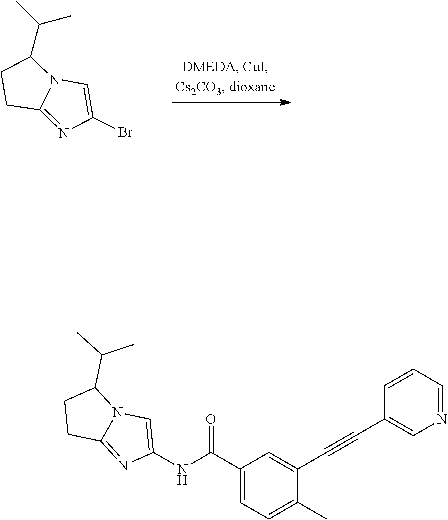 [SMILES: CC(C)C1CCc2nc(Br)cn21 ; Cc1ccc(C(=O)Nc2cn3c(n2)CCC3C(C)C)cc1C#Cc1cccnc1]

Intermediate 68 (150 mg, 0.65 mmol, 1.0 eq), Intermediate 12 (155 mg, 0.65 mmol, 1.0 eq), copper (I) iodide (125 mg, 0.65 mmol, 1.0 eq), N,N′-dimethylethylenediamine (58 mg, 0.65 mmol, 1.0 eq) and caesium carbonate (320 mg, 0.98 mmol, 1.5 eq) were suspended in 1,4-dioxane (3 mL) and DMSO (1 mL), degassed with N₂, sealed and heated at 100° C. for 4 h. Further portions of copper (I) iodide (125 mg, 0.65 mmol, 1.0 eq) and N,N′-dimethylethylenediamine (58 mg, 0.65 mmol, 1.0 eq) were added and the reaction heated at 100° C. for 3 h. The reaction was cooled to rt and the volatiles removed in vacuo. The residue was stirred in MeCN (25 mL) and filtered. The solid was stirred in 2M HCl (25 mL), filtered, the filtrate basified with solid K₂CO₃, extracted with EtOAc (2×25 mL), dried (MgSO₄) and concentrated in vacuo. The residue was triturated with tert-butyl methyl ether, then re-triturated with EtOAc and the solid recrystallised from EtOAc to afford the title compound (11 mg, 4%) as a white solid. UPLC (Method A): 3.26 min, 99%, [M+H]⁺=385.3.

### Example 57

### N-(5-isopropyl-6,8-dihydro-5H-imidazo[2,1-c][1,4]oxazin-2-yl)-4-methyl-3-[2-(3-pyridyl)ethynyl]benzamide

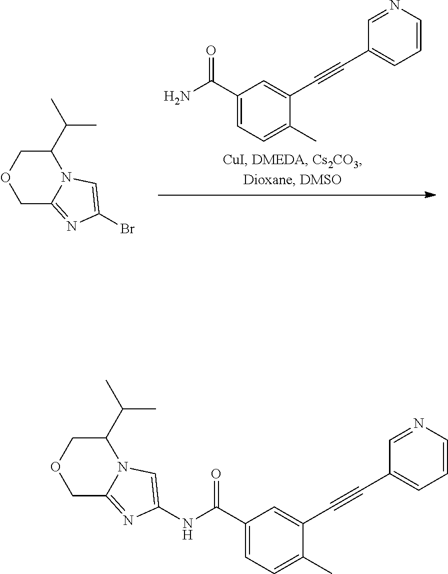 [SMILES: CC(C)C1COCc2nc(Br)cn21 ; Cc1ccc(C(N)=O)cc1C#Cc1cccnc1 ; Cc1ccc(C(=O)Nc2cn3c(n2)COCC3C(C)C)cc1C#Cc1cccnc1]

A mixture of Intermediate 69 (˜20% w/w, 282 mg, 0.23 mmol, 1.0 eq), Intermediate 12 (82 mg, 0.35 mmol, 1.5 eq), CuI (66 mg, 0.35 mmol, 1.5 eq), N,N′-dimethylethylenediamine (37 μL, 0.35 mmol, 1.5 eq) and caesium carbonate (150 mg, 0.46 mmol, 2.0 eq) in dioxane:DMSO (3 mL, 2:1) was heated at 95° C. in a sealed vial for 16 h. A further 1 equivalent of CuI and N,N′-dimethylethylenediamine was added and the reaction heated at 110° C. in a sealed vial for 3 h. The reaction was concentrated in vacuo and partitioned between H₂O (20 mL) and tert-butyl methyl ether (20 mL) and the solid material filtered. The solid and organic layer were combined, absorbed onto silica and purified by normal phase chromatography 1-3% MeCOH/DCM, then by reverse phase chromatography 20-80% MeCN/H₂O (0.1% NH₃ modifier) to afford the title compound (4 mg, 4%) as an orange solid after freeze-drying. UPLC (Method A): 3.15 min, 100%, [M+H]⁺=401.3.

### Example 60

### 4-Methyl-N-(5-methyl-6,7-dihydro-5H-pyrrolo[1,2-c]imidazol-1-yl)-3-[2-(3-pyridyl)ethynyl]benzamide

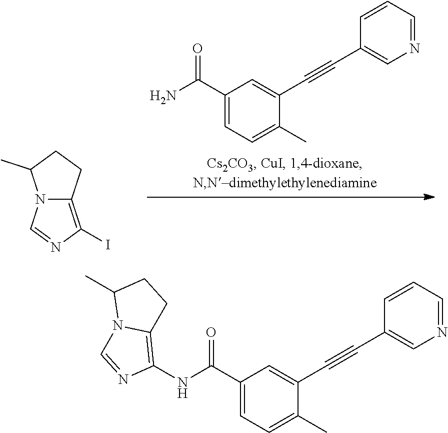

A suspension of Intermediate 12 (70 mg, 0.3 mmol, 1 eq), Intermediate 75 (73 mg, 0.3 mmol, 1 eq), CuI (56 mg, 0.3 mmol, 1 eq), N,N′-dimethylethylenediamine (26 mg, 0.3 mmol, 1 eq) and caesium carbonate (193 mg, 0.6 mmol, 2 eq) in dioxane (6 mL) was degassed with N₂ and heated in a sealed vial at 100° C. for 72 h. The reaction was cooled to rt and concentrated in vacuo. The residue was dissolved in 0.5:5:95 NH₃/MeOH/DCM, filtered through a pad of Celite and concentrated in vacuo. The residue was dissolved in EtOAc, the solid removed by filtration and the filtrate concentrated in vacuo. The crude material was purified by normal phase chromatography 0.2:2:98-0.5:5:95 NH₃/MeOH/DCM to afford the title compound (16 mg, 15%) as a white solid. LCMS (Method D): 2.50 min, 98%, [M+H]⁺=357.1.

### Example 63

### 4-Methyl-3-[2-(3-pyridyl)ethynyl]-N-spiro[5,6-dihydropyrrolo[1,2-b]pyrazole-4,1′-cyclopropane]-2-yl-benzamide

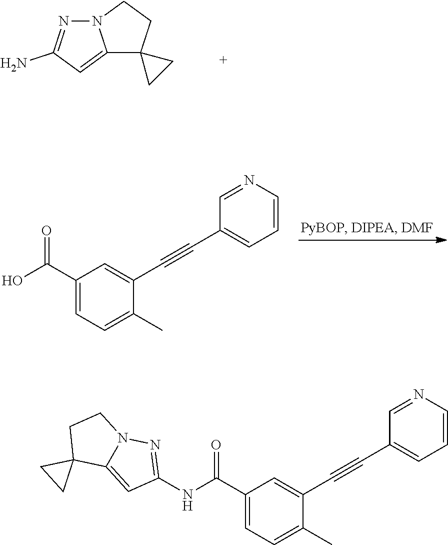 [SMILES: Nc1cc2n(n1)CCC21CC1 ; Cc1ccc(C(=O)O)cc1C#Cc1cccnc1 ; Cc1ccc(C(=O)Nc2cc3n(n2)CCC32CC2)cc1C#Cc1cccnc1]

To a solution of Intermediate 2 (73.0 mg, 0.31 mmol) and Intermediate 78 (46.0 mg, 0.31 mmol) in DMF (2.0 mL) was added PyBOP (241.0 mg, 0.46 mmol) followed by DIPEA (161 μL, 0.92 mmol). The resulting solution was stirred at rt for 1 h and then concentrated in vacuo. The crude residue was purified by column chromatography (Biotage Isolera, reverse phase, 12 g, HP-Sphere C18 ULTRA, 25 μm [residue loaded in DMSO], 5% to 60% MeCN in H₂O, both eluents containing 0.1 Vol % NH₃). The resulting oil was triturated in CH₃CN (with sonication). The resulting solid was filtered and dried in vacuo to yield 4-methyl-3-[2-(3-pyridyl)ethynyl]-N-spiro[5,6-dihydropyrrolo[1,2-b]pyrazole-4,1′-cyclopropane]-2-yl-benzamide (50.8 mg, 44%) as an off-white solid. UPLC (Method A) 3.17 min, 99%, [M+H]⁺=369.3.

### Example 64

### N-(1-isobutylimidazol-4-yl)-4-methyl-3-[2-(3-pyridyl)ethynyl]benzamide

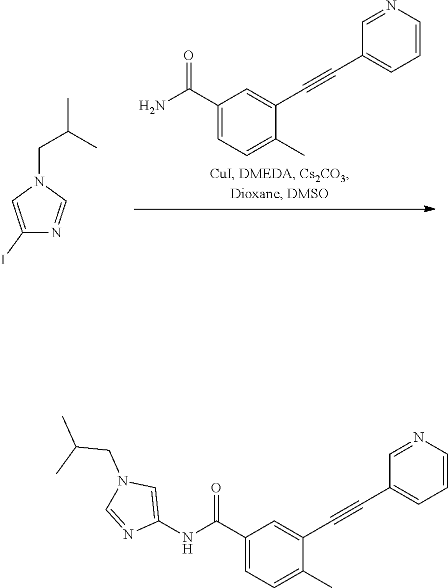

To Intermediate 12 (180 mg, 0.76 mmol, 1.0 eq), caesium carbonate (373 mg, 1.14 mmol, 1.5 eq) and copper (I) iodide (145 mg, 0.76 mmol, 1.0 eq), under a N₂ atmosphere, was added dioxane (8 mL), and N₂ bubbled through the reaction mixture for 5 min. To the mixture was added a solution of 4-iodo-1-isobutyl-imidazole Intermediate 79 (191 mg, 0.76 mmol, 1.0 eq) in DMSO (2 mL), followed by N,N′-dimethylethylenediamine (99 μL, 0.92 mmol, 1.2 eq). The vial was sealed and heated at 95° C. overnight. The reaction was cooled to rt, diluted with DCM (50 mL) and sat. aq. NH₄Cl (20 mL), the phases were separated and the aqueous extracted with DCM (3×30 mL). Combined organics were dried over Na₂SO₄ and concentrated in vacuo. The residue was purified by normal phase chromatography (KP-NH) 0-30% acetone/toluene and the resultant solid was triturated in MeCN (1 mL) to afford the title compound (50 mg, 18%) as a white solid. UPLC (Method A) 3.19 min, 100%, [M+H]⁺=359.3.

### Example 65

### N-(4-ethyl-5,6-dihydro-4H-pyrrolo[1,2-b]pyrazol-2-yl)-4-methyl-3-[2-(3-pyridyl)ethynyl]benzamide

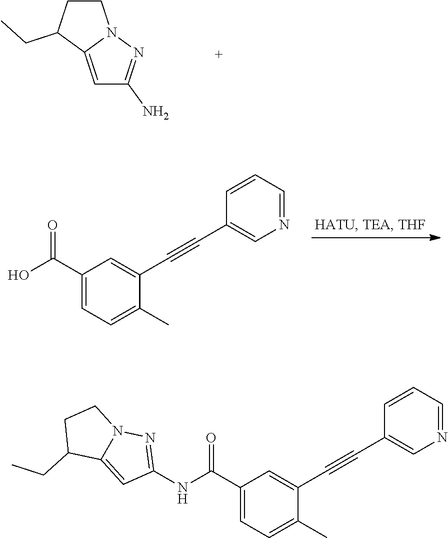 [SMILES: CCC1CCn2nc(N)cc21 ; Cc1ccc(C(=O)O)cc1C#Cc1cccnc1 ; CCC1CCn2nc(NC(=O)c3ccc(C)c(C#Cc4cccnc4)c3)cc21]

To a solution of Intermediate 82 (300 mg, 1.98 mmol), Intermediate 2 (471 mg, 1.98 mmol) and TEA (1.11 mL, 7.94 mmol) in THF (6.6 mL) was added HATU (1.13 g, 2.98 mmol) and the reaction stirred at rt under N₂ overnight. The reaction was quenched with brine (10 mL), diluted with EtOAc (10 mL) and the phases separated. The aqueous phase was extracted with EtOAc (2×20 mL), the combined organics washed with H₂O (10 mL), dried over MgSO₄ and concentrated in vacuo. The crude material was purified by column chromatography (Biotage Isolera, reverse phase, [60 g], HP-Sphere C18 ULTRA, 25 μm [residue loaded in DMSO], 5-100% MeCN/H₂O, both eluents containing 0.1 Vol % NH₃) and re-purified by column chromatography (Biotage Isolera, normal phase, [25 g], KP-NH 40-63 μm/230-400 mesh, 60 Å, residue loaded in DCM, 0-50% EtOAc/Heptane) to afford N-(4-ethyl-5,6-dihydro-4H-pyrrolo[1,2-b]pyrazol-2-yl)-4-methyl-3-[2-(3-pyridyl)ethynyl]benzamide (62.7 mg, 8.5%) as a colourless solid. LCMS (Method D) 2.78 min, 100%, [M+H]⁺=371.1.

### Example 67

### 4-Methyl-N-(5-methyl-5,6,7,8-tetrahydroimidazo[1,2-a]pyridin-2-yl)-3-[2-(3-pyridyl)ethynyl]benzamide

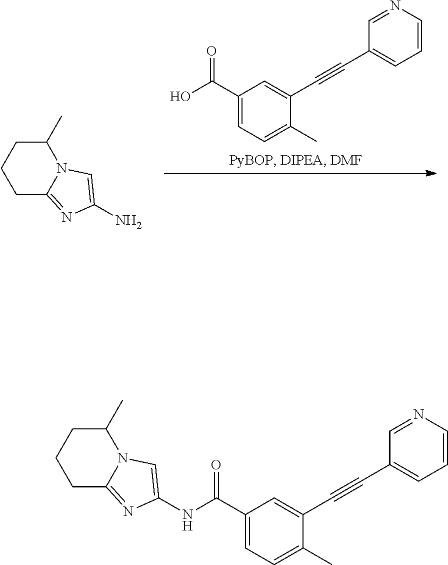 [SMILES: CC1CCCc2nc(N)cn21 ; Cc1ccc(C(=O)O)cc1C#Cc1cccnc1 ; Cc1ccc(C(=O)Nc2cn3c(n2)CCCC3C)cc1C#Cc1cccnc1]

A suspension of Intermediate 2 (106 mg, 0.45 mmol, 1.0 eq), benzotriazol-1-yl-oxytripyrrolidinophosphonium hexafluorophosphate (257 mg, 0.49 mmol, 1.1 eq) and N,N-diisopropylethylamine (120 μL, 0.67 mmol, 1.5 eq) in DMF (1.5 mL) was stirred at rt for 5 min and the resulting solution transferred to a suspension of Intermediate 90 (68 mg, 0.45 mmol, 1.0 eq) in DMF (1.5 mL) and the reaction mixture was stirred at rt for 18 h. The reaction was quenched by pouring into H₂O (10 mL), diluted with DCM (10 mL), the phases were separated and the aqueous extracted with DCM (3×10 mL). The combined organics were dried (Na₂SO₄) and concentrated in vacuo. The crude material was purified by normal phase chromatography (KP-NH) 50% acetone/DCM followed by reverse phase chromatography in 15-100% MeCN/H₂O (0.1% NH₃ modifier) to afford the title compound (7.3 mg, 4%) as a pale yellow solid. UPLC (Method A): 3.10 min, 96%, [M+H]⁺=371.

### Example 69

### N-(1-cyclobutylimidazol-4-yl)-4-methyl-3-[2-(3-pyridyl)ethynyl]benzamide

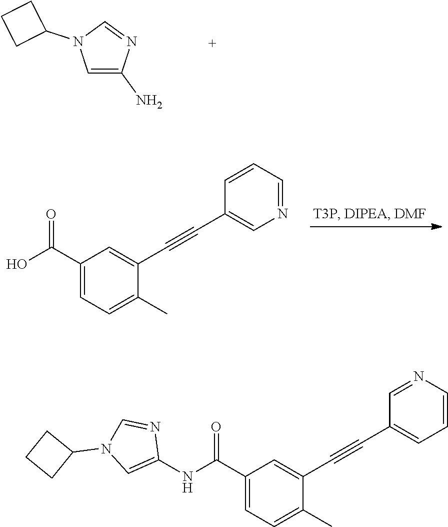 [SMILES: Nc1cn(C2CCC2)cn1 ; Cc1ccc(C(=O)O)cc1C#Cc1cccnc1 ; Cc1ccc(C(=O)Nc2cn(C3CCC3)cn2)cc1C#Cc1cccnc1]

To a suspension of Intermediate 2 (200 mg, 0.84 mmol) in DMF (2 mL) under N₂ was added DIPEA (294 μL, 1.69 mmol). To the resulting clear solution was added 1-propylphosphonic acid cyclic anhydride (1.00 mL, 1.68 mmol) and the reaction stirred for 5 min at rt before addition of 1-cyclobutylimidazol-4-amine hydrochloride (220 mg, 1.26 mmol) and DIPEA (294 μL, 1.69 mmol) as a DMF solution (2 mL). The reaction was heated at 60° C. for 24 h. The reaction was cooled to rt and diluted with DCM (10 mL) washed with phosphate buffer (pH 7, 3×5.0 mL), dried (Na₂SO₄) and concentrated in vacuo. The residue was purified by column chromatography (Biotage Isolera, reverse phase, 12 g, HP-Sphere C18 ULTRA, 25 μm, 10% to 100% MeCN in H₂O, both eluents containing 0.1 Vol % NH₃) to give N-(1-cyclobutylimidazol-4-yl)-4-methyl-3-[2-(3-pyridyl)ethynyl]benzamide (28.7 mg, 96.3%) as a white solid after freeze drying. UPLC (Method G) 3.10 min, 96.9%, [M+H]⁺=357.3.

### Example 78

### N-[1-(2-fluoroethyl)imidazol-4-yl]-4-methyl-3-[2-(3-pyridyl)ethynyl]benzamide

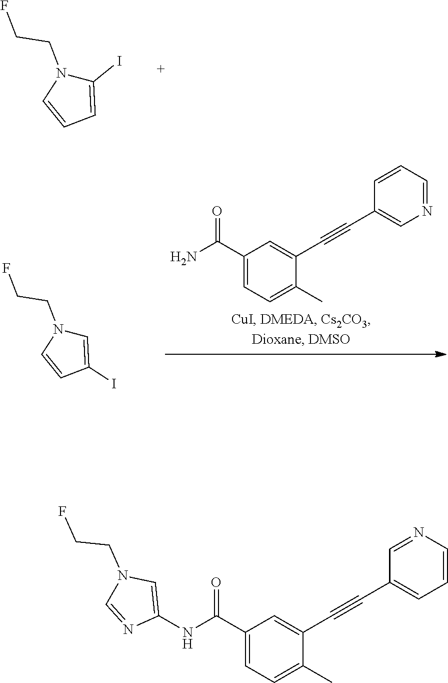 [SMILES: FCCn1cccc1I ; FCCn1ccc(I)c1 ; Cc1ccc(C(N)=O)cc1C#Cc1cccnc1 ; Cc1ccc(C(=O)Nc2cn(CCF)cn2)cc1C#Cc1cccnc1]

To a degassed suspension of Intermediate 12 (196 mg, 0.83 mmol, 1.0 eq), copper (I) iodide (159 mg, 0.83 mmol, 1.0 eq) and caesium carbonate (407 mg, 1.25 mmol, 1.5 eq) in 1,4-dioxane (8 mL) under N2 was added a solution of an inseparable isomeric mixture of Intermediates 104A and 104B (200 mg, 0.83 mmol, 1.0 eq) in DMSO (2 mL) followed by N,N′-dimethylethylenediamine (0.11 mL, 1.00 mmol, 1.2 eq) and the whole mixture degassed for a further 5 min before heating at 95° C. for 3 h. The reaction was cooled to rt, diluted with DCM (50 mL) and sat. aq. NH₄Cl (50 mL), the phases separated, and the aqueous phase extracted with DCM (2×50 mL). Combined organics were washed with brine (50 mL), dried (MgSO₄) and concentrated in vacuo. The residue was purified by normal phase chromatography, 5% MeOH/EtOAc and the resulting solid triturated with EtOAc (5 mL) to afford the title compound (84 mg, 29%) as a colourless solid. UPLC (Method A): 2.82 min, 100%, [M+H]⁺=349.2.

### Example 82

### 4-Methyl-N-(5-methyl-6,7-dihydro-5H-pyrrolo[1,2-a]imidazol-2-yl)-3-[2-(3-pyridyl)ethynyl]benzamide

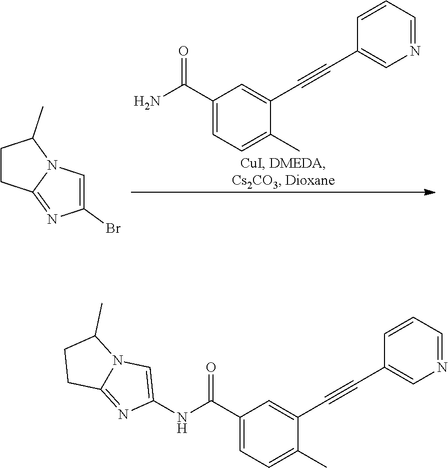

In a vial was combined Intermediate 103 (40 mg, 0.20 mmol, 1.00 eq), caesium carbonate (130 mg, 0.40 mmol, 2.00 eq) and copper (I) iodide (2 mg, 0.01 mmol, 0.05 eq) followed by a solution of N,N′-dimethylethylenediamine (4 mg, 0.04 mmol, 0.20 eq) in dry dioxane (0.5 mL) and Intermediate 12 (47 mg, 0.20 mmol, 1.00 eq) in dry dioxane (2 mL) and the vial was sealed and heated at 100° C. overnight. Further portions of copper (I) Iodide (2 mg, 0.01 mmol, 0.05 eq) and N,N′-dimethylethylenediamine (4 mg, 0.04 mmol, 0.20 eq) were added to the reaction, which was heated for an additional 24 h. The reaction was then cooled to rt, diluted with EtOAc (10 mL), H₂O (2 mL) and 35% aq. ammonia solution (1 mL) and the phases were separated. The aqueous was extracted with EtOAc (15 mL), combined organics were washed with water/ammonia solution (35% aq), 14:1 (15 mL) and sat. NH₄Cl (10 mL), dried (Na₂SO₄) and concentrated in vacuo. The crude material was purified by reverse phase chromatography 20-95% MeCN/H₂O (0.1% NH₃ modifier) to afford the title compound (14 mg, 20%) as a white solid. UPLC (Method A): 2.99 min, 98%, [M+H]⁺=357.3.

### Example 88

### 4-Methyl-N-(4-oxo-6,7-dihydro-5H-pyrazolo[1,5-a]pyridin-2-yl)-3-[2-(3-pyridyl)ethynyl]benzamide

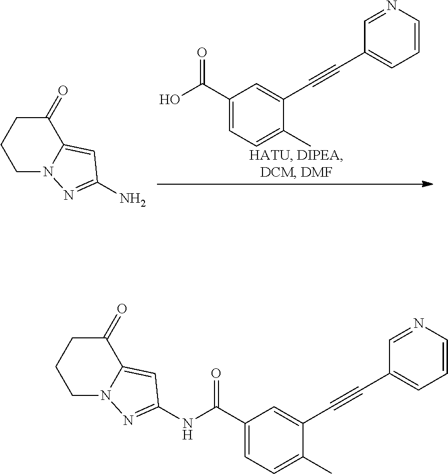 [SMILES: Nc1cc2n(n1)CCCC2=O ; Cc1ccc(C(=O)O)cc1C#Cc1cccnc1 ; Cc1ccc(C(=O)Nc2cc3n(n2)CCCC3=O)cc1C#Cc1cccnc1]

To a suspension of Intermediate 100 (80 mg, 0.53 mmol, 1.0 eq) and Intermediate 2 (126 mg, 0.53 mmol, 1.0 eq) in DCM (4 mL) was added (1-[bis(dimethylamino)methylene]-1H-1,2,3-triazolo[4,5-b]pyridinium 3-oxid hexafluorophosphate (242 mg, 0.63 mmol, 1.2 eq), N,N-diisopropylethylamine (0.28 mL, 1.59 mmol, 3.0 eq) and DMF (4 mL) and the reaction stirred at rt for 72 h. The mixture was quenched by addition of sat. aq. NH₄Cl (20 mL) and extracted with DCM (3×15 mL). The combined organics were washed with sat. aq. NH₄Cl (2×20 mL), dried (Na₂SO₄ followed by phase separator) and concentrated in vacuo. The crude residue was purified by reverse phase chromatography 20-95% MeCN/H₂O (0.1% NH₃ modifier) to afford the title compound (67 mg, 34%) as a white solid after freeze drying. UPLC (Method A) 3.23 min, 97%, [M+H]⁺=371.2.

### Example 89

### 4-Methyl-N-(5-methyl-6,8-dihydro-5H-imidazo[2,1-c][1,4]oxazin-2-yl)-3-[2-(3-pyridyl)ethynyl]benzamide

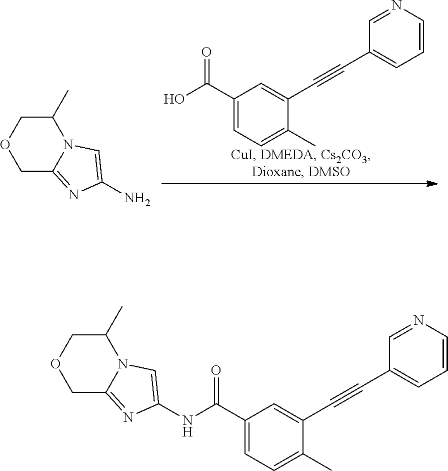 [SMILES: CC1COCc2nc(N)cn21 ; Cc1ccc(C(=O)O)cc1C#Cc1cccnc1 ; Cc1ccc(C(=O)Nc2cn3c(n2)COCC3C)cc1C#Cc1cccnc1]

To a vial containing copper (I) iodide (17.6 mg, 0.09 mmol, 0.2 eq) and caesium carbonate (300.2 mg, 0.92 mmol, 2.0 eq) was added N,N-dimethylethylenediamine (40 μL, 0.37 mmol, 0.8 eq), Intermediate 95 (100.0 mg, 0.46 mmol, 1.0 eq) and degassed dioxane (0.25 mL), then Intermediate 12 (108.9 mg, 0.46 mmol, 1.0 eq) was added in one portion, the flask was rinsed down with degassed dioxane (0.25 mL), the reaction was sealed and heated by microwave irradiation at 100° C. for 1 h. A further portion of copper (I) iodide (17.6 mg, 0.09 mmol, 0.2 eq) and N,N-dimethylethylenediamine (40 μL, 0.37 mmol, 0.8 eq) was added along with dioxane (0.25 mL) and DMSO (0.25 mL) and the reaction was heated by microwave irradiation at 120° C. for 3 h. The reaction was diluted with water (20 mL) and EtOAc (20 mL) and basified with 15% NH₄OH solution to pH 10. The phases were separated, the aqueous was extracted with EtOAc (2×20 mL) and combined organics were washed with brine (50 mL), dried (Na₂SO₄) and concentrated in vacuo. The crude residue was purified by reverse phase chromatography 10-70% MeCN/H₂O (0.1% NH₃ modifier) to afford the title compound (9.5 mg, 5%) as a beige solid after freeze drying. UPLC (Method A) 2.89 min, 95%, [M+H]⁺=373.3.

### Example 94

### N-[1-(cyclobutylmethyl)imidazol-4-yl]-4-methyl-3-[2-(3-pyridyl)ethynyl]benzamide

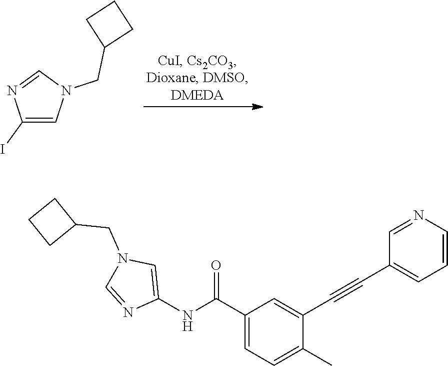 [SMILES: Ic1cn(CC2CCC2)cn1 ; Cc1ccc(C(=O)Nc2cn(CC3CCC3)cn2)cc1C#Cc1cccnc1]

In a microwave vial was added Intermediate 12 (180 mg, 0.76 mmol, 1.0 eq) Cs₂CO₃ (373 mg, 1.14 mmol, 1.5 eq) and CuI (145 mg, 0.76 mmol, 1.0 eq) and the atmosphere purged and replaced with N2 (×3). To the mixture was added dioxane (8 mL) and N2 bubbled through the reaction mixture for 5 min before addition of a solution of Intermediate 19 (200 mg, 0.76 mmol, 1.0 eq) in DMSO (2 mL), followed by DMEDA (99 μL, 0.92 mmol, 1.2 eq) and the reaction vessel sealed and heated to 95° C. overnight. The reaction was allowed to cool to rt and diluted with DCM (100 mL) and sat. aq. NH₄Cl (50 mL). The phases were separated and the aqueous extracted with DCM (3×30 mL). Combined organics were dried over Na₂SO₄, concentrated in vacuo and the residue purified by reverse phase column chromatography (Biotage Isolera, MeCN:H₂O (0.1% NH₃), 1:19 to 4:1 over 10 CV) to furnish the title compound (30.8 mg, 11%) as a white solid after freeze drying. UPLC (Method A) 3.26 min, 99%, [M+H]⁺=371.3.

### Example 95

### 4-Methyl-3-[2-(3-pyridyl)ethynyl]-N-spiro[6,7-dihydro-5H-pyrazolo[1,5-a]pyridine-4,1′-cyclopropane]-2-yl-benzamide

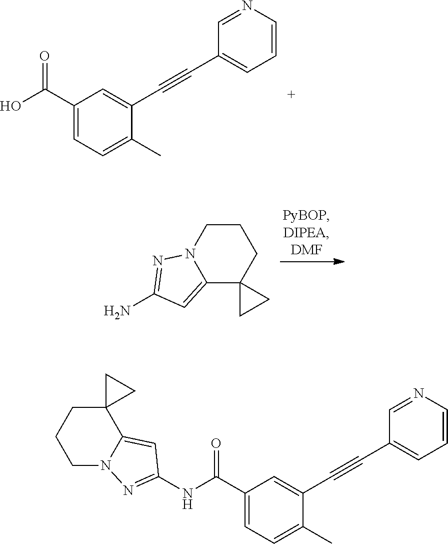 [SMILES: Cc1ccc(C(=O)O)cc1C#Cc1cccnc1 ; Nc1cc2n(n1)CCCC21CC1 ; Cc1ccc(C(=O)Nc2cc3n(n2)CCCC32CC2)cc1C#Cc1cccnc1]

To a solution of 4-methyl-3-[2-(3-pyridyl)ethynyl]benzoic acid Intermediate 2 (116 mg, 0.49 mmol), Intermediate 23 (80 mg, 0.49 mmol) and N,N-diisopropylethylamine (256 μL, 1.47 mmol) in DMF (2.0 mL) at rt was added (benzotriazol-1-yloxy)tripyrrolidinophosphonium hexafluorophosphate (383 mg, 0.74 mmol) and the reaction stirred at rt for 4 h. The reaction solution was loaded onto a reverse phase cartridge and purified by reverse phase column chromatography (Biotage Isolera, reverse phase, 30 g, HP-Sphere C18 ULTRA, 25 μm, 5% to 80% MeCN in H₂O, both eluents containing 0.1 Vol % NH₃). The fractions containing product was concentrated in vacuo and freeze-dried overnight to yield the title product (50.8 mg, 26.9%) as an off-white solid. UPLC (Method A) 3.53 min, 99.3%, [M+H]⁺=383.3.

### Example 96

### N-(6,6-dimethyl-5,8-dihydroimidazo[2,1-c][1,4]oxazin-2-yl)-4-methyl-3-[2-(3-pyridyl)ethynyl]benzamide

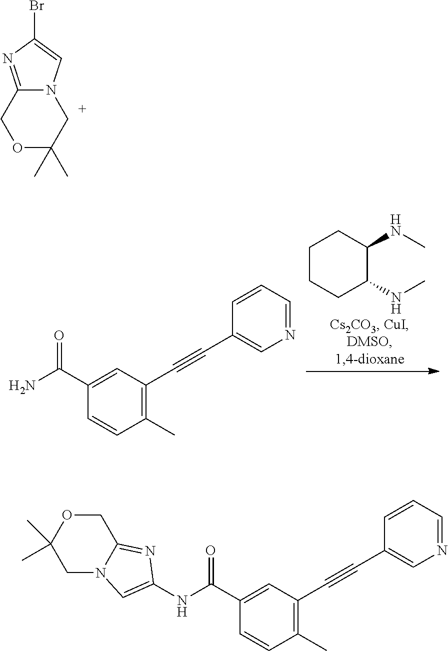

An N₂ purged stirring solution of Intermediate 29 (70.0 mg, 0.30 mmol), Cs₂CO₃ (148.0 mg, 0.45 mmol) and Intermediate 12 (71.6 mg, 0.30 mmol) in 1,4-dioxane (2.4 mL) and DMSO (0.6 mL) was degassed for 10 min. CuI (57.7 mg, 0.30 mmol) and trans N,N′-dimethylcyclohexane-1,2-diamine (57.3 μL, 0.36 mmol) were then added and the reaction was stirred at 95° C. for 2 h. The reaction was stirred for a further 1.5 h and then concentrated in vacuo. The oil was dissolved in sat. NH₄Cl solution (10 mL) and extracted with DCM:IPA, 4:1 (6×5 mL). The combined organics were dried (MgSO₄) and concentrated in vacuo to a crude dark green oil (890 mg). The residue was purified by column chromatography (Biotage Isolera, reverse phase, 30 g, HP-Sphere C18 ULTRA, 25 μm, 10% to 70% MeCN in H₂O, both eluents containing 0.1 Vol % NH₃) to afford the title compound (10.8 mg, 9.0%) as an off-white solid. UPLC (Method A) 3.06 min, 97%, [M+H]⁺=387.3.

### Example 97

### N-(4,4-difluoro-6,7-dihydro-5H-pyrazolo[1,5-a]pyridin-2-yl)-4-methyl-3-[2-(3-pyridyl)ethynyl]benzamide

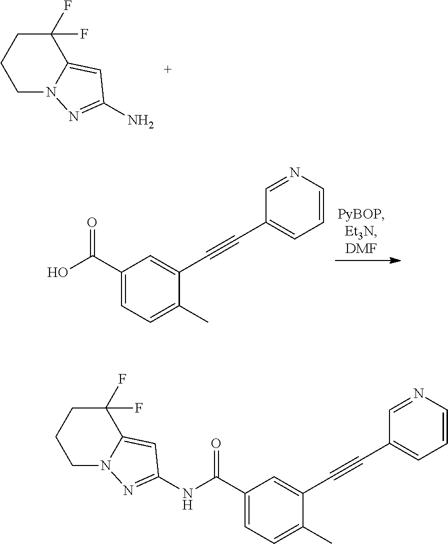 [SMILES: Nc1cc2n(n1)CCCC2(F)F ; Cc1ccc(C(=O)O)cc1C#Cc1cccnc1 ; Cc1ccc(C(=O)Nc2cc3n(n2)CCCC3(F)F)cc1C#Cc1cccnc1]

To a solution of Intermediate 37 (106 mg, 0.61 mmol, 1 eq) in DMF (3 mL) was added Intermediate 2 (145 mg, 0.61 mmol, 1 eq), Et₃N (256 μL, 1.84 mmol, 3 eq) and PyBOP (318 mg, 0.61 mmol, 1 eq) and the reaction stirred at rt overnight. The resulting orange reaction mixture was concentrated in vacuo and the resulting oil dissolved in DCM (30 mL), washed with H₂O (10 mL) and the organics dried (Na₂SO₄), filtered and the solvent removed in vacuo to yield the crude product as an orange oil. This was purified by Biotage Isolera (30 g C18 column) MeCN:H₂O, 1:19 (3 CV) then MeCN:H₂O, 35:65 to 80:20 (over 25 CV) with 0.1% NH₃ and flow rate=25 mL/min to furnish the title compound (95 mg, 40%) as a white solid. UPLC (Method A) 3.28 min, 98%, [M+H]⁺=393.

### Example 104

### N-(4,4-difluoro-3-methyl-6,7-dihydro-5H-pyrazolo[1,5-a]pyridin-2-yl)-4-methyl-3-[2-(3-pyridyl)ethynyl]benzamide

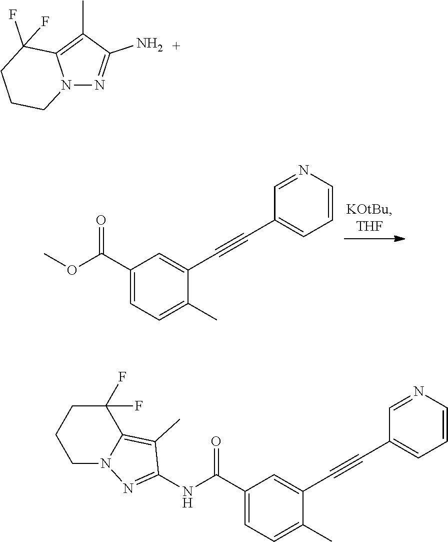 [SMILES: Cc1c(N)nn2c1C(F)(F)CCC2 ; COC(=O)c1ccc(C)c(C#Cc2cccnc2)c1 ; Cc1ccc(C(=O)Nc2nn3c(c2C)C(F)(F)CCC3)cc1C#Cc1cccnc1]

To a solution of Intermediate 112 (60.0 mg, 0.32 mmol) and Intermediate 1 (101.0 mg, 0.40 mmol) in dry THF (3.0 mL) was added tert-butoxypotassium (0.71 mL, 1.21 mmol, 1.7 M in THF) dropwise at r.t. The resultant orange suspension was stirred at r.t. for 1.5 h. To the reaction mixture was added aq. NH₄Cl (10 mL), the product extracted with EtOAc (2×15 mL) and washed with water (15 mL), dried over MgSO₄, filtered, absorbed onto silica and purified by column chromatography (manual column, normal phase, silica gel 40-63 μm/230-400 mesh, 60 Å) eluting with DCM-EtOAc 1:1. The product N-(4,4-difluoro-3-methyl-6,7-dihydro-5H-pyrazolo[1,5-a]pyridin-2-yl)-4-methyl-3-[2-(3-pyridyl)ethynyl]benzamide (23.9 mg, 18%) was isolated as a white solid. UPLC (Method E) 3.17 min, 99%, [M+H]⁺=407.2.

### Example 105

### N-(3-ethyl-4,4-difluoro-6,7-dihydro-5H-pyrazolo[1,5-a]pyridin-2-yl)-4-methyl-3-[2-(3-pyridyl)ethynyl]benzamide

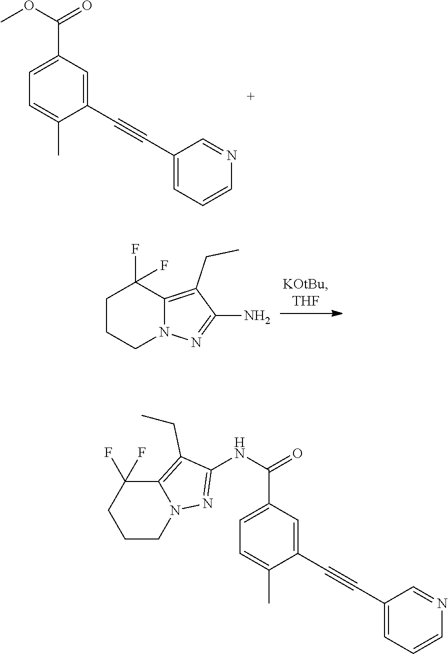 [SMILES: COC(=O)c1ccc(C)c(C#Cc2cccnc2)c1 ; CCc1c(N)nn2c1C(F)(F)CCC2 ; CCc1c(NC(=O)c2ccc(C)c(C#Cc3cccnc3)c2)nn2c1C(F)(F)CCC2]

To a solution of Intermediate 1 (66.8 mg, 0.27 mmol) and Intermediate 115 (54.0 mg, 0.24 mmol) in THF (5.0 mL) was added KOtBu (1.7 M solution in THF, 0.57 mL, 0.97 mmol) dropwise and the mixture stirred at r.t. for 0.5 h under nitrogen. The reaction was quenched with water (10 mL) and extracted with EtOAc (3×10 mL). Combined organics were washed with brine (10 mL), dried over Na₂SO₄ and concentrated to yield crude product. The crude was further purified via column chromatography (Biotage Isolera, reverse phase, 40 g, Siliasep, 20% to 80% MeCN in H₂O both eluents containing 0.1 Vol % NH₃). The fractions containing the product were concentrated and the residue was further purified via column chromatography (manual column, normal phase, silica gel 40-63 μm/230-400 mesh, 60 Å, 20% to 80% EtOAc in heptane) to yield the desired product, N-(3-ethyl-4,4-difluoro-6,7-dihydro-5H-pyrazolo[1,5-a]pyridin-2-yl)-4-methyl-3-[2-(3-pyridyl)ethynyl]benzamide (26.0 mg, 25%) as a colourless solid after freeze-drying. UPLC (Method A) 3.42 min, 97.5%, ES⁺: 421.2 [M+H]⁺.

### Example 106

### 4-Methyl-N-[1-[2-(4-methylpiperazin-1-yl)ethyl]-5-(trifluoromethyl)pyrazol-3-yl]-3-[2-(3-pyridyl)ethynyl]benzamide

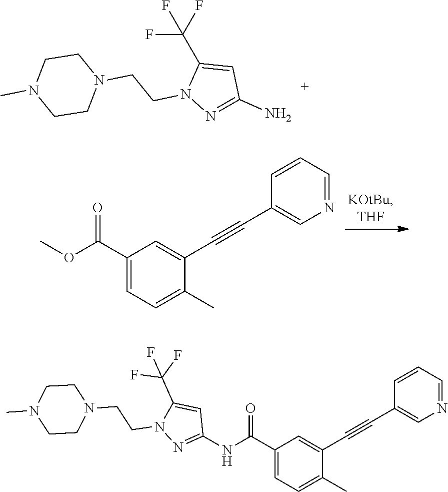

To a solution of Intermediate 1 (113 mg, 0.45 mmol) and Intermediate 116 (100 mg, 0.36 mmol) in THF (4.0 mL) was added KOtBu (20% w/w solution in THF, 0.80 mL, 1.44 mmol) dropwise and the mixture stirred at rt for 0.5 h. The reaction mixture was quenched by addition of water (20 mL) and saturated brine solution (20 mL) and extracted with TBME (2×30 mL). The combined organic layers were washed with 10% K₂CO₃ solution (20 mL), dried over MgSO₄ and concentrated to give an orange oily residue. The residue was purified by column chromatography (Biotage Isolera, reverse phase, 12 g, Sfar C18D Duo 100 Å, 30 μm, 55% to 100% MeCN in H₂O, both eluents containing 0.1 Vol % NH₃) to give 4-methyl-N-[1-[2-(4-methylpiperazin-1-yl)ethyl]-5-(trifluoromethyl)pyrazol-3-yl]-3-[2-(3-pyridyl)ethynyl]benzamide (23.0 mg, 13%) as a colourless solid after freeze-drying. UPLC (Method A) 3.34 min, 99.8%, ES⁺: 497.3 [M+H]⁺.

### Comparative Example 1

### N-(5-tert-butylisoxazol-3-yl)-3-(2-imidazo[1,2-a]pyridin-3-ylethynyl)-4-methyl-benzamide

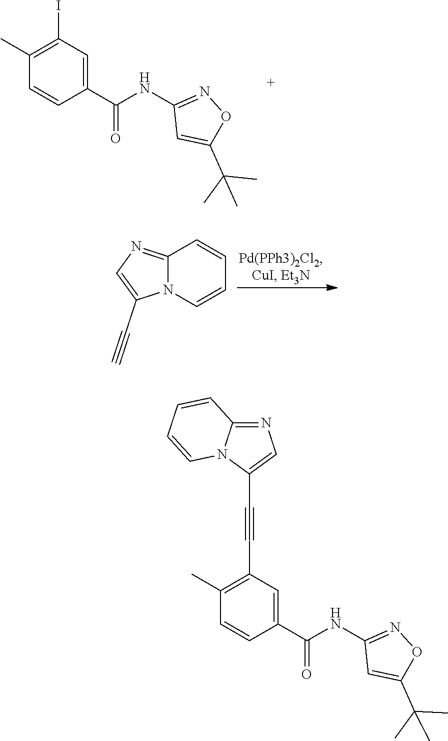

A solution of Intermediate 18 (280 mg, 0.70 mmol, 1 eq), Intermediate 17 (100 mg, 0.7 mmol, 1 eq), bis(triphenylphosphine)palladium(II) dichloride (12.3 mg, 17.6 μmol, 0.025 eq), copper(I) iodide (4.7 mg, 24.6 μmol, 0.035 eq) and TEA (118 μL, 844 μmol, 1.2 eq) in MeCN (7.0 mL) was heated at reflux for 2 h. The reaction mixture was filtered and the filter cake washed with MeCN (20 mL) and DCM (20 mL) and combined organics were concentrated in vacuo. The residue was purified by column chromatography (normal phase, [24 g], RediSep silica gel, 35-60 μm (230-400 mesh), 35 mL per min, gradient 0% to 100% EtOAc in iso-hexanes. The product was dried in a vacuum oven at 50° C. overnight to give N-(5-tert-butylisoxazol-3-yl)-3-(2-imidazo[1,2-a]pyridin-3-ylethynyl)-4-methyl-benzamide (179 mg, 64%) as a yellow solid. LCMS (Method C) 5.50 min, 100%, [M+H]⁺=399.1.

### Comparative Example 2

### 3-(imidazo[1,2-b]pyridazin-3-ylethynyl)-4-methyl-N-[4-[(4-methyl-1-piperazinyl)methyl]-3-(trifluoromethyl)phenyl]benzamide (Ponatinib, CAS: 943319-70-8)

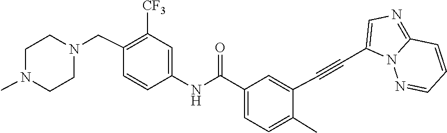 [SMILES: Cc1ccc(C(=O)Nc2ccc(CN3CCN(C)CC3)c(C(F)(F)F)c2)cc1C#Cc1cnc2cccnn12]

Ponatinib was purchased commercially from AK Scientific, Inc.

### Comparative Example 3

### 4-methyl-N-[3-(4-methylimidazol-1-yl)-5-(trifluoromethyl)phenyl]-3-[(4-pyridin-3-ylpyrimidin-2-yl)amino]benzamide (Nilotinib, CAS: 641571-10-0)

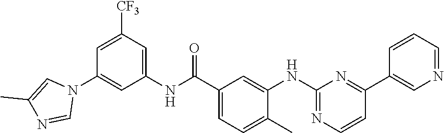 [SMILES: Cc1cn(-c2cc(NC(=O)c3ccc(C)c(Nc4nccc(-c5cccnc5)n4)c3)cc(C(F)(F)F)c2)cn1]

Nilotinib was purchased commercially from Medchem Tronica.

### Comparative Example 4

### 3-[2-(3-Amino-6-fluoro-4-isoquinolyl)ethynyl]-4-methyl-N-[1-(2-morpholinoethyl)pyrazol-3-yl]benzamide

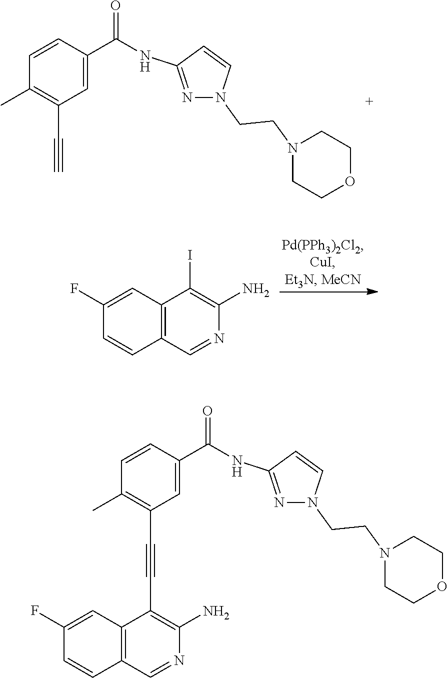 [SMILES: C#Cc1cc(C(=O)Nc2ccn(CCN3CCOCC3)n2)ccc1C ; Nc1ncc2ccc(F)cc2c1I ; Cc1ccc(C(=O)Nc2ccn(CCN3CCOCC3)n2)cc1C#Cc1c(N)ncc2ccc(F)cc12]

Intermediate 42 (185.0 mg, 0.59 mmol, 91.7% pure), Intermediate 40 (160.0 mg, 0.45 mmol, 96% pure), Et₃N (75.9 μL, 0.54 mmol), bis(triphenylphosphine)palladium(II) dichloride (8.0 mg, 11.3 μmol) and CuI (3.0 mg, 15.9 μmol) were added to MeCN (5.0 mL) and the reaction heated under reflux at 82° C. for 3 h. The mixture was filtered through Celite and concentrated in vacuo. The residue was purified by reverse phase HPLC (ACE-5AQ, 100×21.2 mm, 5 μm, 25 mL per min, gradient 0% to 100% (over 7 min) then 100% (3 min) MeCOH in 10% MeCOH/water). The residue was then repurified by reverse phase HPLC (Phenomenex Synergi Hydro-RP 80A AXIA, 100×21.2 mm, 4 μm, 25 mL per min, gradient 20% to 100% (over 7 min) then 100% (3 min) MeCOH in 10% MeCOH/water) [1% formic acid]). The material was de-salted by treating with sat. aq. NaHCO₃ and extracted into DCM and concentrated to dryness in vacuo. The residue was then repurified by reverse phase HPLC (ACE-5AQ, 100×21.2 mm, 5 μm, 25 mL per min, gradient 0% to 100% (over 7 min) then 100% (3 min) MeCOH in 10% MeCOH/water) then dried in a vacuum oven at 50° C. overnight to give the title compound (15.5 mg, 6.8%) as a yellow solid. LC-MS (Method H) 4.57 min, 98%, [M+H]⁺=498.5.

### Comparative Example 5

### N-[3,3-difluoro-1-(4-methylpiperazin-1-yl)indan-5-yl]-3-(2-imidazo[1,2-b]pyridazin-3-ylethynyl)-4-methyl-benzamide

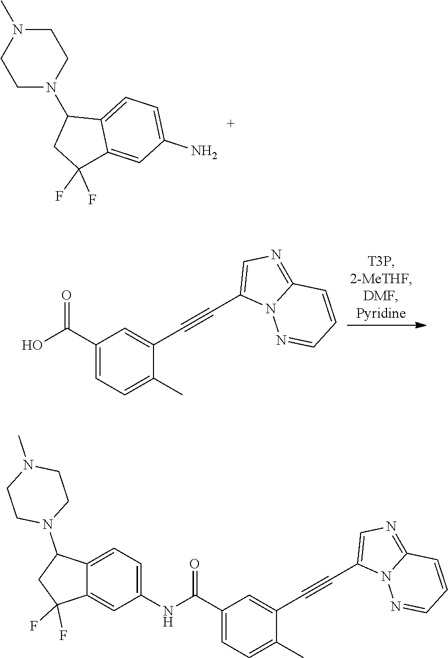 [SMILES: CN1CCN(C2CC(F)(F)c3cc(N)ccc32)CC1 ; Cc1ccc(C(=O)O)cc1C#Cc1cnc2cccnn12 ; Cc1ccc(C(=O)Nc2ccc3c(c2)C(F)(F)CC3N2CCN(C)CC2)cc1C#Cc1cnc2cccnn12]

To a solution of Intermediate 52 (80 mg, 0.30 mmol) and Intermediate 45 (119 mg, 0.30 mmol, 70% purity) in 2-MeTHF (10 mL) and DMF (5 mL) was added pyridine (100 μL, 1.20 mmol) followed 1-propylphosphonic acid cyclic anhydride (400 μL, 0.60 mmol). The reaction mixture was then heated at 70° C. overnight. Separately, to a solution of Intermediate 52 (23 mg, 0.09 mmol) and Intermediate 45 (34 mg, 0.09 mmol, 70% purity) in 2-MeTHF (3 mL) and DMF (1 mL) was added pyridine (30 μL, 0.34 mmol) followed 1-propylphosphonic acid cyclic anhydride (100 μL, 0.17 mmol). The reaction mixture was then heated at 70° C. overnight. The two reactions were then cooled to rt and combined. To the resulting mixture was added EtOAc (50 mL), followed by aq. K₂CO₃ (2M, 50 mL) and the phases separated. The aqueous phase was extracted with EtOAc (2×50 mL) and combined organics were washed with brine (50 mL), dried (Na₂SO₄) and concentrated in vacuo to give the crude product as a brown oil. The crude product was purified on silica using DCM/NH₃ (7M in MeCOH) (99:1 to 95:5), further purified by column chromatography (manual column, normal phase, KP-NH silica, 10-20% acetone in DCM) and repurified using column chromatography (Biotage Isolera, reverse phase, 4 g, HP-Sphere C18 ULTRA, 25 μm [residue loaded in DMSO], 10-80% THF in H₂O, both eluents containing 0.1 Vol % NH₃) and the material freeze-dried for 72 h to yield the title compound (26 mg, 13%) as an off white solid. UPLC (Method A) 3.24 min, 99.5%, [M+H]⁺=527.3.

### Comparative Example 6

### N-[6-(4-isopropyl-1,2,4-triazol-3-yl)-2-pyridyl]-3-[2-(3-pyridyl)ethynyl]benzamide

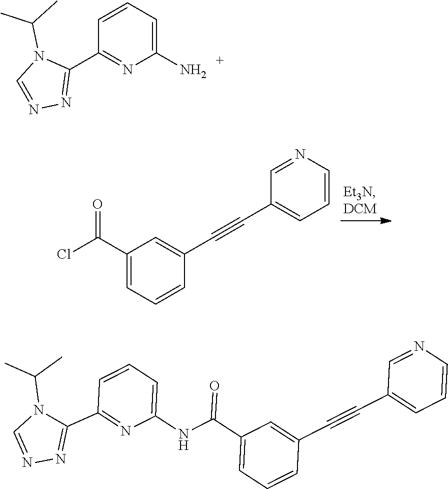 [SMILES: CC(C)n1cnnc1-c1cccc(N)n1 ; O=C(Cl)c1cccc(C#Cc2cccnc2)c1 ; CC(C)n1cnnc1-c1cccc(NC(=O)c2cccc(C#Cc3cccnc3)c2)n1]

To a mixture of Intermediate 119 (50 mg, 0.25 mmol) and triethylamine (40 μL, 0.32 mmol) in DCM (3.0 mL), a solution of Intermediate 121 (119 mg, 0.49 mmol) in DCM (2.0 mL) was added dropwise and the reaction mixture was stirred at rt overnight under nitrogen. The reaction mixture was quenched with water (10 mL) and extracted with DCM (3×10 mL). The combined organics were washed with brine (15 mL), dried over Na₂SO₄ and concentrated in vacuo. The residue was purified by column chromatography (Biotage Isolera, Reverse phase, 25 g, Siliasep, 20% to 80% MeCN in H₂O both eluents containing 0.1 Vol % NH₃), to give the desired product N-[6-(4-isopropyl-1,2,4-triazol-3-yl)-2-pyridyl]-3-[2-(3-pyridyl)ethynyl]benzamide (8.0 mg, 7.9%) as an off-white solid. UPLC (Method A) 2.97 min, 99.3%, ES⁺: 409.3 [M+H]⁺.

### Comparative Example 7

### N-(5-ethyl-1,3,4-thiadiazol-2-yl)-4-pyrazol-1-yl-3-[2-(3-pyridyl)ethynyl]benzamide

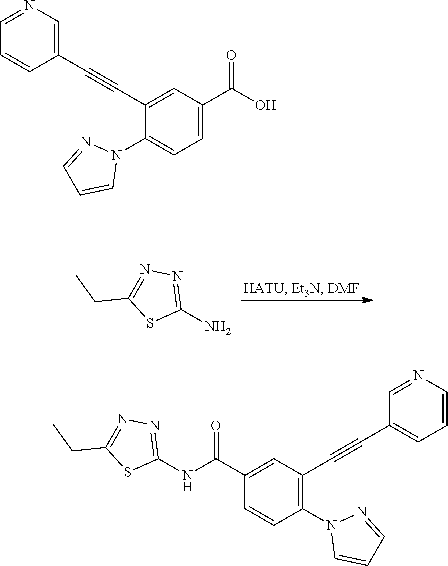

To a solution of Intermediate 124 (79.0 mg, 0.25 mmol) and 2-amino-5-ethyl-1,3,4-thiadiazole (38.0 mg, 0.29 mmol) in DMF (2.0 mL) was added HATU (120.0 mg, 0.32 mmol) and triethylamine (0.10 mL, 0.72 mmol) under a N₂ atmosphere and the reaction stirred at rt overnight. The reaction was quenched with sat. aq. NaHCO₃ (10 mL) and diluted with EtOAc (30 mL), the phases separated and the aqueous extracted with EtOAc (3×30 mL). The combined organics were dried over Na₂SO₄, filtered and concentrated. The crude material was purified by cationic exchange resin SCX-2 (non-endcapped propylsulfonic acid functionalized silica, 50 μM, 60 Å, 1 g), residue loaded in DCM, washed with MeCOH and eluted with NH₃ (2 M in MeCOH). The resulting solid was triturated from water:MBTE, collected on a frit and dried to give N-(5-ethyl-1,3,4-thiadiazol-2-yl)-4-pyrazol-1-yl-3-[2-(3-pyridyl)ethynyl]benzamide (41 mg, 41%) as an off-white solid. UPLC (Method A) 2.02 min, 98.9%, ES⁺: 401.2 [M+H]⁺.

### Biological Data

### Ba/F3 Cell Titer Glo Assay

The CellTiter-Glo luminescent cell viability assay is a homogeneous method of determining the number of viable cells in culture based on quantification of the ATP present. Briefly, IL-3 dependent Ba/F3 cells are modified to express BCR-ABL. Activity of the transformed kinase overrides IL3 dependency for cellular proliferation and survival. Test compounds that specifically inhibit kinase activity lead to programmed cell death which can be measured through the addition of CellTiter-Glo reagent. In this assay Ba/F3 cells expressing BCR-ABL (Advanced Cellular Dynamics) or parental Ba/F3 (control) cells were prepared at 5×10⁴/mL in RPMI 1640 containing 10% FBS, 1×Glutamax and 750 ng/mL puromycin. Test compounds were dispensed into 384 well plates using the Tecan D300e at a top final assay concentration of 10 μM with dosing normalised to 0.1% DMSO in 50 μL volume. 50 μL cells were added to each well of the prepared 384 well plates and the plates spun at 1000 rpm for 1 min prior to incubation at 37° C., 5% CO₂ for 48 h. After 48 h 15 μL CellTiterGlo reagent was added to each well in the plate. Following a 60 min incubation at rt luminescence was read on the Pherastar FS reader.

The exemplified compounds of the invention were tested in the Ba/F3 Cell Titer Glo Assay and the pIC50 data is shown in the table below. pIC₅₀ data are calculated as the −log₁₀(IC₅₀ in Molar) Those data show that the compounds of the invention can inhibit c-Abl.

| Example | pIC₅₀ |
|---|---|
| Comp. Ex. 1 | 7.4 |
| Comp. Ex. 2 | 8.0 |
| Comp. Ex. 3 | 7.6 |
| Comp. Ex. 4 | 6.4 |
| Comp. Ex. 5 | 8.4 |
| Comp. Ex 6 | <5.0 |
| Comp. Ex 7 | <5.0 |
| 1 | 6.1 |
| 2 | 6.4 |
| 3 | 7.3 |
| 4 | 6.5 |
| 5 | 6.7 |
| 6 | 6.4 |
| 7 | 7.1 |
| 8 | 6.8 |
| 9 | 7.0 |
| 10 | 7.7 |
| 11 | 7.4 |
| 12 | 7.3 |
| 13 | 7.0 |
| 14 | 6.8 |
| 15 | 7.4 |
| 16 | 7.1 |
| 17 | 7.5 |
| 18 | 6.8 |
| 19 | 7.0 |
| 20 | 6.8 |
| 21 | 7.0 |
| 22 | 7.0 |
| 23 | 6.8 |
| 24 | 7.3 |
| 25 | 6.8 |
| 26 | 7.3 |
| 27 | 6.5 |
| 28 | 6.7 |
| 29 | 6.9 |
| 30 | 6.2 |
| 31 | 6.3 |
| 32 | 6.2 |
| 33 | 6.8 |
| 34 | 7.5 |
| 35 | 7.4 |
| 36 | 7.4 |
| 37 | 7.3 |
| 38 | 7.2 |
| 39 | 6.9 |
| 40 | 6.9 |
| 41 | 6.9 |
| 42 | 6.8 |
| 43 | 6.8 |
| 44 | 6.7 |
| 45 | 6.8 |
| 46 | 6.8 |
| 47 | 6.7 |
| 48 | 6.7 |
| 49 | 6.7 |
| 50 | 6.7 |
| 51 | 6.7 |
| 52 | 6.7 |
| 53 | 6.7 |
| 54 | 6.6 |
| 55 | 6.6 |
| 56 | 6.5 |
| 57 | 6.6 |
| 58 | 6.6 |
| 59 | 6.6 |
| 60 | 6.5 |
| 61 | 6.6 |
| 62 | 6.6 |
| 63 | 6.5 |
| 64 | 6.4 |
| 65 | 6.5 |
| 66 | 6.5 |
| 67 | 6.5 |
| 68 | 6.5 |
| 69 | 6.4 |
| 70 | 6.4 |
| 71 | 6.3 |
| 72 | 6.2 |
| 73 | 6.3 |
| 74 | 6.2 |
| 75 | 6.2 |
| 76 | 6.2 |
| 77 | 6.2 |
| 78 | 6.1 |
| 79 | 6.1 |
| 80 | 6.1 |
| 81 | 6.1 |
| 82 | 6.0 |
| 83 | 6.1 |
| 84 | 6.1 |
| 85 | 6.1 |
| 86 | 6.0 |
| 87 | 6.0 |
| 88 | 6.1 |
| 89 | 6.0 |
| 90 | 6.0 |
| 91 | 6.0 |
| 92 | 6.0 |
| 93 | 6.0 |
| 94 | 6.1 |
| 95 | 6.8 |
| 96 | 6.2 |
| 97 | 7.3 |
| 98 | 6.1 |
| 99 | 6.5 |
| 100 | 6.5 |
| 101 | 6.6 |
| 102 | 7.1 |
| 103 | 7.1 |
| 104 | 7.2 |
| 105 | 6.9 |
| 106 | 7.5 |

### Determination of CNS Penetration In Vivo

Male Sprague Dawley Rats 300-350 g (Charles River, UK) were group housed, n=3, under a 12 hour light/dark cycle with food and water available ad libitum. At 17:00 on the day prior to dosing all food was removed. On the day of dosing animals were weighed, tail marked and dosed via oral gavage with compound at 3 mg/kg in a volume of 5 mL/kg. Animals were culled at 30 min, 1 h and 4 h post dose via intra-peritoneal administration of pentobarbital. Post mortem blood was withdrawn via cardiac puncture, and briefly stored in K2 EDTA blood tubes on ice before being spun at 14,000 g for 4 min at 4° C. Plasma was withdrawn into a 96 well plate, placed on dry ice and stored at −80° C. Brains were quickly dissected and placed on dry ice before storage at −80° C.

Following dosing of test compound (Oral) to Male Sprague-Dawley Rats, animals are sacrificed at three timepoints. Plasma is isolated from whole blood following cardiac exsanguination by centrifugal blood fractionation and whole brains isolated. Samples are stored on-ice and transferred to the Bioanalytical lab storage at −80° C. Bioanalysis of plasma and brain samples is performed as detailed below. Methods were prepared with guidance from industry standard documents.^{2,3}

### Plasma Bioanalysis

A 10 mM DMSO stock is used to prepare spiking solutions of test compound in the range of 10-100,0000 ng/mL in diluent (MeCN:H₂O, 1;1). Calibration lines are prepared in control male Sprague-Dawley Rat plasma at known concentrations in the range of 1-10000 ng/mL by spiking 2.5 μL of calibration spiking solution into 25 μL control plasma. Experimental samples are thawed to rt and 25 μL aliquots are extracted alongside the calibration lines using protein precipitation (agitation for at least 5 min at rt with 400 μL of MeCN containing 25 ng/mL tolbutamide as an internal standard). Protein precipitates are separated from the extracted test compound by centrifugation at 4000 rpm for 5 min, 4° C. The resulting supernatants are diluted in a ratio of 1:2 with a relevant diluent (e.g. 0.1% formic acid in H₂O or 1:1 MeOH:H₂O).

Samples are analysed by UPLC-MS/MS on either an API6500 QTrap or Waters TQS mass spectrometer using previously optimised analytical MRM (multiple reaction monitoring) methods, specific to the test compound.

The concentration of test compound in isolated samples is determined following analysis of the samples against the two replicates of the calibration line, injected before and after the sample set with an appropriate regression and weighting used. Only samples within 20% of the expected test concentration are included in the calibration line and any samples that fall outside of the limits of the calibration line will be deemed to be less than or above the limit of quantification (LLoQ/ALoQ).

### Brain Bioanalysis

A 10 mM DMSO stock is used to prepare spiking solutions of test compound in the range of 10-100,0000 ng/mL in diluent (1:1 MeCN:H₂O). Calibration lines are prepared in control male Sprague-Dawley Rat brain homogenate at known concentrations in the range of 3-30000 ng/g by spiking 2.5 μL of calibration spiking solution into 25 μL control homogenate.

To prepare control and experimental brain homogenates, brains are thawed, weighed and a volume of diluent added (H₂O) in the ratio of 2 mL per gram of brain. Homogenisation of brains is performed by bead-beater homogenisation using Precellys Evolution and CK14 7 mL small ceramic bead homogenisation tubes.

Aliquots of 25 μL experimental sample are extracted alongside the calibration lines using protein precipitation (agitation for at least 5 min at rt with 400 μL of MeCN containing 25 ng/mL tolbutamide as an internal standard). Protein precipitates are separated from the extracted test compound by centrifugation at 4000 rpm for 5 min, 4° C. The resulting supernatants are diluted in a ratio of 1:2 with a relevant diluent (e.g. 0.1% formic acid in H₂O or 1:1 MeOH:H₂O).

Samples are analysed by UPLC-MS/MS on either an API6500 QTrap or Waters TQS mass spectrometer using previously optimised analytical MRM (multiple reaction monitoring) methods, specific to the test compound.

The concentration of test compound in isolated samples is determined following analysis of the samples against the two replicates of the calibration line, injected before and after the sample set with an appropriate regression and weighting used. Only samples within 20% of the expected test concentration are included in the calibration line and any samples that fall outside of the limits of the calibration line will be deemed to be less than or above the limit of quantification (LLoQ/ALoQ).

### Determination of Brain to Plasma Ratio and Free Brain Concentrations.

Total CNS penetrance is calculated by dividing the concentration in the brain by the concentration in plasma for each timepoint. The mean brain to plasma ratio (Br:PI) is calculated by averaging these ratios (defining which timepoints are used).

The free drug hypothesis states that only unbound compound is able to interact with and elicit a pharmacological effect. Therefore it is desirable for compounds to have a high free brain concentration. To calculate the free concentrations in each matrix, the determined concentrations are multiplied by the % free value as determined by plasma protein binding and brain tissue binding studies using rapid equilibrium dialysis⁵. These values are then converted to molar concentrations to give a nanomolar free result at each timepoint.⁷

The K_{pu,u} or K_{p,brain} is calculated as the ratio of free drug fraction unbound in brain to free drug unbound in plasma.^{4,5,6,7}

The table below shows the free brain level at Cₘₐₓ for compounds of the invention. The examples of the invention have much improved free brain levels at Cₘₐₓ compared to the comparative examples.

| | | | | Free |
|---|---|---|---|---|
| | | | | Brain |
| | | | | level at |
| | | | Dose | Cₘₐₓ |
| Example | Formulation | Route | [mg/mL] | (nM) |
| Comparative | 30% HPBC solution, pH 3.5 | PO | 3 | 0.3 |
| Example 1 | | | | |
| Comparative | 5% NMP in 30% HPBC in water | IV | 0.5 | <0.1 |
| Example 2 | pH5.7 | | | |
| Comparative | 20% Cremophor/10% DMA/21% | PO | 3 | <0.0168 |
| Example 3 | HPBC Solution, pH 6.5 | | | |
| Comparative | 30% HPBC Solution, pH 4.0 | PO | 3 | <2.26 |
| Example 5 | | | | |
| 1 | 30% HPBC pH 3.9 | PO | 3 | 3.2 |
| 2 | 30% HPBC pH 4.8 | PO | 3 | 3.3 |
| 3 | 30% HPBC suspension pH 3.0 | PO | 3 | 11.3 |
| 4 | 30% HPBC Solution, pH3.0 | PO | 3 | 15.2 |
| 5 | 30% HPBC, pH 3.0 suspension | PO | 3 | 9.8 |
| 6 | 30% HPBC, pH 4.0 solution | PO | 3 | 11.3 |
| 7 | 30% HPBC Solution, pH3.0 | PO | 3 | 2.7 |
| 8 | 30% HPBC Solution, pH3.0 | PO | 3 | 4.9 |
| 9 | 30% HPBC pH 3.5 | PO | 3 | 3.5 |
| 10 | 30% HPBC pH 3.5 | PO | 3 | 6.2 |
| 11 | 30% HPBC pH 3.0 | PO | 3 | 6.9 |
| 12 | 30% HPBC, pH 4.0 solution | PO | 3 | 3 |
| 13 | 30% HPBC, pH 5.0 solution | PO | 3 | 6.6 |
| 14 | 30% HPBC suspension pH 3.5 | PO | 3 | 2.2 |
| 15 | 30% HPBC pH 3.0 | PO | 3 | 15.1 |
| 16 | 30% HPBC pH 4.0 | PO | 3 | 5.2 |
| 17 | 30% HPBC pH 4.0 | PO | 3 | 3.2 |
| 18 | 30% HPBC Solution, pH3.0 | PO | 3 | 4.2 |
| 19 | 30% HPBC Solution pH3.0 | PO | 3 | 7.8 |
| 20 | 30% HPBC Solution, pH3.0 | PO | 3 | 2 |
| 21 | 30% HPBC Solution pH3.0 | PO | 3 | 6.2 |
| 22 | 30% HPBC in water, solution, pH 4.0 | PO | 3 | 10 |
| 23 | 30% HPBC in water, solution, pH 4.0 | PO | 3 | 26.1 |
| 24 | 5% NMP in 30% HPBC, pH 4.5 | PO | 3 | 11.1 |
| 25 | 5% NMP in 30% HPBC, pH 4.8 | PO | 3 | 11.1 |
| 26 | 5% NMP in 30% HPBC, pH 3.5 | PO | 3 | 4.1 |
| 27 | 30% HPBC pH 3.7 | PO | 3 | 2.7 |
| 28 | 30% HPBC pH 3.2 | PO | 3 | 7.5 |
| 29 | 30% HPBC pH 3.0 | PO | 3 | 2.9 |
| 30 | 30% HPBC pH 3.8 | PO | 3 | 1.8 |
| 31 | 30% HPBC Solution, pH3.5 | PO | 3 | 4.4 |
| 32 | 30% HPBC Solution, pH4.0 | PO | 3 | 6.6 |
| 33 | 30% HPBC Solution, pH3.0 | PO | 3 | 1.5 |
| 34 | 30% HPBC in water Solution, pH 4.0 | PO | 3 | 2.51 |
| 36 | 30% HPBC in water Solution, pH 3.5 | PO | 3 | 18 |
| 37 | 30% HPBC Solution, pH 3.0 | PO | 3 | 9.89 |
| 38 | 30% HPBC Solution, pH 4.0 | PO | 3 | 6.77 |
| 39 | 30% HPBC Solution in water, pH 4.0 | PO | 3 | 5.35 |
| 44 | 30% HPBC Solution, pH 4.0 | PO | 3 | 3.31 |
| 48 | 30% HPBC Solution, pH 6.2 | PO | 3 | 10.5 |
| 58 | 30% HPBC Solution in water, pH 3.5 | PO | 3 | 14.8 |
| 62 | 30% HPBC Solution in water, pH 3.5 | PO | 3 | 6.41 |
| 84 | 30% HPBC Solution in water, pH 3.5 | PO | 3 | 18.4 |

Formation of Reactive Metabolites of Ponatinib by Extrahepatic CYP1A1

Serious side-effects including hepatotoxicity caused the brief withdrawal of ponatinib from the market. It was postulated that the hepatotoxicity may be caused by the formation of a reactive metabolite of Ponatinib, which was demonstrated by De Lin and colleagues (2017)¹ via an extrahepatic CYP1A1 mediated epoxide formation.

### Method

Investigations into the potential for test compound to form a reactive metabolite were performed using an adapted method detailed by De Lin et al. Briefly, test compound was pre-incubated (n=2; 50 μM substrate concentration) at 37° C. for 5 min with mixed-gender human liver cytosol (1 mg/mL total protein concentration) in 100 mM phosphate buffer (pH 7.4) with 5 mM MgCl₂ in the presence of recombinant human P450 enzyme 1A1 (100 nM), in the presence and absence of 5 mM glutathione. At the end of the pre-incubation period, 2 mM NADPH was added to each sample and the mixture incubated at 37° C. for 60 min. Immediately following the addition of NADPH and after the 60 min incubation period, aliquots of each sample were mixed with MeCN (containing internal standard) to terminate the reaction and precipitate the proteins. Incubations with control bactosomes were also performed alongside to confirm the requirement for metabolic activation.

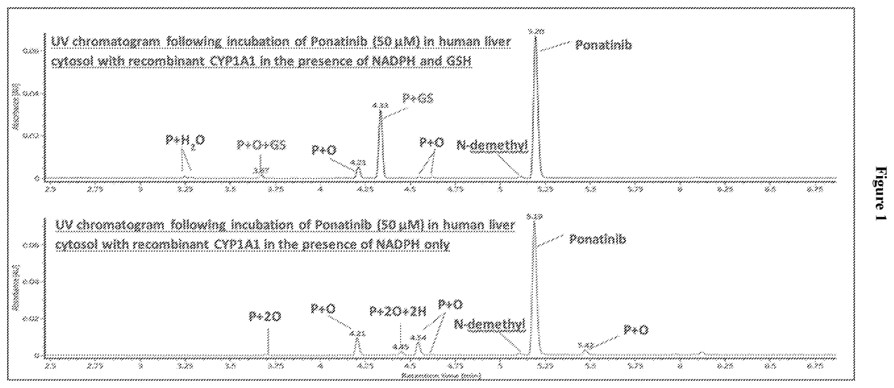

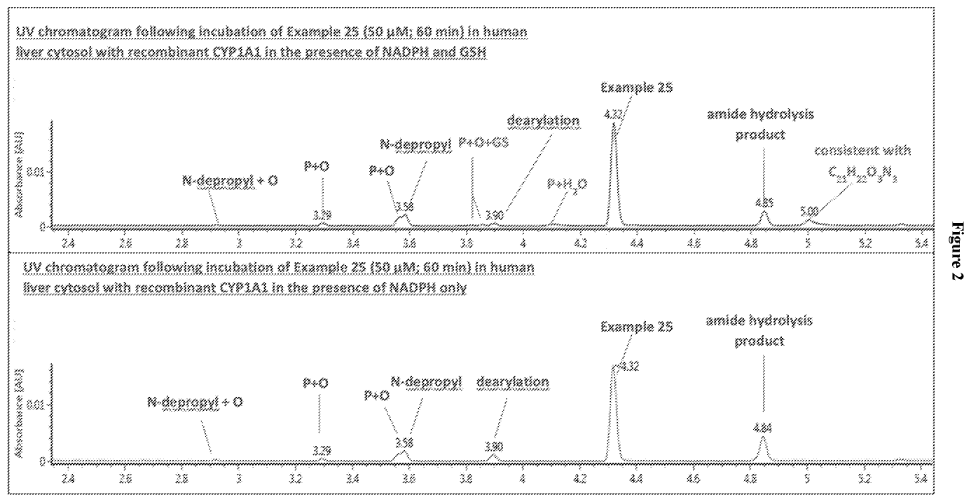

Samples were mixed, centrifuged and following appropriate dilution, supernatants were analysed by LC-MS utilising a high-resolution mass spectrometer to perform metabolite identification and characterisation analysis. The resultant traces are shown in FIGS. 1 and 2.

### Results

Following incubation with human liver cytosol and recombinant CYP1A1 in the presence of NADPH and GSH, ponatinib forms a significant direct glutathione adduct. This metabolite does not form following incubation with control bactosomes indicating that metabolic activation by CYP1A1 is required (postulated to be through biotransformation to an epoxide). Additionally, both NADPH and GSH are required for formation of the glutathione adduct to occur.

Following incubation of Example 25 with human liver cytosol and recombinant CYP1A1 in the presence of NADPH and GSH, no products of direct glutathione conjugation were observed indicating that Example 25 has a significantly reduced risk of forming reactive metabolites in vivo. Two very minor metabolites were identified as products of oxidation and glutathione conjugation however these were very minor and therefore unlikely to be significant.

These results indicate that the compounds of the invention do not form potentially toxic reactive metabolites, such as those observed for Ponatinib.

Human iAstrocyte—Murine Hb9-GFP+ Motor Neuron Co-Culture

### Materials and Methods

iNPCs (induced Neuronal Progenitor Cells) were derived from ALS patient fibroblasts as described previously (Meyer et al. 20148). iNPCs were differentiated into iAstrocytes by culturing in iAsrocyte media for at least 5 days. Murine motor neurons expressing the green fluorescent protein (GFP) under the Hb9 motor neuron-specific promoter (called from now on Hb9-GFP+) were differentiated from murine embryonic stem cells via embryoid bodies (EBs), as previously described (Haidet-Phillips et al. 20119, Wichterle et al. 200210).

### Co-Culture Procedure:

Day0—iNPC Splitting and mESC Splitting

iNPCs and mESC were split into iAstrocyte media and mEB media respectively on the same day, such that iAstrocytes and motor neurons will have both differentiated for 7 days when seeded together in co-culture.

Day3—Media Change iAstrocytes

Changed media on iAstrocytes, and split using accutase if 90-100% confluent 3 days after seeding from iNPCs. iAstrocytes were left a further 2 days in iAstrocyte media until seeded onto 384-well plates.

Day5—iAstrocyte Seeding

Diluted fibronection 1:400 in PBS, and 5 μL added per well. The plate was incubated with fibronectin at room temp for at least 5 mins.

The media was removed from iAstrocytes, and washed in PBS. 1 mL accutase per 10 cm plate was added, and incubated at 37° C. for 4 mins. The plate was tapped to dislodge any remaining iAstrocytes. The iAstrocytes were resuspended in iAstro media, and centrifuged at 200×g for 4 mins. The supernatant was removed, the falcon was flicked to vortex the cells, and resuspended the cells in an appropriate amount of iAstrocyte media. Counted cells using the haemocytometer, and diluted cells to an appropriate dilution for seeding. Seeded 1-2,000 iAstrocytes in 35 μL media on fibronectin-coated 384-well plates. Centrifuged 384-well plates at 1,760×rpm for 60 s using a PK120 (ALC) centrifuged (in neurogenetics) to collect media and cells to base of wells. Plates were left for 24 h for iAstrocytes to adhere to plate.

### Day6—Drug Treatment

Drugs were delivered in 100% drug-grade DMSO to iAstrocyte media using an Echo550 liquid handler (Labcyte). 384-well plates were centrifuged at 1,760×rpm for 60 s using a PK120 (ALC) centrifuge.

### Day7—EB Dissociation and Murine GFP+ Motor Neuron Seeding

2 plates of EBs were collected in a 50 mL tube and centrifuged for 4 mins at 200×g. The supernatant was removed from EBs after centrifugation, and washed in 10 mL PBS then centrifuged again for 4 mins at 200×g, and PBS wash removed.

For each 50 mL tube, 2.75 mL EB dissociation buffer was added and then 250 μL 200 U/mL (10×) Papain. The solution was gently pipetted up and down 10 times, using a P1000 pipette, against the side of the falcon (EB pellet was not pipetted directly). 50 mL tube was put in 37° C. water bath and incubated for 5 mins. Tube removed every 3 mins and gently shaken. After 5 mins, additional 2 mL of EB dissociation and 100 μL 200 U/mL (10×) Papain were added, which was then pipette again 5 times with P1000 pipette, and returned to the water bath for another 5 mins. It was returned to water bath as before, and incubated for no longer than 15-20 mins.

Centrifuged for 5 mins at 300×g. Each 50 mL tube, 2.7 mL EB dissociation was prepared, to which 300 μL FBS and 150 μL 0.5 mg/mL DNaseI was added. The supernatant was removed from dissociated EBs and 3 mL of the FBS/DNaseI mix was added, pipetted up and down with P1000 pipette about 5 times. 5 mL FBS was added very slowly to the bottom of the falcon containing the dissociated EBs. The supernatant was removed and the cells very gently resuspended in about 3 mL MN media (more used if the pellet is large) and filtered through a 40 μm filter. 1 mL extra MN media was added to wash the filter.

2,500 murine Hb9-GFP+ motor neurons were seeded per well in 10 μL motor neuron media on top of the pre-treated iAstrocytes. 384-well plates were centrifuged at 1,760×rpm for 60 s using a PK120 (ALC) centrifuge.

Day8 15 μL motor neuron media were added per well. Hb9-GFP+ motor neurons were imaged using an INCELL analyser 2000 (GE Healthcare)—day 1 of co-culture.

Day9 Hb9-GFP+ motor neurons were imaged using an INCELL analyser 2000 (GE Healthcare)—day 2 of co-culture (imaging is optional on this day).

Day10 Hb9-GFP+ motor neurons were imaged using an INCELL analyser 2000 (GE Healthcare)—day 3 of co-culture.

### Motor Neuron Viability Assessment:

The number of viable motor neurons (defined as GFP+ motor neurons with at least 1 axon) that survive after 72 hours is counted using the Columbus analyser software.

### Results

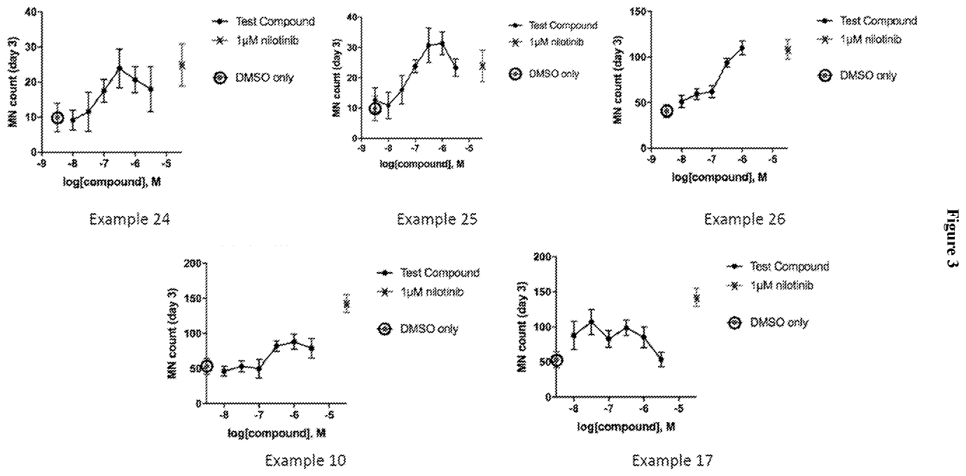

The results in FIG. 3 show that Examples 10, 17, 24, 25 and 26 rescue motor neuron survival in co-culture with patient-derived iAstrocytes. This effect is dose dependant, with maximum responses close to that of the positive control (1 μM Nilotinib and minimum responses close to that of the negative control (DMSO). Some compounds (for example 24, 25 and 17) show a reduction in efficacy at very high concentrations, potentially due to compound toxicity at this range. The efficacy of these compounds in this model demonstrates the utility of the compounds of the invention in the treatment of ALS.

### Modulation of α-Synuclein Aggregation

Compounds were assessed for their ability to modulate α-synuclein aggregation. ReNcell VM Neuronal cells were transduced with α-synuclein encoded adenovirus, and compounds were added after 24 hours. After 72 hours, compound was refreshed. After an additional 72 h, cells were fixated (a total of 6 days of compound treatment). Two distinct α-synuclein antibodies were added (Syn205 and MJF-14), imaging was performed on IN Cell 2200 at 10× magnification. Immunoreactivity is quantified by a high content algorithm. Compound data is normalized to a negative control (0.1% DMSO) and a positive control (10 μM KU0063794; CAS 938440-64-3).

### Results

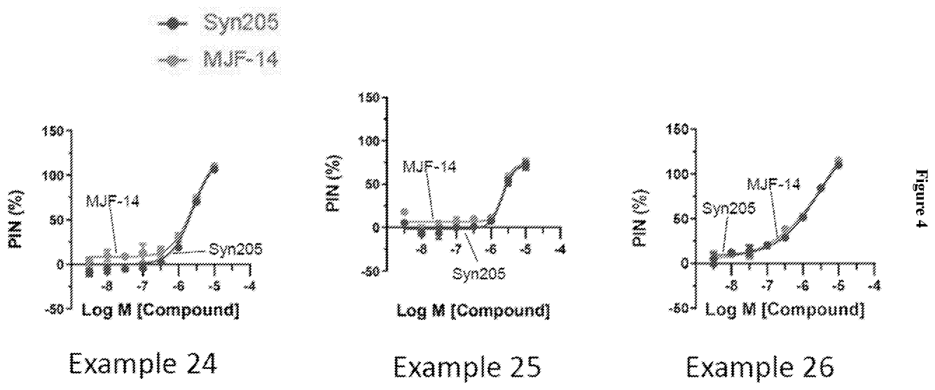

The results in FIG. 4 show that Examples 24, 25 and 26 have a dose dependant reduction on the amount of α-synuclein present in the ReNcell VM neuronal cells. The reduction is consistent with both antibodies, and all compounds show IC₅₀s in the range of 1-10 μM, where 100% inhibition is a reduction equal to 10 μM KU0063794 and 0% is a reduction equal to 0.1% DMSO. The efficacy of these compounds in this model demonstrates the utility of the compounds of the invention in the treatment of Parkinson's Disease.

### In Vitro Safety Panel Data

### HUVEC Cell Viability (CV) Assay Procedure

Cells are seeded at 10,000 cells/well in 96-well plates in HUVEC specific cell culture media. Cells are plated and cultured overnight (16-24 h) at 37° C. After the overnight culture, cells are washed and fed with the Assay Medium. Test compounds are applied and incubated with the cells for the designated time period, after which the cell viability is measured by alamarBlue method. Compounds are tested in duplicate at 8 concentrations (0.03, 0.1, 0.3, 1, 3, 10, 30, and 100 μM by default) for IC₅₀ determinations. The final DMSO concentration is 1%.

Fluorescence readings are recorded after the test compound and alamarBlue incubation. The excitation and emission wavelengths are 530 nm and 590 nm, respectively.

The percent of control is calculated by comparing the readings in the presence of the test compound to the vehicle control. Subsequently, the percent inhibition is calculated by subtracting the percent control activity from 100. IC50 values (concentration causing a half-maximal inhibition of the control value) are determined by non-linear regression analysis of the concentration-response curves using the Hill equation.

The reference compound used for this assay is Staurosporine.

### HUVEC Tube Formation Assay Procedure

HUVEC (human umbilical venous endothelial cells, 1.5×104/well, ATCC CRL-1730) are placed in an earlier prepared 96-well plate. Test substance and/or vehicle is then added to each well in a final concentration of 0.4% DMSO in growth medium under an atmosphere of 5% CO₂ at 37° C. After an 18 h incubation period, morphology of the endothelial cell tubes, which resemble a capillary-like network, are evaluated by a confocal high-content imaging system. Disruption (anti-angiogenesis) of total tube length is measured from each photograph and determined relative to the vehicle control group. Minimum inhibitory concentration (MIC, >30%) was noted as a significant response and GI50 (GI=Growth Inhibition) is determined. Compounds are screened at 30, 3, 0.3, 0.03 and 0.003 μM (or μg/mL) in triplicate.

The reference compound used in this experiment is Suramin with an expected GI50 of 15 μM.

The table below shows HUVEC data for compounds of the invention and comparative examples.

| | Cell viability | Tube formation GI50 |
|---|---|---|
| Example | IC50 (μM) | (μM) |
| Ex. 97 | 3.6 | >10 |
| Ex. 25 | 45 | >10 |
| Ex. 39 | 21 | >30 |
| Comp. Ex. 2 | 0.32 | 1.2 |
| Comp. Ex. 3 | 3.6 | >10 |

### Cardiomyocyte Assay (CRL) Procedure

### Experimental Procedure

All measurements were performed at physiological temperature in a tissue culture incubator (37° C.; 5% CO₂). Test article, positive controls and vehicle were added in a sterile tissue culture hood.

At least 10 h before compound addition SC-hCMs were exposed to the fresh culture medium. Before compound addition a one-minute recording of field potentials and impedance signals (baseline) was obtained. Test articles, vehicle and positive controls were added as a 2× concentration. One-minute recordings were obtained 0.5, 1, 2, 24 and 48 h after test article addition.

At the time of drug application, culture medium was added to an independent 96 well plate that was incubated in a tissue culture incubator for 48 h. This culture medium was used as the blank of the cTnI release assay.

After the 48 h exposure recording, 150 μL of each well was centrifuged to remove debris. The supernatant was collected and frozen for later cTnI content analysis using an ELISA plate according the manufacturer's instructions (Abcam, cat #ab200016).

Data was stored on the Charles River Laboratories computer network for off-line analysis. Data acquisition was performed using RTCA Cardio Software.

### Data Analysis

Data analysis was performed using RTCA Cardio Software, Microsoft Excel and Matlab scripts. Data were reported as mean±SEM.

The following field potential parameters were quantitated: Sodium Spike amplitude, Sodium Spike Rate, field potential duration (FPD), Bazett corrected field potential duration (FPDcB), and dysrhythmic events if present.

The following impedance parameters were quantitated: Cell Index, peak amplitude, relaxation from peak to 50% amplitude (Rel50) and twitch duration.

The sodium spike is the voltage signal produced by the propagation of the depolarizing wave (positive peak) and the local activation of sodium channels (negative peak). The sodium spike amplitude is the voltage difference (in ρV) between the positive and negative peaks of the sodium spike. The Sodium Spike Rate is a measure of the rate of sodium spike generation (spikes/min) produced by the spontaneous beating of stem cell cardiomyocytes.

Abnormal rhythm activity was characterized by the presence of the following proarrhythmic markers:

Triggered activity: Triggered activity (TA) is activity characterized by the presence of voltage oscillations during the repolarization period (Early After Depolarizations, EADs) or after the repolarization is completed (Delayed After Depolarizations, DADs). They are produced by different mechanisms. EADs are due to reactivation of sodium and calcium currents due to prolongation of the action potential. DADs are due to transient releases of calcium from intracellular calcium stores. Both EADs and DADs can trigger action potentials (ectopic beats, EB).

Impedance instability: Impedance instability (II) consist of small oscillations of the impedance signal during the slower relaxation phase of the impedance twitch sometimes associated with small oscillations of the field potential (FPO). II is observed in wells exposed to drugs that prolong the field potential duration. II informs about the possibility of drug-induced delayed repolarization setting the background where more robust proarrhythmic markers like EADs and DADs occur.

The time course of the effects was summarized in tables containing 1) the raw data, 2) the percentage change with respect to baseline calculated using the following equation: Δ %=[(Parameter at Dose x−Parameter at baseline)×100/Parameter at baseline] and 3) the percentage change with respect to baseline corrected by the changes observed in time matched controls according the following equation ΔΔ=Δ % treatment −Δ % control. The SEM of the difference was calculated according to the following equation ΔΔSEM=(SEMtreatment2+SEMcontrol2)0.5.

The statistical analysis includes 1) a Student paired t-test to evaluate the changes respect to baseline and, 2) a multi-comparison analysis (Dunnett's test) of A % changes respect to time matched controls. An alpha probability of 0.05 was considered significant.

The table below shows iPSC Cardiomyocyte data for compounds of the invention and comparative examples.

| | ΔΔ Na Spike | cTnl release at 10 μM | Proarrhythmic |
|---|---|---|---|
| | Amp at 10 μM, | relative to vehicle | Markers at |
| Example | 48 h (%) | (pg/well) | 10 μM, 48 h |
| Ex. 97 | 2.20 | 0 | Not Observed |
| Ex. 39 | −43.3 | 0 | Not Observed |
| Comp. Ex. 2 | Not Measured | 111.2 | Q and RV |
| | (cells quiescent) | | |
| Comp. Ex. 3 | −96.2 | 0 | II and TA |

Q = quiescence;
RV = rhythmic variability;
II = impedance instability;
TA = triggered activity

hERG

hERG IC50 data was supplied by Metrion using their standard assay protocol briefly detailed below.

hERG ion channel screening was assayed in CHO cells stably expressing the human ether-á-go-go related gene, using the QPatch 48 automated, chip-based planar patch clamp device. A Gigaohm seal between the cell membrane and treated silicon surface was obtained and specific external and internal buffered solutions applied to cells prior to recording. Following baseline vehicle treatment, compounds were applied in increasing concentrations (8 pt CRC), effects on hERG tail current amplitudes were measured in two-minute recordings.

The table below shows hERG IC50 data for compounds of the invention and comparative examples.

| Example | hERG IC50 (μM) |
|---|---|
| Ex. 97 | >10 |
| Ex. 25 | >10 |
| Ex. 39 | >10 |
| Comp. Ex. 2 | >10 |
| Comp. Ex. 3 | 3.8 |

### Discussion of Safety Panel Data

Ponatinib (Comparative Example 2) is associated with severe adverse events in the clinic, and is marketed with a black box warning for arterial occlusive events. An in vitro angiogenesis model using human umbilical vein endothelial cells (HUVECs) has been previously used to investigate vascular adverse events for protein tyrosine kinase inhibitors¹¹. Using this model, ponatinib was shown to reduce HUVEC viability and inhibit HUVEC tube formation. To evaluate the antiangiogenic activity of compounds of the invention relative to ponatinib, tube formation and viability assays were performed in HUVECs. The data shown in the table above indicates that ponatinib has a more significant inhibitory effect on tube formation, with a G150 which indicates potency at least 10-fold greater than the compounds of the invention. Ponatinib also demonstrated around a 10-fold (or greater) effect on cell viability compared to the compounds of the invention. The compounds of the invention would therefore be expected to have a less significant anti-angiogenic activity than Ponatinib, according to this model.

Both ponatinib (Comparative Example 2) and nilotinib (Comparative Example 3) are associated with serious cardiovascular adverse events in the clinic. Human-induced pluripotent stem cell-derived cardiomyocytes have been used in the literature to model the cardiotoxic effect of TKIs such as ponatinib and nilotinib¹². In accordance with the literature, it was found that ponatinib is able to induce structural cardiac toxicity as shown by a number of parameters in the table above, including increased troponin (cTnI) secretion (where cTnI is a known marker of cardiac injury). By comparison, the compounds of the invention did not demonstrate any significant cTnI release at 10 μM (48 h), relative to vehicle. Impedance measurements 48 h after treatment with ponatinib at 10 μM demonstrated that sodium spike amplitude could not be measured for ponatinib, due to the damaging or detrimental effect on cardiac cell health which caused the cells to become quiescent (lack of activity). The greatest measured effect on sodium spike amplitude was seen for nilotinib (as previously reported in the literature), additionally significant proarrhythmic markers were observed including triggered activity and impedance instability¹³. A significant reduction in sodium spike amplitude (96.2% vs. control) was observed at 10 μM after 48 hours for nilotinib. Example 39 only gave a 43% reduction, whereas Example 97 had no significant effect on sodium spike amplitude (2.2% vs. control).

It can also be seen from the data in the table above that compounds of the invention have reduced hERG inhibitory activity compared to nilotinib.

The safety panel data clearly demonstrates that the compounds of the invention have an improved safety profile compared to the ponatinib and nilotinib.

### High Resolution Mass Spectrometry Data

| | | | positive | negative | MIM calculated from | MIM calculated from |
|---|---|---|---|---|---|---|
| Ex. | MWt | MIM | ion m/z | ion m/z | positive ion* | negative ion* |
| 1 | 356.42 | 356.16 | 357.1707 | NA | 356.1634 | NA |
| 2 | 370.44 | 370.17 | 371.1864 | 369.1717 | 370.1791 | 370.179 |
| 3 | 345.4 | 345.16 | 346.1658 | 344.1519 | 345.1585 | 345.1592 |
| 4 | 384.35 | 384.12 | 385.1262 | 383.1124 | 384.1189 | 384.1197 |
| 5 | 398.38 | 398.14 | 399.142 | 397.1278 | 398.1347 | 398.1351 |
| 6 | 358.44 | 358.18 | 359.1859 | NA | 358.1786 | NA |
| 7 | 356.42 | 356.16 | 357.1706 | NA | 356.1633 | NA |
| 8 | 398.38 | 398.14 | 399.1418 | 397.1279 | 398.1345 | 398.1352 |
| 9 | 356.42 | 356.16 | 357.1705 | 355.1565 | 356.1632 | 356.1638 |
| 11 | 372.46 | 372.2 | 373.202 | 371.1875 | 372.1947 | 372.1948 |
| 12 | 372.46 | 372.2 | 373.2018 | 371.1875 | 372.1945 | 372.1948 |
| 13 | 356.42 | 356.16 | 357.1706 | 355.1566 | 356.1633 | 356.1639 |
| 14 | 366.36 | 366.13 | 367.1371 | NA | 366.1298 | NA |
| 15 | 372.46 | 372.2 | 373.202 | 371.1878 | 372.1947 | 372.1951 |
| 16 | 378.37 | 378.13 | 379.1363 | 377.1221 | 378.129 | 378.1294 |
| 17 | 370.45 | 370.18 | 371.1863 | 369.1718 | 370.179 | 370.1791 |
| 18 | 370.44 | 370.17 | 371.1863 | 369.1722 | 370.179 | 370.1795 |
| 19 | 345.39 | 345.15 | 346.1659 | NA | 345.1586 | NA |
| 20 | 358.43 | 358.17 | 359.1862 | 357.172 | 358.1789 | 358.1793 |
| 21 | 382.36 | 382.12 | 383.1304 | 381.117 | 382.1231 | 382.1243 |
| 22 | 380.39 | 380.14 | 381.1516 | 379.1375 | 380.1443 | 380.1448 |
| 23 | 358.43 | 358.17 | 359.1861 | 357.1725 | 358.1788 | 358.1798 |
| 24 | 344.41 | 344.16 | 345.1705 | 343.1563 | 344.1632 | 344.1636 |
| 25 | 344.41 | 344.16 | 345.1707 | NA | 344.1634 | NA |
| 26 | 370.45 | 370.17 | 371.1863 | NA | 370.179 | NA |
| 27 | 356.42 | 356.16 | 357.1706 | NA | 356.1633 | NA |
| 28 | 384.35 | 384.12 | 385.1265 | 383.1119 | 384.1192 | 384.1192 |
| 29 | 410.39 | 410.14 | 411.1422 | 409.1281 | 410.1349 | 410.1354 |
| 30 | 368.43 | 368.16 | 369.1709 | NA | 368.1636 | NA |
| 31 | 344.41 | 344.16 | 345.1708 | NA | 344.1635 | NA |
| 32 | 342.39 | 342.15 | 343.1552 | NA | 342.1479 | NA |
| 34 | 359.42 | 359.16 | 360.1703 | 358.1561 | 359.163 | 359.1634 |
| 35 | 345.39 | 345.14 | 346.1546 | 344.1402 | 345.1473 | 345.1475 |
| 36 | 346.38 | 346.15 | 347.161 | NA | 346.1537 | NA |
| 37 | 359.42 | 359.17 | 360.1813 | NA | 359.174 | NA |
| 38 | 358.44 | 358.18 | 359.1863 | NA | 358.179 | NA |
| 39 | 386.45 | 386.17 | 387.1808 | NA | 386.1735 | NA |
| 46 | 370.45 | 370.18 | 371.1865 | NA | 370.1792 | NA |
| 41 | 382.46 | 382.18 | 383.1862 | NA | 382.1789 | NA |
| 42 | 370.45 | 370.18 | 371.1866 | NA | 370.1793 | NA |
| 43 | 366.36 | 366.12 | 367.136 | 365.1216 | 366.1287 | 366.1289 |
| 44 | 370.45 | 370.18 | 371.1863 | NA | 370.179 | NA |
| 45 | 359.42 | 359.16 | 360.17 | 358.156 | 359.1627 | 359.1633 |
| 46 | 346.38 | 346.14 | 347.1496 | 345.1354 | 346.1423 | 346.1427 |
| 47 | 358.44 | 358.18 | 359.186 | NA | 358.1787 | NA |
| 48 | 384.35 | 384.11 | 385.1263 | 383.1122 | 384.119 | 384.1195 |
| 49 | 386.44 | 386.17 | 387.181 | 385.1678 | 386.1737 | 386.1751 |
| 50 | 344.41 | 344.16 | 345.1703 | 343.1564 | 344.163 | 344.1637 |
| 51 | 370.45 | 370.18 | 371.1863 | 369.1718 | 370.179 | 370.1791 |
| 52 | 414.38 | 414.13 | 415.1368 | 413.1232 | 414.1295 | 414.1305 |
| 53 | 366.36 | 366.12 | 367.1358 | 365.1215 | 366.1285 | 366.1288 |
| 54 | 380.39 | 380.14 | 381.1515 | 379.1374 | 380.1442 | 380.1447 |
| 55 | 358.44 | 358.18 | 359.1861 | NA | 358.1788 | NA |
| 56 | 384.47 | 384.2 | 385.202 | NA | 384.1947 | NA |
| 58 | 358.44 | 358.18 | 359.1862 | NA | 358.1789 | NA |
| 59 | 367.35 | 367.12 | 368.1311 | 366.1171 | 367.1238 | 367.1244 |
| 61 | 372.46 | 372.19 | 373.2019 | 371.1874 | 372.1946 | 372.1947 |
| 62 | 342.39 | 342.15 | 343.1549 | 341.1407 | 342.1476 | 342.148 |
| 63 | 368.43 | 368.16 | 369.1704 | 367.1564 | 368.1631 | 368.1637 |
| 64 | 358.44 | 358.18 | 359.1862 | NA | 358.1789 | NA |
| 65 | 370.45 | 370.18 | 371.1863 | NA | 370.179 | NA |
| 66 | 344.41 | 344.16 | 345.1705 | NA | 344.1632 | NA |
| 68 | 370.44 | 370.17 | 371.1862 | 369.1718 | 370.1789 | 370.1791 |
| 69 | 356.42 | 356.16 | 357.1706 | NA | 356.1633 | NA |
| 76 | 370.44 | 370.17 | 371.1865 | 369.1717 | 370.1792 | 370.179 |
| 71 | 398.38 | 398.13 | 399.1432 | 397.1278 | 398.1359 | 398.1351 |
| 72 | 380.39 | 380.14 | 381.1513 | 379.1372 | 380.144 | 380.1445 |
| 73 | 378.37 | 378.13 | 379.1357 | 377.122 | 378.1284 | 378.1293 |
| 74 | 372.46 | 372.19 | 373.2019 | 371.1879 | 372.1946 | 372.1952 |
| 75 | 366.36 | 366.13 | 367.1359 | 365.1217 | 366.1286 | 366.129 |
| 76 | 398.38 | 398.14 | 399.1419 | 397.1279 | 398.1346 | 398.1352 |
| 77 | 370.45 | 370.18 | 371.1862 | 369.1721 | 370.1789 | 370.1794 |
| 78 | 348.37 | 348.14 | 349.1456 | NA | 348.1383 | NA |
| 79 | 358.43 | 358.17 | 359.1861 | 357.1724 | 358.1788 | 358.1797 |
| 80 | 358.44 | 358.18 | 359.1858 | NA | 358.1785 | NA |
| 81 | 385.41 | 385.15 | 386.1606 | 384.1466 | 385.1533 | 385.1539 |
| 82 | 356.42 | 356.16 | 357.1706 | NA | 356.1633 | NA |
| 83 | 398.38 | 398.13 | 399.1423 | 397.1278 | 398.135 | 398.1351 |
| 84 | 356.42 | 356.16 | 357.1706 | 355.1563 | 356.1633 | 356.1636 |
| 85 | 359.42 | 359.17 | 360.1812 | NA | 359.1739 | NA |
| 86 | 352.33 | 352.11 | 353.1201 | 351.106 | 352.1128 | 352.1133 |
| 87 | 380.39 | 380.14 | 381.1513 | 379.1375 | 380.144 | 380.1448 |
| 88 | 370.4 | 370.14 | 371.1495 | 369.1355 | 370.1422 | 370.1428 |
| 89 | 372.42 | 372.16 | 373.1654 | NA | 372.1581 | NA |
| 96 | 378.85 | 378.12 | 379.1316 | 377.1175 | 378.1243 | 378.1248 |
| 91 | 370.45 | 370.18 | 371.1864 | 369.1719 | 370.1791 | 370.1792 |
| 92 | 372.46 | 372.19 | 373.2018 | NA | 372.1945 | NA |
| 93 | 374.44 | 374.17 | 375.1809 | 373.1669 | 374.1736 | 374.1742 |
| 94 | 370.45 | 370.18 | 371.1864 | NA | 370.1791 | NA |
| 95 | 382.46 | 382.18 | 383.1862 | NA | 382.1789 | NA |
| 96 | 386.45 | 386.17 | 387.1812 | NA | 386.1739 | NA |
| 97 | 392.4 | 392.14 | 393.1511 | 391.1376 | 392.1438 | 392.1449 |
| | | 487 | | | | |
| 98 | 348.37 | 348.13 | 349.1454 | 347.1312 | 348.1381 | 348.1385 |
| 99 | 356.42 | 356.16 | 357.1706 | 355.1566 | 356.1633 | 356.1639 |
| 190 | 357.4 | 357.14 | 358.154 | 356.1402 | 357.1467 | 357.1475 |
| 101 | 357.41 | 357.14 | 358.1541 | 356.1403 | 357.1468 | 357.1476 |
| 102 | 393.39 | 393.12 | 394.1357 | 392.1215 | 393.1284 | 393.1288 |
| 103 | 360.41 | 360.16 | 361.1652 | 359.1513 | 360.1579 | 360.1586 |
| 104 | 406.43 | 406.16 | 407.1669 | 405.1531 | 406.1596 | 406.1604 |
| 106 | 496.53 | 496.22 | 497.2266 | 495.2121 | 496.2193 | 496.2194 |
| Comp. | 398.46 | 398.17 | 399.1809 | 397.1666 | 398.1736 | 398.1739 |
| Ex. 1 | | | | | | |
| Comp. | 532.56 | 532.22 | 533.2267 | 531.212 | 532.2194 | 532.2193 |
| Ex. 2 | | | | | | |
| Comp. | 529.52 | 529.18 | 530.1906 | 528.176 | 529.1833 | 529.1833 |
| Ex. 3 | | | | | | |
| Comp. | 498.55 | 498.22 | 499.2262 | 497.2122 | 498.2189 | 498.2195 |
| Ex. 4 | | | | | | |

### REFERENCES

1. De Lin et al. (2017) Novel Pathways of Ponatinib Disposition Catalyzed by CYP1A1 Involving Generation of Potentially Toxic Metabolites. J Pharmacol Exp Ther 363:12-19
2. Food and Drug Administration. (2018). Bioanalytical Method Validation: Guidance for Industry.
3. Whitmire M, Ammerman J, de Lisio P, Killmer J, Kyle D (2011) LC-MS/MS Bioanalysis Method Development, Validation, and Sample Analysis: Points to Consider When Conducting Nonclinical and Clinical Studies in Accordance with Current Regulatory Guidances. J Anal Bioanal Techniques S4:001. doi:10.4172/2155-9872.S4-001
4. Summerfield S, Stevens A J, Cutler L, Del Carmen Osuna M, Hammond B, Tang S P, Hersey A, Spalding D J, and Jeffrey P (2006) Improving the in vitro prediction of in vivo CNS penetration: integrating permeability, Pgp efflux and free fractions in blood and brain. J PharmacolExp Ther 316:1282-1290
5. Waters, Nigel J. et al. (2008) Validation of a Rapid Equilibrium Dialysis Approach for the Measurement of Plasma Protein Binding. Journal of Pharmaceutical Sciences: 97 (10) 4586-4595
6. Kalvass J C, Maurer T S, Pollack G M (2007) Use of plasma and brain unbound fractions to assess the extent of brain distribution of 34 drugs: comparison of unbound concentration ratios to in vivo p-glycoprotein efflux ratios. Drug Metab Dispos 35: 660-666.
7. Friden M, Gupta A, Antonsson M, Bredberg U, Hammarlund-Udenaes M (2007) In vitro methods for estimating unbound drug concentrations in the brain interstitial and intracellular fluids. Drug Metab Dispos 35: 1711-1719.
8. Meyer, K., Ferraiuolo, L., Miranda, C. J., Likhite, S., McElroy, S., Renusch, S., Ditsworth, D., Lagier-Tourenne, C., Smith, R. A., Ravits, J., Burghes, A. H., Shaw, P. J., Cleveland, D. W., Kolb, S. J., and Kaspar, B. K. (2014) Direct conversion of patient fibroblasts demonstrates non-cell autonomous toxicity of astrocytes to motor neurons in familial and sporadic ALS. PNAS 111, 829-832.
9. Haidet-Phillips, A. M., Hester, M. E., Miranda, C. J., Meyer, K., Braun, L., Frakes, A., Song, S., Likhite, S., Murtha, M. J., Foust, K. D., Rao, M., Eagle, A., Kammesheidt, A., Christensen, A., Mendell, J. R., Burghes, A. H. M., and Kaspar, B. K. (2011) Astrocytes from familial and sporadic ALS patients are toxic to motor neurons. Nature Biotechnology 29, 824-828.
10. Wichterle, H., Lieberam, I., Porter, J. A. and Jessell, T. M. (2002) Directed differentiation of embryonic stem cells into motor neurons. Cell 110, 385-397.
11. Leukemia & Lymphoma, 2017, Vol. 58, No. 6, 1455-1467.
12. Toxicological Sciences, 143(1), 2015, 147-155.
13. Toxicology & Applied Pharmacology, 272(1), 2013, 245-255.

1. A compound of Formula (I) 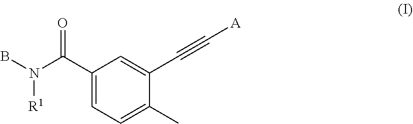
or a pharmaceutically acceptable salt, solvate, hydrate, tautomer, optical isomer, N-oxide, and/or prodrug thereof, wherein
A is an unsubstituted pyridyl;
B is a substituted 5-membered heteroaryl; and
R¹ is H or is selected from the group consisting of
(i) C₁-C₆ alkyl, C₂-C₆ alkenyl, and C₂-C₆ alkynyl, each of which is optionally substituted with one or more substituents independently selected from —NR^{a}R^{b}, —OR^{c}, halo, and oxo; and
(ii) C₆-C₁₀ aryl, C₁-C₉ heteroaryl, C₁-C₉ heterocycle, each of which is optionally substituted with one or more substituents independently selected from halo and C₁-C₆ alkyl, wherein the C₁-C₆ alkyl is optionally substituted with one or more halo atoms,
wherein
each R^{a} and R^{b} are independently selected from H and C₁-C₆ alkyl wherein the C₁-C₆ alkyl is optionally substituted with one or more halo atoms, or R^{a} and R^{b}, can be taken together with the nitrogen atom to which they are attached to form a 5- or 6-membered saturated, partially saturated, or unsaturated ring; and
each R^{c} is independently selected from H and C₁-C₆ alkyl wherein the C₁-C₆ alkyl is optionally substituted with one or more halo atoms.

2. The compound according to claim 1, wherein the compound is a compound of Formula (II)

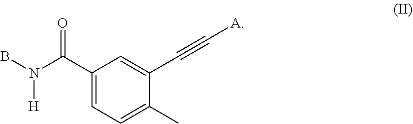

3. The compound according to claim 1 or claim 2, wherein the 5-membered heteroaryl of group B is substituted with one or more substituents independently selected from the group consisting of
(i) C₁-C₆ alkyl, C₂-C₆ alkenyl, C₂-C₆ alkynyl, and C₁-C₆ alkoxy, each of which is optionally substituted with one or more substituents independently selected from —NR^{d}R^{e}, —OR^{f}, halo, and oxo;
(ii) halo, —CN, —C(O)NR^{g}R^{h}, —NR^{g}R^{h}, —(O)ORⁱ, —(O)Rⁱ, and —ORⁱ; and
(iii) C₆-C₁₀ aryl, C₁-C₉ heteroaryl, and C₁-C₉ heterocycle, each of which is optionally substituted with one or more substituents independently selected from halo and C₁-C₆ alkyl, wherein the C₁-C₆ alkyl is optionally substituted with one or more halo atoms;
and/or wherein an alkylene group is attached to two adjacent atoms of the 5-membered heteroaryl of group B to form a 5-, 6-, or 7-membered (preferably 5- or 6-membered) unsaturated, partially saturated or saturated ring (preferably a partially saturated or saturated ring), which is fused to the 5-membered heteroaryl of group B,
optionally wherein one or two carbon atoms of the alkylene group are independently replaced with a heteroatom, optionally wherein when the heteroatom is nitrogen then said nitrogen is substituted with C₁-C₆ alkyl, or —C(O)O—(C₁-C₆ alkyl) wherein the C₁-C₆ alkyl is optionally substituted with one or more halo atoms, and optionally wherein when the heteroatom is sulfur then said sulfur forms a thionyl or sulfonyl group;
optionally wherein one or more of the carbon atoms of the alkylene group is substituted with one or more substituents independently selected from halo, —C(O)O—(C₁-C₆ alkyl), C₁-C₆ alkyl, and oxo, wherein said alkyl groups are optionally independently substituted with one or more halo atoms; and/or
optionally wherein two hydrogen atoms attached to the same carbon of the alkylene group are replaced with carbon atoms which, together with the carbon atom to which they are attached, form a C₃-C₆ cyclic alkyl group, wherein said cyclic alkyl group is optionally substituted with one or more halo atoms, and/or one carbon is replaced with a heteroatom, preferably O or N;
wherein R^{d}, R^{e}, R^{g}, and R^{h} are independently selected from H and C₁-C₆ alkyl wherein the C₁-C₆ alkyl is optionally substituted with one or more halo atoms, or R^{d} and R^{e}, and/or R^{g} and R^{h}, can be taken together with the nitrogen atom to which they are attached to form a 5- or 6-membered saturated, partially saturated, or unsaturated ring which ring is optionally substituted with one or more groups selected from halo and C₁-C₃ alkyl, wherein the C₁-C₃ alkyl is optionally substituted with one or more halo atoms; and
wherein R^{f} and Rⁱ are independently selected from H and C₁-C₆ alkyl, wherein the C₁-C₆ alkyl is optionally substituted with one or more halo atoms.

4. The compound according to claim 3, wherein the 5-membered heteroaryl of group B is substituted with one or more substituents independently selected from the group consisting of
(i) C₁-C₆ alkyl optionally substituted with one or more substituents selected from —NR^{d}R^{e}, —OR^{f}, halo, and oxo;
(ii) halo, —C(O)NR^{g}R^{h}, —C(O)ORⁱ; and
(iii) —ORⁱ,
and/or wherein an alkylene group is attached to two adjacent atoms of the 5-membered heteroaryl of group B to form a 5-, 6-, or 7-membered (preferably 5- or 6-membered) unsaturated, partially saturated or saturated ring (preferably a partially saturated or saturated ring), which is fused to the 5-membered heteroaryl of group B,
optionally wherein one or two carbon atoms of the alkylene group are independently replaced with a heteroatom, optionally wherein when the heteroatom is nitrogen then said nitrogen is substituted with C₁-C₆ alkyl, or —C(O)O—(C₁-C₆ alkyl) wherein the C₁-C₆ alkyl is optionally substituted with one or more halo atoms, and optionally wherein when the heteroatom is sulfur then said sulfur forms a thionyl or sulfonyl group;
optionally wherein one or more of the carbon atoms of the alkylene group is substituted with one or more substituents independently selected from halo, —C(O)O—(C₁-C₆ alkyl), C₁-C₆ alkyl, and oxo, wherein said C₁-C₆ alkyl is optionally independently substituted with one or more halo atoms; and/or
optionally wherein two hydrogen atoms attached to the same carbon of the alkylene group are replaced with carbon atoms which, together with the carbon atom to which they are attached, form a C₃-C₅ cyclic alkyl group, wherein said cyclic alkyl group is optionally substituted with one or more halo atoms, and/or one carbon is replaced with a heteroatom, preferably O or N.

5. The compound according to any preceding claim, wherein an alkylene group is attached to two adjacent atoms of the 5-membered heteroaryl of group B to form a 5-, 6-, or 7-membered (preferably 5- or 6-membered) unsaturated, partially saturated or saturated ring (preferably a partially saturated or saturated ring), which is fused to the 5-membered heteroaryl of group B,
optionally wherein one or two carbon atoms of the alkylene group are independently replaced with a heteroatom, optionally wherein when the heteroatom is nitrogen then said nitrogen is substituted with C₁-C₆ alkyl, or —C(O)O—(C₁-C₆ alkyl) wherein the C₁-C₆ alkyl is optionally substituted with one or more halo atoms, and optionally wherein when the heteroatom is sulfur then said sulfur forms a thionyl or sulfonyl group;
optionally wherein one or more of the carbon atoms of the alkylene group is substituted with one or more substituents independently selected from halo, —C(O)O—(C₁-C₆ alkyl), C₁-C₆ alkyl, and oxo, wherein said C₁-C₆ alkyl is optionally independently substituted with one or more halo atoms; and/or
optionally wherein two hydrogen atoms attached to the same carbon of the alkylene group are replaced with carbon atoms which, together with the carbon atom to which they are attached, form a C₃-C₆ cyclic alkyl group, wherein said cyclic alkyl group is optionally substituted with one or more halo atoms, and/or one carbon is replaced with a heteroatom, preferably O or N.

6. The compound according to claim 4, wherein the 5-membered heteroaryl of group B is substituted with one or more substituents independently selected from the group consisting of
(i) C₁-C₆ alkyl optionally substituted with one or more substituents selected from halo, and —OR^{f};
(ii) halo, —C(O)ORⁱ; and
(iii) —ORⁱ,
(preferably a C₁-C₆ alkyl, isopropyl group or t-butyl group, more preferably an isopropyl group or t-butyl group, most preferably a t-butyl group), and/or wherein an alkylene group is attached to two adjacent atoms of the 5-membered heteroaryl of group B to form a 5- or 6-membered partially saturated or saturated ring, which is fused to the 5-membered heteroaryl of group B,
optionally wherein one or more of the carbon atoms of the alkylene group is substituted with one or more substituents independently selected from halo, C₁-C₆ alkyl and oxo, wherein said alkyl groups are optionally independently substituted with one or more halo atoms, and/or
optionally wherein two hydrogen atoms attached to the same carbon of the alkylene group are replaced with carbon atoms which, together with the carbon atom to which they are attached, form a C₃-C₆ cyclic alkyl group, wherein said cyclic alkyl group is optionally substituted with one or more halo atoms.

7. The compound according to any preceding claim, wherein the substituted 5-membered heteroaryl of group B is a substituted pyrrole, pyrazole, imidazole, triazole, tetrazole, isoxazole, or thiazole, preferably a substituted pyrazole, imidazole, triazole, tetrazole, isoxazole or thiazole, more preferably a substituted pyrazole, triazole or imidazole group, most preferably a substituted pyrazole or imidazole group.

8. The compound according to any preceding claim, wherein group B is selected from one of the following groups 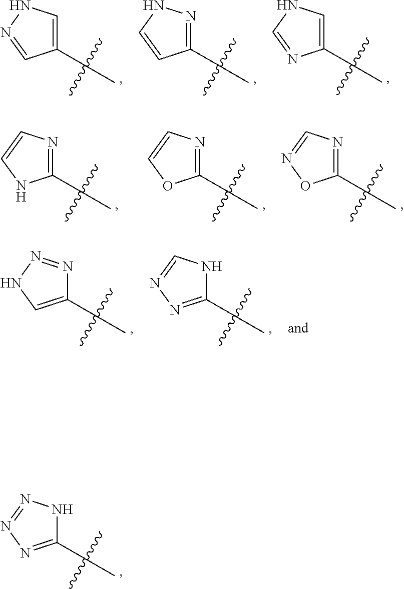
preferably 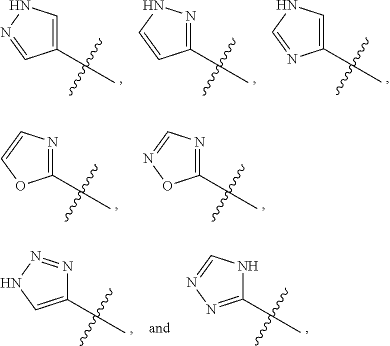
or a tautomer thereof, and each group being substituted.

9. The compound according to claim 1 or 2, wherein group B is selected from optionally substituted group (V) and optionally substituted group (W) 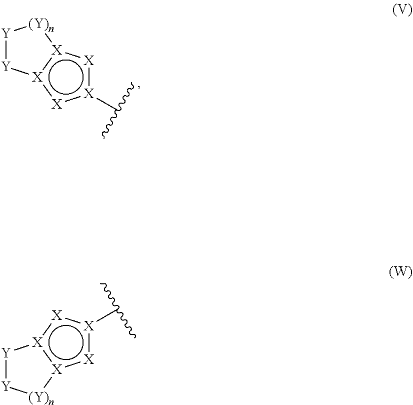
wherein each X and Y is independently selected from C, S, O, and N,
at least one X is N;
at least two X are C;
at least two Y are C;
n=1, 2 or 3, preferably 1 or 2;
wherein
each X is optionally independently substituted with one or more substituents selected from halo, —CN, —C(O)OH, —C(O)O—(C₁-C₆ alkyl), and C₁-C₆ alkyl, preferably halo, —C(O)OH, —C(O)O—(C₁-C₆ alkyl), and C₁-C₆ alkyl, most preferably C₁-C₆ alkyl, wherein said alkyl groups are optionally substituted with one or more halo atoms; and
each Y is optionally independently substituted with one or more substituents selected from halo, —C(O)OH, —C(O)O—(C₁-C₆ alkyl), C₁-C₆ alkyl, and oxo, preferably halo, —C(O)O—(C₁-C₆ alkyl), C₁-C₆ alkyl, and oxo, most preferably C₁-C₆ alkyl and oxo, wherein said alkyl groups are optionally substituted with one or more halo atoms; and/or
optionally wherein two hydrogen atoms attached to the same Y are replaced with carbon atoms which, together with the carbon atom to which they are attached, form a C₃-C₆ cyclic alkyl group, wherein said cyclic alkyl group is optionally substituted with one or more halo atoms,
preferably the compounds are of Formulae (II-V) or (II-W).

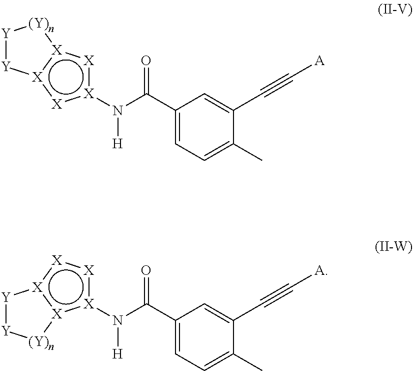

10. The compound according to claim 9, wherein group B is selected from one of the following groups 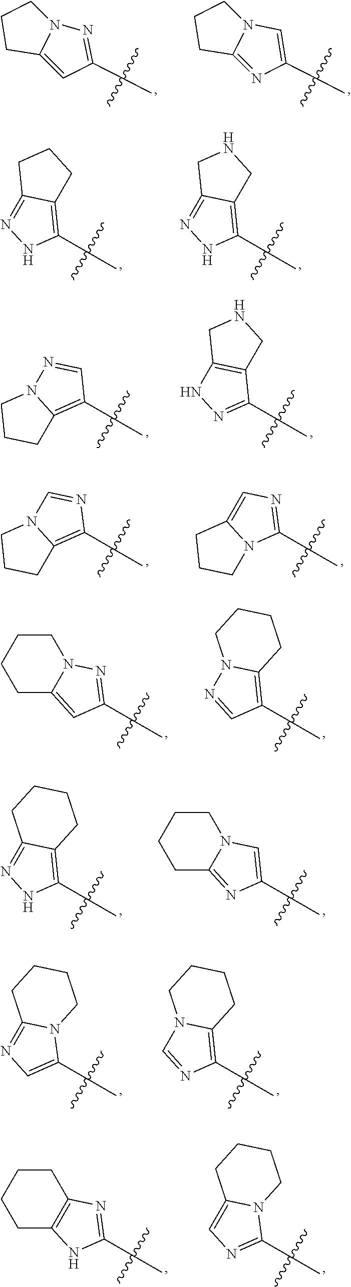 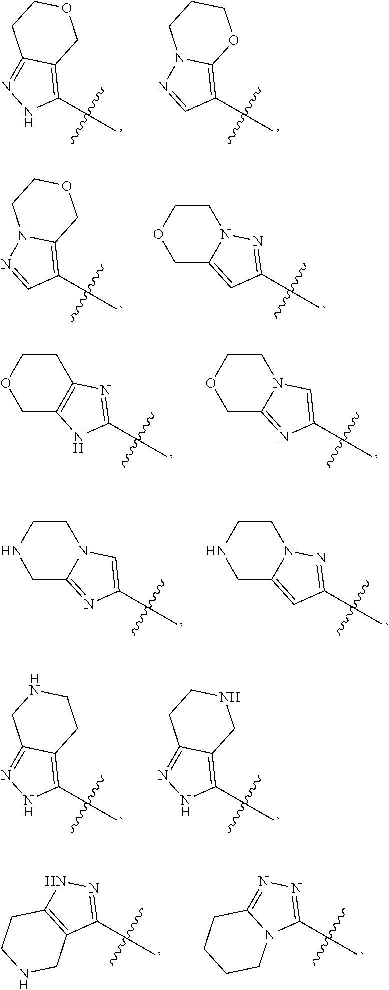 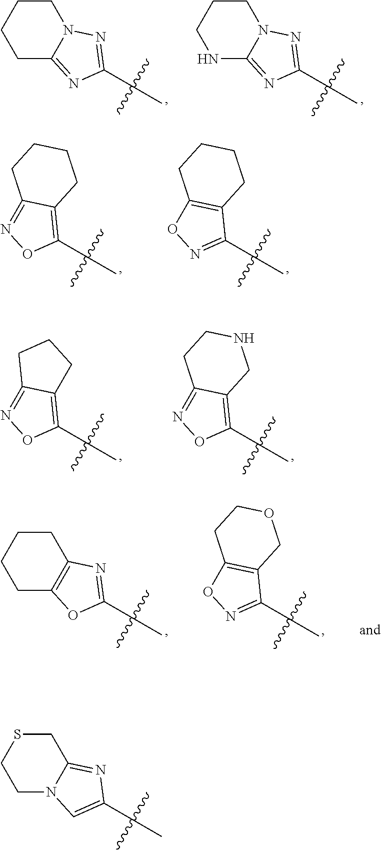
preferably group B is selected from one of the following groups 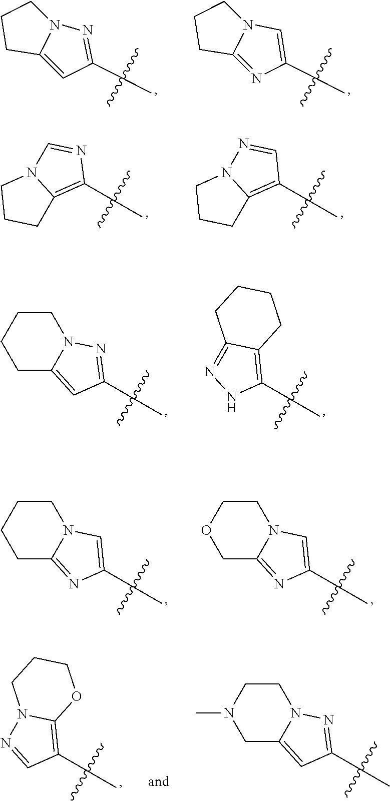
or a tautomer thereof, each group being optionally substituted.

11. The compound according to any preceding claim, wherein A is selected from 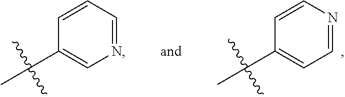 [SMILES: *c1cccnc1 ; "and" ; *c1ccncc1]
preferably A is

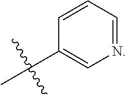 [SMILES: *c1cccnc1]

12. The compound according to any preceding claim, wherein
A is 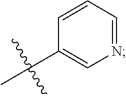 [SMILES: *c1cccnc1]
B is selected from the group consisting of 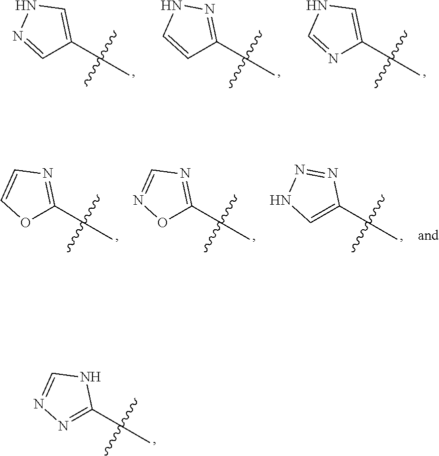
and is substituted with one or more substituents independently selected from the group consisting of
(i) C₁-C₆ alkyl optionally substituted with one or more substituents selected from halo, and —OR^{f};
(ii) halo, —C(O)ORⁱ; and
(iii) —ORⁱ,
(preferably a C₁-C₆ alkyl, isopropyl group or t-butyl group, more preferably an isopropyl group or t-butyl group, most preferably a t-butyl group), and/or wherein an alkylene group is attached to two adjacent atoms of the 5-membered heteroaryl of group B to form a 5-, 6-membered partially saturated or saturated ring, which is fused to the 5-membered heteroaryl of group B,
optionally wherein one or more of the carbon atoms of the alkylene group is substituted with one or more substituents independently selected from halo, C₁-C₆ alkyl and oxo (preferably substituted alpha- to a bridgehead atom with a di-C₁-C₆ alkyl group, more preferably forming a gem-dimethyl group), wherein said alkyl groups are optionally independently substituted with one or more halo atoms; and/or
optionally wherein two hydrogen atoms attached to the same carbon of the alkylene group are replaced with carbon atoms which, together with the carbon atom to which they are attached, form a C₃-C₆ cyclic alkyl group, wherein said cyclic alkyl group is optionally substituted with one or more halo atoms.

13. The compound according to any preceding claim, wherein the compound is
4-methyl-N-{4H,5H,6H,7H-pyrazolo[1,5-a]pyridin-2-yl}-3-[2-(pyridin-3-yl)ethynyl]benzamide;
4-methyl-N-(2-methyl-4,5,6,7-tetrahydro-2H-indazol-3-yl)-3-[2-(pyridin-3-yl)ethynyl]benzamide;
4-methyl-N-[1-(propan-2-yl)-1H-1,2,3-triazol-4-yl]-3-[2-(pyridin-3-yl)ethynyl]benzamide;
4-methyl-3-[2-(pyridin-3-yl)ethynyl]-N-[1-(2,2,2-trifluoroethyl)-1H-pyrazol-3-yl]benzamide;
4-methyl-3-[2-(pyridin-3-yl)ethynyl]-N-[1-(3,3,3-trifluoropropyl)-1H-pyrazol-4-yl]benzamide;
N-(1-tert-butyl-1H-pyrazol-3-yl)-4-methyl-3-[2-(pyridin-3-yl)ethynyl]benzamide;
N-(5-cyclopropyl-1-methyl-1H-pyrazol-3-yl)-4-methyl-3-[2-(pyridin-3-yl)ethynyl]benzamide;
4-methyl-N-[5-methyl-1-(2,2,2-trifluoroethyl)-1H-pyrazol-3-yl]-3-[2-(pyridin-3-yl)ethynyl]benzamide;
N-(1-cyclobutyl-1H-pyrazol-4-yl)-4-methyl-3-[2-(pyridin-3-yl)ethynyl]benzamide;
N-(1-tert-butyl-1H-pyrazol-4-yl)-4-methyl-3-[2-(pyridin-3-yl)ethynyl]benzamide;
N-(3-tert-butyl-1-methyl-1H-pyrazol-5-yl)-4-methyl-3-[2-(pyridin-3-yl)ethynyl]benzamide;
N-[1-(2,2-dimethylpropyl)-1H-pyrazol-4-yl]-4-methyl-3-[2-(pyridin-3-yl)ethynyl]benzamide;
N-[1-(cyclopropylmethyl)-1H-pyrazol-4-yl]-4-methyl-3-[2-(pyridin-3-yl)ethynyl]benzamide;
N-[5-(difluoromethyl)-1-methyl-1H-pyrazol-3-yl]-4-methyl-3-[2-(pyridin-3-yl)ethynyl]benzamide;
4-methyl-N-[5-methyl-1-(2-methylpropyl)-1H-pyrazol-4-yl]-3-[2-(pyridin-3-yl)ethynyl]benzamide;
N-[1-(2,2-difluorocyclopropyl)-1H-pyrazol-4-yl]-4-methyl-3-[2-(pyridin-3-yl)ethynyl]benzamide;
N-[1-(1-cyclopropylethyl)-1H-pyrazol-4-yl]-4-methyl-3-[2-(pyridin-3-yl)ethynyl]benzamide;
N-(1-cyclobutyl-5-methyl-1H-pyrazol-4-yl)-4-methyl-3-[2-(pyridin-3-yl)ethynyl]benzamide;
4-methyl-N-[1-(propan-2-yl)-1H-1,2,4-triazol-3-yl]-3-[2-(pyridin-3-yl)ethynyl]benzamide;
4-methyl-N-[1-(2-methylpropyl)-1H-pyrazol-4-yl]-3-[2-(pyridin-3-yl)ethynyl]benzamide;
N-[5-(difluoromethoxy)-1-methyl-1H-pyrazol-3-yl]-4-methyl-3-[2-(pyridin-3-yl)ethynyl]benzamide;
N-[1-(2,2-difluoroethyl)-5-methyl-1H-pyrazol-4-yl]-4-methyl-3-[2-(pyridin-3-yl)ethynyl]benzamide;
4-methyl-N-[5-methyl-1-(propan-2-yl)-1H-pyrazol-4-yl]-3-[2-(pyridin-3-yl)ethynyl]benzamide;
N-(1-isopropylpyrazol-4-yl)-4-methyl-3-[2-(3-pyridyl)ethynyl]benzamide;
N-(1-isopropylimidazol-4-yl)-4-methyl-3-[2-(3-pyridyl)ethynyl]benzamide;
N-(4,4-dimethyl-5,6-dihydropyrrolo[1,2-b]pyrazol-2-yl)-4-methyl-3-[2-(3-pyridyl)ethynyl]benzamide;
N-[1-(cyclopropylmethyl)imidazol-4-yl]-4-methyl-3-[2-(3-pyridyl)ethynyl]benzamide;
4-Methyl-3-[2-(3-pyridyl)ethynyl]-N-[1-(2,2,2-trifluoroethyl)imidazol-4-yl]benzamide;
4-methyl-3-[2-(3-pyridyl)ethynyl]-N-[5-(trifluoromethyl)-6,7-dihydro-5H-pyrrolo[1,2-a]imidazol-2-yl]benzamide;
4-Methyl-3-[2-(3-pyridyl)ethynyl]-N-spiro[6,7-dihydropyrrolo[1,2-a]imidazole-5,1′-cyclopropane]-2-yl-benzamide;
4-methyl-N-(1-propylimidazol-4-yl)-3-[2-(3-pyridyl)ethynyl]benzamide;
N-(1-cyclopropylimidazol-4-yl)-4-methyl-3-[2-(3-pyridyl)ethynyl]benzamide;
4-methyl-3-[2-(3-pyridyl)ethynyl]-N-[5-(trifluoromethyl)-5,6,7,8-tetrahydroimidazo[1,2-a]178yridine-2-yl]benzamide;
N-(4-tert-butyl-1,3-oxazol-2-yl)-4-methyl-3-[2-(pyridin-3-yl)ethynyl]benzamide;
4-methyl-N-[4-(propan-2-yl)-1,3-oxazol-2-yl]-3-[2-(pyridin-3-yl)ethynyl]benzamide;
4-methyl-N-[2-(propan-2-yl)-2H-1,2,3,4-tetrazol-5-yl]-3-[2-(pyridin-3-yl)ethynyl]benzamide;
N-(1-tert-butyl-1H-1,2,4-triazol-3-yl)-4-methyl-3-[2-(pyridin-3-yl)ethynyl]benzamide;
N-(1-tert-butylimidazol-4-yl)-4-methyl-3-[2-(3-pyridyl)ethynyl]benzamide;
N-(5,5-dimethyl-6,8-dihydroimidazo[2,1-c][1,4]oxazin-2-yl)-4-methyl-3-[2-(3-pyridyl)ethynyl]benzamide;
N-(6,6-dimethyl-5,7-dihydropyrrolo[1,2-c]imidazol-1-yl)-4-methyl-3-[2-(3-pyridyl)ethynyl]benzamide;
4-methyl-3-[2-(3-pyridyl)ethynyl]-N-spiro[5,6-dihydropyrrolo[1,2-b]pyrazole-4,1′-cyclobutane]-2-yl-benzamide;
N-(1-cyclopentylimidazol-4-yl)-4-methyl-3-[2-(3-pyridyl)ethynyl]benzamide;
N-[1-(difluoromethyl)-5-methyl-1H-pyrazol-4-yl]-4-methyl-3-[2-(pyridin-3-yl)ethynyl]benzamide;
N-(5,5-dimethyl-6,7-dihydropyrrolo[1,2-a]imidazol-2-yl)-4-methyl-3-[2-(3-pyridyl)ethynyl]benzamide;
4-methyl-N-[4-(2-methylpropyl)-1,3-oxazol-2-yl]-3-[2-(pyridin-3-yl)ethynyl]benzamide;
4-methyl-N-[3-(propan-2-yl)-1,2,4-oxadiazol-5-yl]-3-[2-(pyridin-3-yl)ethynyl]benzamide;
N-[1-(butan-2-yl)-1H-pyrazol-3-yl]-4-methyl-3-[2-(pyridin-3-yl)ethynyl]benzamide;
4-methyl-3-[2-(pyridin-3-yl)ethynyl]-N-[1-(2,2,2-trifluoroethyl)-1H-pyrazol-4-yl]benzamide;
N-{6,6-dimethyl-5H,6H,7H-pyrazolo[3,2-b][1,3]oxazin-3-yl}-4-methyl-3-[2-(pyridin-3-yl)ethynyl]benzamide;
4-methyl-N-(1-propyl-1H-pyrazol-4-yl)-3-[2-(pyridin-3-yl)ethynyl]benzamide;
N-(3-cyclobutyl-1-methyl-1H-pyrazol-5-yl)-4-methyl-3-[2-(pyridin-3-yl)ethynyl]benzamide;
N-[3-methoxy-1-(2,2,2-trifluoroethyl)-1H-pyrazol-4-yl]-4-methyl-3-[2-(pyridin-3-yl)ethynyl]benzamide;
N-[1-(2,2-difluoroethyl)-1H-pyrazol-4-yl]-4-methyl-3-[2-(pyridin-3-yl)ethynyl]benzamide;
N-[1-(2,2-difluoroethyl)-5-methyl-1H-pyrazol-3-yl]-4-methyl-3-[2-(pyridin-3-yl)ethynyl]benzamide;
4-methyl-N-[5-methyl-1-(propan-2-yl)-1H-pyrazol-3-yl]-3-[2-(pyridin-3-yl)ethynyl]benzamide;
N-(5-isopropyl-6,7-dihydro-5H-pyrrolo[1,2-a]imidazol-2-yl)-4-methyl-3-[2-(3-pyridyl)ethynyl]benzamide;
N-(5-isopropyl-6,8-dihydro-5H-imidazo[2,1-c][1,4]oxazin-2-yl)-4-methyl-3-[2-(3-pyridyl)ethynyl]benzamide;
4-methyl-N-[4-methyl-1-(propan-2-yl)-1H-pyrazol-3-yl]-3-[2-(pyridin-3-yl)ethynyl]benzamide;
N-[5-(difluoromethyl)-1-methyl-1H-1,2,3-triazol-4-yl]-4-methyl-3-[2-(pyridin-3-yl)ethynyl]benzamide;
4-methyl-N-(5-methyl-6,7-dihydro-5H-pyrrolo[1,2-c]imidazol-1-yl)-3-[2-(3-pyridyl)ethynyl]benzamide;
4-methyl-N-[1-methyl-3-(2-methylpropyl)-1H-pyrazol-5-yl]-3-[2-(pyridin-3-yl)ethynyl]benzamide;
N-(1-cyclopropyl-1H-pyrazol-4-yl)-4-methyl-3-[2-(pyridin-3-yl)ethynyl]benzamide;
4-methyl-3-[2-(3-pyridyl)ethynyl]-N-spiro[5,6-dihydropyrrolo[1,2-b]pyrazole-4,1′-cyclopropane]-2-yl-benzamide;
N-(1-isobutylimidazol-4-yl)-4-methyl-3-[2-(3-pyridyl)ethynyl]benzamide;
N-(4-ethyl-5,6-dihydro-4H-pyrrolo[1,2-b]pyrazol-2-yl)-4-methyl-3-[2-(3-pyridyl)ethynyl]benzamide;
4-methyl-N-[1-(propan-2-yl)-1H-pyrazol-3-yl]-3-[2-(pyridin-3-yl)ethynyl]benzamide;
4-methyl-N-(5-methyl-5,6,7,8-tetrahydroimidazo[1,2-a]pyridin-2-yl)-3-[2-(3-pyridyl)ethynyl]benzamide;
N-[1-(cyclobutylmethyl)-1H-pyrazol-4-yl]-4-methyl-3-[2-(pyridin-3-yl)ethynyl]benzamide;
N-(1-cyclobutylimidazol-4-yl)-4-methyl-3-[2-(3-pyridyl)ethynyl]benzamide;
N-[3-(cyclopropylmethyl)-1-methyl-1H-pyrazol-5-yl]-4-methyl-3-[2-(pyridin-3-yl)ethynyl]benzamide;
4-methyl-N-[3-methyl-1-(2,2,2-trifluoroethyl)-1H-pyrazol-4-yl]-3-[2-(pyridin-3-yl)ethynyl]benzamide;
N-[1-(2,2-difluoroethyl)-3-methyl-1H-pyrazol-4-yl]-4-methyl-3-[2-(pyridin-3-yl)ethynyl]benzamide;
N-[1-(2,2-difluorocyclopropyl)-1H-pyrazol-3-yl]-4-methyl-3-[2-(pyridin-3-yl)ethynyl]benzamide;
4-methyl-N-[3-methyl-1-(2-methylpropyl)-1H-pyrazol-4-yl]-3-[2-(pyridin-3-yl)ethynyl]benzamide;
N-[1-(2,2-difluoroethyl)-1H-pyrazol-3-yl]-4-methyl-3-[2-(pyridin-3-yl)ethynyl]benzamide;
4-methyl-N-[4-methyl-1-(2,2,2-trifluoroethyl)-1H-pyrazol-3-yl]-3-[2-(pyridin-3-yl)ethynyl]benzamide;
N-(1-cyclobutyl-3-methyl-1H-pyrazol-4-yl)-4-methyl-3-[2-(pyridin-3-yl)ethynyl]benzamide;
N-[1-(2-fluoroethyl)imidazol-4-yl]-4-methyl-3-[2-(3-pyridyl)ethynyl]benzamide;
4-methyl-N-[3-methyl-1-(propan-2-yl)-1H-pyrazol-4-yl]-3-[2-(pyridin-3-yl)ethynyl]benzamide;
4-methyl-N-[1-(2-methylpropyl)-1H-pyrazol-3-yl]-3-[2-(pyridin-3-yl)ethynyl]benzamide;
4-methyl-N-{5-methyl-4-oxo-4H,5H,6H,7H-pyrazolo[1,5-a]pyrazin-2-yl}-3-[2-(pyridin-3-yl)ethynyl]benzamide;
4-methyl-N-(5-methyl-6,7-dihydro-5H-pyrrolo[1,2-a]imidazol-2-yl)-3-[2-(3-pyridyl)ethynyl]benzamide;
N-[1,4-dimethyl-3-(trifluoromethyl)-1H-pyrazol-5-yl]-4-methyl-3-[2-(pyridin-3-yl)ethynyl]benzamide;
N-(3-cyclopropyl-1-methyl-1H-pyrazol-5-yl)-4-methyl-3-[2-(pyridin-3-yl)ethynyl]benzamide;
4-methyl-N-[1-methyl-3-(propan-2-yl)-1H-1,2,4-triazol-5-yl]-3-[2-(pyridin-3-yl)ethynyl]benzamide;
N-[1-(difluoromethyl)-1H-pyrazol-4-yl]-4-methyl-3-[2-(pyridin-3-yl)ethynyl]benzamide;
N-[1-(2,2-difluoroethyl)-4-methyl-1H-pyrazol-3-yl]-4-methyl-3-[2-(pyridin-3-yl)ethynyl]benzamide;
4-methyl-N-(4-oxo-6,7-dihydro-5H-pyrazolo[1,5-a]pyridin-2-yl)-3-[2-(3-pyridyl)ethynyl]benzamide;
4-methyl-N-(5-methyl-6,8-dihydro-5H-imidazo[2,1-c][1,4]oxazin-2-yl)-3-[2-(3-pyridyl)ethynyl]benzamide;
N-[4-chloro-1-(propan-2-yl)-1H-pyrazol-3-yl]-4-methyl-3-[2-(pyridin-3-yl)ethynyl]benzamide;
N-(3-cyclopropyl-1-ethyl-1H-pyrazol-5-yl)-4-methyl-3-[2-(pyridin-3-yl)ethynyl]benzamide;
4-methyl-N-[5-methyl-1-(2-methylpropyl)-1H-pyrazol-3-yl]-3-[2-(pyridin-3-yl)ethynyl]benzamide;
N-[1-(1-methoxypropan-2-yl)-1H-pyrazol-3-yl]-4-methyl-3-[2-(pyridin-3-yl)ethynyl]benzamide;
N-[1-(cyclobutylmethyl)imidazol-4-yl]-4-methyl-3-[2-(3-pyridyl)ethynyl]benzamide;
4-Methyl-3-[2-(3-pyridyl)ethynyl]-N-spiro[6,7-dihydro-5H-pyrazolo[1,5-a]pyridine-4,1′-cyclopropane]-2-yl-benzamide;
N-(6,6-dimethyl-5,8-dihydroimidazo[2,1-c][1,4]oxazin-2-yl)-4-methyl-3-[2-(3-pyridyl)ethynyl]benzamide;
N-(4,4-difluoro-6,7-dihydro-5H-pyrazolo[1,5-a]pyridin-2-yl)-4-methyl-3-[2-(3-pyridyl)ethynyl]benzamide;
N-[1-(2-fluoroethyl)-1H-pyrazol-4-yl]-4-methyl-3-[2-(pyridin-3-yl)ethynyl]benzamide;
4-methyl-N-{4H,5H,6H,7H-pyrazolo[1,5-a]pyridin-3-yl}-3-[2-(pyridin-3-yl)ethynyl]benzamide;
4-methyl-3-[2-(pyridin-3-yl)ethynyl]-N-(4,5,6,7-tetrahydro-1,2-benzoxazol-3-yl)benzamide;
4-methyl-3-[2-(pyridin-3-yl)ethynyl]-N-(4,5,6,7-tetrahydro-2,1-benzoxazol-3-yl)benzamide;
N-(5,5-difluoro-4,5,6,7-tetrahydro-1,2-benzoxazol-3-yl)-4-methyl-3-[2-(pyridin-3-yl)ethynyl]benzamide; or
N-(5-tert-butyl-1,3,4-oxadiazol-2-yl)-4-methyl-3-[2-(pyridin-3-yl)ethynyl]benzamide,
or a pharmaceutically acceptable salt, solvate, hydrate, tautomer, optical isomer, N-oxide, and/or prodrug thereof.

14. A pharmaceutical composition comprising a compound according to any preceding claim and a pharmaceutically acceptable carrier, excipient, and/or diluent.

15. The compound according to any one of claims 1 to 13, or the pharmaceutical composition of claim 14, for use in therapy.

16. The compound according to any one of claims 1 to 13, or the pharmaceutical composition of claim 14, for use in the treatment or prevention of a neurodegenerative disorder, a cancer, a prion disease, a viral infection, diabetes, an inflammatory disease, or a skeletal or muscular dystrophy, preferably a neurodegenerative disorder or a cancer.

17. Use of the compound according to any one of claims 1 to 13 for the manufacture of a medicament for the treatment or prevention of a neurodegenerative disorder, a cancer, a prion disease, a viral infection, diabetes, an inflammatory disease, or a skeletal or muscular dystrophy, preferably a neurodegenerative disorder or a cancer.

18. A method for the treatment or prevention of a disease or condition responsive to c-ABL inhibition comprising administering a therapeutically effective amount of the compound according to any one of claims 1 to 13, or the pharmaceutical composition of claim 14, to a subject.

19. The method of claim 18, wherein the disease or condition is a neurodegenerative disorder, a cancer, a prion disease, a viral infection, diabetes, an inflammatory disease, or a skeletal or muscular dystrophy, preferably a neurodegenerative disorder or a cancer.

20. The compound or pharmaceutical composition for use according to claim 16, the use of the compound according to claim 17, or the method of claim 19, wherein the neurodegenerative disorder is selected from Alzheimer disease, Down's syndrome, frontotemporal dementia, progressive supranuclear palsy, Pick's disease, Niemann-Pick disease, Parkinson's disease, Huntington's disease (HD), dentatorubropallidoluysian atrophy, Kennedy's disease, and spinocerebellar ataxia, fragile X (Rett's) syndrome, fragile XE mental retardation, Friedreich's ataxia, myotonic dystrophy, spinocerebellar ataxia type 8, and spinocerebellar ataxia type 12, Alexander disease, Alper's disease, amyotrophic lateral sclerosis (ALS), ataxia telangiectasia, Batten disease, Canavan disease, Cockayne syndrome, corticobasal degeneration, Creutzfeldt-Jakob disease, ischemia stroke, Krabbe disease, Lewy body dementia, multiple sclerosis, multiple system atrophy, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis.

21. The compound for use, use of the compound, or method, of claim 20, wherein the neurodegenerative disorder is amyotrophic lateral sclerosis (ALS) or Parkinson's disease, preferably ALS.

22. The compound or pharmaceutical composition for use according to claim 16, the use of the compound according to claim 17, or the method of claim 19, wherein the cancer is leukaemia, preferably chronic myeloid leukaemia (CML), acute lymphoblastic leukaemia (ALL), acute myelogenous leukaemia (AML), or mixed-phenotype acute leukaemia (MPAL), or any central nervous system (CNS) metastases thereof, preferably CML or ALL.
